(19) United States
(12) Patent Application Publication
(10) Pub. No.: US 2003/0044845 A1
(43) Pub. Date: Mar. 6, 2003

(54) Novel therapeutic agents for membrane transporters

(76) Inventors: Thomas E. Jenkins, La Honda, CA (US); Burton G. Christensen, Alamo, CA (US); John H. Griffin, Atherton, CA (US); J. Kevin Judice, El Granada, CA (US)

Correspondence Address: THERAVANCE, INC., 901 GATEWAY BOULEVARD, SOUTH SAN FRANCISCO, CA, 94080, US

(21) Appl. No.: 10/075,017

(22) Filed: Feb. 13, 2002

Related U.S. Application Data
(63) Continuation of application No. 09/499,176, filed on Feb. 7, 2000, now abandoned.
Application No. 09/499,176 is a continuation of application No. 09/327,096, filed on Jun. 7, 1999, now abandoned.
(60) Provisional application No. 60/088,465, filed on Jun. 8, 1998.
(60) Provisional application No. 60/093,068, filed on Jul. 16, 1998.

(51) Int. Cl. G01N 33/53; C 07D 4 1/02

(52) U.S. Cl. 435/7.1; 546/140

(57) ABSTRACT

Novel multi-binding compounds (agents) are disclosed which bind cell membrane transporters including ion channels, molecular transporters and ion pumps. The compounds of this invention comprise from 2 to 10 ligands each of which can bind to such cellular transporters to modulate the biological processes/functions thereof. Each of the ligands is covalently attached to a linker (framework) to provide for a multi-binding compound. The linker is selected such that the multi-binding compound exhibits increased modulation of the biological processes/functions of the transporter as compared to the aggregate of the individual ligand units made available for binding to the transporter.

### CROSS REFERENCE TO RELATED APPLICATIONS

This application claims the benefit of U.S. Provisional Application Serial Nos. 60/088,465, filed Jun. 8, 1998, and 60/093,068, filed Jul. 16, 1998, the contents of which are herein incorporated by reference in their entirety.

### BACKGROUND OF THE INVENTION

### Field of the Invention

This invention relates to novel multi-binding compounds which bind to cell membrane transporters and modulate their activity. The compounds of this invention comprise at least two ligands covalently connected by a linker or linkers, wherein the ligands in their monovalent (i.e. unlinked) state bind to and are capable of modulating the activity of one or more transporters. The manner of linking the ligands together is such that the multi-binding agents thus formed demonstrate a biologic and/or therapeutic effect related to membrane transport processes which is greater than that of the same number of unlinked ligands made available for binding to the transporter. The invention also relates to methods of using such compounds and to methods of preparing them.

These multi-binding compounds are particularly useful in treating diseases and conditions of mammals and avians that are mediated by the cellular transporters targeted by the ligands. Accordingly, this invention also relates to pharmaceutical compositions comprising a pharmaceutically acceptable excipient and an effective amount of a compound of this invention.

This invention is also directed to general synthetic methods for generating large libraries of diverse multimeric compounds capable of binding cell membrane transporters which multimeric compounds are candidates for possessing multibinding properties. The diverse multimeric compound libraries provided by this invention are synthesized by combining a linker or linkers with a ligand or ligands to provide for a library of multimeric compounds wherein the linker and ligand each have complementary functional groups permitting covalent linkage. The library of linkers is preferably selected to have diverse properties such as valency, linker length, linker geometry and rigidity, hydrophilicity or hydrophobicity, amphiphilicity, acidity, basicity, polarizability and polarization. The library of ligands is preferably selected to have diverse attachment points on the same ligand, different functional groups at the same site of otherwise the same ligand, and the like.

### State of the Art

Cellular membranes form a selective permeability barrier to ions and small molecules thereby maintaining intracellular concentrations that are compatible with and optimized for physiological processes. This permeability barrier is comprised of a lipid bilayer, which restricts the passage of polar, charged and hydrophilic molecules and ions, and of cell membrane transporters. Cell membrane transporters include proteinaceous structures that open in a controlled manner to allow selected ions to flow passively into or out of the cell (e.g., ion channels), and proteins that actively pump ions and molecules into or out of the cell against a concentration gradient (e.g., H⁺/K⁺ ATPases, MDR efflux pumps, neurotransmitter transporters).

Ion channels are formed by the association of integral membrane proteins into structures having a central hydrophilic pore. Channel pores allow ions to equilibrate across membranes in response to their electrochemical gradients and at rates that are diffusion-limited. Ion channels are characterized by their selectivity and gating properties. Selectivity refers to the rate at which different ion species pass through an open channel under standard conditions. Gating is the process that regulates the opening and closing of an ion channel. Thus, voltage-regulated ion channels respond to changes in membrane potential; ligand-regulated channels respond to the binding of particular neurotransmitters (e.g., acetylcholine, glycine, GABA, 5-hydroxytryptamine, glutamate) or intracellular messengers (e.g., cyclic nucleotides, Ca⁺⁺); and mechanosensitive channels respond to mechanical deformation (e.g., stretch). Ion channels exist in resting (closed), open or inactivated (i.e., desensitized) states. Voltage-gated ion channels in the open state typically transition to an inactivated state, and must reacquire the ability to respond to an external stimulus during a recovery period. This may also be true of ligand-gated channels, particularly after prolonged exposure to an agonist. Certain channels are gated by more than one type of stimulus (e.g., an inward rectifying voltage-regulated potassium channel in cardiac muscle is activated by acetylcholine). Ion channels serve a variety of important cellular functions, including excitability, neuronal signalling, excitation- secretion coupling, volume regulation and so on. Ion channels are implicated in a variety of pathophysiological disorders, including hypertension, cardiac arrhythmogenesis, non-insulin dependent diabetes mellitus, and seizures, and mediate the transmission of pain impulses by peripheral nerves. Defective ion channel proteins are responsible for a variety of heritable diseases (see, e.g., Ackerman & Clapham, Ion Channels-Basic Science and Clinical Disease, NEJM 336: 1575 (1997).

The ABC transporters comprise a superfamily that shares a highly conserved ATP-binding cassette. See, e.g., Higgins, ABC Transporters: From Microorganisms to Man. Annu. Rev. Cell Biol. 8: 67-113 (1992) These transporters typically use ATP hydrolysis as a source of energy to pump diverse classes of molecules (e.g., sugars, peptides, inorganic ions, amino acids, oligopeptides, polysaccharides, proteins) across membranes against a concentration gradient. Each transporter is highly selective for a particular substrate and pumps unidirectionally . Some members of the ABC transporter family have ion channel activity. For example, the cystic fibrosis transmembrane regulator (CFTR), a cAMP- and protein kinase A-regulated Cl⁻ channel, uses ATP hydrolysis as a gating mechanism. P-glycoprotein (MDR) appears to be bifunctional, possessing drug transport as well as chloride channel activities; the latter is cell-volume regulated and requires the binding, but not the hydrolysis, of ATP. In both prokaryotes and eukaryotes, ABC transporters function in nutrient uptake, protein export and drug resistance (e.g., erythromycin resistance in Staphylococcus, daunomycin resistance in Streptomyces, chloroquine resistance in Plasmodium, and multidrug resistance in cancers).

Ion pumps are also involved in the active transport of ions across membranes. Ion pumps are members of the ion-transporting P-type ATPase family which couple ion transport to a cycle of phosphorylation and dephosphorylation of an ATPase enzyme. In mammalian cells, this class includes the Ca⁺⁺ATPases, the Na⁺/K⁺ ATPases, and the H⁺/K⁺ ATPases. The H⁺/ K⁺ ATPases are involved in acid secretion in the stomach and are clinically important targets in peptic ulcer disease, gastroesophageal reflux disease (GERD) and gastric hyperacidity. The Ca⁺⁺ and Na⁺/K⁺ ATPases are therapeutic targets in the treatment of heart failure. See, e.g., Chapter 34, in: Goodman & Gilman's “The Pharmacological Basis of Therapeutics”, Ninth Edition,McGraw-Hill (1996).

Another class of transporters belongs to the neurotransmitter transporter gene family. The proteins in this family are involved, inter alia, in the ion-dependent active uptake of amino acid (e.g., GABA) and amine neurotransmitters (e.g., norepinephrine, dopamine and 5-HT) at presynaptic nerve terminals. The neurotransmitters are taken up into cells against a concentration gradient by coupling the transport to the flow of Na⁺ and Cl⁻ down their transmembrane gradients. These transporters are clinically important targets for drugs useful in treating anxiety and depression. See, e.g., Chapter 12, in: Goodman & Gilman's “The Pharmacological Basis of Therapeutics”, Ninth Edition,McGraw-Hill (1996).

Cell membrane transporters are comprised of two or more homologous transmembrane protein domains/subunits which associate to form a translocation channel for ions and molecules. Cell membrane transporters are integral membrane proteins and thus are constrained in their rotational and translational mobility. They can be grouped into a small number of superfamilies on the basis of shared structural features (e.g., amino acid sequence homologies), may operate by similar molecular mechanisms, and in many cases may have a common evolutionary origin. Nevertheless, within a given superfamily there may be numerous subtypes with different tissue specificities resulting from combinations of different types and numbers of subunits.

FIG. 1 illustrates in cross-sectional view the transmembrane domain/subunit organization of various transporter molecules, as it is presently understood by those working in the field of transport physiology. It should be understood that, for purposes of simplification, other subunits that may be involved in or required for transporter activity have been omitted from the diagram.

Referring to FIG. 1, voltage-gated ion channels and related proteins are tetrameric structures formed by the noncovalent association of individual subunits (1),(2), or by the interaction of homologous domains of a monomeric protein (3). The channels differ as well in the number of transmembrane segments per subunit or per domain. Inward-rectifier type K⁺ channels and P_{2X} purinergic channels have two transmembrane-segments in each subunit, Shaker-type K⁺ channels have six transmembrane segments per subunit and Na⁺ and Ca⁺⁺ channels have six transmembrane segments per domain. Neurotransmitter-gated ion channels such as those shown in (4) are organized as pentamers, with each of the subunits having four transmembrane segments/domains. Lastly, the ion pumps and transporters (not shown here) contain 10-12 transmembrane segments in various structural arrangements, including pseudodimers having two nonidentical homologous domains on a single protein, (e.g., MDR, CFTR) (Higgins, Annu. Rev. Cell Biol. 8: 67-113 (1992)) and dimers (e.g., H⁺/K⁺ ATPase) (Hall et al, Biochim. Biophys. Acta 1077: 173-179 (1991).

Because of their central roles in ion homeostasis and in cell signalling events, ion transporters are involved in a wide variety of physiological activities, e.g., muscle contraction, cardiovascular function, renal function, neurotransmission, insulin secretion, immunomodulation and the like. See, e.g., Chapters 19, 35 and 37, In: Goodman & Gilman's “The Pharmacological Basis of Therapeutics”, Ninth Edition,McGraw-Hill (1996); Aidley & Stanfield, “Ion Channels”, Cambridge University Press (1996); Z. W. Hall, Chapter 3, in: “An Introduction to Molecular Neurobiology”, Sinauer Associates Inc. (1992); Cahalan and Chandy, Curr Opin in Biotech 8, 749 (1997).

Membrane-associated transporters play an important role in a wide variety of pathologic conditions. In some instances, these are traceable to genetic alterations in the transporters (see, e.g., Ackerman and Clapham, NEJM 336: 1575-1586 (1997). In diseased tissues, one or more transporter activities may be involved in pathogenic mechanisms, e.g., Ca⁺⁺, Na⁺ and K⁺ channels in cardiac arrhythmias (see, e.g., Task Force of the Working Group on Arrhythmias of the European Society of Cardiology, Circulation 84: 1831 (1991); H+pumps in gastroesophageal reflux disease (GERD) (see, e.g., Kahrilas, JAMA 276: 983-988 (1996)). In still others, molecular transporters contribute to drug resistance of pathogens and tumor cells (e.g., bacterial efflux pumps, Nikaido, Science 264: 382 (1994); mammalian MDR pumps; Gottesman & Pastan, Annu. Rev. Biochem. 62: 385 (1993)).

Not surprisingly, cell membrane transporters are recognized as important targets for drug therapy. For example, sodium channels are targeted by local anesthetics, certain antiarrhythmic drugs and anticonvulsants; potassium channels are targeted by certain antidiabetic, antihypertensive and antiarryhthmic drugs; calcium channels are targeted by certain antihypertensive, antiarrhythmic and antianginal drugs; ligand-gated Cl⁻ channels (invertebrates) are targeted by certain antiparasitic drugs; the proton channel of the influenza virus is targeted by certain antiviral drugs; H⁺/K⁺-ATPase is targeted by drugs used in gastric acidity disorders and peptic ulcer disease; and monoamine transporters are targeted for the treatment of depression.

Nevertheless, the clinical shortcomings of drugs in current usage (e.g., low potency, poor safety profile, lack of selectivity for the intended therapeutic target, and suboptimal duration of action) suggest that there continues to exist a need for new types of drugs having improved therapeutic activities.

### SUMMARY OF THE INVENTION

This invention addresses the above-mentioned needs by providing novel multi-binding compounds that bind cell membrane transporters in biological systems. The binding of these compounds to such cell membrane transporters can be used to treat diseases and conditions mediated by such cells.

Accordingly, in one embodiment, this invention is directed to a multi-binding compound comprising 2 to 10 ligands which may be the same or different and which are covalently attached to a linker or linkers, which may be the same or different, each of said ligands comprising a ligand domain capable of binding to a cell membrane transporter with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

The multi-binding compounds of this invention are preferably represented by formula I:

(L)ₚ(X)_{q} I

where each L is a ligand that may be the same or different at each occurrence;

X is a linker that may be the same or different at each occurrence;

p is an integer of from 2 to 10; and

q is an integer of from 1 to 20;

wherein each of said ligands comprises a ligand domain capable of binding to a cell membrane transporter, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

Preferably q is less than p.

Preferably, the binding of the multibinding compound to a cell membrane transporter in a mammal or avian modulates diseases and conditions mediated by the transporter.

In a second embodiment, this invention is directed to a pharmaceutical composition comprising a pharmaceutically acceptable excipient and a therapeutically effective amount of one or more multi-binding compounds (or pharmaceutically acceptable salts thereof) comprising 2 to 10 ligands which may be the same or different and which are covalently attached to a linker or linkers, which may be the same or different, each of said ligands comprising a ligand domain capable of binding to a cell membrane transporter of a cell mediating mammalian or avian diseases or conditions, thereby modulating the diseases or conditions, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

In a third embodiment, this invention is directed to a pharmaceutical composition comprising a pharmaceutically acceptable excipient and a therapeutically effective amount of one or more multi-binding compounds represented by formula I,

(L)ₚ(X)_{q} I

or pharmaceutically acceptable salts thereof,

where each L is a ligand that may be the same or different at each occurrence;

X is a linker that may be the same or different at each occurrence;

p is an integer of from 2 to 10; and

q is an integer of from 1 to 20;

wherein each of said ligands comprises a ligand domain capable of binding to a cell membrane transporter of a cell mediating mammalian or avian diseases or conditions, thereby modulating the diseases or conditions, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

Preferably q is less than p.

In a fourth embodiment, this invention is directed to a method for modulating the activity of a cell membrane transporter in a biologic tissue, which method comprises contacting a tissue having a cell membrane transporter with a multi-binding compound (or pharmaceutically acceptable salts thereof) under conditions sufficient to produce a change in transporter activity in said tissue, wherein the multi-binding compound comprises 2 to 10 ligands which may be the same or different and which are covalently attached to a linker or linkers, which may be the same or different, each of said ligands comprising a ligand domain capable of binding to a cell membrane transporter, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

In a fifth embodiment, this invention is directed to a method for treating a disease or condition in a mammal or avian resulting from an activity of a cell membrane transporter, which method comprises administering to said mammal or avian a therapeutically effective amount of a pharmaceutical composition comprising a pharmaceutically acceptable excipient and one or more multibinding compounds (or pharmaceutically acceptable salts thereof) comprising 2 to 10 ligands which may be the same or different and which are covalently attached to a linker or linkers, which may be the same or different, each of said ligands comprising a ligand domain capable of binding to a cell membrane transporter of a cell mediating mammalian or avian diseases or conditions, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

In a sixth embodiment, this invention is directed to a method for treating a disease or condition in a mammal or avian resulting from an activity of a cell membrane transporter, which method comprises administering to said mammal or avian a therapeutically effective amount of a pharmaceutical composition comprising a pharmaceutically acceptable excipient and one or more multibinding compounds represented by formula I,

(L)ₚ(X)_{q} I

and pharmaceutically acceptable salts thereof,

where each L is a ligand that may be the same or different at each occurrence;

X is a linker that may be the same or different at each occurrence;

p is an integer of from 2 to 10; and

q is an integer of from 1 to 20;

wherein each of said ligands comprises a ligand domain capable of binding to a cell membrane transporter of a cell mediating mammalian or avian diseases or conditions, with the following provisos:

(a) when each ligand is a 1,2,3,4 tetrahydroisoquinoline, then the linker is at least two atoms;

(b) neither the compound nor the ligand is a bis-quinolinium cyclophane; and

(c) the multibinding compound does not comprise homomeric phenylsulfonamide ligands;

(d) the multibinding compound does not comprise a dimeric 1,4 dihydropyridine compound linked through carboxylic ester groups.

Preferably q is less than p.

In a seventh embodiment, this invention relates to processes for preparing the multi-binding agents of Formula I.

This invention is also directed to general synthetic methods for generating large libraries of diverse multimeric compounds which multimeric compounds are candidates for possessing multibinding properties. The diverse multimeric compound libraries provided by this invention are synthesized by combining a linker or linkers with a ligand or ligands to provide for a library of multimeric compounds wherein the linker and ligand each have complementary functional groups permitting covalent linkage. The library of linkers is preferably selected to have diverse properties such as valency, linker length, linker geometry and rigidity, hydrophilicity or hydrophobicity, amphiphilicity, acidity, basicity, polarizability and polarization. The library of ligands is preferably selected to have diverse attachment points on the same ligand, different functional groups at the same site of otherwise the same ligand, and the like.

This invention is also directed to libraries of diverse multimeric compounds which multimeric compounds are candidates for possessing multibinding properties. These libraries are prepared via the methods described above and permit the rapid and efficient evaluation of what molecular constraints impart multibinding properties to a ligand or a class of ligands targeting a receptor.

Accordingly, in one of its method aspects, this invention is directed to a method for identifying multimeric ligand compounds possessing multibinding properties which method comprises:

(a) identifying a ligand or a mixture of ligands wherein each ligand contains at least one reactive functionality;

(b) identifying a library of linkers wherein each linker in said library comprises at least two functional groups having complementary reactivity to at least one of the reactive functional groups of the ligand;

(c) preparing a multimeric ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands; and

(d) assaying the multimeric ligand compounds produced in (c) above to identify multimeric ligand compounds possessing multibinding properties.

In another of its method aspects, this invention is directed to a method for identifying multimeric ligand compounds possessing multibinding properties which method comprises:

(a) identifying a library of ligands wherein each ligand contains at least one reactive functionality;

(b) identifying a linker or mixture of linkers wherein each linker comprises at least two functional groups having complementary reactivity to at least one of the reactive functional groups of the ligand;

(c) preparing a multimeric ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands; and

(d) assaying the multimeric ligand compounds produced in (c) above to identify multimeric ligand compounds possessing multibinding properties.

The preparation of the multimeric ligand compound library is achieved by either the sequential or concurrent combination of the two or more stoichiometric equivalents of the ligands identified in (a) with the linkers identified in (b). Sequential addition is preferred when a mixture of different ligands is employed to ensure heterodimeric or multimeric compounds are prepared. Concurrent addition of the ligands occurs when at least a portion of the multimer comounds prepared are homomultimeric compounds.

The assay protocols recited in (d) can be conducted on the multimeric ligand compound library produced in (c) above, or preferably, each member of the library is isolated by preparative liquid chromatography mass spectrometry (LCMS).

In one of its composition aspects, this invention is directed to a library of multimeric ligand compounds which may possess multivalent properties which library is prepared by the method comprising:

(a) identifying a ligand or a mixture of ligands wherein each ligand contains at least one reactive functionality;

(b) identifying a library of linkers wherein each linker in said library comprises at least two functional groups having complementary reactivity to at least one of the reactive functional groups of the ligand; and

(c) preparing a multimeric ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands.

In another of its composition aspects, this invention is directed to a library of multimeric ligand compounds which may possess multivalent properties which library is prepared by the method comprising:

(a) identifying a library of ligands wherein each ligand contains at least one reactive functionality;

(b) identifying a linker or mixture of linkers wherein each linker comprises at least two functional groups having complementary reactivity to at least one of the reactive functional groups of the ligand; and

(c) preparing a multimeric ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands.

In a preferred embodiment, the library of linkers employed in either the methods or the library aspects of this invention is selected from the group comprising flexible linkers, rigid linkers, hydrophobic linkers, hydrophilic linkers, linkers of different geometry, acidic linkers, basic linkers, linkers of different polarization and/or polarizabiltiy and amphiphilic linkers. For example, in one embodiment, each of the linkers in the linker library may comprise linkers of different chain length and/or having different complementary reactive groups. Such linker lengths can preferably range from about 2 to 100 Å.

In another preferred embodiment, the ligand or mixture of ligands is selected to have reactive functionality at different sites on said ligands in order to provide for a range of orientations of said ligand on said multimeric ligand compounds. Such reactive functionality includes, by way of example, carboxylic acids, carboxylic acid halides, carboxyl esters, amines, halides, pseudohalides, isocyanates, vinyl unsaturation, ketones, aldehydes, thiols, alcohols, anhydrides, boronates and precursors thereof. It is understood, of course, that the reactive functionality on the ligand is selected to be complementary to at least one of the reactive groups on the linker so that a covalent linkage can be formed between the linker and the ligand.

In other embodiments, the multimeric ligand compound is homomeric (i.e., each of the ligands is the same, although it may be attached at different points) or heteromeric (i.e., at least one of the ligands is different from the other ligands).

In addition to the combinatorial methods described herein, this invention provides for an iterative process for rationally evaluating what molecular constraints impart multibinding properties to a class of multimeric compounds or ligands targeting a receptor. Specifically, this method aspect is directed to a method for identifying multimeric ligand compounds possessing multibinding properties which method comprises:

(a) preparing a first collection or iteration of multimeric compounds which is prepared by contacting at least two stoichiometric equivalents of the ligand or mixture of ligands which target a receptor with a linker or mixture of linkers wherein said ligand or mixture of ligands comprises at least one reactive functionality and said linker or mixture of linkers comprises at least two functional groups having complementary reactivity to at least one of the reactive functional groups of the ligand wherein said contacting is conducted under conditions wherein the complementary functional groups react to form a covalent linkage between said linker and at least two of said ligands;

(b) assaying said first collection or iteration of multimeric compounds to assess which if any of said multimeric compounds possess multibinding properties;

(c) repeating the process of (a) and (b) above until at least one multimeric compound is found to possess multibinding properties;

(d) evaluating what molecular constraints imparted multibinding properties to the multimeric compound or compounds found in the first iteration recited in (a)-(c) above;

(e) creating a second collection or iteration of multimeric compounds which elaborates upon the particular molecular constraints imparting multibinding properties to the multimeric compound or compounds found in said first iteration;

(f) evaluating what molecular constraints imparted enhanced multibinding properties to the multimeric compound or compounds found in the second collection or iteration recited in (e) above;

(g) optionally repeating steps (e) and (f) to further elaborate upon said molecular constraints.

Preferably, steps (e) and (f) are repeated at least two times, more preferably at from 2-50 times, even more preferably from 3 to 50 times, and still more preferably at least 5-50 times.

### BRIEF DESCRIPTION OF THE DRAWINGS

FIG. 1 is a highly schematic illustration of the transmembrane organizations of representative transporters (cross-sectional view).

FIG. 2 illustrates a method for optimizing the linker geometry for presentation of ligands (filled circles) in bivalent compounds:

A. phenyldiacetylene core structure

B. cyclohexane dicarboxylic acid core structure

FIG. 3 illustrates the use of the technique of FIG. 2 for presentation of ligands in trivalent compounds.

FIG. 4 illustrates examples of multi-binding compounds comprising (A) 2 ligands, (B) 3 ligands, (C) 4 ligands, and (D) >4 ligands attached in different formats to a linker.

FIG. 5 illustrates several ligands for use in preparing multi-binding compounds. Potentially modifiable positions are indicated by arrows: (1) amiodarone; (2) amlodipine; (3) 5-pentyl, 5-phenyl hydantoin; (4) lidocaine; and (5) ondansetron.

FIGS. 6-11 show representative multi-binding local anesthetic compounds, where:

FIG. 6 shows bivalent structures with alkylene/substituted alkylene frameworks;

FIG. 7 shows bivalent structures having alkylalkoxy frameworks;

FIG. 8 shows bivalent structures having aromatic groups in the framework;

FIG. 9 shows structures having heterocyclic frameworks;

FIG. 10 shows trivalent and higher-order valency structures with various linker types; and

FIG. 11 shows bivalent and higher-order valency structures having various types of linkers with quaternary amine centers.

### DETAILED DESCRIPTION OF THE INVENTION

Biological systems in general are controlled by molecular interactions between bioactive ligands and their receptors, in which the receptor “recognizes” a molecule or a portion thereof (i.e., a ligand domain) to produce a biological effect. Thus, the cellular transport of ions and molecules involves “recognition” and interaction of these substrates with specific membrane-associated transporters. Accordingly, diseases or conditions that involve, or are mediated by, cell membrane transporters can be treated with pharmacologically active ligands that interact with such transporters to initiate, modulate or abrogate transporter activity.

The interaction of a cell membrane transporter and a ligand may be described in terms of “affinity” and “specificity”. The “affinity” and “specificity” of any given ligand-cell membrane transporter interaction is dependent upon the complementarity of molecular binding surfaces and the energetic costs of complexation (i.e., the net difference in free energy ΔG between bound and free states). Affinity may be quantified by the equilibrium constant of complex formation, the ratio of on/off rate constants, and/or by the free energy of complex formation. Specificity relates to the difference in binding affinity of a ligand for different receptors.

The net free energy of interaction of a ligand with a cell membrane transporter is the difference between energetic gains (enthalpy gained through molecular complementarity and entropy gained through the hydrophobic effect) and energetic costs (enthalpy lost through decreased solvation and entropy lost through reduced translational, rotational and conformational degrees of freedom).

The compounds of this invention are comprised of 2 to 10 ligands covalently linked together and capable of acting as multi-binding agents. Without wishing to be bound by theory, the surprising activity of these compounds is believed to arise at least in part from their ability to bind in a multivalent manner with the cell membrane transporter, which gives rise to a more favorable net free energy of binding. Multivalent binding interactions are characterized by the concurrent interaction of at least two ligands of a multi-binding compound with multiple ligand binding sites on a cell membrane transporter. Multivalent interactions differ from collections of individual monovalent interactions by being capable of providing enhanced biologic and/or therapeutic effect. Monovalent and multivalent binding is schematically illustrated in the diagram below. Multivalent binding can amplify binding affinities and differences in binding affinities, resulting in enhanced binding specificity as well as affinity.

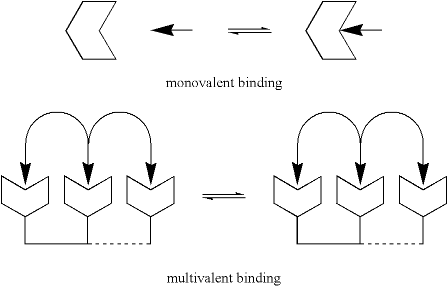

### DEFINITIONS

As used herein:

The term “alkyl” refers to a monoradical branched or unbranched saturated hydrocarbon chain, preferably having from 1 to 40 carbon atoms, preferably 1-10 carbon atoms, more preferably 1-6 carbon atoms, such as methyl, ethyl, n-propyl, isopropyl, n-butyl, secondary butyl, tert-butyl, n-hexyl, n-octyl, n-decyl, n-dodecyl, 2-ethyldodecyl, tetradecyl, and the like, unless otherwise indicated.

The term “substituted alkyl” refers to an alkyl group as defined above having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and and are chosen from hydrogen, optionally substituted alkyl, cycloalkyl, alkenyl, cycloalkenyl, alkynyl, aryl, heteroaryl and heterocyclic.

The term “alkylene” refers to a diradical of a branched or unbranched saturated hydrocarbon chain, preferably having from 1 to 40 carbon atoms, preferably 1-10 carbon atoms, more preferably 1-6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

The term “substituted alkylene” refers to an alkylene group as defined above having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocyclooxy, nitro, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Additionally, such substituted alkylene groups include those where 2 substituents on the alkylene group are fused to form one or more cycloalkyl, substituted cycloalkyl, cycloalkenyl, substituted cycloalkenyl, aryl, heterocyclic or heteroaryl groups fused to the alkylene group.

The term “alkaryl” or “aralkyl” refers to the groups -alkylene-aryl and-substituted alkylene-aryl in which alkylene and aryl are as defined herein. Such alkaryl groups are exemplified by benzyl, phenethyl and the like.

The term “alkoxy” refers to the groups alkyl-O—, alkenyl-O—, cycloalkyl-O—, cycloalkenyl-O—, and alkynyl-O—, where alkyl, alkenyl, cycloalkyl, cycloalkenyl, and alkynyl are as defined herein. Preferred alkoxy groups are alkyl-O— and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like

The term “substituted alkoxy” refers to the groups substituted alkyl-O—, substituted alkenyl-O—, substituted cycloalkyl-O—, substituted cycloalkenyl-O—, and substituted alkynyl-O—where substituted alkyl, substituted alkenyl, substituted cycloalkyl, substituted cycloalkenyl and substituted alkynyl are as defined herein.

The term “alkylalkoxy” refers to the groups -alkylene-O-alkyl, alkylene-O-substituted alkyl, substituted alkylene-O-alkyl and substituted alkylene-O-substituted alkyl wherein alkyl, substituted alkyl, alkylene and substituted alkylene are as defined herein. Examples of such groups are methylenemethoxy (—CH₂OCH₃), ethylenemethoxy (—CH₂CH₂OCH₃), n-propylene-iso-propoxy (—CH₂CH₂CH₂OCH(CH₃)₂), methylene-t-butoxy (-CH₂-O-C(CH₃)₃) and the like.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein. Preferred alkylthioalkoxy groups are alkylene-S-alkyl and include, by way of example, methylenethiomethoxy (—CH₂SCH₃), ethylenethiomethoxy (—CH₂CH₂SCH₃), n-propylene-iso-thiopropoxy (—CH₂CH₂CH₂SCH(CH₃)₂), methylene-t-thiobutoxy (-CH₂SC(CH₃)₃) and the like.

“Alkenyl” refers to a monoradical of a branched or unbranched unsaturated hydrocarbon preferably having from 2 to 40 carbon atoms, preferably 2-10 carbon atoms, more preferably 2-6 carbon atoms, and preferably having 1-6 double bonds. This term is further exemplified by such radicals as vinyl, prop-2-enyl, pent-3-enyl, hex-5-enyl, 5-ethyldodec-3,6-dienyl, and the like.

The term “substituted alkenyl” refers to an alkenyl group as defined above having from 1 to 5 substituents selected from the group consisting of alkoxy, substituted alkoxy, acyl, acylamino, acyloxy, amino, aminoacyl, aminoacyloxy, oxyaminoacyl, azido, cyano, halogen, hydroxyl, keto, thioketo, carboxyl, carboxylalkyl, thiol, thioalkoxy, substituted thioalkoxy, aryl, heteroaryl, heterocyclic, aryloxy, thioaryloxy, heteroaryloxy, thioheteroaryloxy, heterocyclooxy, thioheterocyclooxy, nitro, —SO-alkyl, —SO-substituted alkyl, —SO-aryl, —SO-heteroaryl, —SO₂-alkyl, —SO₂-substituted alkyl, —SO₂-aryl, -SO₂-heteroaryl, and. —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

“Alkenylene” refers to a diradical of an unsaturated hydrocarbon, preferably having from 2 to 40 carbon atoms, preferably 2-10 carbon atoms, more preferably 2-6 carbon atoms, and preferably having 1-6 double bonds. This term is further exemplified by such radicals as 1,2-ethenyl, 1,3-prop-2-enyl, 1,5-pent-3-enyl, 1,4-hex-5-enyl, 5-ethyl-1,12-dodec-3,6-dienyl, and the like.

The term “substituted alkenylene” refers to an alkenylene group as defined above having from 1 to 5 substituents, selected from the group consisting of alkoxy, substituted alkoxy, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocyclooxy, nitro, and NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Additionally, such substituted alkenylene groups include those where 2 substituents on the alkenylene group are fused to form one or more cycloalkyl, substituted cycloalkyl, cycloalkenyl, substituted cycloalkenyl, aryl, heterocyclic or heteroaryl groups fused to the alkenylene group.

“Alkynyl” refers to a monoradical of an unsaturated hydrocarbon, preferably having from 2 to 40 carbon atoms, preferably 2-10 carbon atoms, more preferably 2-6 carbon atoms, and preferably having 1-6 triple bonds. This term is further exemplified by such radicals as acetylenyl, prop-2-ynyl, pent-3-ynyl, hex-5-ynyl, 5-ethyldodec-3,6-diynyl, and the like.

The term “substituted alkynyl” refers to an alkynyl group as defined above having from 1 to 5 substituents, selected from the group consisting of alkoxy, substituted alkoxy, acyl, acylamino, acyloxy, amino, aminoacyl, aminoacyloxy, oxyacylamino, azido, cyano, halogen, hydroxyl, keto, thioketo, carboxyl, carboxylalkyl, thiol, thioalkoxy, substituted thioalkoxy, aryl, aryloxy, thioaryloxy, heteroaryl, heteroaryloxy, thioheteroaryloxy, heterocyclic, heterocyclooxy, thioheterocycloxy, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, SO₂-heterocyclic, NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

“Alkynylene” refers to a diradical of an unsaturated hydrocarbon radical, preferably having from 2 to 40 carbon atoms, preferably 2-10 carbon atoms, more preferably 2-6 carbon atoms, and preferably having 1-6 triple bonds. This term is further exemplified by such radicals as 1,3-prop-2-ynyl, 1,5-pent-3-ynyl, 1,4-hex-5-ynyl, 5-ethyl-1,12-dodec-3,6-diynyl, and the like.

The term “acyl” refers to the groups —CHO, alkyl-C(O)—, substituted alkyl-C(O)—, cycloalkyl-C(O)—, substituted cycloalkyl-C(O)—, cycloalkenyl-C(O)—, substituted cycloalkenyl-C(O)—, aryl-C(O)—, heteroaryl-C(O)— and heterocyclic-C(O)—where alkyl, substituted alkyl, cycloalkyl, substituted cycloalkyl, cycloalkenyl, substituted cycloalkenyl, aryl, heteroaryl and heterocyclic are as defined herein.

The term “acylamino” refers to the group —C(O)NRR where each R is independently hydrogen, alkyl, substituted alkyl, aryl, heteroaryl, heterocyclic or where both R groups are joined to form a heterocyclic group (e.g., morpholine) wherein alkyl, substituted alkyl, aryl, heteroaryl and heterocyclic are as defined herein.

The term “aminoacyl” refers to the group —NRC(O)R where each R is independently hydrogen, alkyl, substituted alkyl, aryl, heteroaryl, or heterocyclic wherein alkyl, substituted alkyl, aryl, heteroaryl and heterocyclic are as defined herein.

The term “aminoacyloxy” refers to the group —NRC(O)OR where each R is independently hydrogen, alkyl, substituted alkyl, aryl, heteroaryl, or heterocyclic wherein alkyl, substituted alkyl, aryl, heteroaryl and heterocyclic are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclic-C(O)O— wherein alkyl, substituted alkyl, cycloalkyl, substituted cycloalkyl, aryl, heteroaryl, and heterocyclic are as defined herein.

The term “aryl” refers to an unsaturated aromatic carbocyclic group of from 6 to 20 carbon atoms having a single ring (e.g., phenyl) or multiple condensed (fused) rings (e.g., naphthyl or anthryl).

Unless otherwise constrained by the definition for the aryl substituent, such aryl groups can optionally be substituted with from 1 to 5 substituents selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, trihalomethyl, NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Preferred aryl substituents include alkyl, alkoxy, halo, cyano, nitro, trihalomethyl, and thioalkoxy.

The term “aryloxy” refers to the group aryl-O— wherein the aryl group is as defined above including optionally substituted aryl groups as also defined above.

The term “arylene” refers to a diradical derived from aryl or substituted aryl as defined above, and is exemplified by 1,2-phenylene, 1,3-phenylene, 1,4-phenylene, 1,2-naphthylene and the like.

The term “carboxyalkyl” refers to the group “—C(O)Oalkyl” where alkyl is as defined above.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents selected from the group consisting of alkoxy, substituted alkoxy, cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyarnino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

The term “cycloalkenyl” refers to cyclic alkenyl groups of from 4 to 20 carbon atoms having a single cyclic ring or fused rings and at least one point of internal unsaturation. Examples of suitable cycloalkenyl groups include, for instance, cyclobut-2-enyl, cyclopent-3-enyl, cyclooct-3-enyl and the like.

The term “substituted cycloalkenyl” refers to cycloalkenyl groups having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

The term “halo” or “halogen” refers to fluoro, chloro, bromo and iodo.

“Haloalkyl” refers to alkyl as defined above substituted by 1-4 halo groups as defined above, which may be the same or different, such as 3-fluorododecyl, 12,12,12-trifluorododecyl, 2-bromooctyl, -3-bromo-6-chloroheptyl, and the like.

The term “heteroaryl” refers to an aromatic group of from 1 to 15 carbon atoms and 1 to 4 heteroatoms selected from oxygen, nitrogen and sulfur within at least one ring (if there is more than one ring).

Unless otherwise constrained by the definition for the heteroaryl substituent, such heteroaryl groups can be optionally substituted with 1 to 5 substituents selected from the group consisting of acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, aminoacyl, acylamino, alkaryl, aryl, aryloxy, azido, carboxyl, carboxylalkyl, cyano, halo, nitro, heteroaryl, heteroaryloxy, heterocyclic, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl, trihalomethyl, mono-and di-alkylamino, mono-and NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Preferred heteroaryls nclude pyridyl, pyrrolyl and furyl.

The term “heteroaryloxy” refers to the group heteroaryl-O—.

The term “heteroarylene” refers to the diradical group derived from heteroaryl or substituted heteroaryl as defined above, and is exemplified by the groups 2,6-pyridylene, 2,4-pyridiylene, 1,2-quinolinylene, 1,8-quinolinylene, 1,4-benzofuranylene, 2,5-pyridinylene, 1,3-morpholinylene, 2,5-indolenyl, and the like.

The term “heterocycle” or “heterocyclic” refers to a monoradical saturated or unsaturated group having a single ring or multiple condensed rings, from 1 to 40 carbon atoms and from 1 to 10 hetero atoms, preferably 1 to 4 heteroatoms, selected from nitrogen, sulfur, phosphorus, and/or oxygen within the ring.

Unless otherwise constrained by the definition for the heterocyclic substituent, such heterocyclic groups can be optionally substituted with 1 to 5, and preferably 1 to 3 substituents, selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, -SO₂-heteroaryl, and NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic. Such heterocyclic groups can have a single ring or multiple condensed rings.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles.

A preferred class of heterocyclics include “crown compounds” which refers to a specific class of heterocyclic compounds having one or more repeating units of the formula [—(CH₂—)ₘY—] where m is equal to or greater than 2, and Y at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “heterocyclooxy” refers to the group heterocyclic-O—.

The term “thioheterocyclooxy” refers to the group heterocyclic-S—.

The term “heterocyclene” refers to the diradical group derived from a heterocycle as defined herein, and is exemplified by the groups 2,6-morpholino, 2,5-morpholino and the like.

The term “oxyacylamino” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

The term “thiol” refers to the group —SH.

The term “thioalkoxy” refers to the group —S-alkyl.

The term “substituted thioalkoxy” refers to the group —S-substituted alkyl.

The term “thioaryloxy” refers to the group aryl-S— wherein the aryl group is as defined above including optionally substituted aryl groups also defined above.

The term “thioheteroaryloxy” refers to the group heteroaryl-S— wherein the heteroaryl group is as defined above including optionally substituted aryl groups as also defined above.

As to any of the above groups which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

“Alkyl optionally interrupted by 1-5 atoms chosen from O, S, or N” refers to alkyl as defined above in which the carbon chain is interrupted by O, S, or N. Within the scope are ethers, sulfides, and amines, for example 1 -methoxydecyl, 1 -pentyloxynonane, 1-(2-isopropoxyethoxy)4-methylnonane, 1-(2-ethoxyethoxy)dodecyl, 2-(t-butoxy)heptyl, 1-pentylsulfanylnonane, nonylpentylamine, and the like.

“Heteroarylalkyl” refers to heteroaryl as defined above linked to alkyl as defined above, for example pyrid-2-ylmethyl, 8-quinolinylpropyl, and the like. “Optional” or “optionally” means that the subsequently described event or circumstance may or may not occur, and that the description includes instances where said event or circumstance occurs and instances in which it does not. For example, optionally substituted alkyl means that alkyl may or may not be substituted by those groups enumerated in the definition of substituted alkyl.

The term “pharmaceutically acceptable salt” refers to salts which retain the biological effectiveness and properties of the multi-binding compounds of this invention and which are not biologically or otherwise undesirable. In many cases, the multi-binding compounds of this invention are capable of forming acid and/or base salts by virtue of the presence of amino and/or carboxyl groups or groups similar thereto.

Pharmaceutically acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di-and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Examples of suitable amines include, by way of example only, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that other carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

The term “protecting group” or “blocking group” refers to any group which when bound to one or more hydroxyl, thiol, amino or carboxyl groups of the compounds prevents reactions from occurring at these groups and which protecting group can be removed by conventional chemical or enzymatic steps to reestablish the hydroxyl, thiol, amino or carboxyl group. See, generally, T. W. Greene & P. G. M. Wuts “Protective Groups in Organic Synthesis,” 2^{nd} Ed, 1991 , John Wiley and Sons, N.Y.

The particular removable blocking group employed is not critical and preferred removable hydroxyl blocking groups include conventional substituents such as allyl, benzyl, acetyl, chloroacetyl, thiobenzyl, benzylidine, phenacyl, t-butyl-diphenylsilyl and any other group that can be introduced chemically onto a hydroxyl functionality and later selectively removed either by chemical or enzymatic methods in mild conditions compatible with the nature of the product.

Preferred removable amino blocking groups include conventional substituents such as t-butyoxycarbonyl (t-BOC), benzyloxycarbonyl (CBZ), fluorenylmethoxycarbonyl (FMOC), allyloxycarbonyl (ALOC) and the like, which can be removed by conventional conditions compatible with the nature of the product.

Preferred carboxyl protecting groups include esters such as methyl, ethyl, propyl, t-butyl etc. which can be removed by mild hydrolysis conditions compatible with the nature of the product.

As used herein, the terms “inert organic solvent” or “inert solvent” mean a solvent inert under the conditions of the reaction being described in conjunction therewith [including, for example, benzene, toluene, acetonitrile, tetrahydrofuran (“THF”), dimethylformamide (“DMF”), chloroform (“CHCl_{3”}), methylene chloride (or dichloromethane or “CH₂Cl₂”), diethyl ether, ethyl acetate, acetone, methylethyl ketone, methanol, ethanol, propanol, isopropanol, tert-butanol, dioxane, pyridine, and the like]. Unless specified to the contrary, the solvents used in the reactions of the present invention are inert solvents.

The term “library” refers to at least 3, preferably from 10² to 10⁹ and more preferably from 10² to 10⁴ multimeric compounds. Preferably, these compounds are prepared as a multiplicity of compounds in a single solution or reaction mixture which permits facile synthesis thereof. In one embodiment, the library of multimeric compounds can be directly assayed for multibinding properties. In another embodiment, each member of the library of multimeric compounds is first isolated and, optionally, characterized. This member is then assayed for multibinding properties.

The term “collection” refers to a set of multimeric compounds which are prepared either sequentially or concurrently (e.g., combinatorially). The collection comprises at least 2 members; preferably from 2 to 10⁹ members and still more preferably from 10 to 10⁴ members.

The term “multimeric compound” refers to compounds comprising from 2 to 10 ligands covalently connected through at least one linker which compounds may or may not possess multibinding properties (as defined herein).

The term “pseudohalide” refers to functional groups which react in displacement reactions in a manner similar to a halogen. Such functional groups include, by way of example, mesyl, tosyl, azido and cyano groups.

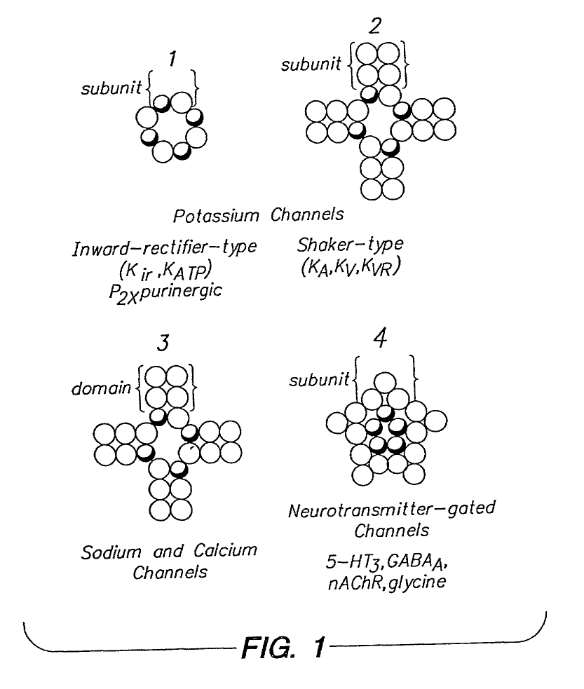

The term “cell membrane transporter” refers to a biomembrane-associated structure that is capable of transporting ions and/or molecules across a lipid membrane which is normally impermeant. Transporters include ion channels, molecular transporters and ion pumps. Examples of cell membrane transporters and ligands (drugs) that bind to them and disease conditions mediated thereby include those found in Tables 1 and 6, and in FIG. 1 (see Appendix).

“Ligand” as used herein denotes a compound that is a binding partner for a receptor, such as a cell membrane transporter, and is bound thereto by complementarity. The specific region or regions of the ligand molecule that is recognized by the ligand binding site of a receptor is designated as the “ligand domain” . A ligand may be either capable of binding to a receptor by itself, or may require the presence of one or more non-ligand components for binding (e.g. Ca²⁺, Mg²⁺ or a water molecule is required for the binding of a ligand to various receptors).

An exemplary list of ligands that target cell membrane transporters is shown in Table 1 (Appendix). For purposes of the present invention, however, it should be understood that portions of the ligand structure that are not essential for specific molecular recognition and binding activity may be varied substantially, replaced with unrelated structures and, in some cases, omitted entirely without affecting the binding interaction. It is further understood that the term ligands is not intended to be limited to compounds known to be useful as cell membrane transporter-binding compounds (e.g., known drugs). Those skilled in the art will understand that the term ligand can equally apply to a molecule that is not normally recognized for its transporter binding properties. The primary requirement for a ligand as defined herein is that it has a ligand domain, as defined above, which is available for binding to a recognition site on a cell membrane transporter. In addition, it should be noted that ligands that exhibit marginal activity or lack useful activity as monomers can be highly active as multibinding compounds, because of the biological benefit conferred by multivalency.

“Multi-binding agent” or “multi-binding compound” refers to a compound that is capable of multivalency as defined below, and which has from 2 to 10 ligands covalently bound to one or more linkers which may be the same or different. The multi-binding agent provides a biologic and/or therapeutic effect greater than the aggregate of unlinked ligands equivalent thereto. That is to say, that the biologic and/or therapeutic effect of the ligands attached to the multi-binding compound is greater than that achieved by the same number of unlinked ligands made available for binding to the receptor or receptors. Examples of greater “biologic and/or therapeutic effect” include increased ligand-receptor binding interactions (e.g., increased affinity; increased agonist, antagonist or modulatory effects; improved kinetics), increased selectivity for the target, increased potency, increased efficacy, decreased toxicity, optimized duration of action, improved bioavailability, improved pharmacokinetics, improved activity spectrum, increased therapeutic index, and the like. The multibinding compounds of this invention will exhibit at least one, and preferably more than one, of the above-mentioned effects.

The term “ligand binding site” as used herein denotes a site on a receptor, such as a cell membrane transporter, that recognizes a ligand domain and provides a binding partner for the ligand. The ligand binding site may be defined by monomeric or multimeric structures. This interaction may be capable of producing a unique biological effect, for example agonism, antagonism, modulation, or may maintain an ongoing biological event, and the like. For purposes of this invention, the ligand, the ligand domain and the ligand binding site cannot both be an antibody, an antibody domain or a fragment of an antibody.

It should be recognized that the ligand binding sites of cell membrane transporters that participate in biological multivalent binding interactions are constrained to varying degrees by their intra- and intermolecular associations. For example, ligand binding sites may be covalently joined in a single structure, noncovalently associated in one or more multimeric structures, embedded in a membrane or biopolymer matrix, and so on, and therefore have less translational and rotational freedom than if the same sites were present as monomers in solution.

The terms “agonism” and “antagonism” are well known in the art. As used herein, the term “agonist” refers to a ligand that when bound to a cell membrane transporter stimulates the functional activity of the transporter. The term “antagonist” refers to a ligand that when bound to a transporter inhibits the functional activity of the transporter.

The term “modulatory effect” is intended to refer to the ability of a ligand to change the activity of a cell membrane transporter through binding to the transporter, regardless of whether the transporter is activated (or inhibited) by an endogenous ligand, or by the occurrence (or non-occurrence) of a physicochemical stimulus (e.g., a change in membrane potential or mechanical deformation in the case of certain classes of ion channels). The “modulatory effect” of compounds of this invention will often result in inhibition of transport processes.

“Potency” as used herein refers to the minimum concentration at which a ligand is able to achieve a desirable biological or therapeutic effect. The potency of a ligand is typically proportional to its affinity for its receptor. In some cases, the potency may be non-linearly correlated with its affinity. In comparing the potency of two drugs, e.g., a multi-binding agent and the aggregate of its unlinked ligand, the dose-response curve of each is determined under identical test conditions (e.g. in an in vitro or in vivo assay, in an appropriate animal model (such as a human patient)). The finding that the multi-binding agent produces an equivalent biologic or therapeutic effect at a lower concentration than the aggregate unlinked ligand (e.g. on a per weight, per mole or per ligand basis) is indicative of enhanced potency.

“Univalency” as used herein refers to a single binding interaction between one ligand with one ligand binding site as defined herein. It should be noted that a compound having multiple copies of a ligand (or ligands) exhibits univalency when only one ligand of that compound interacts with a ligand binding site. Examples of univalent interactions are depicted below.

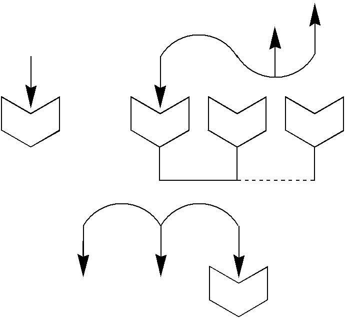

“Multivalency” as used herein refers to the concurrent binding of from 2 to 10 linked ligands (which may be the same or different) and two or more corresponding ligand binding sites, which may be the same or different. For example, two ligands connected by a linker that bind concurrently to two ligand binding sites would be considered bivalent; three concurrently binding ligands thus connected would be trivalent.

It should be understood that not all compounds that contain multiple copies of a ligand attached to a linker necessarily exhibit the phenomena of multi-valency, i.e., that the biologic and/or therapeutic effect of the multi-binding agent is greater than that of the same number of unlinked ligands made available for binding to the receptor or receptors. For multi-valency to occur, the ligands that are connected by a linker or linkers have to be presented to their receptors by the linker(s) in a specific manner in order to bring about the desired ligand-orienting result, and thus produce a multi-binding interaction.

“Selectivity” or “specificity” is a measure of the binding preferences of a ligand for different receptors. The selectivity of a ligand with respect to its target receptor relative to another receptor is given by the ratio of the respective values of K_{d} (i.e., the dissociation constants for each ligand-receptor complex) or, in cases where a biological effect is observed below the K_{d}, the ratio of the respective EC₅₀s (i.e., the concentrations that produce 50 % of the maximum response for the ligand interacting with the two distinct receptors).

The term “treatment” refers to any treatment of a disease or condition in a mammal, particularly a human, or an avian, and includes:

(i) preventing the disease or condition from occurring in a subject which may be predisposed to the condition but has not yet been diagnosed with the condition and, accordingly, the treatment constitutes prophylactic treatment for the pathologic condition;

(ii) inhibiting the disease or condition, i.e., arresting its development;

(iii) relieving the disease or condition, i.e., causing regression of the disease or condition; or

(iv) relieving the symptoms resulting from the disease or condition, e.g., relieving nausea, neuropathic pain, resistance to respiratory airflow, etc. without addressing the underlining disease or condition.

The term “disease or condition which is modulated by treatment with a ligand” covers all disease states and/or conditions (e.g., pain, nausea, resistance to respiratory airflow) that are generally acknowledged in the art to be usefully treated with a ligand for a cell membrane transporter in general, and those disease states and/or conditions that have been found to be usefully treated by a specific multi-binding compound of our invention, i.e., the compounds of Formula I. Such disease states include, by way of example only, hypertension, cardiac arrythmias, myocardial ischemia, Liddle syndrome, insulinoma, cystic fibrosis, myopathies, seizures, neuropathic pain, anxiety, gastric acidity, achalasia, influenza, parasitic diseases, and the like.

The term “therapeutically effective amount” refers to that amount of multi-binding compound that is sufficient to effect treatment, as defined above, when administered to a mammal or avian in need of such treatment. The therapeutically effective amount will vary depending upon the subject and disease condition being treated, the weight and age of the subject, the severity of the disease condition, the manner of administration and the like, which can readily be determined by one of ordinary skill in the art.

The term “pharmaceutically acceptable excipient” is intended to include vehicles and carriers capable of being coadministered with a multi-binding compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present invention.

The term “linker”, identified where appropriate by the symbol X, refers to a group or groups that covalently links from 2 to 10 ligands (as defined above) in a manner that provides for a compound capable of multivalency when in the presence of a cell membrane transporter having 2 or more ligand binding sites. Among other features, the linker is a ligand-orienting entity that permits attachment of multiple copies of a ligand (which may be the same or different) thereto. It may be chiral or achiral. In some cases, the linker may itself be biologically active. The term “linker” does not, however, cover solid inert supports such as beads, glass particles, rods, and the like, but it is to be understood that the multi-binding compounds of this invention can be attached to a solid support if desired, for example, for use in separation and purification processes and for similar applications.

The extent to which multivalent binding is realized depends upon the efficiency with which the linker or linkers that joins the ligands presents them to their array of ligand binding sites. Beyond presenting these ligands for multivalent interactions with ligand binding sites, the linker spatially constrains these interactions to occur within dimensions defined by the linker. Thus the structural features of the linker (valency, geometry, orientation, size, flexibility, chemical composition) are features of multi-binding compounds that play an important role in determining their molecular interactions with receptors and efficacy.

The linkers used in this invention are selected to allow multivalent binding of ligands to any desired ligand binding sites of a cell membrane transporter, whether such sites are located interiorly (e.g., within a channel/translocation pore), both interiorly and on the periphery of a transporter, at the boundary region between the lipid bilayer and the transporter, or at any intermediate position thereof. The distance between the nearest neighboring ligand domains is preferably in the range of about 2 Å to about 100 Å, more preferably in the range of about 2 Å to about 50 Å and even more preferably about 3-15 Å.

The ligands are covalently attached to the linker or linkers using conventional chemical techniques. The reaction chemistries resulting in such linkage are well known in the art and involve the use of reactive functional groups present on the linker and ligand. Preferably, the reactive functional groups on the linker are selected relative to the functional groups available on the ligand for binding or which can be introduced onto the ligand for binding. Again, such reactive functional groups are well known in the art. For example, reaction between a carboxylic acid of either the linker or the ligand and a primary or secondary amine of the ligand or the linker in the presence of suitable well-known activating agents results in formation of an amide bond covalently linking the ligand to the linker; reaction between an amine group of either the linker or the ligand and a sulfonyl halide of the ligand or the linker results in formation of a sulfonamide bond covalently linking the ligand to the linker; and reaction between an alcohol or phenol group of either the linker or the ligand and an alkyl or aryl halide of the ligand or the linker results in formation of an ether bond covalently linking the ligand to the linker. Table 2 (Appendix) illustrates numerous reactive functional groups and the resulting bonds formed by reaction therebetween. Where functional groups are lacking, they can be created by suitable chemistries that are described in standard organic chemistry texts such as J. March, “Advanced Organic Chemistry”, 4^{th} Edition, (Wiley-Interscience (New York), 1992.

The linker is attached to the ligand at a position that retains ligand domain-receptor binding and specifically permits the ligand domain of the ligand to orient itself to bind to the ligand binding site. The determination of where acceptable substitutions can be made on a ligand is typically based on prior knowledge of structure-activity relationships of the ligand and/or congeners and/or structural information about ligand-receptor complexes (e.g., X-ray crystallography, NMR, and the like). Such positions and synthetic protocols for linkage are well known in the art and can be determined by those with ordinary skill in the art. Following attachment of a ligand to the linker or a significant portion thereof (e.g., 2-10 atoms of linker), the linker-ligand conjugate may be tested for retention of activity in a relevant assay system (see Specific Embodiments and Table 3 (Appendix) for representative assays).

The relative orientation in which the ligand domains are displayed to the receptors depends both on the particular point (or points) of attachment of the ligands to the linker, and on the geometry of the linker framework. The term linker embraces everything that is not considered to be part of the ligand, e.g., ancillary groups such as solubilizing groups, lipophilic groups, groups that alter pharmacodynamics or pharmacokinetics, groups that modify the diffusability of the multi-binding compound, groups that attach the ligand to the linker, groups that aid the ligand-orienting function of the linker, for example, by imparting flexibility or rigidity to the linker as a whole, or to a portion thereof, and so on. In some preferred embodiments, the linker comprises a portion of the ligand, as is illustrated below for a bivalent lidocaine compound of Formula I. The N-ethyl groups of a lidocaine molecule that comprise the linker on the left side of the structure are highlighted in bold.

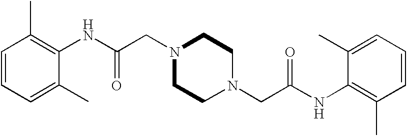

At present, it is preferred that the multi-binding agent is a bivalent compound, i.e., two ligands which are covalently linked to linker X, or a trivalent compound, i.e., three ligands which are covalently linked to linker X.

Suitable linkers are discussed below.

### METHODOLOGY

The linker or linkers, when covalently attached to multiple copies of the ligands, provides a biocompatible, substantially non-immunogenic multi-binding compound. The biological activity of the multi-binding compound is highly sensitive to the geometry, composition, size, flexibility or rigidity, the presence or absence of anionic or cationic charge, the relative hydrophobicity/hydrophilicity, and similar properties of the linker. Accordingly, the linker is preferably chosen to maximize the biological activity of the multi-binding compound. The linker may be biologically “neutral,” i.e., not itself contribute any biological activity to the multi-binding compound, or it may be chosen to enhance the biological activity of the compound. In general, the linker may be chosen from any organic molecule construct that orients two or more ligands to the receptors to permit multivalency. In this regard, the linker can be considered as a “framework” on which the ligands are arranged in order to bring about the desired ligand-orienting result, and thus produce a multi-binding compound.

For example, different orientations of ligands can be achieved by varying the geometry of the framework (linker) by use of mono- or polycyclic groups, such as aryl and/or heteroaryl groups, or structures incorporating one or more carbon-carbon multiple bonds (alkenyl, alkenylene, alkynyl or alkynylene groups). The optimal geometry and composition of frameworks (linkers) used in the multi-binding compounds of this invention are based upon the properties of their intended receptors. For example, it is preferred to use rigid cyclic groups (e.g., aryl, heteroaryl), or non-rigid cyclic groups (e.g., cycloalkyl or crown groups) to reduce conformational entropy when such may be necessary to achieve energetically coupled binding.

Different hydrophobic/hydrophilic characteristics of the linker as well as the presence or absence of charged moieties can readily be controlled by the skilled artisan. For example, the hydrophobic nature of a linker derived from hexamethylene diamine (H₂N(CH₂)₆NH₂) or related polyamines can be modified to be substantially more hydrophilic by replacing the alkylene group with a poly(oxyalkylene) group such as found in the commercially available “Jeffamines” (class of surfactants).

Different frameworks can be designed to provide preferred orientations of the ligands. Such frameworks may be represented by using an array of dots (as shown below) wherein each dot is either an atom, chosen from carbon, hydrogen, oxygen, nitrogen, sulfur, phosphorus, or halogen, or the dot represents a point in space (i.e., an absence of an atom). Only certain atoms on the grid have the ability to act as an attachment point for the ligands, namely, C, O, N, S and P.

To facilitate the understanding of the framework structure, the framework is illustrated as a two dimensional array in the following diagram, although clearly the framework is a three dimensional array in practice.

| | . | . | . | . | . | . | . | . | | |
|---|---|---|---|---|---|---|---|---|---|---|
| | . | . | . | . | . | . | . | . | | |
| | . | . | . | . | . | . | . | . | | |
| | . | . | . | . | . | . | . | . | | |
| | . | . | . | . | . | . | . | . | | |
| 8 | ▪ | ▪ | ▪ | ▪ | ▪ | ▪ | ▪ | ▪ | ▪ | . . . . . |
| 7 | ▪ | ▪ | ▪ | ▪ | ▪ | ▪ | ▪ | ▪ | ▪ | . . . . . |
| 6 | ▪ | ▪ | ▪ | ▪ | ▪ | ▪ | ▪ | ▪ | ▪ | . . . . . |
| 5 | ▪ | ▪ | ▪ | ▪ | ▪ | ▪ | ▪ | ▪ | ▪ | . . . . . |
| 4 | ▪ | ▪ | ▪ | ▪ | ▪ | ▪ | ▪ | ▪ | ▪ | . . . . . |
| 3 | ▪ | ▪ | ▪ | ▪ | ▪ | ▪ | ▪ | ▪ | ▪ | . . . . . |
| 2 | ▪ | ▪ | ▪ | ▪ | ▪ | ▪ | ▪ | ▪ | ▪ | . . . . . |
| 1 | ▪ | ▪ | ▪ | ▪ | ▪ | ▪ | ▪ | ▪ | ▪ | . . . . . |
| 0 | ▪ | ▪ | ▪ | ▪ | ▪ | ▪ | ▪ | ▪ | ▪ | . . . . . |
| | 0 | 1 | 2 | 3 | 4 | 5 | 6 | 7 | 8 | |

Atoms can be connected to each other via bonds (single, double or triple bonds with acceptable resonance and tautomeric forms), with regard to the usual constraints of chemical bonding. Ligands may be attached to the framework via single, double or triple bonds (with chemically acceptable tautomeric and resonance forms). Multiple ligand groups (2 to 10) can be attached to the framework such that the minimal, shortest path distance between adjacent ligand groups does not exceed 100 atoms. Preferably, the linker connections to the ligand is selected such that the maximum distance between two adjacent ligands is no more than 40 Angstroms (Å).

An example of a linker as presented by the grid is shown below for a biphenyl construct.

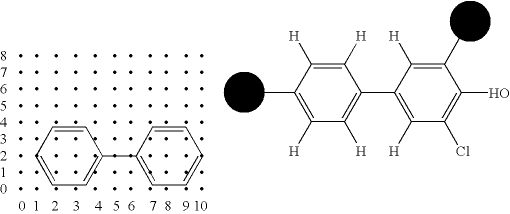

Nodes (1,2), (2,0), (4,4), (5,2), (4,0), (6,2), (7,4), (9,4), (10,2), (9,0), (7,0) all represent carbon atoms. Node (10,0) represents a chlorine atom. All other nodes (or dots) are points in space (i.e., represent an absence of atoms). Nodes (1,2) and (9,4) are attachment points.

Hydrogen atoms are affixed to nodes (2,4), (4,4), (4,0), (2,0), (7,4), (10,2) and (7,0).

Nodes (5,2) and (6,2) are connected by a single bond.

The carbon atoms present are connected by either a single or double bonds, taking into consideration the principle of resonance and/or tautomerism. The intersection of the framework (linker) and the ligand group, and indeed, the framework (linker) itself can have many different bonding patterns. Examples of acceptable patterns of three contiguous atom arrangements are shown in the following diagram:

| | | | | |
|---|---|---|---|---|
| CCC | NCC | OCC | SCC | PCC |
| CCN | NCN | OCN | SCN | PCN |
| CCO | NCO | OCO | SCO | PCO |
| CCS | NCS | OCS | SCS | PCS |
| CCP | NCP | OCP | SCP | PCP |
| CNC | NNC | ONC | SNC | PNC |
| CNN | NNN | ONN | SNN | PNN |
| CNO | NNO | ONO | SNO | PNO |
| CNS | NNS | ONS | SNS | PNS |
| CNP | NNP | ONP | SNP | PNP |
| COC | NOC | OOC | SOC | POC |
| CON | NON | OON | SON | PON |
| COO | NOO | OOO | SOO | POO |
| COS | NOS | OOS | SOS | POS |
| COP | NOP | OOP | SOP | POP |
| CSC | NSC | OSC | SSC | PSC |
| CSN | NSN | OSN | SSN | PSN |
| CSO | NSO | OSO | SSO | PSO |
| CSS | NSS | OSS | SSS | PSS |
| CSP | NSP | OSP | SSP | PSP |
| CPC | NPC | OPC | SPC | PPC |
| CPN | NPN | OPN | SPN | PPN |
| CPO | NPO | OPO | SPO | PPO |
| CPS | NPS | OPS | SPS | PPS |
| CPP | NPP | OPP | SPP | PPP |

One skilled in the art would be able to identify bonding patterns that would produce multivalent compounds. Methods for producing these bonding arrangements are described in March, “Advanced Organic Chemistry”, 4th Edition, Wiley-Interscience, New York, N.Y. (1992). These arrangements are described in the grid of dots shown in the scheme above. All of the possible arrangements for the five most preferred atoms are shown. Each atom has a variety of acceptable oxidation states. The bonding arrangements underlined are less acceptable and are not preferred.

Examples of molecular structures in which the above bonding patterns could be employed as components of the linker are shown below.

| | | | | |
|---|---|---|---|---|
| 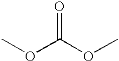 | 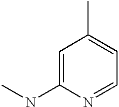 | 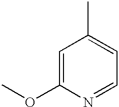 | 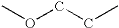 | 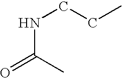 |
| 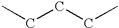 | 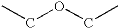 | 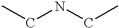 | 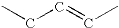 | 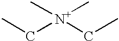 |
| 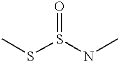 | 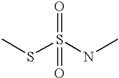 | 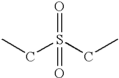 | 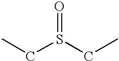 | 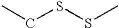 |
| 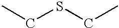 | 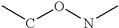 | 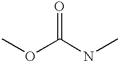 | 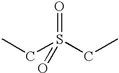 | 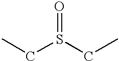 |
| 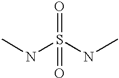 | 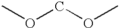 | 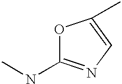 | 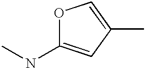 | 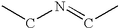 |
| 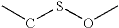 | 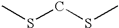 | 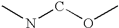 | 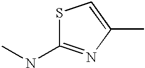 | 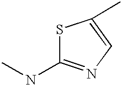 |
| 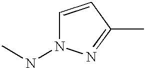 | 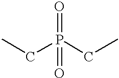 | 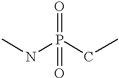 | 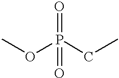 | 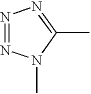 |

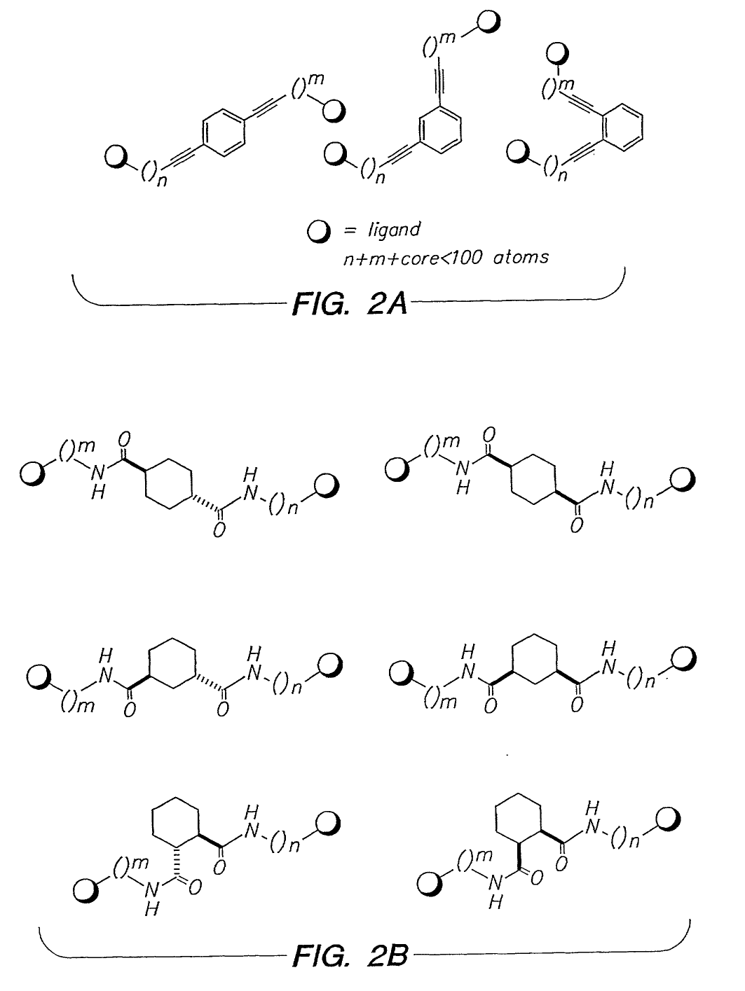

The identification of an appropriate framework geometry for ligand domain presentation is an important first step in the construction of a multi-binding agent with enhanced activity. Systematic spatial searching strategies can be used to aid in the identification of preferred frameworks through an iterative process. FIG. 2 (Appendix) illustrates a useful strategy for determining an optimal framework display orientation for ligand domains and can be used for preparing the bivalent compounds of this invention. Various alternative strategies known to those skilled in the art of molecular design can be substituted for the one described here. As shown in FIG. 2, the ligands (shown as filled circles) are attached to a central core structure such as phenyldiacetylene (Panel A) or cyclohexane dicarboxylic acid (Panel B). The ligands are spaced apart from the core by an attaching moiety of variable lengths m and n. If the ligand possesses multiple attachment sites (see discussion below), the orientation of the ligand on the attaching moiety may be varied as well. The positions of the display vectors around the central core structures are varied, thereby generating a collection of compounds. Assay of each of the individual compounds of a collection generated as described will lead to a subset of compounds with the desired enhanced activities (e.g., potency, selectivity). The analysis of this subset using a technique such as Ensemble Molecular Dynamics will suggest a framework orientation that favors the properties desired.

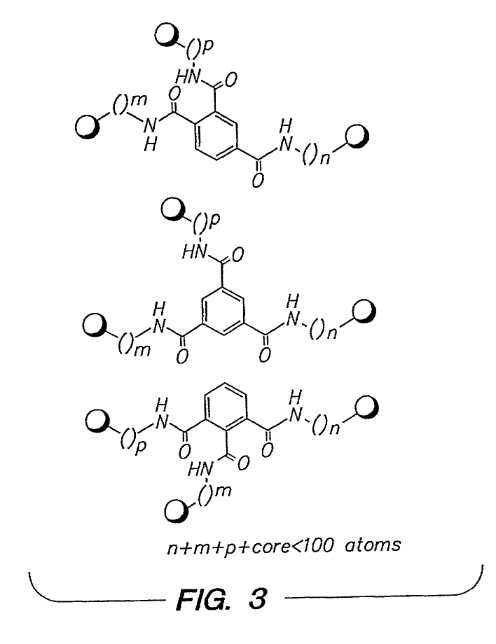

The process may require the use of multiple copies of the same central core structure or combinations of different types of display cores. It is to be noted that core structures other than those shown here can be used for determining the optimal framework display orientation of the ligands. The above-described technique can be extended to trivalent compounds (FIG. 3) and compounds of higher-order valency.

An example of this process for extending the framework from the ligand is presented below for the local anesthetic-type Na⁺ channel blockers. Some of the acceptable possible locations for elaboration into the framework are shown with arrows.

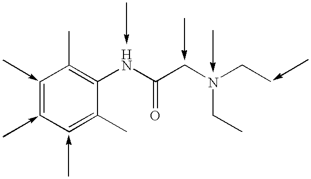

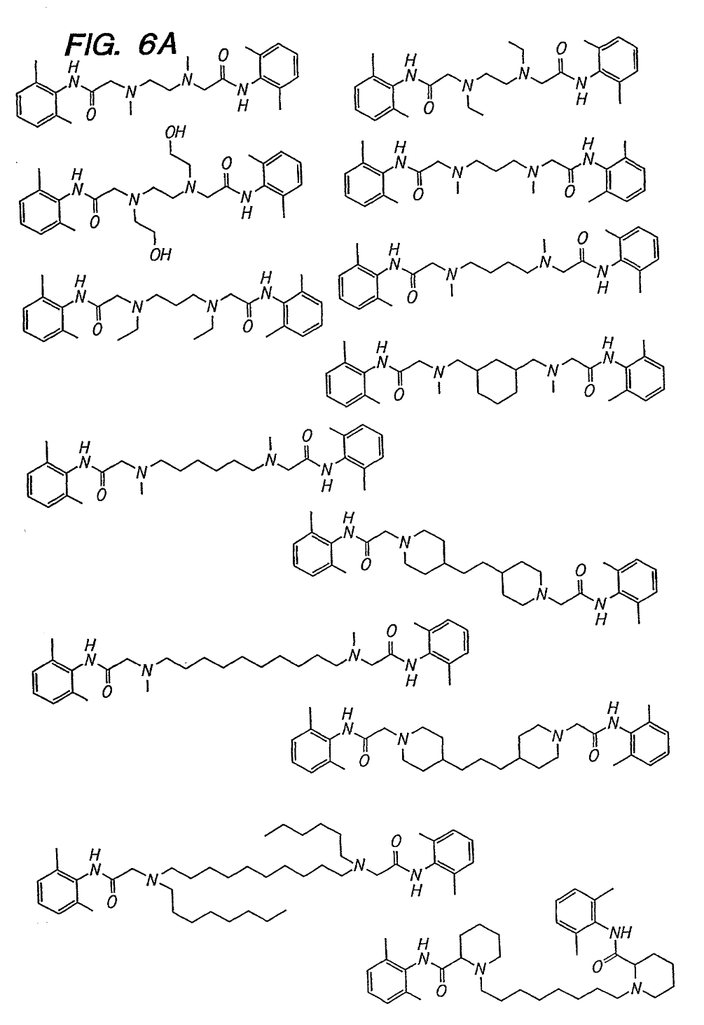

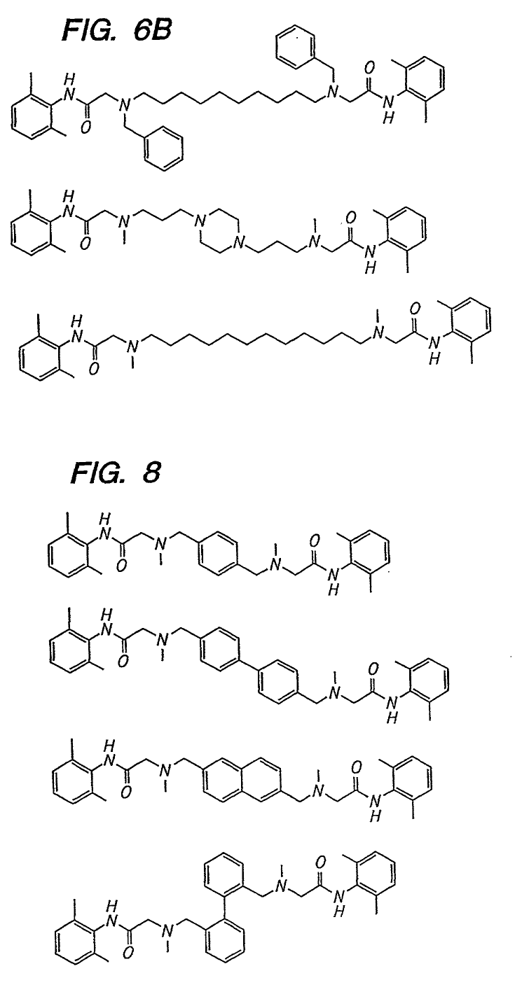

Examples of local anesthetic bivalent compounds are shown below and in FIGS. 6-8, 9 and 11 (Appendix).

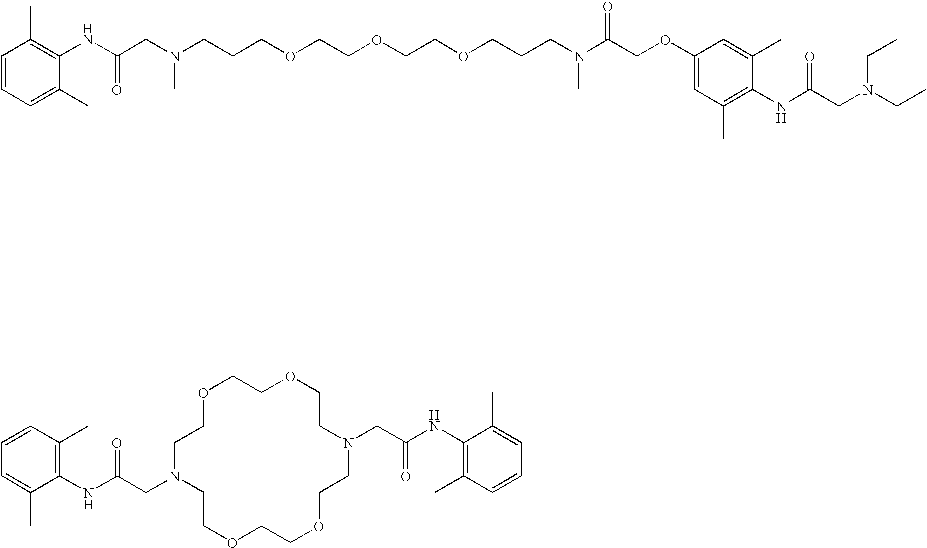

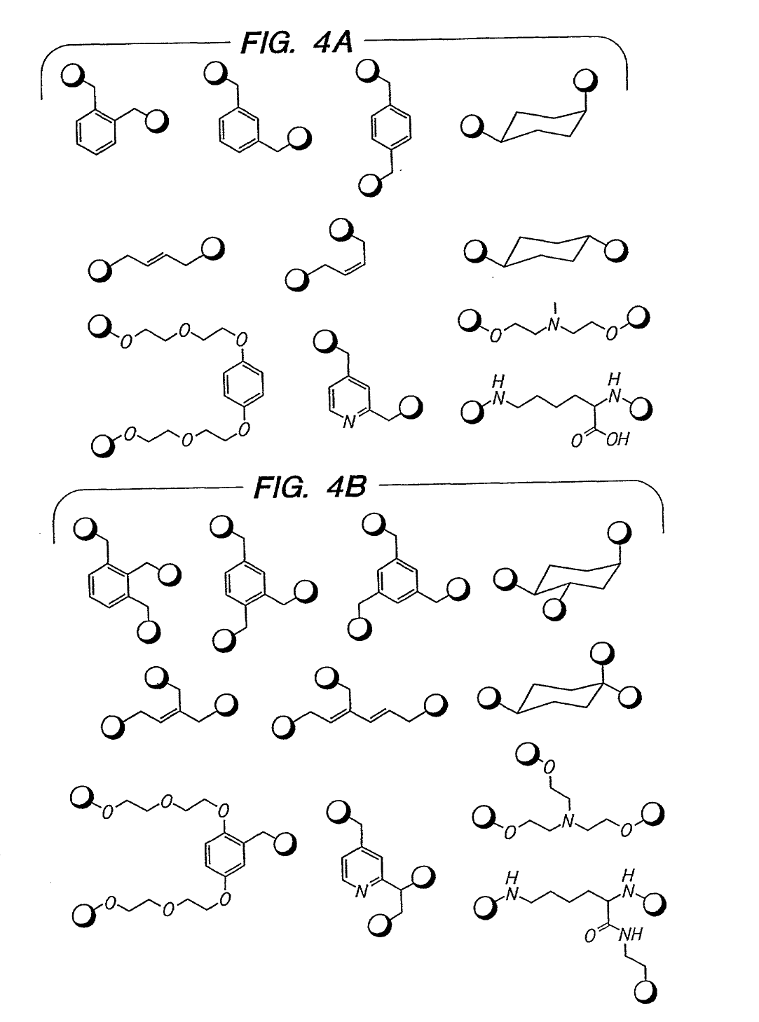

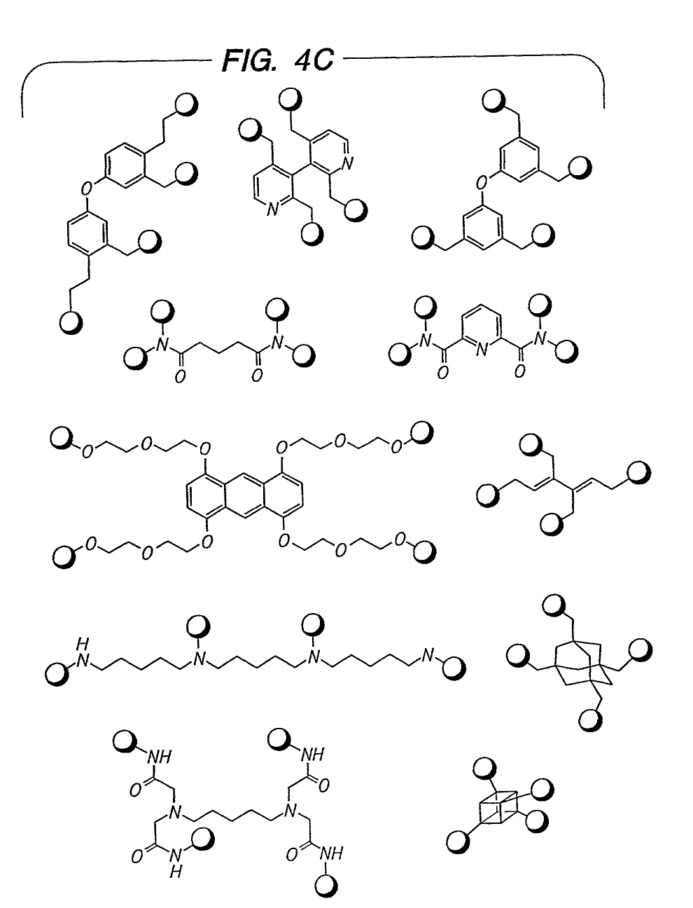

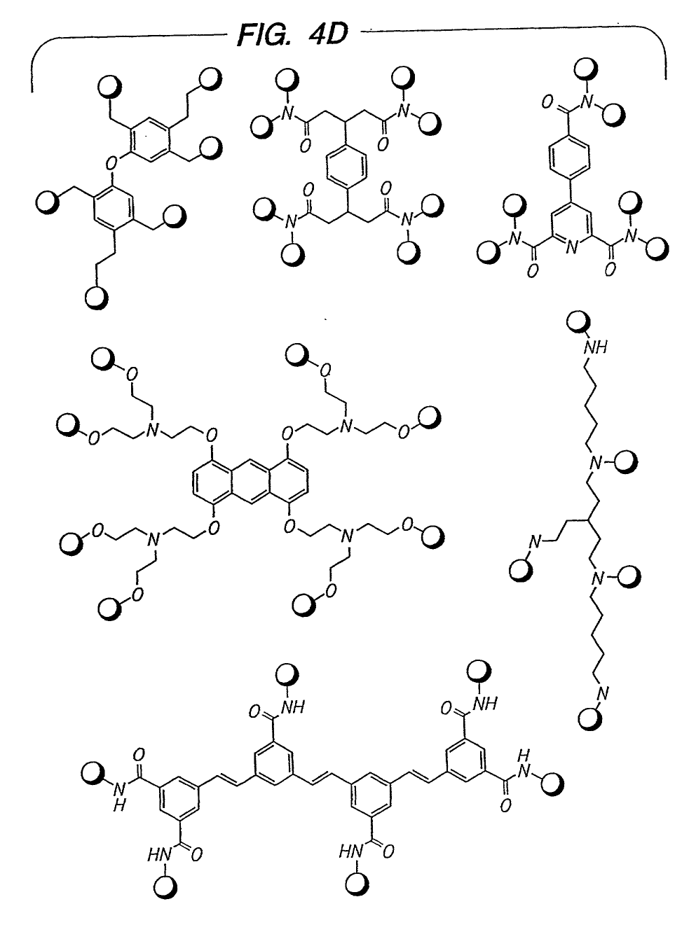

FIG. 4 illustrates the variety of multiligand orientations that are obtained using different types of linkers. It can therefore be seen that there is a plethora of possibilities for the composition of a linker. A wide variety of linkers is commercially available (see, e.g., Chem Sources USA and Chem Sources International; the ACD electronic database; and Chemical Abstracts). Many of the linkers that are suitable for use in this invention fall into this category. Others can be readily synthesized by methods known in the art, and as described below.

Examples of linkers include aliphatic moieties, aromatic moieties, steroidal moieties, peptides, and the like. Specific examples are peptides or polyamides, hydrocarbons, aromatics, heterocyclics, ethers, lipids, cationic or anionic groups, or a combination thereof.

Examples are given below and in Tables 4 and 5, but it should be understood that various changes may be made and equivalents may be substituted without departing from the true spirit and scope of the invention. For example, properties of the linker can be modified by the addition or insertion of ancillary groups into the linker, for example, to change the solubility of the multi-binding compound (in water, fats, lipids, biological fluids, etc.), hydrophobicity, hydrophilicity, linker flexibility, antigenicity, stability, and the like. For example, the introduction of one or more poly(ethylene glycol) (PEG) groups onto the linker enhances the hydrophilicity and water solubility of the multi-binding compound, increases both molecular weight and molecular size and, depending on the nature of the unPEGylated linker, may increase the in vivo retention time. Further, PEG may decrease antigenicity and potentially enhances the overall rigidity of the linker.

Ancillary groups that enhance the water solubility/hydrophilicity of the linker and, accordingly, the resulting multi-binding compounds are useful in practicing this invention. Thus, it is within the scope of the present invention to use ancillary groups such as, for example, small repeating units of ethylene glycols, alcohols, polyols, (e.g., glycerin, glycerol propoxylate, saccharides, including mono-, oligosaccharides, etc.) carboxylates (e.g., small repeating units of glutamic acid, acrylic acid, etc.), amines (e.g., tetraethylenepentamine), and the like to enhance the water solubility and/or hydrophilicity of the multi-binding compounds of this invention. In preferred embodiments, the ancillary group used to improve water solubility/hydrophilicity will be a polyether. In particularly preferred embodiments, the ancillary group will contain a small number of repeating ethylene oxide (—CH₂CH₂O—) units.

The incorporation of lipophilic ancillary groups within the structure of the linker to enhance the lipophilicity and/or hydrophobicity of the compounds of Formula I is also within the scope of this invention. Lipophilic groups useful with the linkers of this invention include, but are not limited to, lower alkyl, aromatic groups and polycyclic aromatic groups. The aromatic groups may be either unsubstituted or substituted with other groups, but are at least substituted with a group which allows their covalent attachment to the linker. As used herein the term “aromatic groups” incorporates both aromatic hydrocarbons and heterocyclic aromatics. Other lipophilic groups useful with the linkers of this invention include fatty acid derivatives which may or may not form micelles in aqueous medium and other specific lipophilic groups which modulate interactions between the multi-binding compound and biological membranes.

Also within the scope of this invention is the use of ancillary groups which result in the compound of Formula I being incorporated into a vesicle, such as a liposome, or a micelle. The term “lipid” refers to any fatty acid derivative that is capable of forming a bilayer or micelle such that a hydrophobic portion of the lipid material orients toward the bilayer while a hydrophilic portion orients toward the aqueous phase. Hydrophilic characteristics derive from the presence of phosphato, carboxylic, sulfato, amino, sulfhydryl, nitro and other like groups well known in the art. Hydrophobicity could be conferred by the inclusion of groups that include, but are not limited to, long chain saturated and unsaturated aliphatic hydrocarbon groups of up to 20 carbon atoms and such groups substituted by one or more aryl, heteroaryl, cycloalkyl, and/or heterocyclic group(s). Preferred lipids are phosphoglycerides and sphingolipids, representative examples of which include phosphatidylcholine, phosphatidylethanolamine, phosphatidylserine, phosphatidylinositol, phosphatidic acid, palmitoyleoyl phosphatidylcholine, lysophosphatidylcholine, lysophosphatidyl-ethanolamine, dipalmitoylphosphatidylcholine, dioleoylphosphatidylcholine, distearoylphosphatidylcholine and dilinoleoylphosphatidylcholine. Other compounds lacking phosphorous, such as sphingolipid and glycosphingolipid families, are also within the group designated as lipid. Additionally, the amphipathic lipids described above may be mixed with other lipids including triglycerides and sterols.

The flexibility of the linker can be manipulated by the inclusion of ancillary groups which are bulky and/or rigid. The presence of bulky or rigid groups can hinder free rotation about bonds in the linker, or bonds between the linker and the ancillary group(s), or bonds between the linker and the functional groups. Rigid groups can include, for example, those groups whose conformational freedom is restrained by the presence of rings and/or π-bonds, for example, aryl, heteroaryl and heterocyclic groups. Other groups which can impart rigidity include polypeptide groups such as oligo- or polyproline chains.

Rigidity can also be imparted electrostatically. Thus, if the ancillary groups are either positively or negatively charged, the similarly charged ancillary groups will force the linker into a configuration affording the maximum distance between each of the like charges. The energetic cost of bringing the like-charged groups closer to each other, which is inversely related to the square of the distance between the groups, will tend to hold the linker in a configuration that maintains the separation between the like-charged ancillary groups. Further, ancillary groups bearing opposite charges will tend to be attracted to their oppositely charged counterparts and potentially may enter into both inter- and intramolecular ionic bonds. This non-covalent mechanism will tend to hold the linker in a conformation which allows bonding between the oppositely charged groups. The addition of ancillary groups which are charged, or alternatively, protected groups that bear a latent charge which is unmasked, following addition to the linker, by deprotection, a change in pH, oxidation, reduction or other mechanisms known to those skilled in the art, is within the scope of this invention.

Bulky groups can include, for example, large atoms, ions (e.g., iodine, sulfur, metal ions, etc.) or groups containing large atoms, polycyclic groups, including aromatic groups, non-aromatic groups and structures incorporating one or more carbon-carbon p-bonds (i.e., alkenes and alkynes). Bulky groups can also include oligomers and polymers which are branched- or straight-chain species. Species that are branched are expected to increase the rigidity of the structure more per unit molecular weight gain than are straight-chain species. In preferred embodiments, rigidity (entropic control) is imparted by the presence of alicyclic (e.g., cycloalkyl), aromatic and heterocyclic groups. In other preferred embodiments, this comprises one or more six-membered rings. In still further preferred embodiments, the ring is an aryl group such as, for example, phenyl or naphthyl, or a macrocyclic ring such as, for example, a crown compound.

In view of the above, it is apparent that the appropriate selection of a linker group providing suitable orientation, entropy and physico-chemical properties is well within the skill of the art.

Eliminating or reducing antigenicity of the multibinding compounds described herein is also within the scope of this invention. In certain cases, the antigenicity of a multi-binding compound may be eliminated or reduced by use of groups such as, for example, poly(ethylene glycol).

As explained above, the multi-binding compounds described herein comprise 2-10 ligands attached covalently to a linker that links the ligands in a manner that allows their multivalent binding to ligand binding sites of cell membrane transporters. The linker spatially constrains these interactions to occur within dimensions defined by the linker. This and other factors increases the biologic and/or therapeutic effect of the multi-binding compound as compared to the same number of ligands used in monobinding form.

The compounds of this invention are preferably represented by the empirical formula (L)ₚ(X)_{q} where L, X, p and q are as defined above. This is intended to include the several ways in which the ligands can be linked together in order to achieve the objective of multivalency, and a more detailed explanation is provided below.

As noted previously, the linker may be considered as a framework to which ligands are attached. Thus, it should be recognized that the ligands can be attached at any suitable position on this framework, for example, at the termini of a linear chain or at any intermediate position thereof.

The simplest and most preferred multi-binding compound is a bivalent compound which can be represented as L-X-L, where L is a ligand and is the same or different and X is the linker. Examples of such bivalent compounds are provided in FIGS. 4A, 6, 7, 8, 9 and 11. A trivalent compound could also be represented in a linear fashion, i.e., as a sequence of repeated units L-X-L-X-L, in which L is a ligand and is the same or different at each occurrence, as is X. However, a trivalent compound can also comprise three ligands attached to a central core, and thus be represented as (L)₃X, where the linker X could include, for example, an aryl or cycloalkyl group. Illustrations of trivalent and tetravalent compounds of this invention are found in FIGS. 4B 4C, and 10. Tetravalent compounds can be represented in a linear array, L-X-L-X-L-X-L, or a branched array,

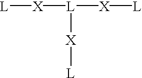

i.e., a branched construct analogous to the isomers of butane (n-butyl, iso-butyl, sec-butyl, and t-butyl). Alternatively, it could be represented as an aryl or cycloalkyl derivative as described above with four (4) ligands attached to the core linker.

The same considerations apply to higher multibinding compounds of this invention containing from 5-10 ligands, as illustrated in FIG. 4(D). However, for multibinding agents attached to a central linker such as an aryl, cycloalkyl or heterocyclyl group, or a crown compound, there is a self-evident constraint that there must be sufficient attachment sites on the linker to accommodate the number of ligands present; for example, a benzene ring could not accommodate more than 6 ligands, whereas a multi-ring linker (e.g., biphenyl) could accommodate a larger number of ligands.

The formula (L)ₚ(X)_{q} is also intended to represent a cyclic compound of formula (-L-X-)ₙ , where n is 2-10.

All of the above variations are intended to be within the scope of the invention defined by the formula (L)ₚ(X)_{q}.

With the foregoing in mind, a preferred linker may be represented by the following formula:

-X′-Z-(Y′-Z)ₘ-Y″-Z-X′-

in which:

m is an integer of from 0 to 20;

X′ at each separate occurrence is —O—, —S—, —S(O)—, —S(O)₂—, —NR—, —N⁺ R R′—, —C(O)—, —C(O)O—, —C(O)NH—, —C(S), —C(S)O—, —C(S)NH— or a covalent bond, where R and R′ at each separate occurrence are as defined below for R′ and R″;

Z is at each separate occurrence selected from alkylene, substituted alkylene, alkylalkoxy, cycloalkylene, substituted cycloalkylene, alkenylene, substituted alkenylene, alkynylene, substituted alkynylene, cycloalkenylene, substituted alkenylene, arylene, substituted arylene, heteroarylene, heterocyclene, substituted heterocyclene, crown compounds, or a covalent bond;

Y′ and Y′ at each separate occurrence are selected from

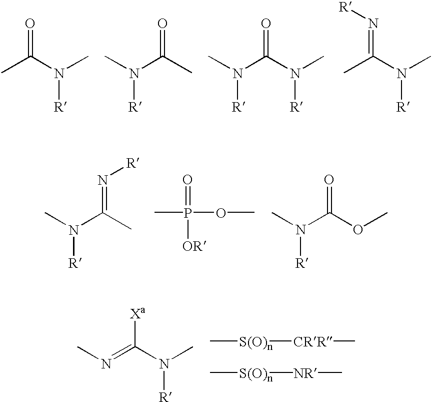

—S—S— or a covalent bond;

in which:

n is 0, 1 or 2; and

R′ and R″ at each separate occurrence are selected from hydrogen, alkyl, substituted alkyl, cycloalkyl, substituted cycloalkyl, alkenyl, substituted alkenyl, alkynyl, substituted alkynyl, aryl, heteroaryl or heterocyclic.

Additionally, the linker moiety can be optionally substituted at any atom therein by one or more alkyl, substituted alkyl, cycloalkyl, substituted cycloalkyl, alkenyl, substituted alkenyl, alkynyl, substituted alkynyl, aryl, heteroaryl and heterocyclic group.

### Preparation of Compounds of Formula I

Accordingly, examples of compounds of Formula I may be prepared as shown below.

As indicated above, the simplest (and preferred) construct is a bivalent compound which can be represented as L-X-L, where L is a ligand that is the same or different at each occurrence, and X is the linker. Accordingly, an example of the preparation of a bivalent ligand is given below as an illustration of the manner in which multi-binding compounds of Formula I are obtained. This example is applicable to any ligand that includes or that can be functionalized with amino and/or carboxyl groups, for example, procaine, tetracaine, nifedipine, diltiazem, amlodipine, and others. Examples of different linkers (X) are shown. In the reactions schemes that follow, for ease of understanding of the principles involved, the structure of the ligand is presented as a “box”. Thus, the ligand is illustrated such that carboxyl [C], amino [V], and methylamino [N] groups are shown as examples of connecting points for the linker.

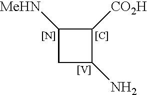

Two ligands are connected by the linker X via a carboxyl group or amino group of a first ligand to any carboxyl group or amino group of a second ligand. Another simplification in the description of the preparations is that, for example, compound (1) is illustrated as a compound of formula H₂N—(CH₂)ₘ—NHCO₂-t-butyl, in which m is an integer of 1-20. However, it should be understood that (CH₂)ₘ is not intended to signify or imply that the scope of this reaction (or of the invention) is limited to straight (i.e. unbranched) alkylene chains, but rather (CH₂)ₘ is intended to include branched alkylenes as defined in the Detailed Description of the Invention, substituted alkylenes, and the like, also as disclosed in the Detailed Description of the Invention. Similarly, the compound of formula (2) is illustrated as ClC(O)— (CH₂)ₙ—C(O)Cl, and (CH₂)ₙ equally is not limited to straight alkylene chains, but includes all those modifications shown above.

Accordingly, bivalent compounds of Formula I where the linkage is from a [C] group of a first ligand to a [C] group of a second ligand, i.e. a [C-C] linkage, may be prepared from intermediates of formula (4), the preparation of which is shown below in Reaction Scheme 1.

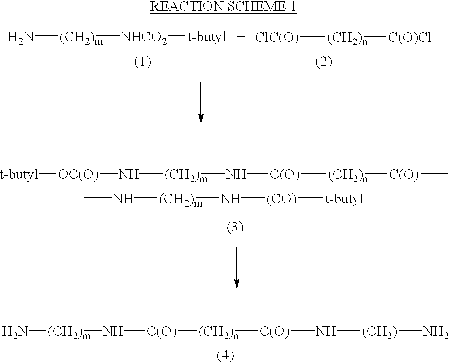

in which m and n are independently at each occurrence integers of 1-20.

### Preparation of Compounds of Formula (3)

As illustrated in Reaction Scheme 1, step 1, about two molar equivalents of an omega-amino carbamic acid ester [formula (1)] is reacted with about one molar equivalent of a dicarboxylic acid halide, preferably chloride, of formula (2). The reaction is conducted in the presence of a non-nucleophilic base, preferably diisopropylethylamine, in an inert solvent, preferably methylene chloride, at a temperature of about 0-5° C. The mixture is then allowed to warm to room temperature. When the reaction is substantially complete, the compound of formula (3) is isolated and purified by conventional means.

### Preparation of Compounds of Formula (4)

As illustrated in Reaction Scheme 1, step 2, the carbamate is removed under acid conditions. In general a preferred acid is trifluoroacetic acid, and the reaction is conducted in an inert solvent, preferably methylene chloride, at about room temperature. When the reaction is substantially complete, the compound of formula (4) is isolated and purified by conventional means.

The compound of formula (4) is then converted into a [C-C] ligand dimer as shown in Reaction Scheme 2 (Appendix).

### Preparation of Compounds of Formula I

In general, about two molar equivalents of ligand is reacted with about one molar equivalent of the compound of formula (4), under conventional amide coupling conditions. Preferably, a hindered base is employed, preferably diisopropylethylamine, in the presence of benzotriazol-1-yloxytripyrrolidinophosphonium hexafluorophosphate (PyBOP) and 1-hydroybenzotriazole. The reaction is conducted in an inert polar solvent, for example, N, N-dimethylformamide (DMF) or dimethyl sulfoxide (DMSO), or preferably a mixture of both, at about room temperature. When the reaction is substantially complete, the compound of Formula I is isolated and purified by conventional means, preferably purified by reverse phase HPLC. Also isolated was a byproduct of formula (5).

Alternatively, compounds of Formula I [C-C] may be prepared from intermediates of formula (8), the preparation of which is shown in Reaction Scheme 3 (Appendix).

### Preparation of Compounds of Formula (7)

As illustrated in Reaction Scheme 3, step 1, ligand is reacted with about 1.1 molar equivalents of a carbamic ester terminated by an alkylamino group [formula (6)]. The ester moiety is chosen for ease of removal under mild conditions in subsequent reactions, and is preferably 9-fluorenylmethyl. Conventional amide coupling conditions are employed, preferably using PyBOP and 1-hydroxybenzotriazole. In general, the reaction is conducted in the presence of a hindered base, preferably diisopropylethylamine, in an inert polar solvent, preferably DMF or DMSO, preferably a mixture of both, at about room temperature. When the reaction is substantially complete, the compound of formula (7) is isolated and purified by conventional means.

### Preparation of Compounds of Formula (8)

As illustrated in Reaction Scheme 3, step 2, the compound of formula (7) is reacted with a mild base to remove the protecting ester groups, which also affords decarboxylation. In general, the base is preferably piperidine, and the reaction is conducted in an inert polar solvent, preferably dimethylformamide, at about room temperature for about 10 minutes to one hour. When the reaction is substantially complete, the compound of formula (8) is isolated and purified by conventional means, preferably using reverse phase HPLC.

The compound of formula (8) is then converted into a [C-C] bivalent compound as shown in Reaction Scheme 4 (Appendix).

### Preparation of Compounds of Formula I

As illustrated in Reaction Scheme 4, the compound of formula (8) is reacted with a dicarboxylic acid. In general, about 3 molar equivalents of the compound of formula (8) is reacted with about 1 molar equivalent of the dicarboxylic acid of formula HO₂C—(CH₂)ₙ—CO₂H, under conventional amide coupling conditions. Preferably, a hindered base is employed, preferably diisopropylethlyamine, in the presence of PyBOP and 1-hydroxybenzotriazole. The reaction is conducted in an inert polar solvent, preferably DMF, at about room temperature for about 1-3 hours. When the reaction is substantially complete, the compound of Formula I is isolated and purified by conventional means, preferably purified by reverse phase of HPLC.

Compounds of Formula I wherein the linkage is [V-V] may be prepared from intermediates of formula (14), the preparation of which is shown in Reaction Scheme 6 (Appendix). The starting material, the compound of formula (11), is prepared as shown in Reaction Scheme 5 (Appendix).

### Preparation of Compounds of Formula (10)

As illustrated in Reaction Scheme 5, step 1, a ligand having an —NH₂ group suitable for linking is reacted with a protected ester-aldehyde of formula (9) to form a Schiff's base. The ester moiety is chosen for ease of removal under mild conditions in subsequent reactions, and is preferably 9-fluorenylmethyl. In general, the reaction is conducted in an inert polar solvent, preferably 3-dimethyl-3,4,5,6-tetrahydro-2(1H)-pyrimidinone plus methanol, at about 50-100° C., preferably about 70° C., for about 30 minutes to 1 hour. The Schiff's base of formula (10) is not isolated, but reacted further immediately as shown below.

### Preparation of Compounds of Formula (11)

As illustrated in Reaction Scheme 5, step 2, the solution of the compound of formula (10) is further reacted with a mild reducing agent. In general, the reducing agent is preferably sodium cyanoborohydride, and the reaction is conducted at about 50-100° C., preferably about 70° C., for about 1-3 hours, preferably about 2 hours. When the reaction is substantially complete, the compound of formula (11) is isolated and purified by conventional means, preferably purified by reverse phase HPLC.

Compounds of Formula I wherein the linkage is [V-V] may then be prepared from intermediates of formula (11a), the preparation of which is shown in Reaction Scheme 6 (Appendix).

### Preparation of Compounds of Formula (14)

As illustrated in Reaction Scheme 6, step 1, the compound of formula (11a), which is a compound of formula (11) in which the carboxyl group has been protected conventionally, for example as an ester, is reacted with a mild base to remove the carbamate. In general, the base is preferably piperidine, and the reaction is conducted in an inert polar solvent, preferably DMF, at about room temperature for about 10 minutes to one hour, preferably about 30 minutes. When the reaction is substantially complete, the compound of formula (14) is isolated and purified by conventional means, preferably using reverse phase HPLC.

### Preparation of Compounds of Formula I

As illustrated in Reaction Scheme 6, step 2, the compound of formula (14) is reacted with a dicarboxylic acid. In general, about 3 molar equivalents of the compound of formula (8) is reacted with about 1 molar equivalent of the dicarboxylic acid of formula HO₂C—(CH₂)ₙ—CO₂H, under conventional amide coupling conditions. Preferably, a hindered base is employed, preferably diisopropylethylamine, in the presence of PyBOP and 1-hydroxybenzotriazole. The reaction is conducted in an inert polar solvent, preferably DMF, at about room temperature for about 1-3 hours. When the reaction is substantially complete, the protecting group R, preferably and ester, is removed conventionally, and the [V-V] compound of Formula I is isolated and purified by conventional means, preferably purified by reverse phase HPLC.

Compounds of Formula I wherein the linkage is [C-V] may be prepared from intermediates of formula (23), the preparation of which is shown in Reaction Scheme 7 (Appendix). The starting material, the compound of formula (8), is prepared as previously shown.

### Preparation of Compounds of Formula (22)

As illustrated in Reaction Scheme 7, step 1, the compound of formula (8) is reacted with an acid in the same manner as shown above, for example in Reaction Scheme 11, to form an amide of formula (22).

### Preparation of Compounds of Formula (23)

As illustrated in Reaction Scheme 7, step 2, the compound of formula (22) is hydrolyzed with a base, for example piperidine, in the same manner as shown above, to form a compound of formula (23).

The compound of formula (23) is then converted into a [C-V] bivalent compound of Formula I by reaction with a compound of formula (17), prepared as shown previously, as shown in Reaction Scheme 8 (Appendix).

### Preparation of Compounds of Formula I

As illustrated in Reaction Scheme 8, the compound of formula (23) is reacted with a compound of formula (17) in a typical coupling reaction as shown previously, to give a compound of Formula I [C-V].

Compounds of Formula I wherein the linkage is [C-N] may be prepared from intermediates of formula (26), the preparation of which is shown below in Reaction Scheme 9 (Appendix).

### Preparation of Compounds of Formula (24)

As illustrated in Reaction Scheme 9, step 1, ligand is reacted with a protected aminoaldehyde in the presence of an amount of base sufficient to direct the reaction of the aldehyde to the [N] position. The Schiff's base thus formed is reduced in the same manner as shown in Reaction Scheme 5 to form a compound of formula (24).

### Preparation of Compounds of Formula (25)

As illustrated in Reaction Scheme 9, step 2, the compound of formula (24) is reacted with an amine in a coupling reaction in the same manner as shown previously, for example in Reaction Scheme 10, to form an amide of formula (25).

### Preparation of Compounds of Formula (26)

As illustrated in Reaction Scheme 9, step 3, the protecting group FM is removed conventionally from the compound of formula (25) with a mild base to form a compound of formula (26).

The compound of formula (26) is then converted into a [C-N] bivalent compound of Formula I by reaction with a compound of formula (23), prepared as shown previously, as shown in Reaction Scheme 10 (Appendix).

### Preparation of Compounds of Formula I

As illustrated in Reaction Scheme 10, the compound of formula (26) is reacted with a compound of formula (23) in a typical coupling reaction as shown previously, for example in Reaction Scheme 6, to give a compound of Formula I [C-N].

Compounds of Formula I wherein the linkage is [N-V] may be prepared by a reaction of a compound of formula (26) with a compound of formula (19), as shown in Reaction Scheme 11 (Appendix).

### Preparation of Compounds of Formula I

As illustrated in Reaction Scheme 11, the compound of formula (26) is reacted with a compound of formula (19) in a typical coupling reaction as shown above, to give a compound of Formula I [N-V].

Compounds of Formula I wherein the linkage is [N-N] may be prepared by reaction of a compound of formula (26) with a dicarboxylic acid of formula HO₂—C—(CH₂)ₙ—CO₂H, as shown in Reaction Scheme 12 (Appendix).

### Preparation of Compounds of Formula I

As illustrated in Reaction Scheme 12, the compound of formula (26) is reacted with a dicarboxylic acid of formula HO₂C—(CH₂)ₙ—CO₂H in the same manner as shown previously in Reaction Scheme 4, to give a compound of Formula I [N-N].

### Preparation of Higher Valency Compounds of Formula I

Trivalent compounds may be prepared from bivalent compounds, for example by reacting the free carboxyl group of a bivalent compound of Formula I as prepared in Reaction Scheme 8 [C-N] with an appropriate linker, which is in turn reacted with a ligand, thus giving three ligands linked together (i.e. of the type L-X-L-X-L). Alternatively, the bivalent compound of Formula I as prepared in Reaction Scheme II could be reacted further with an appropriate linker and ligand to give a trivalent compound.

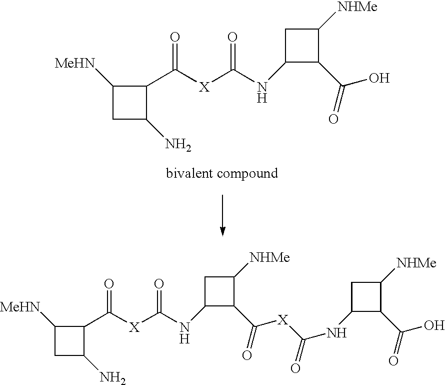

### Trivalent Compound where —X—C(O)—is a linker

Clearly, other compounds of Formula I may be similarly prepared as multibinding agents, i.e. tetravalent, pentavalent, hexavalent, heptavalent, octavalent, nonavalent, and decavalent compounds.

Alternatively, reaction of three molecules of a ligand bearing a carboxyl group with one molecule of a cyclic linker, such as 1,3,5-tris-(aminomethyl)benzene, yields a trimeric compound that is potentially trivalent. Other cyclic oligomers can be similarly prepared.

Where X represents —CH₂NH—; and L is a ligand.

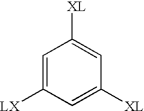

Tetravalent compounds may be prepared as shown above. An alternative configuration for a tetravalent compound would be obtained by dimerizing a bivalent compound by connecting two molecules of a bivalent compound via its linker. This could be represented as follows:

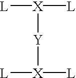

Where L is a ligand that is the same or different at each occurrence;

X is a linker; and

Y is a linker that is the same as X or different.

### Alternative Methods of Linking Ligands via Amino Groups

Ligands having terminal amino groups may also be linked by reaction with aldehyde linkers to form a Schiff's base, which can be used as such as a linker or reduced to give a saturated linker. Alternatively, reaction with a disulfonyl halide would give a sulfonamide linker. Another alternative synthesis encompasses reaction of an amine with a diiminoester, which would afford an amidine linker.

### Linking Ligands via Hydroxy Groups

Ligands that include a free hydroxy group in their structure (an alcohol or phenolic hydroxy) may be connected using those hydroxy groups as linkage points by means well known in the art. For example, one synthetic strategy that could be used for linking ligands with free hydroxy groups involves treating the ligand with t-butyl bromoacetate in the presence of a suitable base (e.g. potassium carbonate, NaH) to convert the —OH group to an —O—CH₂CO₂-t-But group, which can be hydrolyzed to an O—CH₂CO₂H group using trifluoracetic acid. The oxyacetic group can then be used as the linking point for two ligands by making use of the linking strategies shown previously for carboxylic acids. For example, reaction of two molar equivalents of the ligand with a diamine of the formula H₂N—(CH₂)ₙ—NH₂, where n is an integer of 1-20, leads to two ligands being connected by a linker of the formula —CH₂CONH—(CH₂)ₙ—NHCOCH_{2.}

Alternatively, treating an alkoxy-bearing ligand with BOC—NHCH₂CH₂Br effectively converts the —OH group to an O—CH₂CH₂NH—BOC group, which can by deprotected to give an O—CH₂CH₂NH₂ group using trifluoracetic acid. The oxyethylamino group can then be used as the linking point for two ligands by making use of the linking strategies shown above for amines. For example, reaction of two molar equivalents of the ligand with a dicarboxylic acid of the formula HO₂C—(CH₂)ₙ—CO₂H where n is an integer of 1-20, leads to two ligands being connected by a linker of the formula:

—CH₂CH₂NHCO—(CH₂)ₙ—CONHCH₂CH₂—.

Amiodarone is an example of a ligand that includes a hydroxy group suitable for this linking strategy.

Alternatively, converting the hydroxy-group to a leaving group, for example by treatment with mesyl chloride or tosyl chloride, or converting the hydroxy group to a halide by means well known in the art, the ligand can then be linked directly by reaction with a diamine.

The Mannich reaction can be used to link ligands that have an “active” hydrogen in their molecular structure. Examples of such active hydrogens are hydrogens that are adjacent to an electron withdrawing group such as a ketone, aldehyde, acid or ester, nitrile, and nitro group, and the like. The Mannich reaction is well known to those skilled in the art, and many reviews in the chemical literature and textbooks on the Mannich reaction are available. Of particular value is that a linker could be constructed on a ligand having an aromatic moiety, providing there is an active hydrogen. As those of skill in the art will appreciate, other art-recognized chemistries can be used to provide ligands with suitable functional groups for attachment to a linker.

In view of the above, the chemistry for attaching ligands to linkers is well established in the art and is well within the level of ordinary skill in the art.

### Specific Embodiments

The following examples are used to specifically illustrate the attachment of several classes of ligands to linkers according to this invention. The specific ligands employed are for illustrative purposes and should not be construed as a limitation for this application.

### Inhibitors of Sodium Ion Channels

Included among compounds of Formula I are compounds comprising ligands that inhibit the activity of sodium ion channels. Not surprisingly, these ligands are used for a variety of clinical conditions, for example: ligands such as lidocaine, bupivacaine, benzocaine, etidocaine, mepivacaine, prilocaine, ropivacaine, tetracaine, and the like have local anesthetic properties and are used for the treatment of pain, and for other types of applications, e.g., protection and recovery from ischemia (Lantos et al, Arch. Int. Pharmacodyn. Ther. 331: 179 (1996), asthma (Hunt et al., Mayo Clin. Proc. 71: 361 (1996), rapid heartbeat (Gorgels et al. Am. J. Cardiol. 78: 43 (1996)) and natriuresis (Wyeth et al, Life Sci. 60: 473 (1997)); ligands such as carbamazepine, phenytoin, lamotrigine and valproic acid are used to treat seizures; ligands such as disopyramide, procainamide, quinidine, lidocaine, mexiletine, phenytoin, tocainide, encainide, flecainide, and propafenone are used as antiarrhythmic drugs; and amiloride is used conjunctively with other drugs to improve mucociliary clearance in cystic fibrosis and as a diuretic.

### Local Anesthetic-type Compounds

Local anesthetic compounds inhibit the neuronal voltage-gated Na⁺ion channels and thereby block conduction of pain impulses. There exists a clinical need for local anesthetics with improved safety profiles (e.g., the CNS effects of current agents include restlessness, convulsions, myocardial toxicity, allergic reactions), improved selectivity for sensory vs. motor block, prolonged duration of action and greater potency (e.g., the principal disadvantage of existing agents for infiltration anesthesia is that the required tissue concentration is too high to provide anesthesia for large volumes of tissue).

The duration of action of a local anesthetic is proportional to the time during which the anesthetic is in contact with the nerve, or, more precisely, the ion channel(s). The effect of currently used local anesthetics tends to be short-lived as a result of dissociation from and diffusion away from the intended site of action. Therefore, repeated doses or a slow-release formulation must be administered to achieve a prolonged effect. Undesired side effects of local anesthetics, such as those mentioned above, are largely a function of systemic concentrations of the drug resulting from such diffusion. Because of these serious side effects, the quantity of drug administered and its localization to non-systemic compartments must be carefully controlled.

Consequently, methods that localize a local anesthetic to its intended site of action and provide reduced off-rate would be useful for prolonging the duration of action, hence the clinical utility of local anesthetics in pain management and mitigating their untoward toxic effects resulting from systemic levels of the drug.

A multi-binding local anesthetic compound comprises from 2-10 ligands linked covalently for multivalency. One group of preferred ligands includes conventional local anesthetics such as those described above. Also included in this class of compounds are ligands that share common structural features with conventional local anesthetics such as those listed above, i.e., a benzene ring linked via an ester or amide linkage to an aliphatic group having a terminal tertiary or quaternary amine. The length of the linkage, i.e. the distance between the benzene ring and the amine, is typically about 6-9 Å. Preferred multi-binding local anesthetic compounds include at least two such local anesthetic-type binding groups.

The modular, repeated tetrameric molecular structure of the sodium ion channel presents a plurality of cation binding sites. Accordingly, quaternary amines, guanidines and amidines are a second preferred class of binding group to be employed in a multi-binding local anesthetic compound. These positively charged amine moieties may comprise the terminal amine moiety of a local anesthetic-type binding group, or may be otherwise linked to a local anesthetic-type binding group, or to the linker framework (described further below) of the multi-binding local anesthetic compound. Also contemplated, in addition to tetraalkyl ammonium ions, are other amine functionalities which are cationic at physiological pH, such as guanidines and amidines.

In addition to these interactions, the binding of lipophilic groups in the cell membrane proximate to the channel can be useful for increasing the duration of action of multi-binding local anesthetics. A third type of preferred binding group, therefore, is a lipophilic group such as a long chain alkyl group or an aromatic hydrocarbon. Such a group may be linked to the local anesthetic-type binding group, or it may comprise part of a quaternary alkyl amine, or it may be linked to the linker framework of the multi-binding local anesthetic.

The ligands, as described above, are linked together for multivalent binding interactions with ligand binding sites of a voltage-gated Na⁺ ion channel. The multi-binding compound includes at least two local anesthetic-type binding groups, and preferably additional groups from the second and third classes described above.

One type of preferred linker comprises a linear, branched or cyclic chain having from two to fifty, and preferably two to twenty-four, non-hydrogen atoms, where the bonds making up the chain are selected from alkyl (carbon-carbon), alkenyl (double bonded carbon-carbon), alkyl ether (carbon-oxygen), thioether (carbon-sulfur), alkyl amino (carbon-nitrogen) linkages, or a combination thereof. The chain may further include one or more groups selected from cycloalkyl, heterocyclyl, aryl, heteroaryl, carbonyl, carboxy ester, carboxy amide, and sulfonyl, intervening between these bonds.

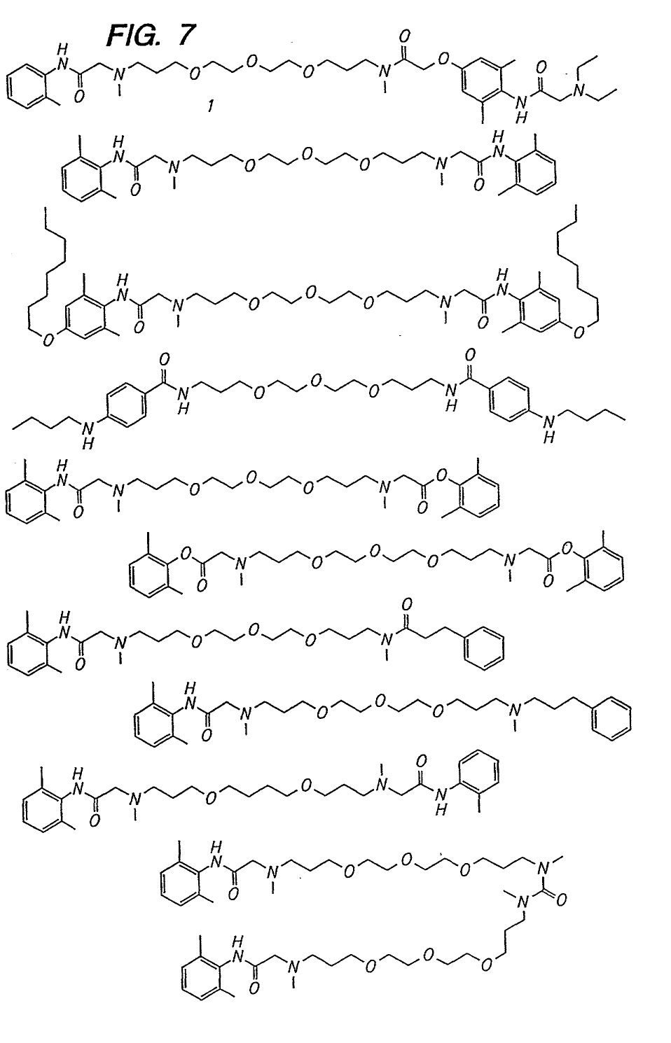

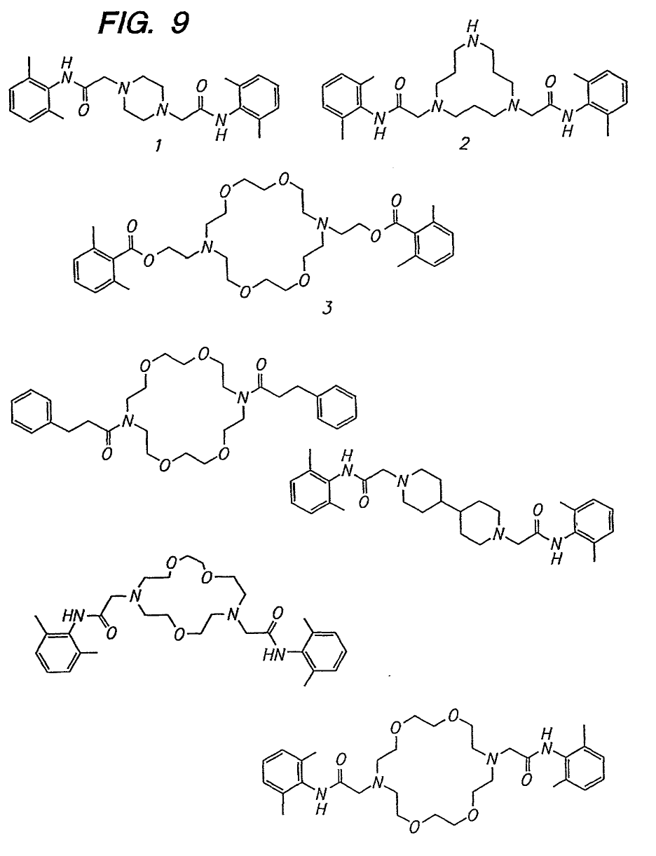

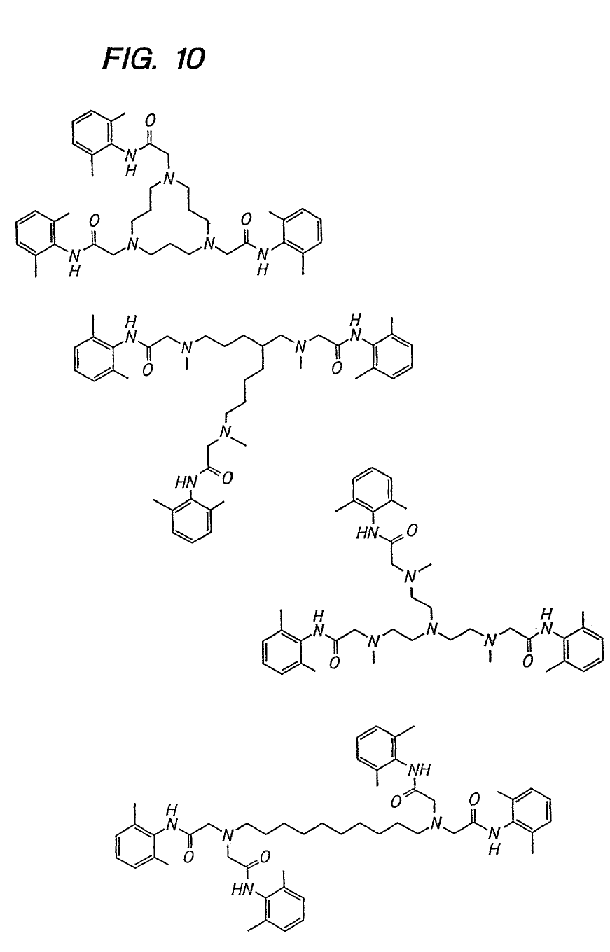

In some preferred compounds, the linker includes three to twelve atoms between amine moieties of the ligands, e.g. between the aliphatic amine groups of local anesthetic-type binding groups. In other preferred compounds, the nitrogen atoms of one or more groups may form part of the linker, as discussed above and shown, for example, in FIGS. 6-11, which illustrate differently substituted bivalent, trivalent and higher-order valency multi-binding compounds containing local anesthetic-type ligands. The ligands may be linked via a linear linker, as in, for example, those shown in FIGS. 6 and 7, or form a macrocyclic linker, as in certain crown compounds shown in FIGS. 9-11. The linker may combine linear and cyclic structures, as also shown in FIG. 9.

Local anesthetic-type ligands may be linked to the linker at different attachment points to achieve different orientations of the ligand domains, thereby to facilitate multivalency, as was discussed previously. This is illustrated below for lidocaine-based compounds of Formula I. Lidocaine is preferably linked via the aliphatic amine moieties, via substituents on the benzene ring, or via the carbon which is alpha to the amide carbonyl group. The lidocaine moieties may be linked to form various combinations of C—, N— and Aryl-linked bivalent compounds. The linker shown below is one of several classes of preferred linkers for local anesthetic-type compounds of this invention, as discussed previously. Other preferred linkers are exemplified in FIGS. 6-11 (Appendix).

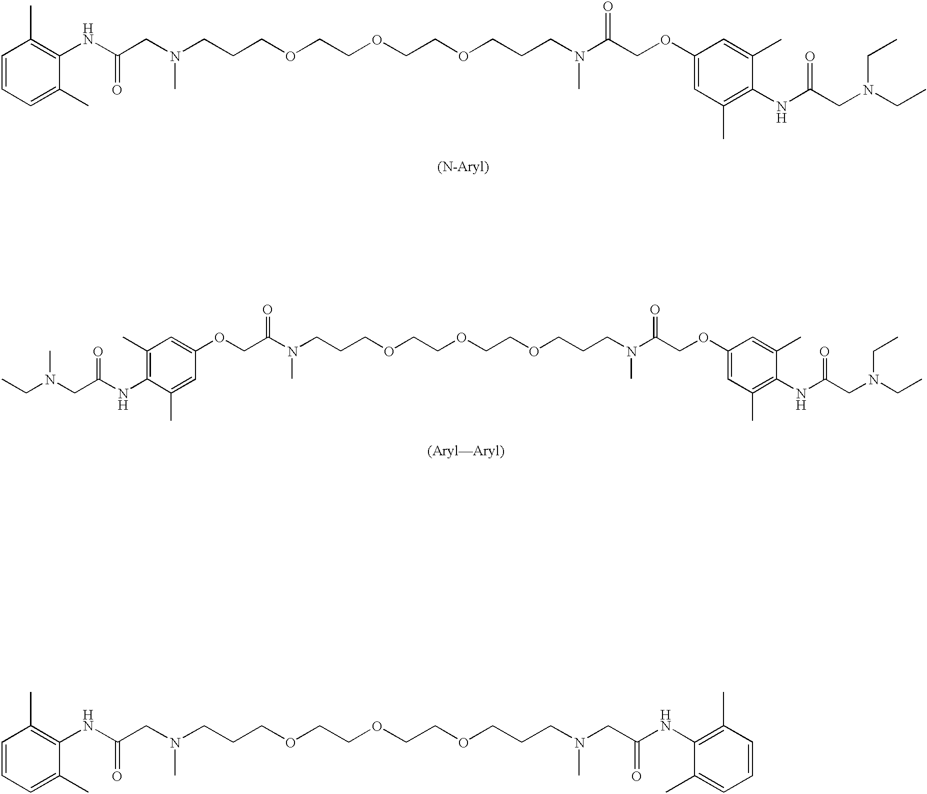

### N—N

Linear linkers may include oligomers having side chains comprising local anesthetic-type binding groups. Any of these compounds may further include binding groups of the second and third classes, i.e. quaternary ammonium ions and/or lipophilic groups, as described above. The compounds may also include one or more chiral centers, such as exists in the monobinding local anesthetic bupivacaine, and may be optically active at such centers, such as the monobinding anesthetic ropivacaine. The centers may be present in the linker of the compound, or in the other types of binding groups, as well as in the local anesthetic-type ligand. Chirality may also be present in compounds lacking asymmetric atoms, e.g., in allene, biphenyl, spirane and helical compounds.

In one embodiment, particularly useful for more potent anesthetic compounds, the framework comprises ester linkages which are degradable by plasma esterases. The presence of such linkages limits the lifetime of the compound in systemic circulation, thereby reducing systemic toxicity. Such linkages may also be included in the binding groups themselves.

### Preparation of Local Anesthetic Compounds of Formula I

Representative synthetic methods for preparation of the multi-binding local anesthetic compounds of this invention are shown in Reaction Schemes 14-17 (Appendix).

Bivalent, trivalent and higher-order valency compounds can be prepared according to the general method shown in Reaction Scheme 14. The starting material is a bi-, tri- or multi-functional amine. A large variety of such amines is commercially available, or they may be prepared by synthetic methods well known in the art. As described above, linkers may incorporate alkylene, alkalkoxy, alkthioalkoxy, amine, carbonyl, ester, or amide linkages, and ring structures.

As illustrated in Reaction Scheme 14, three molar equivalents of an appropriately substituted N-aryl-2-haloacetamide is reacted with a slight excess of a triamine or triamine salt, preferably in an ethanolic solution containing diisopropylethylamine under an inert atmosphere with mild heating, e.g. 80° C., for several hours until the reaction is complete. The haloacetamide can be prepared by reaction of a substituted aniline with haloacetyl chloride (see Example 7 below). For reaction of secondary amines, the solution is preferably heated at reflux.

The trivalent compound is isolated and purified by conventional means, preferably by precipitation and filtration. For preparation of compounds of higher-order valency, a similar procedure is followed, using an appropriate number of moles of halocompound.

According to another route, a local anesthetic-type ligand having a free amine is reacted with a framework precursor having multiple leaving groups. In one example (Example 10 below), approximately two moles of 2′, 6′-dimethyl-2-piperidinecarboxanilide were reacted with an α,ω-dibromide to produce a compound having the structure shown in FIG. 9 (1).

Compounds incorporating a quaternary amine may be prepared by a similar route, shown in Reaction Scheme 15 and described in Example 9 below. N-methyl amines, for example, can be conveniently prepared from primary amines by synthesizing the t-butoxycarbonyl derivative and reducing it with a hydride reducing agent such as LiAlH₄ (LAH). Reaction of the secondary amine with a substituted α-chloroacetamide, as above gives a multivalent compound having a tertiary amine linkage to the framework. This linkage may be quaternized by reaction with a suitable alkylating agent such as methyl iodide, as shown.

Cyclic or macrocyclic compounds may be prepared from the corresponding cyclic polyamines. These amines include, e.g., piperazine (see, e.g., FIG. 9 (1) to larger structures such as 1,5,9-triazacyclododecane (see, e.g., FIG. 9 (2), 1,4,7,10-tetraazacyclododecane (cyclen), 1,4,8,11 -tetraazacyclotetradecane (cyclam), 1,4,7,10,13,16-hexaazacyclooctadecane (hexacyclen), and the like. The rings may also contain other linkages, such as ether linkages, as in e.g. 1,4,10,13-tetraoxa-7,16-diazacyclooctadecane (see, e.g., FIG. 9 (3)). Such cyclic compounds are frequently commercially available, as are those named above, and can be synthesized by conventional methods.

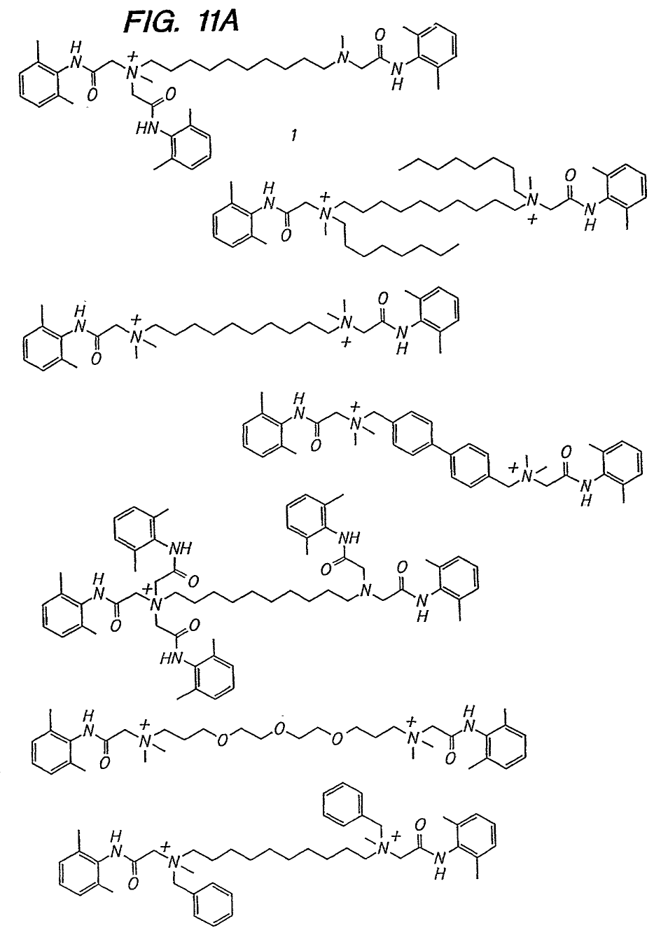

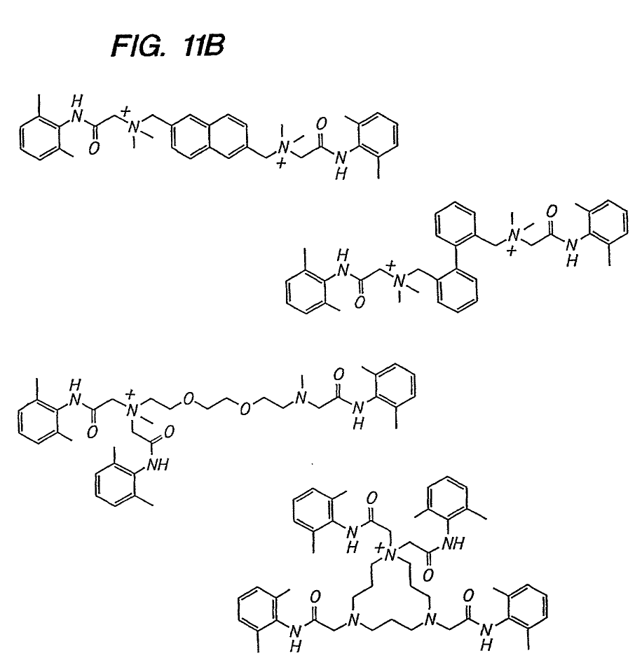

Compounds having two or three local anesthetic-type binding groups attached to a single quaternary nitrogen on a framework (see, e.g., FIG. 11 (1)), can be formed by using a large excess of the α-chloroacetamide component. Such compounds are also typically formed in small quantities as byproducts of the standard alkylation reactions described above. Similarly, “underalkylated” compounds can be formed by using correspondingly less of the α-chloroacetamide component relative to the polyamine.

Local anesthetic-type ligands linked via the aromatic ring, may be prepared as shown in Reaction Scheme 16 and Example 11. A substituted phenol is nitrated and reacted with a bifunctional (or higher functionality) bromide, forming multiple ether linkages, as shown. The nitro groups are reduced, and the resulting amino groups are condensed with α-chloroacetyl chloride to form amide linkages. The aliphatic chlorine is then displaced by an amine, e.g. diethyl amine, to give the multi-binding compound.

Local anesthetic-type ligands may be linked “head-to-tail” (N-Aryl) as shown in Reaction Scheme 17.

When it is desired to incorporate degradable ester linkages into the multivalent compound, this may be accomplished by the use of a local anesthetic-type binding group having an ester rather than an amide linkage. Alternatively, one or more ester linkages may be incorporated into the linking framework (see, e.g., FIG. 7 (1)).

### Combinatorial Libraries

The methods described above lend themselves to combinatorial approaches for identifying multimeric compounds which possess multibinding properties.

Specifically, factors such as the proper juxtaposition of the individual ligands of a multibinding compound with respect to the relevant array of binding sites on a target or targets is important in optimizing the interaction of the multibinding compound with its target(s) and to maximize the biological advantage through multivalency. One approach is to identify a library of candidate multibinding compounds with properties spanning the multibinding parameters that are relevant for a particular target. These parameters include: (1) the identity of ligand(s), (2) the orientation of ligands, (3) the valency of the construct, (4) linker length, (5) linker geometry, (6) linker physical properties, and (7) linker chemical functional groups.

Libraries of multimeric compounds potentially possessing multibinding properties (i.e., candidate multibinding compounds) and comprising a multiplicity of such variables are prepared and these libraries are then evaluated via conventional assays corresponding to the ligand selected and the multibinding parameters desired. Considerations relevant to each of these variables are set forth below:

### Selection of Ligand(s)

A single ligand or set of ligands is (are) selected for incorporation into the libraries of candidate multibinding compounds which library is directed against a particular biological target or targets. The only requirement for the ligands chosen is that they are capable of interacting with the selected target(s). Thus, ligands may be known drugs, modified forms of known drugs, substructures of known drugs or substrates of modified forms of known drugs (which are competent to interact with the target), or other compounds. Ligands are preferably chosen based on known favorable properties that may be projected to be carried over to or amplified in multibinding forms. Favorable properties include demonstrated safety and efficacy in human patients, appropriate PK/ADME profiles, synthetic accessibility, and desirable physical properties such as solubility, logP, etc. However, it is crucial to note that ligands which display an unfavorable property from among the previous list may obtain a more favorable property through the process of multibinding compound formation; i.e., ligands should not necessarily be excluded on such a basis. For example, a ligand that is not sufficiently potent at a particular target so as to be efficacious in a human patient may become highly potent and efficacious when presented in multibinding form. A ligand that is potent and efficacious but not of utility because of a non-mechanism-related toxic side effect may have increased therapeutic index (increased potency relative to toxicity) as a multibinding compound. Compounds that exhibit short in vivo half-lives may have extended half-lives as multibinding compounds. Physical properties of ligands that limit their usefulness (e.g. poor bioavailability due to low solubility, hydrophobicity, hydrophilicity) may be rationally modulated in multibinding forms, providing compounds with physical properties consistent with the desired utility.

### Orientation: Selection of Ligand Attachment Points and Linking Chemistry

Several points are chosen on each ligand at which to attach the ligand to the linker. The selected points on the ligand/linker for attachment are functionalized to contain complementary reactive functional groups. This permits probing the effects of presenting the ligands to their receptor(s) in multiple relative orientations, an important multibinding design parameter. The only requirement for choosing attachment points is that attaching to at least one of these points does not abrogate activity of the ligand. Such points for attachment can be identified by structural information when available. For example, inspection of a co-crystal structure of a protease inhibitor bound to its target allows one to identify one or more sites where linker attachment will not preclude the enzyme:inhibitor interaction. Alternatively, evaluation of ligand/target binding by nuclear magnetic resonance will permit the identification of sites non-essential for ligand/target binding. See, for example, Fesik, et al., U.S. Pat. No. 5,891,643. When such structural information is not available, utilization of structure-activity relationships (SAR) for ligands will suggest positions where substantial structural variations are and are not allowed. In the absence of both structural and SAR information, a library is merely selected with multiple points of attachment to allow presentation of the ligand in multiple distinct orientations. Subsequent evaluation of this library will indicate what positions are suitable for attachment.

It is important to emphasize that positions of attachment that do abrogate the activity of the monomeric ligand may also be advantageously included in candidate multibinding compounds in the library provided that such compounds bear at least one ligand attached in a manner which does not abrogate intrinsic activity. This selection derives from, for example, heterobivalent interactions within the context of a single target molecule. For example, consider a receptor antagonist ligand bound to its target receptor, and then consider modifying this ligand by attaching to it a second copy of the same ligand with a linker which allows the second ligand to interact with the same receptor molecule at sites proximal to the antagonist binding site, which include elements of the receptor that are not part of the formal antagonist binding site and/or elements of the matrix surrounding the receptor such as the membrane. Here, the most favorable orientation for interaction of the second ligand molecule with the receptor/matrix may be achieved by attaching it to the linker at a position which abrogates activity of the ligand at the formal antagonist binding site. Another way to consider this is that the SAR of individual ligands within the context of a multibinding structure is often different from the SAR of those same ligands in momomeric form.

The foregoing discussion focused on bivalent interactions of dimeric compounds bearing two copies of the same ligand joined to a single linker through different attachment points, one of which may abrogate the binding/activity of the monomeric ligand. It should also be understood that bivalent advantage may also be attained with heterodimeric constructs bearing two different ligands that bind to common or different targets. For example, a 5HT₄ receptor antagonist and a bladder-selective muscarinic M₃ antagonist may be joined to a linker through attachment points which do not abrogate the binding affinity of the monomeric ligands for their respective receptor sites. The dimeric compound may achieve enhanced affinity for both receptors due to favorable interactions between the 5HT₄ ligand and elements of the M₃ receptor proximal to the formal M₃ antagonist binding site and between the M₃ ligand and elements of the 5HT₄ receptor proximal to the formal 5HT₄ antagonist binding site. Thus, the dimeric compound may be more potent and selective antagonist of overactive bladder and a superior therapy for urinary urge incontinence.

Once the ligand attachment points have been chosen, one identifies the types of chemical linkages that are possible at those points. The most preferred types of chemical linkages are those that are compatible with the overall structure of the ligand (or protected forms of the ligand) readily and generally formed, stable and intrinsically inocuous under typical chemical and physiological conditions, and compatible with a large number of available linkers, Amide bonds, ethers, amines, carbamates, ureas, and sulfonamides are but a few examples of preferred linkages.

### Linkers: Spanning Relevant Multibinding Parameters through Selection of Valency, Linker Length, Linker Geometry, Rigidity, Physical Properties, and Chemical Functional Groups

In the library of linkers employed to generate the library of candidate multibinding compounds, the selection of linkers employed in this library of linkers takes into consideration the following factors:

Valency. In most instances the library of linkers is initiated with divalent linkers. The choice of ligands and proper juxtaposition of two ligands relative to their binding sites permits such molecules to exhibit target binding affinities and specificities more than sufficient to confer biological advantage. Furthermore, divalent linkers or constructs are also typically of modest size such that they retain the desirable biodistribution properties of small molecules.

Linker length. Linkers are chosen in a range of lengths to allow the spanning of a range of inter-ligand distances that encompass the distance preferable for a given divalent interaction. In some instances the preferred distance can be estimated rather precisely from high-resolution structural information of targets, typically enzymes and soluble receptor targets. In other instances where high-resolution structural information is not available (such as 7TM G-protein coupled receptors), one can make use of simple models to estimate the maximum distance between binding sites either on adjacent receptors or at different locations on the same receptor. In situations where two binding sites are present on the same target (or target subunit for multisubunit targets), preferred linker distances are 2-20 Å, with more preferred linker distances of 3-12 Å. In situations where two binding sites reside on separate (e.g., protein) target sites, preferred linker distances are 20-100 Å, with more preferred distances of 30-70 Å.

Linker geometry and rigidity. The combination of ligand attachment site, linker length, linker geometry, and linker rigidity determine the possible ways in which the ligands of candidate multibinding compounds may be displayed in three dimensions and thereby presented to their binding sites. Linker geometry and rigidity are nominally determined by chemical composition and bonding pattern, which may be controlled and are systematically varied as another spanning function in a multibinding array. For example, linker geometry is varied by attaching two ligands to the ortho, meta, and para positions of a benzene ring, or in cis- or trans- arrangements at the 1,1- vs. 1,2- vs. 1,3- vs. 1,4-positions around a cyclohexane core or in cis- or trans-arrangements at a point of ethylene unsaturation. Linker rigidity is varied by controlling the number and relative energies of different conformational states possible for the linker. For example, a divalent compound bearing two ligands joined by 1,8-octyl linker has many more degrees of freedom, and is therefore less rigid than a compound in which the two ligands are attached to the 4,4′ positions of a biphenyl linker.

Linker physical properties. The physical properties of linkers are nominally determined by the chemical constitution and bonding patterns of the linker, and linker physical properties impact the overall physical properties of the candidate multibinding compounds in which they are included. A range of linker compositions is typically selected to provide a range of physical properties (hydrophobicity, hydrophilicity, amphiphilicity, polarization, acidity, and basicity) in the candidate multibinding compounds. The particular choice of linker physical properties is made within the context of the physical properties of the ligands they join and preferably the goal is to generate molecules with favorable PK/ADME properties. For example, linkers can be selected to avoid those that are too hydrophilic or too hydrophobic to be readily absorbed and/or distributed in vivo.

Linker chemical functional groups. Linker chemical functional groups are selected to be compatible with the chemistry chosen to connect linkers to the ligands and to impart the range of physical properties sufficient to span initial examination of this parameter.

### Combinatorial Synthesis

Having chosen a set of n ligands (n being determined by the sum of the number of different attachment points for each ligand chosen) and m linkers by the process outlined above, a library of (n!)m candidate divalent multibinding compounds is prepared which spans the relevant multibinding design parameters for a particular target. For example, an array generated from two ligands, one which has two attachment points (A1, A2) and one which has three attachment points (B1, B2, B3) joined in all possible combinations provide for at least 15 possible combinations of multibinding compounds:

| | | | | | | | |
|---|---|---|---|---|---|---|---|
| A1-A1 | A1-A2 | A1-B1 | A1-B2 | A1-B3 | A2-A2 | A2-B1 | A2- |
| | | | | | | | B2 |
| A2-B3 | B1-B1 | B1-B2 | B1-B3 | B2-B2 | B2-B3 | B3-B3 | |

When each of these combinations is joined by 10 different linkers, a library of 150 candidate multibinding compounds results.

Given the combinatorial nature of the library, common chemistries are preferably used to join the reactive functionalies on the ligands with complementary reactive functionalities on the linkers. The library therefore lends itself to efficient parallel synthetic methods. The combinatorial library can employ solid phase chemistries well known in the art wherein the ligand and/or linker is attached to a solid support. Alternatively and preferably, the combinatorial libary is prepared in the solution phase. After synthesis, candidate multibinding compounds are optionally purified before assaying for activity by, for example, chromatographic methods (e.g., HPLC).

### Analysis of Array by Biochemical, Analytical, Pharmacological, and Computational Methods

Various methods are used to characterize the properties and activities of the candidate multibinding compounds in the library to determine which compounds possess multibinding properties. Physical constants such as solubility under various solvent conditions and logD/clogD values can be determined. A combination of NMR spectroscopy and computational methods is used to determine low-energy conformations of the candidate multibinding compounds in fluid media. The ability of the members of the library to bind to the desired target and other targets is determined by various standard methods, which include radioligand displacement assays for receptor and ion channel targets, and kinetic inhibition analysis for many enzyme targets. In vitro efficacy, such as for receptor agonists and antagonists, ion channel blockers, and antimicrobial activity, can also be determined. Pharmacological data, including oral absorption, everted gut penetration, other pharmacokinetic parameters and efficacy data can be determined in appropriate models. In this way, key structure-activity relationships are obtained for multibinding design parameters which are then used to direct future work.

The members of the library which exhibit multibinding properties, as defined herein, can be readily determined by conventional methods. First those members which exhibit multibinding properties are identified by conventional methods as described above including conventional assays (both in vitro and in vivo).

Second, ascertaining the structure of those compounds which exhibit multibinding properties can be accomplished via art recognized procedures. For example, each member of the library can be encrypted or tagged with appropriate information allowing determination of the structure of relevant members at a later time. See, for example, Dower, et al., International Patent Application Publication No. WO 93/06121; Brenner, et al., Proc. Natl. Acad. Sci., USA, 89:5181 (1992); Gallop, et al., U.S. Pat. No. 5,846,839; each of which are incorporated herein by reference in its entirety. Alternatively, the structure of relevant multivalent compounds can also be determined from soluble and untagged libaries of candidate multivalent compounds by methods known in the art such as those described by Hindsgaul, et al., Canadian Patent Application No. 2,240,325 which was published on Jul. 11, 1998. Such methods couple frontal affinity chromatography with mass spectroscopy to determine both the structure and relative binding affinities of candidate multibinding compounds to receptors.

The process set forth above for dimeric candidate multibinding compounds can, of course, be extended to trimeric candidate compounds and higher analogs thereof.

### Follow-up Synthesis and Analysis of Additional Array(s)

Based on the information obtained through analysis of the initial library, an optional component of the process is to ascertain one or more promising multibinding “lead” compounds as defined by particular relative ligand orientations, linker lengths, linker geometries, etc. Additional libraries are generated around these leads to provide for further information regarding structure to activity relationships. These arrays typically bear more focused variations in linker structure to further optimize target affinity and/or activity at the target (antagonism, partial agonism, etc.), and/or alter physical properties. By iterative redesign/analysis using the novel principles of multibinding design along with classical medicinal chemistry, biochemistry, and pharmacology approaches, one is able to prepare and identify optimal multibinding compounds that exhibit biological advantage towards their targets and as therapeutic agents.

To further elaborate upon this procedure, suitable divalent linkers include, by way of example only, those derived from dicarboxylic acids, disulfonylhalides, dialdehydes, diketones, dihalides, dipseudohalides, diisocyanates, diamines, diols, diboronates, mixtures of carboxylic acids, sulfonylhalides, aldehydes, ketones, halides, pseudohalides, isocyanates, amines, boronates, and alcohols. In each case, the carboxylic acid, sulfonylhalide, aldehyde, ketone, halide, pseudohalide, isocyanate, amine, boronate and alcohol functional group is reacted with a complementary functionality on the ligand to form a covalent linkage. Such complementary functionality is well known in the art as illustrated in the following table:

COMPLEMENTARY BINDING CHEMISTRIES

| First Reactive Group | Second Reactive Group | Linkage |
|---|---|---|
| hydroxyl | isocyanate | urethane |
| amine | epoxide | β-hydroxyamine |
| sulfonyl halide | amine | sulfonamide |
| carboxyl acid | amine | amide |
| hydroxyl | alkyl/aryl halide | ether |
| aldehyde | amine/NaCNBH₄ | amine |
| ketone | amine/NaCNBH₄ | amine |
| amine | isocyanate | urea |

Exemplary linkers include the following linkers identified as X-1 through X-418 as set forth below:

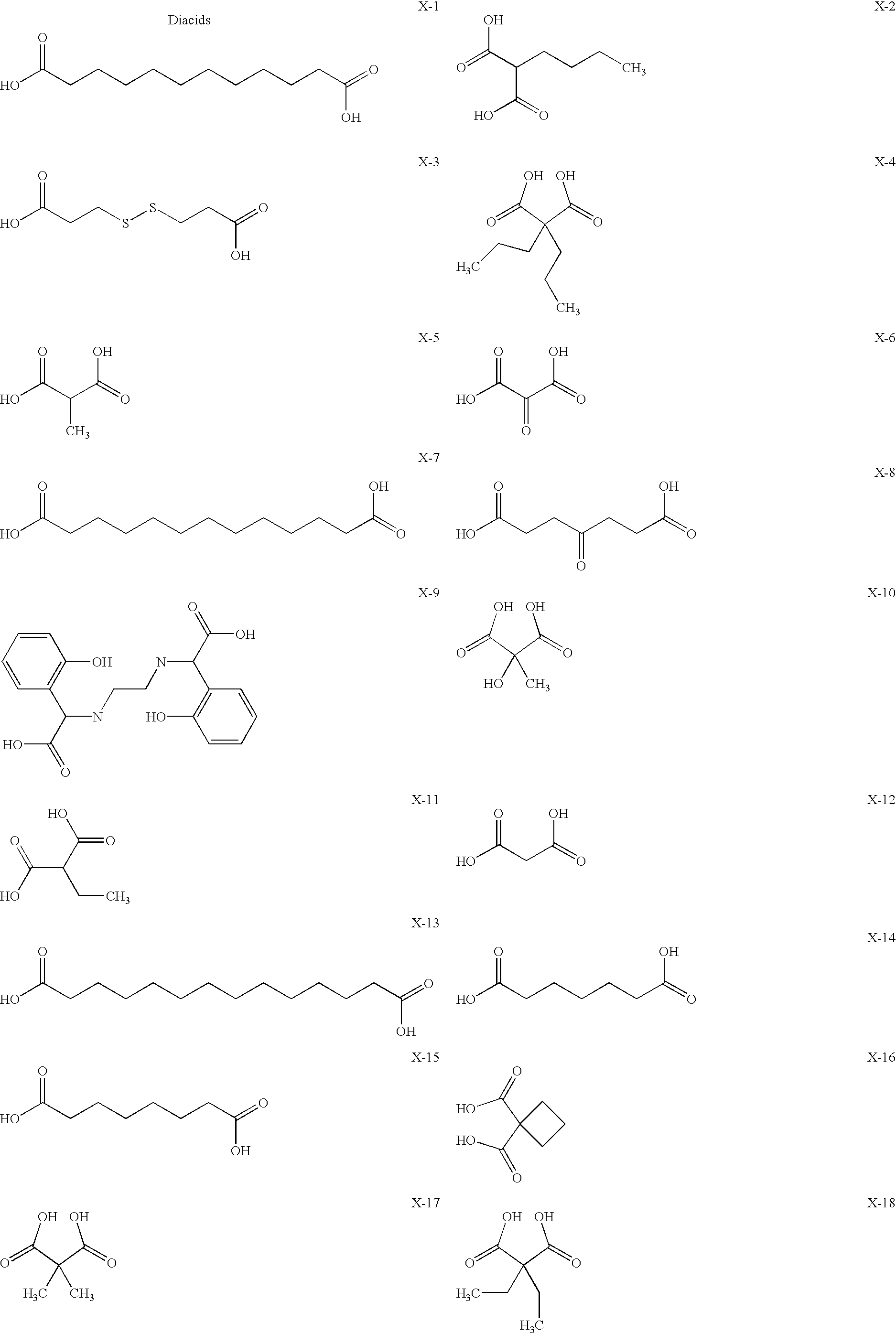

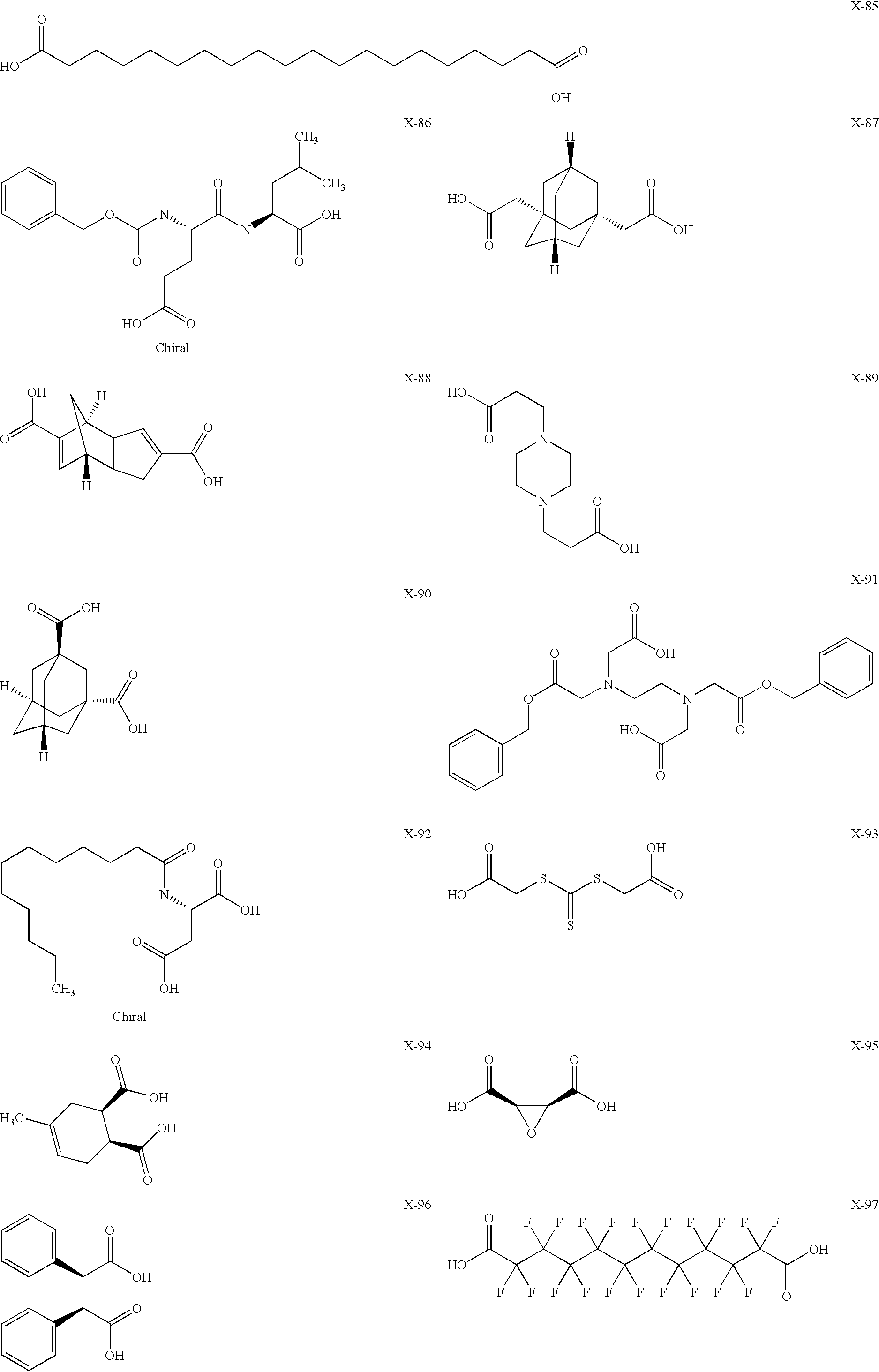

Representative ligands for use in this invention include, by way, of example, L-1 as identified and defined above and in Table 6 (see Appendix).

Combinations of ligands (L) and linkers (X) per this invention include, by way example only, homo- and hetero-dimers wherein a first ligand is selected from L-1 above and the second ligand, also independently selected from Table 6, and linker is selected from the following:

| | | | | | |
|---|---|---|---|---|---|
| L-1/X-1- | L-1/X-2- | L-1/X-3- | L-1/X-4- | L-1/X-5- | L-1/X-6- |
| L-1/X-7- | L-1/X-8- | L-1/X-9- | L-1/X-10- | L-1/X-11- | L-1/X-12- |
| L-1/X-13- | L-1/X-14- | L-1/X-15- | L-1/X-16- | L-1/X-17- | L-1/X-18- |
| L-1/X-19- | L-1/X-20- | L-1/X-21- | L-1/X-22- | L-1/X-23- | L-1/X-24- |
| L-1/X-25- | L-1/X-26- | L-1/X-27- | L-1/X-28- | L-1/X-29- | L-1/X-30- |
| L-1/X-31- | L-1/X-32- | L-1/X-33- | L-1/X-34- | L-1/X-35- | L-1/X-36- |
| L-1/X-37- | L-1/X-38- | L-1/X-39- | L-1/X-40- | L-1/X-41- | L-1/X-42-L- |
| 1/X-43- | L-1/X-44- | L-1/X-45- | L-1/X-46- | L-1/X-47- | L-1/X-48- |
| L-1/X-49- | L-1/X-50- | L-1/X-51- | L-1/X-52- | L-1/X-53- | L-1/X-54- |
| L-1/X-55- | L-1/X-56- | L-1/X-57- | L-1/X-58- | L-1/X-59- | L-1/X-60- |
| L-1/X-61- | L-1/X-62- | L-1/X-63- | L-1/X-64- | L-1/X-65- | L-1/X-66- |
| L-1/X-67- | L-1/X-68- | L-1/X-69- | L-1/X-70- | L-1/X-71- | L-1/X-72- |
| L-1/X-73- | L-1/X-74- | L-1/X-75- | L-1/X-76- | L-1/X-77- | L-1/X-78- |
| L-1/X-79- | L-1/X-80- | L-1/X-81- | L-1/X-82- | L-1/X-83- | L-1/X-84- |
| L-1/X-85- | L-1/X-86- | L-1/X-87- | L-1/X-88- | L-1/X-89- | L-1/X-90- |
| L-1/X-91- | L-1/X-92- | L-1/X-93- | L-1/X-94- | L-1/X-95- | L-1/X-96- |
| L-1/X-97- | L-1/X-98- | L-1/X-99- | L-1/X-100- | L-1/X-101- | L-1/X-102- |
| L-1/X-103- | L-1/X-104- | L-1/X-105- | L-1/X-106- | L-1/X-107- | L-1/X-108- |
| L-1/X-109- | L-1/X-110- | L-1/X-111- | L-1/X-112- | L-1/X-113- | L-1/X-114- |
| L-1/X-115- | L-1/X-116- | L-1/X-117- | L-1/X-118- | L-1/X-119- | L-L/X-120- |
| L-1/X-121- | L-1/X-122- | L-1/X-123- | L-1/X-124- | L-1/X-125- | L-1/X-126- |
| L-1/X-127- | L-1/X-128- | L-1/X-129- | L-1/X-130- | L-1/X-131- | L-1/X-132- |
| L-1/X-133- | L-1/X-134- | L-1/X-135- | L-1/X-136- | L-1/X-137- | L-1/X-138- |
| L-1/X-139- | L-1/X-140- | L-1/X-141- | L-1/X-142- | L-1/X-143- | L-1/X-144- |
| L-1/X-145- | L-1/X-146- | L-1/X-147- | L-1/X-148- | L-1/X-149- | L-1/X-150- |
| L-1/X-151- | L-1/X-152- | L-1/X-153- | L-1/X-154- | L-1/X-155- | L-1/X-156- |
| L-1/X-157- | L-1/X-158- | L-1/X-159- | L-1/X-160- | L-1/X-161- | L-1/X-162- |
| L-1/X-163- | L-1/X-164- | L-1/X-165- | L-1/X-166- | L-1/X-167- | L-1/X-168- |
| L-1/X-169- | L-1/X-170- | L-1/X-171- | L-1/X-172- | | |
| L-1/X-173- | L-1/X-174- | L-1/X-175- | L-1/X-176- | L-1/X-177- | L-1/X-178- |
| L-1/X-179- | L-1/X-180- | L-1/X-181- | L-1/X-182- | L-1/X-183- | L-1/X-184- |
| L-1/X-185- | L-1/X-186- | L-1/X-187- | L-1/X-188- | L-1/X-189- | L-1/X-190- |
| L-1/X-191- | L-1/X-192- | L-1/X-193- | L-1/X-194- | L-1/X-195- | L-1/X-196- |
| L-1/X-197- | L-1/X-198- | L-1/X-199- | L-1/X-200- | L-1/X-201- | L-1/X-202- |
| L-1/X-203- | L-1/X-204- | L-1/X-205- | L-1/X-206- | L-1/X-207- | L-1/X-208- |
| L-1/X-209- | L-1/X-210- | L-1/X-211- | L-1/X-212- | L-1/X-213- | L-1/X-214- |
| L-1/X-215- | L-1/X-216- | L-1/X-217- | L-1/X-218- | L-1/X-219- | L-1/X-220- |
| L-1/X-221- | L-1/X-222- | L-1/X-223- | L-1/X-224- | L-1/X-225- | L-1/X-226- |
| L-1/X-227- | L-1/X-228- | L-1/X-229- | L-1/X-230- | L-1/X-231- | L-1/X-232- |
| L-1/X-233- | L-1/X-234- | L-1/X-235- | L-1/X-236- | L-1/X-237- | L-1/X-238- |
| L-1/X-239- | L-1/X-240- | L-1/X-241- | L-1/X-242- | L-1/X-243- | L-1/X-244- |
| L-1/X-245- | L-1/X-246- | L-1/X-247- | L-1/X-248- | L-1/X-249- | L-1/X-250- |
| L-1/X-251- | L-1/X-252- | L-1/X-253- | L-1/X-254- | L-1/X-255- | L-1/X-256- |
| L-1/X-257- | L-1/X-258- | L-1/X-259- | L-1/X-260- | L-1/X-261- | L-1/X-262- |
| L-1/X-263- | L-1/X-264- | L-1/X-265- | L-1/X-266- | L-1/X-267- | L-1/X-268- |
| L-1/X-269- | L-1/X-270- | L-1/X-271- | L-1/X-272- | L-1/X-273- | L-1/X-274- |
| L-1/X-275- | L-1/X-276- | L-1/X-277- | L-1/X-278- | L-1/X-279- | L-1/X-280- |
| L-1/X-281- | L-1/X-282- | L-1/X-283- | L-1/X-284- | L-1/X-285- | L-1/X-286- |
| L-1/X-287- | L-1/X-288- | L-1/X-289- | L-1/X-290- | L-1/X-291- | L-1/X-292- |
| L-1/X-293- | L-1/X-294- | L-1/X-295- | L-1/X-296- | L-1/X-297- | L-1/X-298- |
| L-1/X-299- | L-1/X-300- | L-1/X-301- | L-1/X-302- | L-1/X-303- | L-1/X-304- |
| L-1/X-305- | L-1/X-306- | L-1/X-307- | L-1/X-308- | L-1/X-309- | L-1/X-310- |
| L-1/X-311- | L-1/X-312- | L-1/X-313- | L-1/X-314- | L-1/X-315- | L-1/X-316- |
| L-1/X-317- | L-1/X-318- | L-1/X-319- | L-1/X-320- | L-1/X-321- | L-1/X-322- |
| L-1/X-323- | L-1/X-324- | L-1/X-325- | L-1/X-326- | L-1/X-327- | L-1/X-328- |
| L-1/X-329- | L-1/X-330- | L-1/X-331- | L-1/X-332- | L-1/X-333- | L-1/X-334- |
| L-1/X-335- | L-1/X-336- | L-1/X-337- | L-1/X-338- | L-1/X-339- | L-1/X-340- |
| L-1/X-341- | L-1/X-342- | L-1/X-343- | L-1/X-344- | L-1/X-345- | L-1/X-346- |
| L-1/X-347- | L-1/X-348- | L-1/X-349- | L-1/X-350- | L-1/X-351- | L-1/X-352- |
| L-1/X-353- | L-1/X-354- | L-1/X-355- | L-1/X-356- | L-1/X-357- | L-1/X-358- |
| L-1/X-359- | L-1/X-360- | L-1/X-361- | L-1/X-362- | L-1/X-363- | L-1/X-364- |
| L-1/X-365- | L-1/X-366- | L-1/X-367- | L-1/X-368- | L-1/X-369- | L-1/X-370- |
| L-1/X-371- | L-1/X-372- | L-1/X-373- | L-1/X-374- | L-1/X-375- | L-1/X-376- |
| L-1/X-377- | L-1/X-378- | L-1/X-379- | L-1/X-380- | L-1/X-381- | L-1/X-382- |
| L-1/X-383- | L-1/X-384- | L-1/X-385- | L-1/X-386- | L-1/X-387- | L-1/X-388- |
| L-1/X-389- | L-1/X-390- | L-1/X-391- | L-1/X-392- | L-1/X-393- | L-1/X-394- |
| L-1/X-395- | L-1/X-396- | L-1/X-397- | L-1/X-398- | L-1/X-399- | L-1/X-400- |
| L-1/X-401- | L-1/X-402- | L-1/X-403- | L-1/X-404- | L-1/X-405- | L-1/X-406- |
| L-1/X-407- | L-1/X-408- | L-1/X-409- | L-1/X-410- | L-1/X-411- | L-1/X-412- |
| L-1/X-413- | L-1/X-414- | L-1/X-415- | L-1/X-416- | L-1/X-417- | L-1/X-418- |

### Activity Tests

Local anesthetics can be tested for activity in various well-known assays (e.g.,. the batrachotoxin (BTX) displacement assay (McNeal et al, J. Med. Chem. 28: 381 (1985)), patch clamp method (see, generally, Neher and Sakmann, “The Patch Clamp Technique,” Scientific American pp. 44-51 (1992); Hamill et al, Pfügers Arch. 391: 85 (1981); intact isolated nerve assay, e.g., isolated frog sciatic nerve (see Example 13 described below); blockage of the cutaneous trunci muscle reflex (CTMR) in guinea pigs (Bulbring et al, J. Pharmacol. Exp. Therap. 85: 78-84 (1945); Blight et al, J. Compar. Neurology 296: 614-633 (1990); Choi et al, Life Sci. 61: PL177-84 (1997)). Evaluation of motor and sympathetic function during sciatic nerve block is described, e.g., in Grant et al, Anesth. Analg. 75: 889-94 (1992), and Thalhammer et al., Anesthesiology 82: 1013-25 (1995).

The multi-binding compounds prepared as described above were screened for Na⁺ ion channel binding and functional activities as exemplified in Examples 11-13 below.

### Anticonvulsant Compounds

The anticonvulsant drugs are used for the treatment of all types of seizures, including epilepsy. As noted previously, this class of drugs includes, for example, diphenyl hydantoin (Dilantin), and other related ligands with binding affinity for the sodium channel comparable to that of Dilantin.

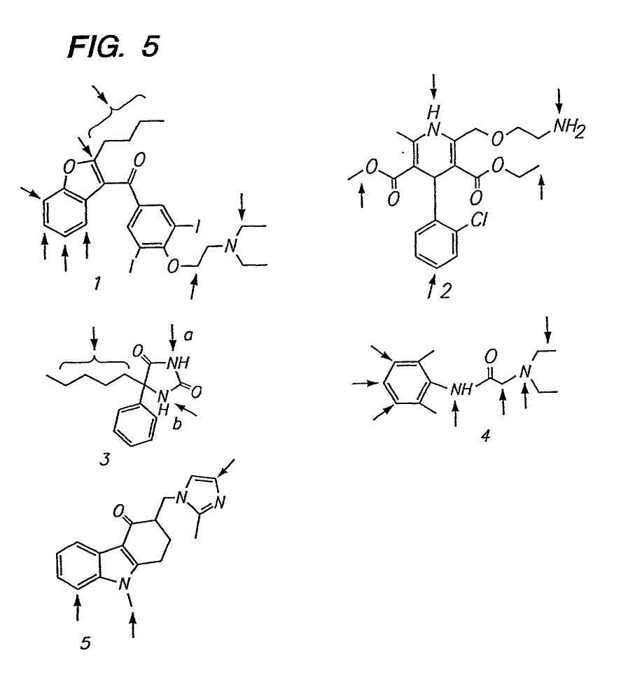

The preparation of multi-binding anticonvulsant compounds of this invention makes use of ligands such as the 5-pentyl-5-phenyl hydantoin shown in FIG. 5 (3). Positions that are potentially available for attachment of a linker include either of the imide nitrogens (i.e., (a) or (b)) and positions on the pentyl chain, as indicated by the arrows.

### Preparation of a Bivalent Phenylhydantoin Compound

The preparation of a bivalent phenyl hydantoin compound is shown, for example, in Reaction Scheme 18 (Appendix). The starting material, an aliphatic hydroxyketone derivative of phenyl hydantoin, can be prepared by the Bucherer-Berg procedure from the appropriate ketones (see Brown et al, J. Med. Chem. 40: 602-607 (1997)). Standard well-known peptide coupling conditions are then used to attach the ligands to an appropriate diamine linker, as shown.

Compounds of higher-order valency can be prepared by judicious choice of linkers and by appropriately varying the concentrations of the ligands and reagents.

### Activity Tests

Anticonvulsant activity is screened in rodents using the maximal electroshock (MES) test and the subcutaneous pentylenetetrazol (scMET) test ( see, e.g., Flaherty et al, J. Med. Chem. 39: 1509-1513 (1996)). Sodium channel binding is performed by the BTX displacement assay as described above for local anesthetics.

### Drugs that Affect Potassium Ion Channels

Included among compounds of Formula I are compounds comprising ligands that either activate or inhibit potassium ion channels. Not surprisingly, these ligands are used for a variety of clinical conditions, for example: channel blocking ligands such as glipizide, glyburide and tolazamide are used for the treatment of diabetes; diazoxide and minoxidil are used as antihypertensive drugs; and amiodarone, clofilium, dofetilide and N-acetylprocainamide are used as antiarrythmic drugs. Ligands that open potassium channels include levcromakalin, pinacidil, nicorandil, aprikalim, and diazoxide. The channel openers act on ATP-gated potassium channels in vascular smooth muscle and decreate vascular tone. They are potentially useful as cardioprotective agents.

### Anti-arrhythmic Compounds

The anti-arrhythmic potassium channel blockers of this invention are exemplified by amiodarone. Amiodarone is classified as a Class III anti-arrhythmic drug (i.e., a drug that prolongs the cardiac action potential duration).

Amiodarone is an iodine-containing benzofuran derivative that is used in the treatment of life-threatening ventricular arrhythmias that are unresponsive to other types of anti-arrhythmic drugs. Amiodarone and analogs thereof can be used to prepare multi-binding anti-arrhythmic compounds of Formula I. The structure of amiodarone is given in FIG. 5 (1). Positions that are potentially available for attachment of a linker based on published SAR are indicated by the arrows.

### Preparation of a Bivalent Amiodarone Compound

The preparation of a bivalent amiodarone compound is shown, for example, in Reaction Scheme 19 (Appendix). The reactions can be performed using conventional procedures known to those of ordinary skill in the art.

Compounds of higher-order valency can be prepared by judicious choice of linkers and by appropriately varying the concentrations of the ligands and reagents.

### Activity Tests

The activity of anti-arrhythmic potassium channel blockers of this invention is assessed by measuring potassium currents in cardiac myocytes using the patch clamp technique (see, e.g., Kamp et al, PflüArch. 391: 85 (1981)). In vivo anti-arrhythmic activity is measured by testing the ability of compounds to suppress symptoms in dogs with subacute myocardial infarctions or induced ventricular arrhythmia as described by Abdollah et al, J. Cardiovasc. Pharm. 15: 799 (1990)).

### Inhibitors of Calcium Channels

Included in the compounds of Formula I are compounds comprising ligands that inhibit calcium ion channels. These ligands are used for the treatment of various cardiovascular diseases, such as hypertension (e.g., amlodipine, diltiazem, flodipine, isradipine, nifedipine, nicardipine, verapamil), arrhythmias (e.g., diltiazem, bepridil, verapamil), and angina (e.g., amlodipine, diltiazem, felodipine, nifedipine, verapamil).

At least two types of cardiac calcium channels have been identified, the L type (found in all cardiac tissues) and the T type (found in pacemaker tissues) (Pelzer et al, Rev. Physiol. Biochem. Pharmacol. 114: 107 (1990)). The α₁ subunits of L- and non-L type calcium channels are structurally homologous. Calcium channels are found also in vascular smooth muscle, adrenal chromaffin cells, other endocrine cells and the brain. These factors may contribute to the undesirable side effects of currently used calcium channel blockers (e.g., hypotension and arrhythmia). Thus there is a continuing clinical need for safer, more selective calcium channel blocking compounds.

### Anti-hypertensive Compounds

The anti-hypertensive calcium channel blockers of this invention are exemplified by amlodipine (structure shown below).

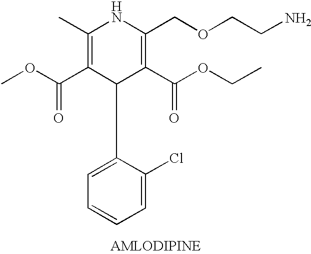

This drug is classified as a dihydropyridine-based calcium channel blocker. Amlodipine is dihydropyridine calcium antagonist with an 2-methoxyalkylamine group, which provides for its prolonged anti-hypertensive activity (Arrowsmith et al, J. Med. Chem. 29: 1696 (1986)).

FIG. 5 (2) shows amlodipine with arrows pointing to positions that are potentially available for attachment of a linker.

### Preparation of a Bivalent Amlodipine Compound

The preparation of a bivalent amlodipine compound is shown, for example, in Reaction Scheme 20 (Appendix). The reactions can be performed using conventional procedures known to those of ordinary skill in the art.

Compounds of higher-order valency can be prepared by judicious choice of linkers and by appropriately varying the concentrations of the ligands and reagents.

### Activity Tests

The activity of anti-arrhythmic calcium channel blockers of this invention is determined by assaying their vasorelaxant and antihypertensive properties. The concentration of a compound required to produce 50% vasorelaxation in KCl-contracted rabbit thoracic aorta strips in the presence of calcium is measured (Brittain et al, Physiologist 28: 325 (1985)). Anti-hypertensive activity is determined in male spontaneously hypertensive rats by measurement of mean arterial blood pressure (Rovnyak et al, J. Med. Chem. 35: 3254-3263 (1992)).

### Isolation and Purification of the Compounds

Isolation and purification of the compounds and intermediates described herein can be effected, if desired, by any suitable separation or purification such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the Examples herein below. However, other equivalent separation or isolation procedures could, of course, also be used.

### Pharmaceutical Formulations

When employed as pharmaceuticals, the compounds of formula I are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions which contain, as the active ingredient, one or more of the compounds of formula I above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g. about 40 mesh. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions are preferably formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Preferably, the compound of formula I above is employed at no more than about 20 weight percent of the pharmaceutical composition, more preferably no more than about 15 weight percent, with the balance being pharmaceutically inert carrier(s).

The active compound is effective over a wide dosage range and is generally administered in a pharmaceutically effective amount. It, will be understood, however, that the amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as corn oil, cottonseed oil, sesame oil, coconut oil, or peanut oil, as well as elixirs and similar pharmaceutical vehicles. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect.

Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices which deliver the formulation in an appropriate manner.

The following formulation examples illustrate representative pharmaceutical compositions of the present invention.

### Formulation Example 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### Formulation Example 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### Formulation Example 3

A dry powder inhaler formulation is prepared containing the following components:

| Ingredient | Weight % |
|---|---|
| Active Ingredient | 5 |
| Lactose | 95 |

The active ingredient is mixed with the lactose and the mixture is added to a dry powder inhaling appliance.

### Formulation Example 4

Tablets, each containing 30 mg of active ingredient, are prepared as follows:

| | Quantity |
|---|---|
| Ingredient | (mg/tablet) |
| Active Ingredient | 30.0 mg |
| Starch | 45.0 mg |
| Microcrystalline cellulose | 35.0 mg |
| Polyvinylpyrrolidone | 4.0 mg |
| (as 10% solution in sterile water) | |
| Sodium carboxymethyl starch | 4.5 mg |
| Magnesium stearate | 0.5 mg |
| Talc | 1.0 mg |
| Total | 120 mg |

The active ingredient, starch and cellulose are passed through a No. 20 mesh U.S. sieve and mixed thoroughly. The solution of polyvinylpyrrolidone is mixed with the resultant powders, which are then passed through a 16 mesh U.S. sieve. The granules so produced are dried at 50° C. to 60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 120 mg.

### Formulation Example 5

Capsules, each containing 40 mg of medicament are made as follows:

| | Quantity |
|---|---|
| Ingredient | (mg/capsule) |
| Active Ingredient | 40.0 mg |
| Starch | 109.0 mg |
| Magnesium stearate | 1.0 mg |
| Total | 150.0 mg |

The active ingredient, starch, and magnesium stearate are blended, passed through a No. 20 mesh U.S. sieve, and filled into hard gelatin capsules in 150 mg quantities.

### Formulation Example 6

Suppositories, each containing 25 mg of active ingredient are made as follows:

| Ingredient | Amount |
|---|---|
| Active Ingredient | 25 mg |
| Saturated fatty acid glycerides to | 2,000 mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### Formulation Example 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water to | 5.0 | mL |

The active ingredient, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### Formulation Example 8

| | Quantity |
|---|---|
| Ingredient | (mg/capsule) |
| Active Ingredient | 15.0 mg |
| Starch | 407.0 mg |
| Magnesium stearate | 3.0 mg |
| Total | 425.0 mg |

The active ingredient, starch, and magnesium stearate are blended, passed through a No. 20 mesh U.S. sieve, and filled into hard gelatin capsules in 425.0 mg quantities.

### Formulation Example 9

A subcutaneous formulation may be prepared as follows:

| Ingredient | Quantity | |
|---|---|---|
| Active Ingredient | 5.0 | mg |
| Corn Oil | 1.0 | mL |

### Formulation Example 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, herein incorporated by reference. Such patches may be constructed for continuous, pulsatile, or on demand delivery of pharmaceutical agents.

Frequently, it will be desirable or necessary to introduce the pharmaceutical composition to the brain, either directly or indirectly. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is herein incorporated by reference.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

Other suitable formulations for use in the present invention can be found in Remington's Pharmaceutical Sciences, Mace Publishing Company, Philadelphia, Pa., 17th ed. (1985) and “Modern Pharmaceutics”, Marcel Dekker, Inc., 3^{rd} ed. (G. S. Banker & C. T. Rhodes, eds.)

### Utility

The multibinding agents of this invention are useful in modulating the activity of cell membrane transporters. They will be typically be used for the treatment of diseases and conditions in mammals and avians that involve, or are mediated by, cell membrane transporters. As well, the multibinding agents of this invention are expected to be useful as imaging and diagnostic agents, in research or industrial processes (e.g., separation and purification), as research tools (e.g., for detection and isolation of specific transporters in biological samples), and for agricultural applications such as crop protection (e.g., as insecticides and pesticides).

In order to further illustrate the present invention and advantages thereof, the following specific examples are given but are not meant to limit the scope of the claims in any way.

### EXAMPLES

In the examples below, all temperatures are in degrees Celsius (unless otherwise indicated) and all percentages are weight percentages (also unless otherwise indicated). Additionally, the following abbreviations have the following meanings. If an abbreviation is not defined, it has its generally accepted meaning.

### EXAMPLE 1

### Preparation of [C—C] Compounds of Formula I (1) Preparation of a Compound of Formula (3) in which m is 2 and n is 3

To a solution of tert-butyl N-(2-aminoethyl)carbamate (2.3 g, 14.4 mmol) and N,N-diisopropylethylamine (2.5 ml, 14.3 mmol) in 15 mL methylene chloride at 0° C. was added glutaryl dichloride (0.6 mL, 4.7 mmol) in 15 mL methylene chloride dropwise. The resulting mixture was allowed to warm to room temperature with stirring while adding water (15 mL). The methylene chloride was removed under reduced pressure and more water was added (30 mL). The resulting suspension was filtered and washed sequentially with 10% potassium hydrogen sulfate, water, saturated sodium bicarbonate, and water. The solid was dried under vacuum yielding 1.3 g (3.1 mmol, 66%) of pentanedioic acid bis-[(2-t-butoxycarbonylaminoethyl)amide], a compound of formula (3).

Similarly, varying the composition of m and n, other compounds of formula (3) are prepared.

### (2) Preparation of a Compound of Formula (4) in which m is 2 and n is 3

Pentanedioic acid bis-[(2-t-butoxycarbonylaminoethyl)amide], a compound of formula (3) (1.3 g, 3.1 mmol) was suspended in 15 mL methylene chloride. 15 mL of trifluoroacetic acid was added at room temperature giving (with effervescence) a solution that was stirred for 40 minutes, then evaporated in vacuo. The residue was dissolved in methanol and treated with 3 mL 4N hydrogen chloride in dioxane followed by diethyl ether, giving a gum. The liquids were decanted and the gum dried under vacuum yielding 1.0 g (3.4 mmol) of pentanedioic acid bis-[(2-aminoethyl)amide], a compound of formula (4).

Similarly, varying m and n, other compounds of formula (4) are prepared.

### (3) Preparation of a Compound of Formula I

At room temperature a carboxy-bearing ligand (2.3 mmol) is dissolved in 36 mL of dimethylsulfoxide. To this solution is added pentanedioic acid bis-[(2-aminoethyl)amide], a compound of formula (4) (1.0 g, 3.4 mmol suspended in 27 mL N,N-dimethyIformamide) followed by N,N-diisopropylethylamine (2.4 mL, 13.8 mmol). The resulting suspension is stirred at room temperature for several hours until it is mostly soluble. Then a solution of PyBOP (1.3 g, 2.5 mmol) and 1-hydroxybenzotriazole (310 mg, 2.3 mmol) in 9 mL N,N-dimethylformamide is added rapidly dropwise. The mixture is stirred at room temperature for 1 hour and then added dropwise to 600 mL of acetonitrile, giving a precipitate that is filtered, washed with acetonitrile ,then diethyl ether, and dried under vacuum. The crude product is purified by reverse phase HPLC to yield a salt of a compound of Formula I.

Accordingly, following the procedures of Example 1, steps 1-3, other [C-C] compounds of Formula I are prepared.

### EXAMPLE 2

Alternative Preparation of [C—C] Compounds of Formula I

### (1) Preparation of a Compound of Formula (7) in which m is 2

At room temperature a carboxy-bearing ligand (4.7 mmol) is dissolved in 75 mL of dimethylsulfoxide. To this solution is added N,N-diisopropylethylamine (4.1 mL, 23.5 mmol) followed by 9-fluorenylmethyl N-(2-aminoethyl)carbamate hydrochloride (1.8 g, 5.6 mmol). To the resulting solution at room temperature is added rapidly dropwise a solution of PyBOP (2.7 g, 5.2 mmol) and 1-hydroxybenzotriazole (630 mg, 4.7 mmol) in 75 mL 1,3-dimethyl-3,4,5,6-tetrahydro-2(1H)-pyrimidinone. The resulting solution is stirred at room temperature for 2 hours, then poured into 800 mL diethyl ether, giving a gum. The diethyl ether is decanted and the gum washed with additional diethyl ether to give a compound of formula (7).

### (2) Preparation of a Compound of Formula (8) in which m is 2

The gum of formula (7) is then taken up in 40 mL of N,N-dimethylformamide, to which 10 mL of piperidine is added and the solution left to stand at room temperature for 20 minutes. The solution is then added dropwise to 450 mL of acetonitrile giving a precipitate. Centrifugation is followed by decantation of the acetonitrile and the residue washed twice with 450 mL of acetonitrile, once with 450 mL of diethyl ether and air dried. The residue is taken up in water, acidified to pH <5 with a small amount of 3N hydrochloric acid and purified by reverse-phase HPLC using a gradient of 2-30% acetonitrile in water containing 0.1% trifluoroacetic acid yielding a compound of formula (8).

### (3) Preparation of a Compound of Formula I

Compound (8) (220 μmol) and glutaric acid (10 mg, 76 μmol) are dissolved in 5 mL N,N-dimethylformamide and N,N-diisopropylethylamine (140 μL, 800 μmol) followed by PyBOP (83 mg, 160μmol) and 1-hydroxybenzotriazole (10 mg, 74 μmol) in 500 μL N,N-dimethylformamide. The reaction is stirred for 75 minutes at room temperature then an additional 20 mg of PyBOP was added. 75 minutes later the solution is dripped into 45 mL acetonitrile. The resulting precipitate is collected by centrifugation, washed with ether, air dried and purified by reverse-phase HPLC to give a compound of Formula I as its salt.

### (4) Preparation of other Compounds of Formula I

Accordingly, following the procedures of Example 2, steps 1-3, other [C—C] compounds of Formula I are prepared.

### EXAMPLE 3

### Preparation of a [C—V] Compound of Formula I in which Position C' is Substituted (1) Preparation of a Compound of Formula (22) in which m and n are both 2

To a solution of a 2-aminoethyl ligand carboxamide (26.8 :mol) in DMF (2.0 mL) is added a compound of the formula:

HO₂CCH₂CH₂NHOCCH₂CH₂NHOCCH₂CH₂CH₂-CONHCH₂CH₂CONHCH₂CH₂CO₂FM

(FM refers to 9-Fluorenyl) (20.0 mg, 26.8 :mol), followed by PyBOP (20.9 mg, 40.2 :mol), HOBt (5.40 mg, 40.2 :mol), and Hunig's base (23.3 :L, 134 :mol). The reaction solution is stirred for 1 hour and then added dropwise to 20 mL of acetonitrile giving a precipitate, which is collected by centrifugation. The crude precipitate is dried in air, yielding a compound of formula (22). The compound is used in the next step without further purification.

### (2) Preparation of a Compound of Formula (23) in which m and n are both 2

The compound of formula (22) is dissolved in 1 mL of DMF, and 100 :L of piperidine added to the solution. The solution is allowed to stand at room temperature for 30 minutes, following the course of the reaction by mass spectroscopy. The reaction solution is added dropwise to 20 mL of acetonitrile, giving a precipitate that is collected by centrifugation. The precipitate is purified by reverse phase HPLC. The desired product is identified by mass spectroscopy using an API 300 electrospray mass spectrometer and afterwards lyophilized to afford compound (23).

### (1) Preparation of a Compound of Formula I

The compound of formula (23) prepared above (4.80 :mol) is dissolved in 500 :L of DMF. A compound of formula (17) (4.80 :mol ) is added to the solution, followed by PyBOP (2.50 mg, 4.8 umol), HOBt (0.65 mg, 4.80 :mol) and Hunig's base (6.70 :L, 38.4 :mol). The reaction solution is added dropwise to 20 mL of acetonitrile, giving a precipitate that is collected by centrifugation. The precipitate is purified by reverse phase HPLC. The desired product Is identified by mass spectroscopy using an API 300 electrospray mass spectrometer.

### EXAMPLE 4

Preparation of a [C—N] Compound of Formula I

### (1) Preparation of a Compound of Formula (24) in which m is 2

A ligand having a secondary amine group and a carboxy group (2.60 mmol) is suspended in 40 mL of 1,3-dimethyl-3,4,5 6-tetrahydro-2-(1H)-pyrimidinone and heated to 70° C. for 15 minutes. N-(9-fluorenylmethoxycarbonyl)-aminoacetaldehyde (720 mg, 2.6 mmol) is added and the mixture is heated at 70° C. for one hour. Sodium cyanoborohydride (160 mg, 2.5 mmol) in 2 mL methanol is added and the mixture is heated at 70° C. for 2 hours, then cooled to room temperature. ). The reaction solution is added dropwise to 20 mL of acetonitrile, giving a precipitate that is collected by centrifugation. The precipitate is purified by reverse phase HPLC to yielded [N]-2-[(9-fluorenyl)methoxycarbonylaminoethyl] ligand, a compound of formula (24) as its trifluroacetate salt.

### (2) Preparation of a Compound of Formula (25) in which m is 2 and p is 3

The compound of formula (24) obtained above (150 :mol) is dissolved in 3 mL of DMF. 3-(dimethylamino)propylamine (28.3 :L, 225 :mol) is added, followed by the addition of PyBOP (85.8 mg, 165 :mol), HOBt (20.3 mg, 150 :mol) and Hunig's base (65.0 :L, 375 :mol). The reaction solution is stirred for 1 hour and then added dropwise to 20 mL of acetonitrile giving a precipitate, which is collected by centrifugation, to give compound of formula (25).

### (3) Preparation of a Compound of Formula (26) in which m is 2 and p is 3

The compound of formula (25) obtained above is dissolved in 1 mL of DMF, and 100 :L of piperidine added to the solution. The solution is allowed to stand at room temperature for 30 minutes and the course of the reaction is followed by mass spectroscopy. The reaction solution is added dropwise to 20 mL of acetonitrile, giving a precipitate that is collected by centrifugation. The precipitate is purified by reverse phase HPLC to yield the compound of formula (26) as its salt.

### (2) Preparation of a Compound of Formula I

The compound of formula (26) prepared above (3.14 :mol) is dissolved in 500 :L of DMF. The compound of formula (19) (3.14 :mol) is added to the solution, followed by PyBOP (2.44 mg, 4.8 :mol), HOBt(0.65 mg, 4.8 :mol) and Hunig's base (6.7 :L, 38.4 :mol). ). The reaction solution is added dropwise to 20 mL of acetonitrile, giving a precipitate that is collected by centrifugation. The precipitate is purified by reverse phase HPLC to yield a compound of Formula I as its salt.

### EXAMPLE 5

Preparation of an [N—N] Compound of Formula I

### (1) Preparation of a Compound of Formula I

The compound of formula (26) prepared above (12.7 umol) is dissolved in 500 uL of DMF, and a compound of the formula: HO₂CCH₂CH₂NHOCCH₂CH₂NHOCCH₂CH₂CH₂CONHCH₂CH₂CONHCH₂CH₂CO₂H (2.64 mg, 6.34 :mol) is added, followed by PyBOP (8.24 mg, 15.8 :mol), HOBt( 2.13 mg, 15.8 :mol) and Hunig's base (8.8 :l, 51.0 :mol). The reaction solution is added dropwise to 20 mL of acetonitrile, giving a precipitate that is collected by centrifugation. The precipitate is purified by reverse phase HPLC to yield a compound of formula I as its salt.

### EXAMPLE 6

Preparation of a [C—V] Compound of Formula I

### (1) Preparation of a Compound of Formula (27) in which m is 2

A ligand bearing an amino group (3.2 mmol) is suspended in 40 mL of 1,3-dimethyl-3,4,5,6-tetrahydro-2-(1H)-pyrimidinone and heated at 70° C. for 15 minutes. 4-Butoxybenzaldehyde (570 mg, 3.2 mmol) is added and the mixture heated at 70° C. for 1 hour. Sodium cyanoborohydride (241 mg, 3.8 mmol) in 2 mL methanol is added and the mixture heated at 70° C. for 2 hours, then cooled to room temperature. The reaction solution is added dropwise to 20 mL of acetonitrile, giving a precipitate that is collected by centrifugation. The precipitate is purified by reverse phase HPLC to yield a compound of formula (27) as its salt.

### 2. Preparation of a compound of Formula (28) where m is 2

The compound of formula (27) prepared above (45.0 :mol) is dissolved in 2 mL of DMF. Ethylene diamine (13.4 mg, 22.3 ?mol) is added followed by PyBOP (28.0 mg, 54.0 :mol), HOBt (7.2 mg, 54.0 :mol) and Hunig's base (63.0 :L, 360 :mol). The reaction solution is added dropwise to 20 mL of acetonitrile, giving a precipitate that is collected by centrifugation. The precipitate is purified by reverse phase HPLC to yield a compound of formula (28) as its salt.

### 3. Preparation of a [C—V] compound of Formula I in which one V position is substituted, wherein m and n are 2 and p is 3

The compound of formula (28) prepared above (4.9 :mol) is dissolved in 500 :L of DMF. The compound of formula (19) (4.9 :mol) is added to the solution, followed by PyBOP (3.06 mg, 5.9 :mol), HOBt (0.80 mg, 5.9 :mol) and Hunig's base (6.7 uL, 38.4 :mol). The reaction solution is added dropwise to 20 mL of acetonitrile, giving a precipitate that is collected by centrifugation. The precipitate is purified by reverse phase HPLC to yield a compound of Formula I as its salt.

### EXAMPLE 7

### Preparation of N-aryl-2-chloroacetamide

Under nitrogen atmosphere, one equivalent of an appropriately substituted aniline (7.2 mmoles) was dissolved in 50 mL dichloromethane. To this was added 1.3 equivalents of DIEA (9.4 mmoles). The reaction mixture was cooled in an ice bath. Chloroacetyl chloride (7.95 mmoles) dissolved in 50 mL dichloromethane was added dropwise to the reaction mixture. The reaction was stirred at room temperature until reaction was complete (approximately 0.5 hours). The course of the reaction was followed by TLC (50% ethyl acetate and 50% hexanes). The reaction mixture was evaporated to a syrup, and was then partitioned between EtOAc and water. The organic layer was washed with saturated NaHCO₃ (2 times), saturated NaCl, then dried with MgSO₄, filtered and rinsed with EtOAc. After removal of excess solvent, 2-chloro-2′,6′-acetoxylidide was obtained as a white solid (51% yield).

### EXAMPLE 8

### Preparation of an [N—N] bivalent local anesthetic compound of Formula I 1. Preparation of di-Boc-protected diaminobutane

1,4-Diaminobutane (34.0 mmol) was dissolved in 100 mL dichloromethane under nitrogen atmosphere. Di-tert-butyl dicarbonate (Boc₂O) (119.12 mmol) dissolved in 100 mL dichloromethane was added dropwise to the stirred solution and stirring was continued at room temperature until the reaction was complete (4 hours). The course of the reaction was followed by TLC (50% ethyl acetate and 50% hexanes). The reaction mixture was evaporated giving a precipitate that was collected by filtration. The precipitate was rinsed with ether to yield a white solid (9.02 grams; 92% yield).

### 2. Preparation of N,N-dimethyldiaminobutane

Lithium aluminum hydride (LAH) (63.3 mmol) was dissolved in 200 mL tetrahydrofuran (THF) in an ice bath under nitrogen atmosphere. Di-Boc-protected diamine (12.7 mmol) was dissolved in 50 mL THF and added dropwise to the LAH/THF solution. The reaction was stirred with cooling, then warmed to room temperature, placed in an oil bath and the temperature was raised by increments of 10° C. to 85° C. for 30 minutes . The mixture was stirred at reflux for 18 hours, then cooled to room temperature and placed in an ice bath. Sodium sulfate decahydrate was slowly added to quench the excess LAH. The solids were removed by filtration and rinsing with THF. The filtrate was concentrated to a thick syrup and excess solvent removed under vacuum to yield N,N-dimethyldiaminobutane as a viscous oil (3.58 grams; 98% yield).

### 3. Preparation of bivalent lidocaine linked by N,N-dimethyldiaminobutane

N,N-dimethyldiaminobutane (1.95 mmol), 2-chloro-2′,6′-acetoxylidide (3.71 mmol) and diisopropylethylamine (DIEA) (4.89 mmol) were dissolved in 4 mL ethanol. under nitrogen atmosphere. The mixture was stirred at 85° C. until the reaction was complete (12 hours). The course of the reaction was followed by TLC (50% ethyl acetate and 50% hexanes). The mixture was cooled to room temperature for crystallization of the product, then filtered to yield a solid product, which was rinsed with ether, and excess solvent was removed under vacuum to yield the bivalent lidocaine N,N-dimethylaminobutane-linked product as a white solid (0.88 grams; 83% yield).

### 4. Preparation of compounds of Formula I of higher-order valency

Accordingly, following the procedures of Example 8, steps 1-3, using the appropriate molar quantities of Boc₂O and N-aryl-2-chloroacetamide, and a desired multifunctional amine linker, other [N—N] local anesthetic compounds of Formula I are prepared.

### EXAMPLE 9

### Quaternization of a local anesthetic compound of Formula I

One equivalent (0.45 mmole) of bivalent lidocaine compound from Example 8 above was dissolved in 3 mL methyl iodide under a nitrogen atmosphere, heated to 80-85° C. for 1-2 hours or until the reaction is complete, and the reaction is cooled. The excess methyl iodide is removed and the residue washed with hexane and dried under vacuum to afford the di-quaternary salt of the bivalent lidocaine compound as a whate solid (0.42 grams; 98% yield).

Accordingly, following this procedure, other [N—N] local anesthetic compounds of Formula I are prepared as their quaternary salts.

### EXAMPLE 10

### Preparation of bivalent local anesthetic compound attached via piperidine rings to an alkylene linker

2′,6′-Dimethyl-2-piperidinecarboxanilide was prepared as follows: DL-Pipecolinic acid (2-piperidine carboxylic acid) was reacted with a 20% excess of benzyl chloroformate, under basic conditions, to give the N-protected carboxylic acid. This was reacted with 2,6-dimethylaniline in DMF containing DIEA/HATU (N-[(dimethylamino)-1H-1,2,3-triazolo[4,5-b]pyridin-1-ylmethylene]-N-methylmethanaminium hexafluorophosphate N-oxide) (approx. 1.2-1.3 equiv. each), catalyzed by HOAT (3-hydroxy-3H-1,2,3-triazolo[4,5-b]pyridine), to give the amide. The protecting group was removed by catalytic hydrogenation (Pd/C in methanol).

Approx. 1.9 equivalents of the 2′,6′-dimethyl-2-piperidinecarboxanilide were combined with one equivalent 1,10-dibromodecane, and 2.5 equivalents DIEA in ethanol. The mixture was heated and stirred at 8020 C. under nitrogen overnight. The reaction mixture was concentrated, dissolved in 0.1N HCl, diluted with water, and lyophilized to recover the hydrochloride salt of the bivalent compound.

### BIOLOGICAL EXAMPLE

### BTX Displacement Assay

The local anesthetic binding site in the sodium ion channel is allosterically linked to the binding site for batrachotoxin (BTX). The displacement of radiolabeled BTX from Site 2 on synaptosomal membranes correlates with local anesthetic activity.

### 1. Preparation of Synaptosomes

Synaptosomes were prepared using frozen rat brains obtained from Pel-Freez. Ten brains were thawed and the cerebral cortex harvested (cerebellum removed). Approximately 4 to 5 g of cortex were placed in 30 ml of 0.32 M sucrose at 020 C. and homogenized in a Teflon/glass homogenizer (clearance approx. 0.15-0.23 mm, 12 up and down strokes at less than 800 rpm) while the mortar of the homogenizer was kept in an ice/water bath. The crude homogenates were pooled and distributed as 20 ml aliquots into polycarbonate centrifuge bottles. These were centrifuged at 5,100 rpm (ω²t setting 1.57×10⁸ rad²/sec) in a 50.2 Ti rotor for approximately 9 min at 420 C. in a Beckman L8-80M ultracentrifuge. The pellet (P1) was discarded. The supernatant (nominally in 0.32 M sucrose) was removed, layered onto 8 ml of 1.2 M sucrose at 0° C. and spun at 50,000 rpm (ω²t setting 1.6×10¹⁰ rad²/sec) for approximately 10 min in a 50.2 Ti rotor at 4° C. Supernatant (4 ml) was removed from the gradient interface and mixed with 10 ml of 0.32 M sucrose at 0° C. The pellet (P2) and other material was discarded. The diluted interface was layered onto 8 ml of 0.8M sucrose and spun at 50,000 rpm (ω²t setting 1.6×10¹⁰ rad²/sec) for approximately 10 min in a 50.2 Ti rotor at 4° C. The supernatant was discarded, and the pellets (P3) were pooled and resuspended by homogenization at 0° C. with a Wheaton glass homogenizer B type (clearance approx. 0.15-0.23 mm, 12 up and down strokes) in 15 ml synaptosomal storage buffer (130 mM choline Cl, 5.5 mM glucose, 5.4 mM KCl, 0.8 mM MgSO₄), 4.50 mM HEPES, pH adjusted to 7.4 with Tris Base (approx. 22 mM)). The homogenized solution was spun at 50,000 rpm (ω²t setting 1.6×10¹⁰ rad²/sec) for approximately 10 min in a 50.2 Ti rotor at 4° C. The supernatant was discarded, and the pellet (P4) was resuspended in 8 ml synaptosomal storage buffer by homogenization (as in the previous step) at 0° C. The synaptosomes were “snap frozen” in 500 μl aliquots on ethanol/dry ice and stored at −80° C. Total protein concentration was measured after solubilizing membranes in 1.2% (wt/vol) SDS by Lowry's method.

### 2. BTX Displacement Assay

Incubations were carried out in a total volume of 250 μl containing synaptosomes at a final concentration of 0.8 μg total protein/μl 50 nM ³H-BTX-13 (50 Ci/mmol), 1 μM tetrodotoxin, 120 μg/ml Leiurus quinquestriatus venom, in synaptosome storage buffer (see above). Test compound was added at various concentrations, typically from 1 to 100 μg/ml. Systems were incubated for 30 to 45 min at 37° C., after which time incubation was terminated by dilution of the reaction mixture with 250 μl of wash buffer (163 mM choline Cl, 5 mM HEPES, 1.8 mM CaCl₂ and 0.8 mM MgSO₄, pH to 7.4 with Tris base) at 0° C., and solutions were filtered through a Millipore GFC 96 well filter plate. Filtration was accomplished by vacuum filtration through a Millipore 96 well vacuum manifold. Each filter plate was washed three times with wash buffer at 0° C. (250 μl/wash). Filter and filtrate were counted in 25 μl of OptiPhase Supermix (Wallac) on a Wallac scintillation counter (model 1450 Microbeta). Nonspecific binding was determined by parallel experiments in the presence of 300 μM veratridine.

### Whole-Cell Voltage Clamp

The whole cell variant of the patch-clamp method (Hamill et al., Pflügers Arch. 391:85-100, 1981) was used to measure Na+ currents in GH₃ cells. The external solution contained (in mmol) 150 choline Cl, 0.2 CdCl₂, 2 CaCl₂, and 10 hydroxethylpiperazine ethane sulfonic acid (HEPES) adjusted to pH 7.4 with tetramethyl hydroxide. Micropipettes were fabricated and had a tip resistance of ˜1 Mω when filled with an Na+ solution containing (in mmol) 100 NaF, 30 NaCl, 10 EGTA (ethylene glycol-bis(β-aminoethyl ether)- N,N,N′,N′-tetraacetic acid), and 10 hydroxyethyl- piperazineethane sulfonic acid, adjusted to pH 7.2 with CsOH.

The junction potential of electrodes was nulled before seal formation. After the rupture of the patch membrane, the cell was allowed to equilibrate with the pipette solution for at least 15 min at the holding potential of −100 mV. Under these reversed Na+ gradient conditions, outward Na+ currents were activated at approximately −30 mV.

Test compounds, at appropriate concentrations, were applied to cells with a flow rate of about 0.12 ml/min via a series of narrow-bored capillary tubes positioned within 200 μm of the cell. Typically, the more soluble salt form, rather than the free base, was used. Washout of drugs was performed via a tube containing the external solution without drug present. Voltage-clamp protocols were created with pClamp software (Axon Instruments, Inc., Foster City, Calif.). Leak and capacitance were subtracted by a leak and capacity compensator (Hille and Campbell, J. Gen. Physiol. 67:265-93, 1976). Additional compensation was achieved by the patch clamp device (EPC7, List-Electronic, Darmstadt/Eberstadt, Germany). All experiments were performed at room temperature. At the end of the experiments, the drift in the junction potential was generally <2 mV.

### Frog Sciatic Nerve Sucrose-Gap Assay

Large bullfrogs (Rana catesbiana) measuring approximately 5 inches (obtained from Amphibians of North America Co.) were used in the experiments. The sciatic nerves were dissected from rapidly decapitated and pithed animals and maintained in a N-morpholinopropane sulfonate (MOPS)-Ringer's solution. Nerves were stored for up to 2 days at 4° C.

The MOPS-Ringer's solution contained, in mmol, 110 NaCl, 2.5 KCl, 2.0 CaCl₂, 4.0 glucose and 5.0 MOPS buffer. The solution pH was adjusted to 7.2-7.3 using 1 N NaOH. This normal Ringer's solution was used for storing nerves and for the filling of the two stimulating pools (50 μl each) and the recording “intracellular” pool. The solution used for the “test” pool (to begin recording with and for the drug solutions) contained 10 mM tetraethylammonium chloride added to the normal Ringer's solution.

The compounds to be tested for local anesthetic activity were prepared as 1 mM stock solutions in double-deionized water. The solutions were stored at −20° C. to minimize loss of potency from frequent defrosting. The working solutions were prepared by diluting stock solution in TEA-Ringer's immediately prior to their use in an experiment.

Segments of nerve measuring 32-35 mm (tibial or peroneal branch) were desheathed and mounted in a polycarbonate sucrose-gap chamber. In the chamber, nerves were laid across a series of pools and within a cylindrical gap with the proximal nerve end in the “test” pool. The isotonic sucrose (180 mM in double-deionized water) was perfused through a hollow gap. A petroleum jelly (Vaseline, Cheeseborough Pons) was used to create water-tight seals surrounding regions of the nerve passing between adjacent aqueous pools.

The proximal end of the nerve was stimulated by a pair of bipolar Ag/AgCl electrodes inserted into the stimulating pools. The “test” pool (500 μl volume) contained the Ag/AgCl electrode that recorded the extracellular electrical potential. Flowing at 1.5 ml/min, the nonionic sucrose solution prevented the action potential from propagating beyond the test pool. The intracellular potential conducted passively through the sucrose gap to the distal end of the preparation was recorded by a Ag/AgCl (“intracellular”) electrode in Ringer's containing pool.

Using a Grass S48 Stimulator (Grass Instruments, Quincy, Mass.), nerves were stimulated for 50 μsec at three times the intensity required to induce the maximal compound action potential (CAP) (3-4 V). Stimuli were supplied by an isolated stimulation unit (model SIU5A, Grass Instruments, Quincy, Mass.). The electrical signal from the nerve, the compound action potential (CAP) from large myelinated fibers, was amplified 50 times using an amplifier model AK47LN (Metametrics, Cambridge, Mass.). The signal was displayed on a oscilloscope (model 1032AB, Ballantine Laboratories, Inc, Boonton, N.J.), from which it was photographed using Polaroid film for later analysis.

A nerve preparation was considered acceptable if the CAP measured not less than 20 mV, and the experiment was carried out after CAP stabilizing in TEA-Ringer's (not changed more than 2 mV over a 10-20 min period).

Nerves were stimulated at 0.4 Hz during the full experiment time to assess “tonic” block, and “phasic” block was measured by 20 Hz trains applied for 2-4 secs every 5-10 min in drug solutions as well as in the control TEA-Ringer's. As each train of stimuli was administered, the resulting CAPs were recorded. Before testing the drug solutions, the test pool was emptied and refilled 3 times with the same volume of the control TEA-Ringer's. The same procedure preceded the washing period. When washing out the preparation, the TEA-Ringer's was changed 3-4 times more. All data were recorded at room temperature.

The compounds of Formula I were active in the above tests and demonstrated improved properties when compared to monobinding lidocaine.

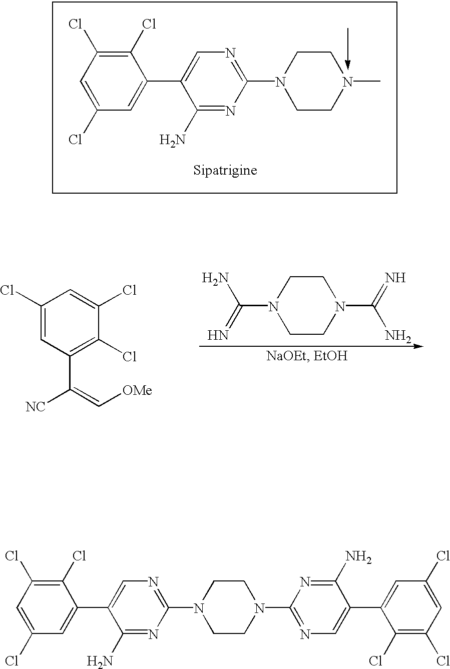

To a solution of NaOEt in ethanol (20 mL) is added piperazinodiformamidine dihydrochloride (8.22 mmol) (CAS 17238-65-2). After stirring for 10 minutes, 2-(2,3,5-trichlorophenyl)-3-methoxyacrylonitrile (19.2 mmol) is added and the mixture is stirred at reflux for 4 hours. The mixture is left standing at room temperature overnight and then filtered. The filtrate is concentrated and the residue is purified by chromatography to afford the desired product.

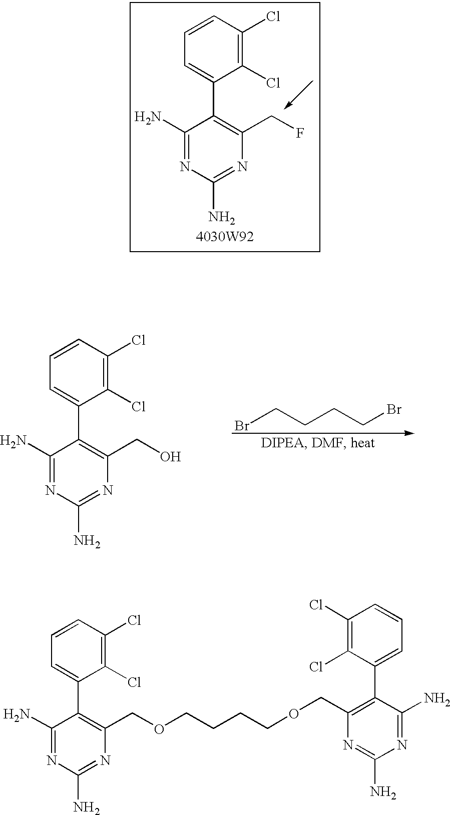

A solution of 20 mmols of the starting compound, which is reported in WO97/09317, in DMF with 10 mmols of 1,4-dibromobutane and 20 mmols of diisopropylethylamine is heated at 80° C. and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

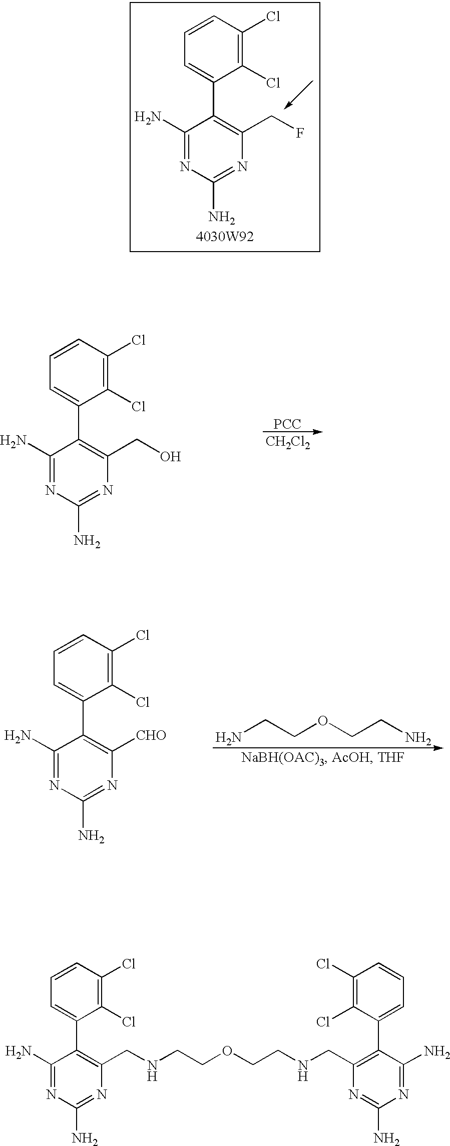

The starting compound (100 mmol), which is reported in WO97/09317, is dissolved in CH₂Cl₂. Pyridinium chloroformate (110 mmol) is added in portions with stirring. The progress of the reaction is monitored by TLC. When judged complete, the solution is filtered through a small plug of silica gel, then evaporated under vacuum. The residue is chromatographed to afford the desired product .

2,2′-Oxybis(ethylamine) dihydrochloride (1 mmol) (CAS 60792-79-2) is dissolved in THF (10 ml). Acetic acid (0.5 ml) is then added and the reaction is heated to reflux. The product from the preceding reaction (2 mmol) dissolved in THF (10 ml) is then added dropwise to the refluxing solution over 60 minutes and the reaction is refluxed for a further 60 minutes. At this point, NaBH(OAc)₃ is added in portions and the reaction is stirred at relux for a further 2 hours. The reaction is allowed to cool and then is quenched with aqueous NH₄Cl solution until the pH of the solution is adjusted to pH 7.0 using either 1 M HCl or 1 M NaOH. The product is extracted from this aqueous phase with EtOAc. The organic layer is dried using Na₂SO₄, the drying agent is then filtered off and the solvent removed in vacuo to provide the crude product. The desired material is purified from this mixture using reverse phase HPLC.

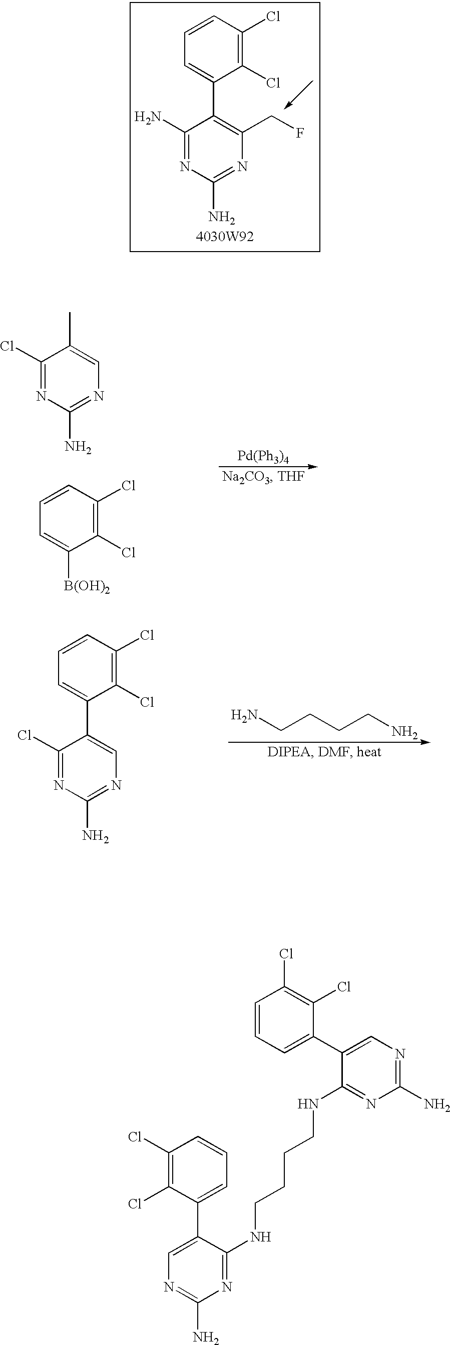

A mixture of the starting compound (30 mmol) (CAS 3993-80-4) in THF and tetrakis(triphenylphosphine)palladium(0) is stirred under nitrogen at room temperature for 10 minutes. 2M aqueous sodium carbonate is added to the mixture followed by a solution of 2,3-dichlorobenzene boronic acid (30 mmol), described in WO 98/38174, in absolute ethanol and the mixture refluxed under nitrogen for 17 hours. A further equivalent of 2,3-dichlorobenzene boronic acid in absolute ethanol is added and the mixture refluxed for an additional 7.50 hours. Finally, another equivalent of 2,3-dichlorobenzene boronic acid in absolute ethanol is added to the mixture and continued refluxing for 17 hours. The cooled mixture is evaporated in vacuo. The residue is dissolved in chloroform, washed with aqueous saturated sodium bicarbonate and water, dried over anhydrous magnesium sulphate, filtered and the filtrate evaporated down in vacuo. The residue is purified by flash chromatography using chloroform/methanol as the eluant to afford the desired product.

A solution of 20 mmols of the product from the preceding reaction in DMF with 10 mmols of 1,4-diaminobutane and 20 mmols of diisopropylethylamine is heated as necessary in a sealed vessel and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

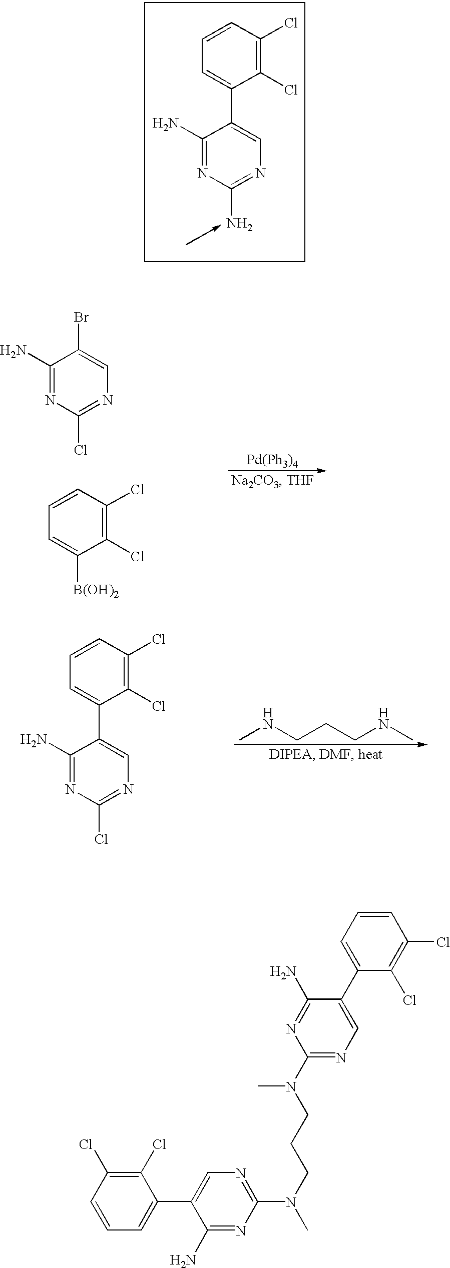

A mixture of the starting compound (30 mmol) (CAS 205672-25-9) in THF and tetrakis(triphenylphosphine)palladium(0) is stirred under nitrogen at room temperature for 10 minutes. 2M aqueous sodium carbonate is added to the mixture followed by a solution of 2,3-dichlorobenzene boronic acid (30 mmol) in absolute ethanol and the mixture refluxed under nitrogen for 17 hours. A further equivalent of 2,3-dichlorobenzene boronic acid in absolute ethanol is added and the mixture refluxed for an additional 7.50 hours. Finally, another equivalent of 2,3-dichlorobenzene boronic acid in absolute ethanol is added to the mixture and continued refluxing for 17 hours. The cooled mixture is evaporated in vacuo. The residue is dissolved in chloroform, washed with aqueous saturated sodium bicarbonate and water, dried over anhydrous magnesium sulphate, filtered and the filtrate evaporated down in vacuo. The residue is purified by flash chromatography using chloroform/methanol as the eluant to afford the desired product.

A solution of 20 mmols of the product from the preceding reaction in DMF with 10 mmols of N,N′-dimethyl-1,3 -propanediamine and 20 mmols of diisopropylethylamine is heated as necessary in a sealed vessel and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

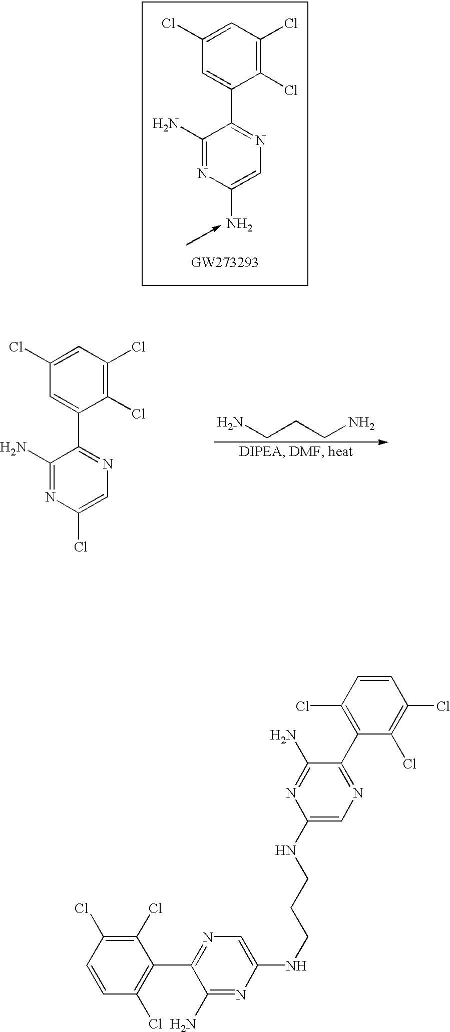

A solution of 72 mmols of 2-amino-6-chloro-3-(2.3,5-trichlorophenyl)pyrazine, which is reported in WO 98/38174, in DMF with 36 mmols of 1,3-diaminopropane and 72 mmols of diisopropylethylamine is heated as necessary in a sealed vessel and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

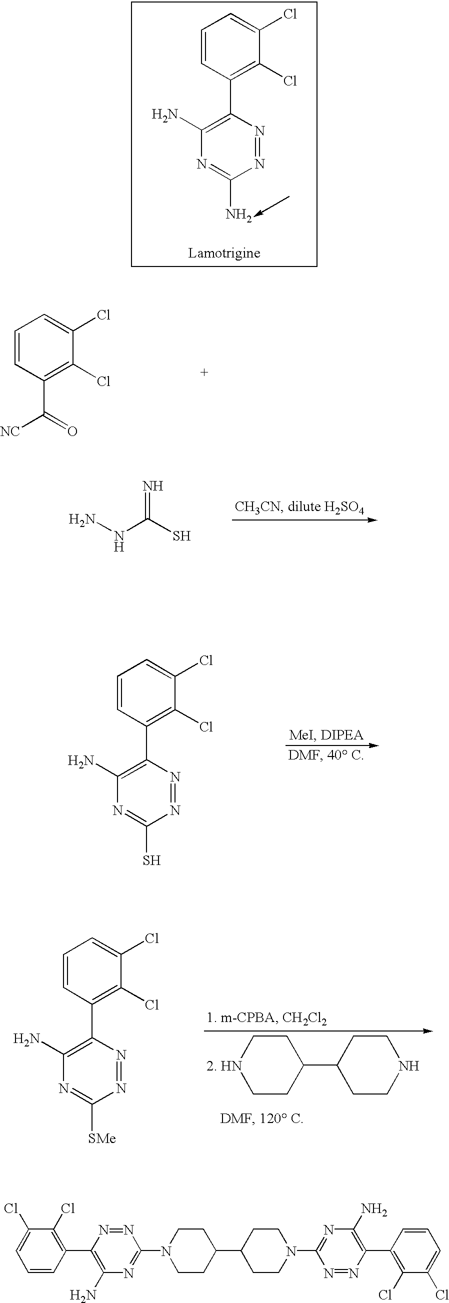

A solution of 2,3-dichlorbenzoyl cyanide (30 mmol), which is reported in EP 0459829(A1), is dissolved in acetonitrile and added dropwise to a suspension of thiosemicarbazide (90 mmol) (CAS 79-19-6) in dilute sulphuric acid. The temperature is maintained below 30° C. The mixture is stirred at room temperature for 3 days. The solid is filtered, washed with water and sucked dry. A suspension of the solid in a 10% solution of sodium hydroxide pellets in water is stirred at room temperature for 1 hour. The solid is filtered, washed with water and dried in vacuo. The solid is refluxed with hot n-propanol for 1.5 hours, filtered and dried in vacuo at 80° C. to afford the desired product.

A solution of 20 mmols of the above product in DMF with 20 mmols of methyl iodide and 20 mmols of diisopropylethylamine is heated at 40° C. for 12 hours. The reaction mixture is concentrated and chromatographed to afford the desired product.

A solution of 10 mmols of the above product in dichloromethane with 20 mmols of m-chloroperoxybenzoic acid is stirred at room temperature for 12 hours. The reaction mixture is concentrated and chromatographed to afford the desired product. A solution of 5.0 mmols of the resulting product in DMF with 2.5 mmols of 4,4′-bipiperidine dihydrochloride is heated at 120° C. for 12 hours in a sealed vessel. The reaction mixture is concentrated and chromatographed to afford the desired product.

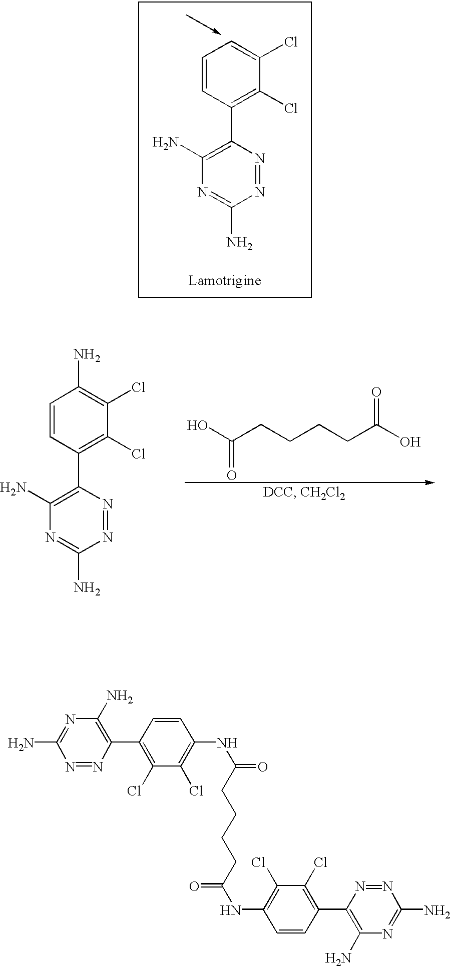

A solution of 3,5-diamino-6-(4-amino-2,3-dichlorophenyl)- 1,2,4-triazine, which is reported in BP 459829(A1), (2 mmols) and adipic acid (1 mmol) in methylene chloride is prepared under argon in a flask equipped with magnetic stirrer and drying tube. To this solution is added dicyclohexylcarbodiimide (solid, 2.1 mmols) while stirring at room temperature. The course of the reaction is followed by thin layer chromatography. When reaction has occurred, the reaction solution is diluted with ethyl acetate and washed with water and with aqueous Na₂CO₃. The organic layer is dried (Na₂SO₄), filtered and concentrated under reduced pressure to give the crude product. The desired compound is obtained by purification of the crude product by use of HPLC.

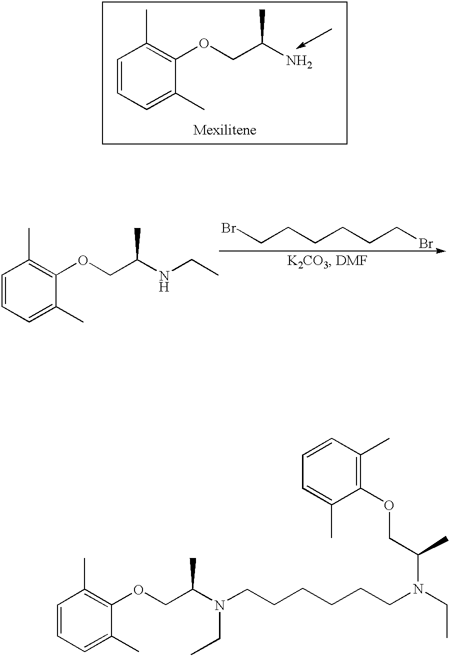

A solution of 20 mmols of (R)-N-ethyl mexiletine, which is reported in WO 97/27169. in DMF with 10 mmols of 1,6-dibromohexane and 20 mmols of potassium carbonate is heated as necessary and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

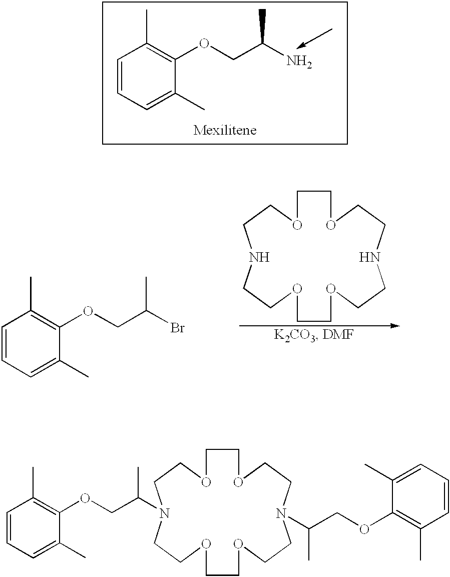

A solution of 20 mmols of 2-(2-bromopropoxy)-1,3-dimethylbenzene (CAS 96656-46-1) in DMF with 10 mmols of 1,4,10,13-tetraoxa-7,16-diazacyclooctadecane (4,13-diaza-18-crown-6) and 20 mmols of potassium carbonate is heated as necessary and the reaction followed by TLC. When judged complete, the mixture is partitioned between ethyl acetate and water and the organic phase washed with water, dried over sodium sulfate and the solvent removed in vacuo. The residue is purified by chromatography and separation of the stereoisomers by known technique to afford the desired product.

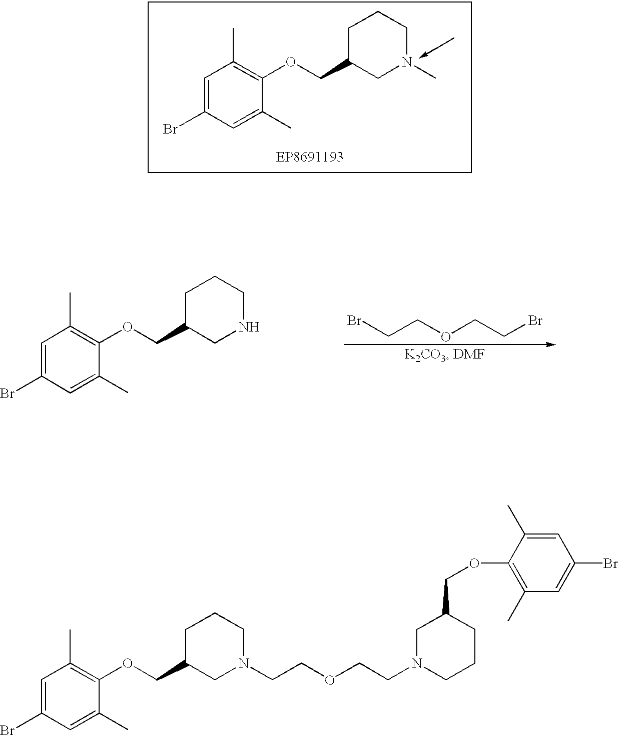

A solution of 20 mmols of (S)-3-(4-bromo-2,6-dimethylphenoxymethyl)piperidine, which is reported in EP 0869119 A1, in DMF with 10 mmols of 2-bromoethyl ether and 20 mmols of potassium carbonate is heated as necessary and the reaction followed by TLC. When judged complete, the mixture is partitioned between ethyl acetate and water and the organic phase washed with water, dried over sodium sulfate and the solvent removed in vacuo. The residue is purified by chromatography to afford the desired product.

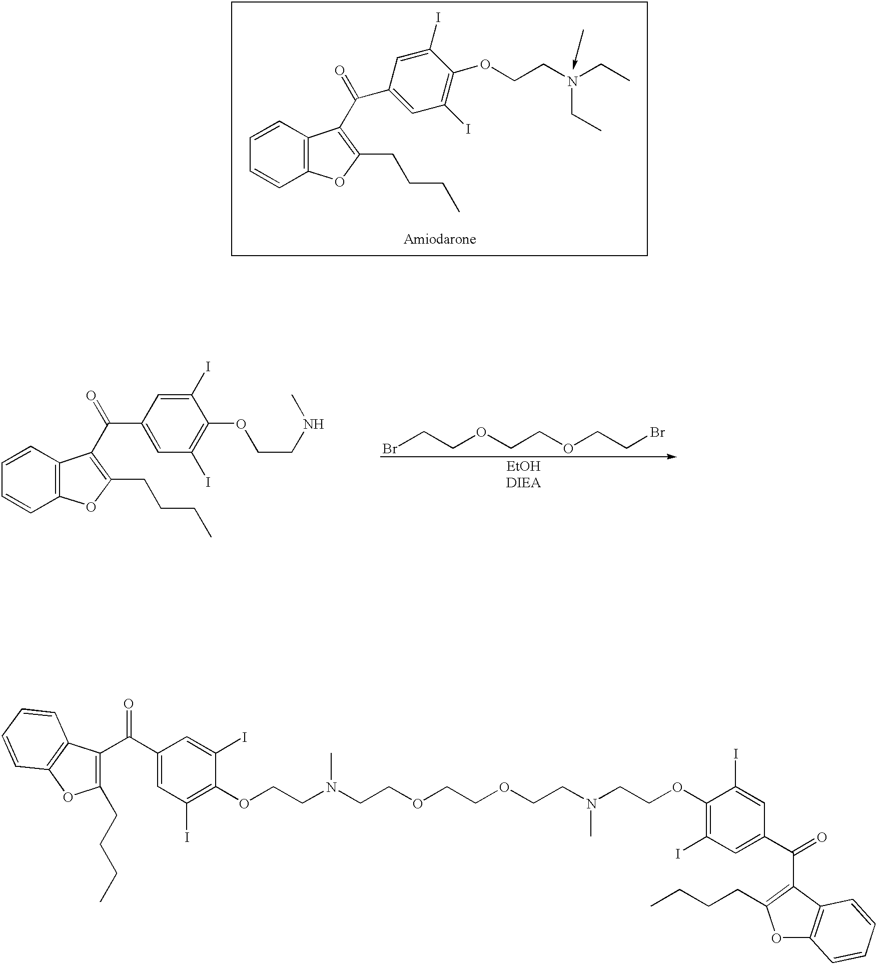

A solution of N-methyl 2-[4-[2-butylbenzofuran-3-ylcarbonyl]-2,6-diiodophenoxy]-ethylamine, prepared according to procedures described in Eur. J. Med Chem., 1974, 19-25, (5 mmol), 1,8-dibromo-3,6-dioxaoctane (2.5 mmol) and diisopropylethylamine (2 mL) in EtOH (25 mL) is maintained at room temperature . The progress of the reaction is followed by tlc. When it is complete, the mixture is poured into water and extracted with CH₂Cl₂. The extract is dried and evaporated, and the residue is chromatographed to afford the desired compound.

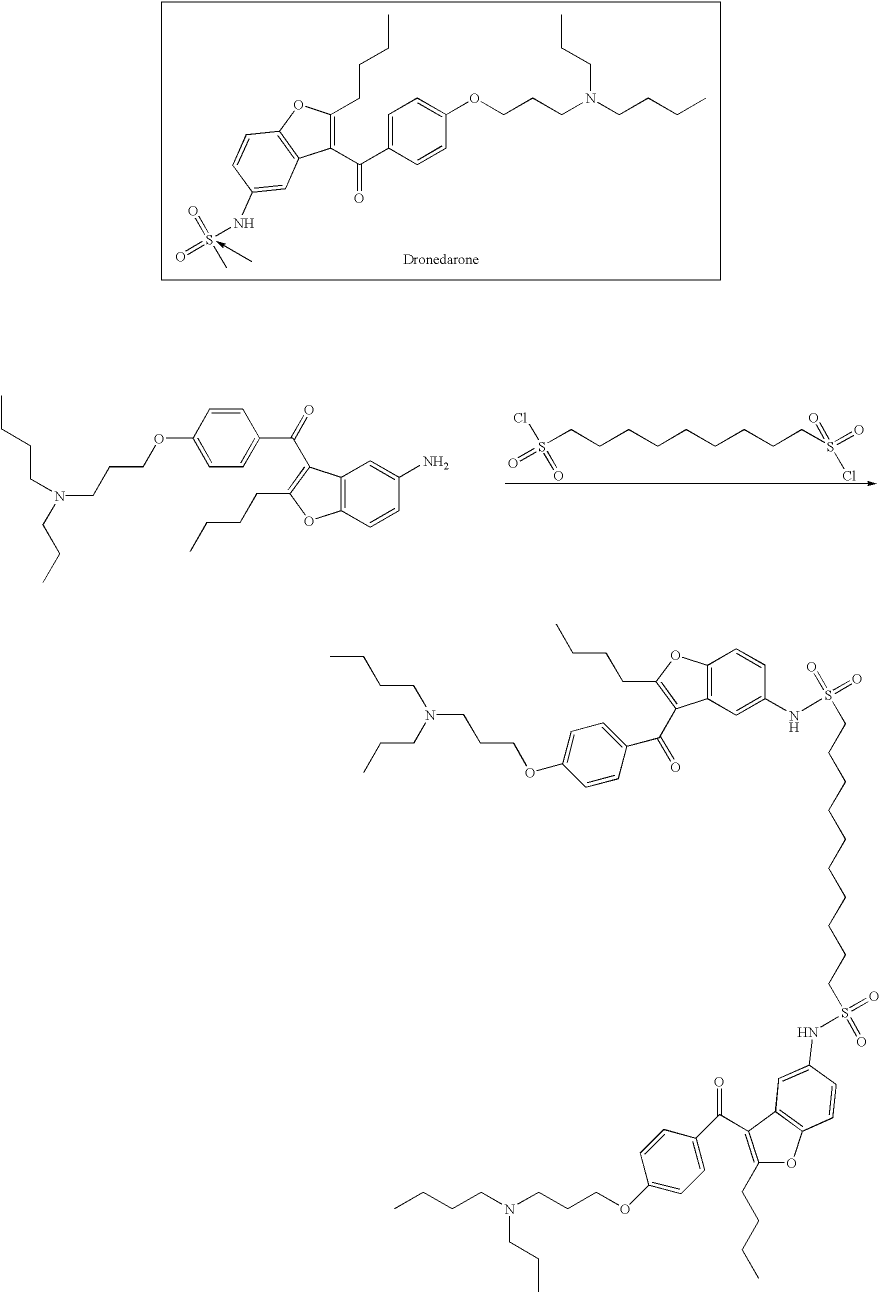

5-Amino-2-butyl-3-[4-(3-dibutylaminopropoxy)benzoyl]benzofuran, prepared as described in EP 0471609, (1 mmol) and 1,10-di(chlorosulfonyl)decane (0.5 mmol) are heated at reflux in CH₂Cl₂ (20 mL). The progress of the reaction is followed by tic. When it is complete, the solution is added to dilute Na₂CO₃. The organic phase is separated, dried and evaporated, and the residue is chromatographed to afford the desired compound.

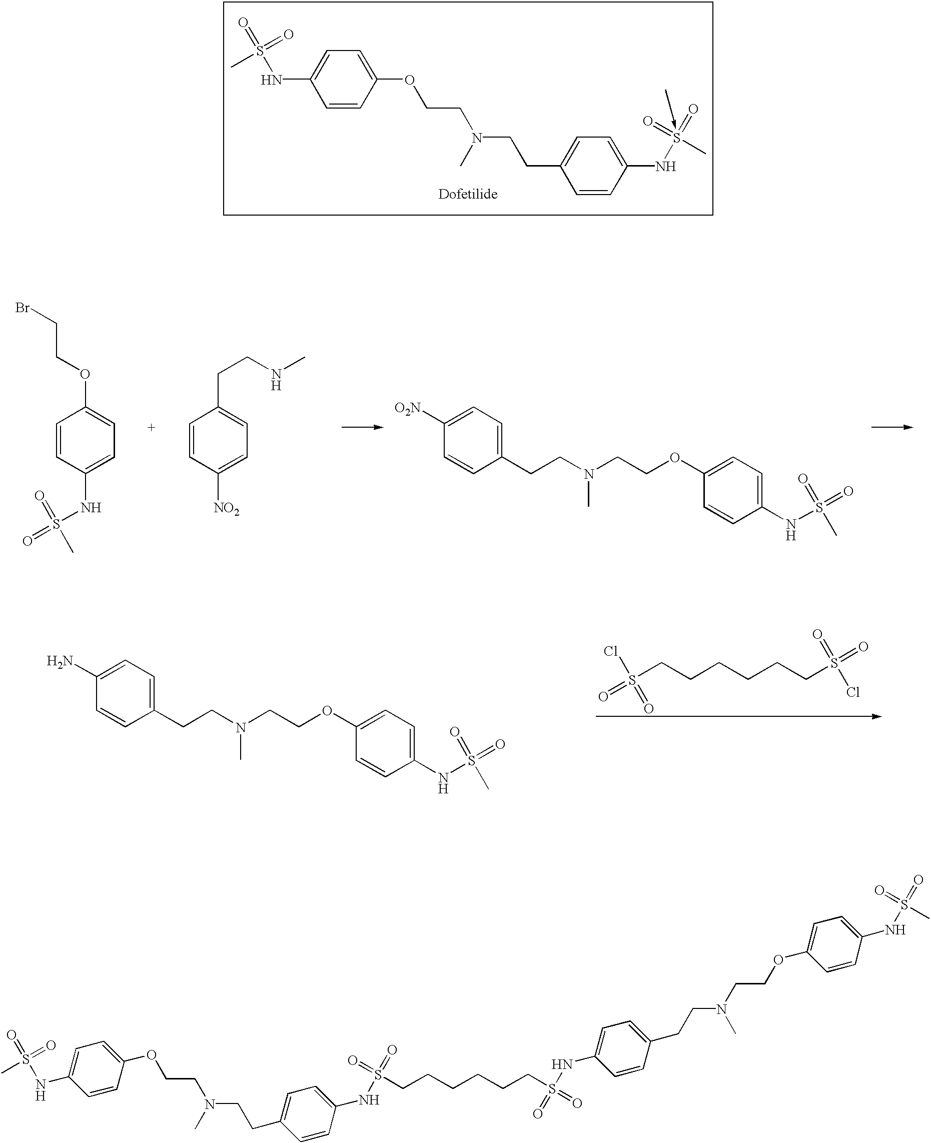

2-[4-(Methylsulfonylamino)phenoxy]ethyl bromide, (10 mmol) and N-methyl 2-(4-nitrophenyl)ethylamine (10 mmol), both prepared as described in J. Med. Chem. 1990, 1151, are heated at reflux in MeCN (100 mL) containing K₂CO₃ (3 g) and Kl (0.2 g). The reaction is monitored by tic. When it is complete, the solution is added to water and extracted with EtOAc. The extract is dried and evaporated and the residue is chromatographed to afford N-methyl N-(4-nitrophenylethyl) 2-[4-(methylsulfonylamino)phenoxy]ethylamine.

The above compound (3 mmol) is dissolved in EtOH (50 mL) and Raney nickel (1 g) is added. The mixture is stirred in a hydrogen atmosphere. The progress of the reaction is monitored by tic. When it is complete, the solution is filtered and then evaporated. The residue is chromatographed to afford N-methyl N-(4-aminophenylethyl) 2-[4-(methyl sulfonylamino)phenoxy]ethylamine.

A solution of hexane- 1,6-disulfonyl chloride (1 mmol), diisopropylethylamine (1 mL) and the above compound (2 mmol) in dry CH₂Cl₂ (25 mL) is maintained at room temperature. The progress of the reaction is monitored by tlc. When it is complete, the solvent is removed under reduced pressure and the residue is chromatographed to afford the desired compound.

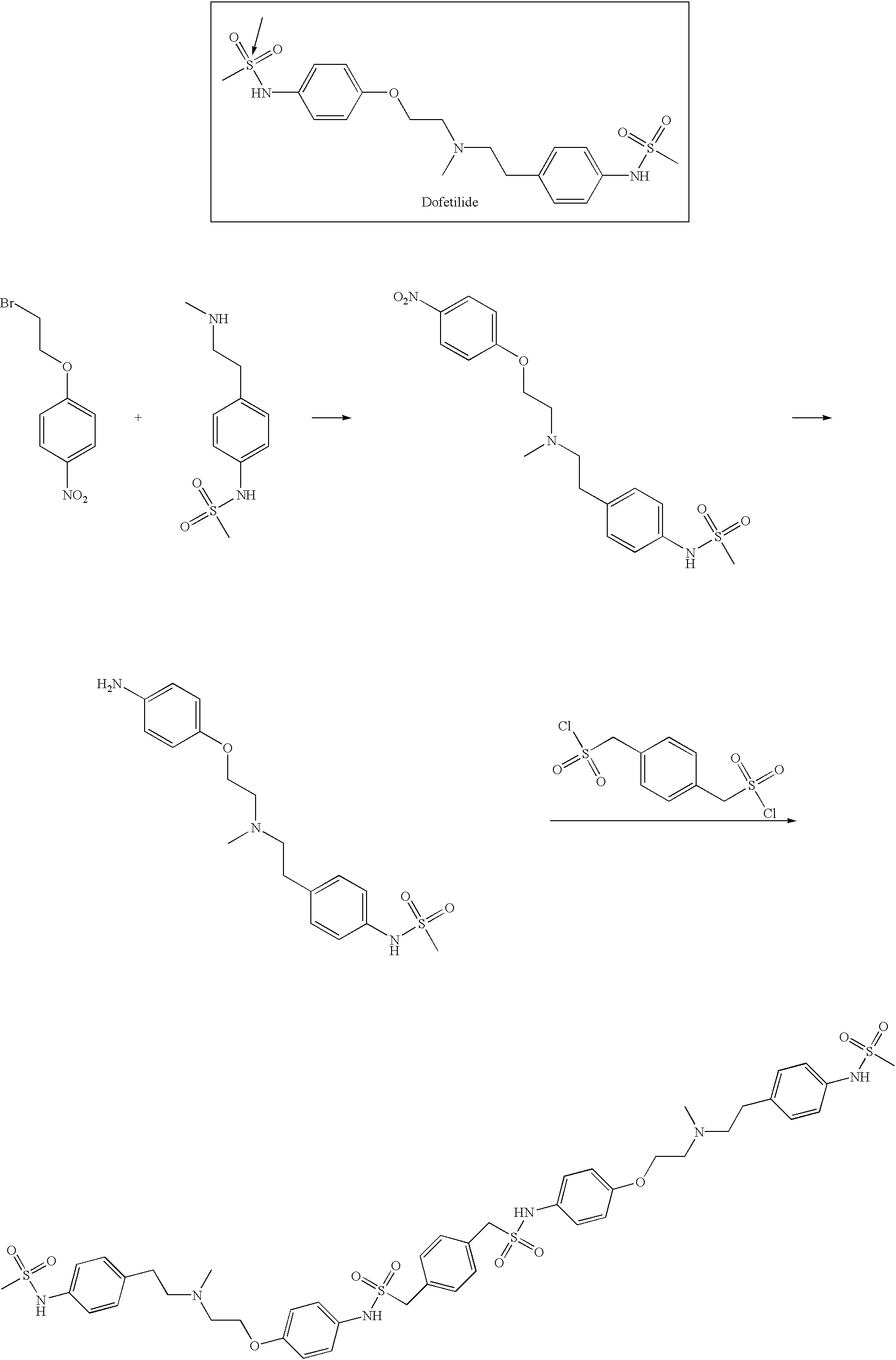

2-(4-Nitrophenoxy)ethyl bromide (10 mmol) and 2-(4-methylsulfonylaminophenyl) N-methylethylamine (10 mmol) are heated at reflux in MeCN (50 mL) containing K₂CO₃ (2 g). The progress of the reaction is monitored by tlc. When it is complete, the solution is added to water and extracted with EtOAc. The extract is dried and evaporated and the residue is chromatographed to afford N-methyl N-(4-(methylsulfonylamino)phenylethyl) 2-(4-aminophenoxy)ethylamine.

The above compound (3 mmol) is dissolved in EtOH (50 mL) and Raney nickel (1 g) is added. The mixture is stirred in a hydrogen atmosphere. The progress of the reaction is monitored by tlc. When it is complete, the solution is filtered and then evaporated. The residue is chromatographed to afford the desired compound.

The above compound (1 mmol) and 1,4-di-(chlorosulfonylmethyl)benzene (0.5 mmol) are dissolved in CH₂Cl₂ (50 mL) . The progress of the reaction is monitored by tlc. When it is complete, the solution is washed with dilute Na₂CO₃, then dried and evaporated. The residue is chromatographed to afford the desired compound

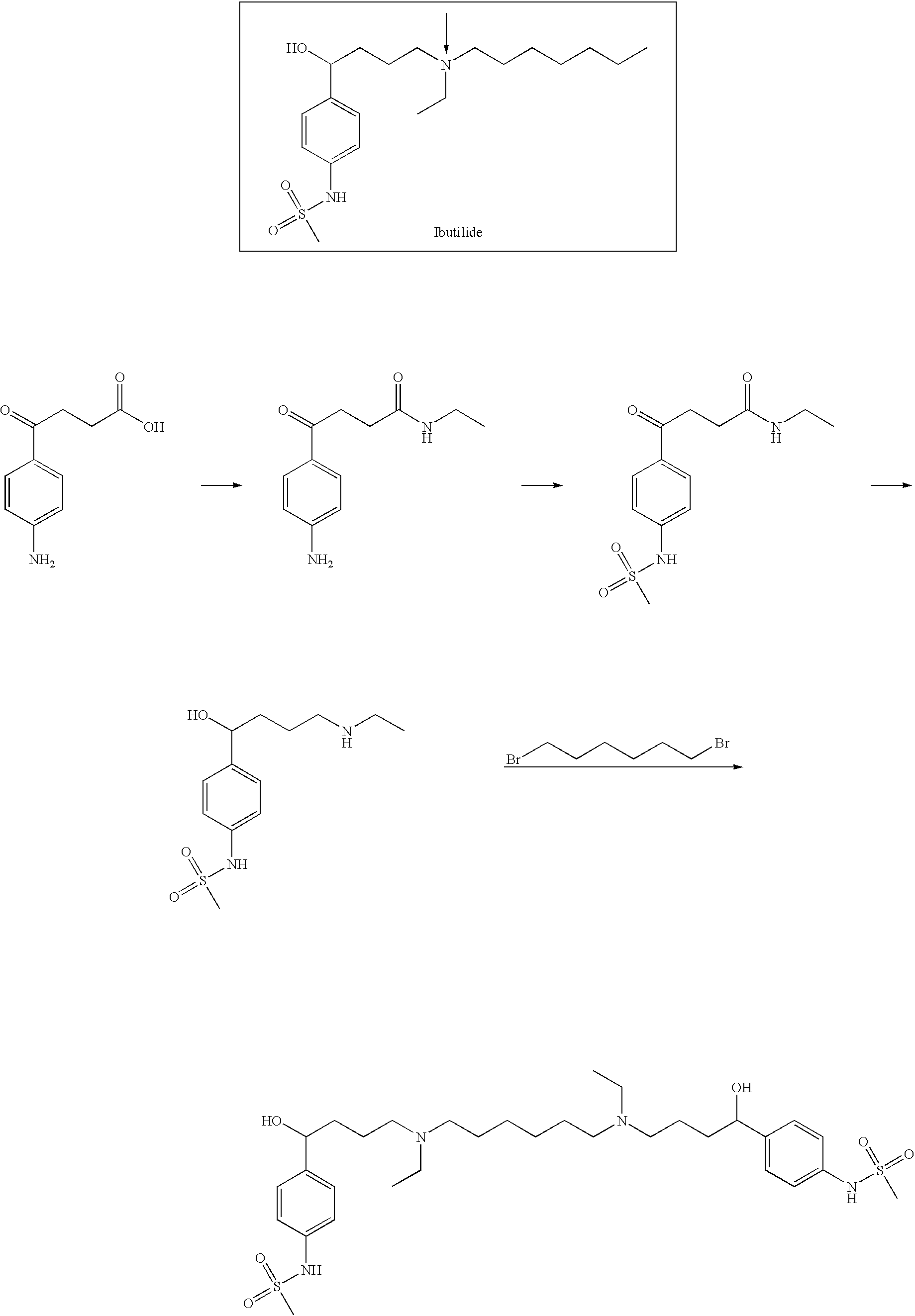

4-(4-Aminophenyl)-4-oxobutanoic acid (5 mmol) is added to a solution of dicyclohexylcarbodiimide (5 mmol) and ethylamine (5 mmol) in THF (50 mL) After 12 hours, the mixture is added to water and extracted with EtOAc. The extract is washed with dilute NaOH, then dried and evaporated. The residue is chromatographed to afford N-ethyl 4-(4-aminophenyl)-4-oxobutanamide.

The product from above (3 mmol) is dissolved in THF (25 mL) and to the solution is added diisopropylethylamine (5 mmol) and methanesulfonyl chloride (3 mmol). The reaction is monitored by tlc. When it is complete, the solution is added to water and extracted with EtOAc. The extract is dried and evaporated and the residue is chromatographed to afford N-ethyl 4-(4-methylsulfonylaminophenyl)-4-oxobutanamide.

The above-described compound (1 mmol) is dissolved in ether (50 mL); the solution is cooled to 0°, and to it is added lithium aluminum hydride (5 mmol). The reaction is monitored by tlc. When it is complete, excess hydride is decomposed by addition of aqueous potassium sodium tartrate. The organic phase is separated, dried and evaporated, and the residue is chromatographed to afford N-ethyl 4-(4-methylsulfonylaminophenyl)-4-hydroxybutylamine.

The above compound (1 mmol), diisopropylethylamine (2 mmol) and 1,6-dibromohexane (0.5 mmol) are dissolved in MeCN (25 mL). The progress of the reaction is monitored by tlc. When it is complete, the mixture is added to dilute Na₂CO₃, and extracted with CH₂Cl₂. The extract is dried and evaporated, and the residue is chromatographed to afford the desired compound.

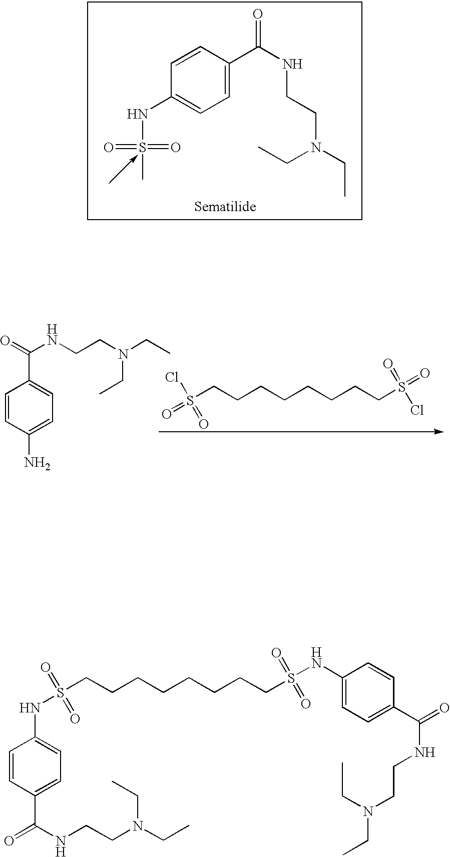

Procainamide (10 mmol) is dissolved in MeCN (50 mL) and 1,8-di-(chlorosulfonyl)octane (5 mmol) is added. The progress of the reaction is monitored by tlc. When it is complete, the solution is added to dilute Na₂CO₃ and extracted with CH₂Cl₂. The extract is dried and evaporated to afford the desired compound.

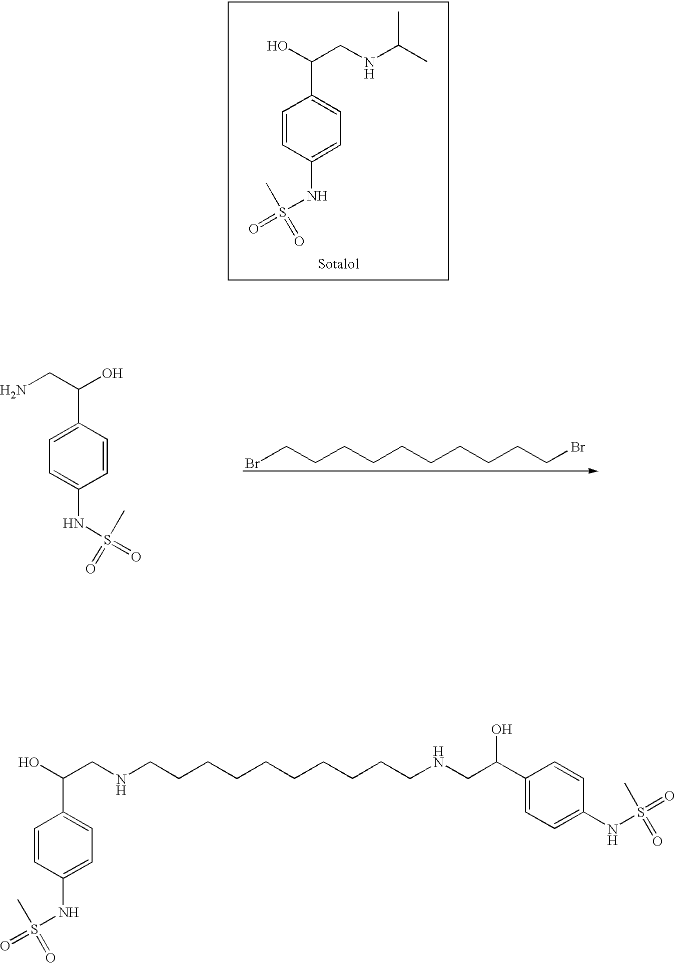

1,10-Dibromodecane (5 mmol), 2-hydroxy-2-(4-methylsulfonylaminophenyl)-ethylamine (10 mmol), prepared as described in European Patent 338793, potassium iodide (0.1 g) and K₂CO₃ (1 g) are stirred in MeCN (50 mL). The reaction is monitored by tlc. When it is complete, the mixture is added to water and extracted with CH₂Cl₂. The extract is dried and evaporated, and the residue is chromatographed to afford the desired compound.

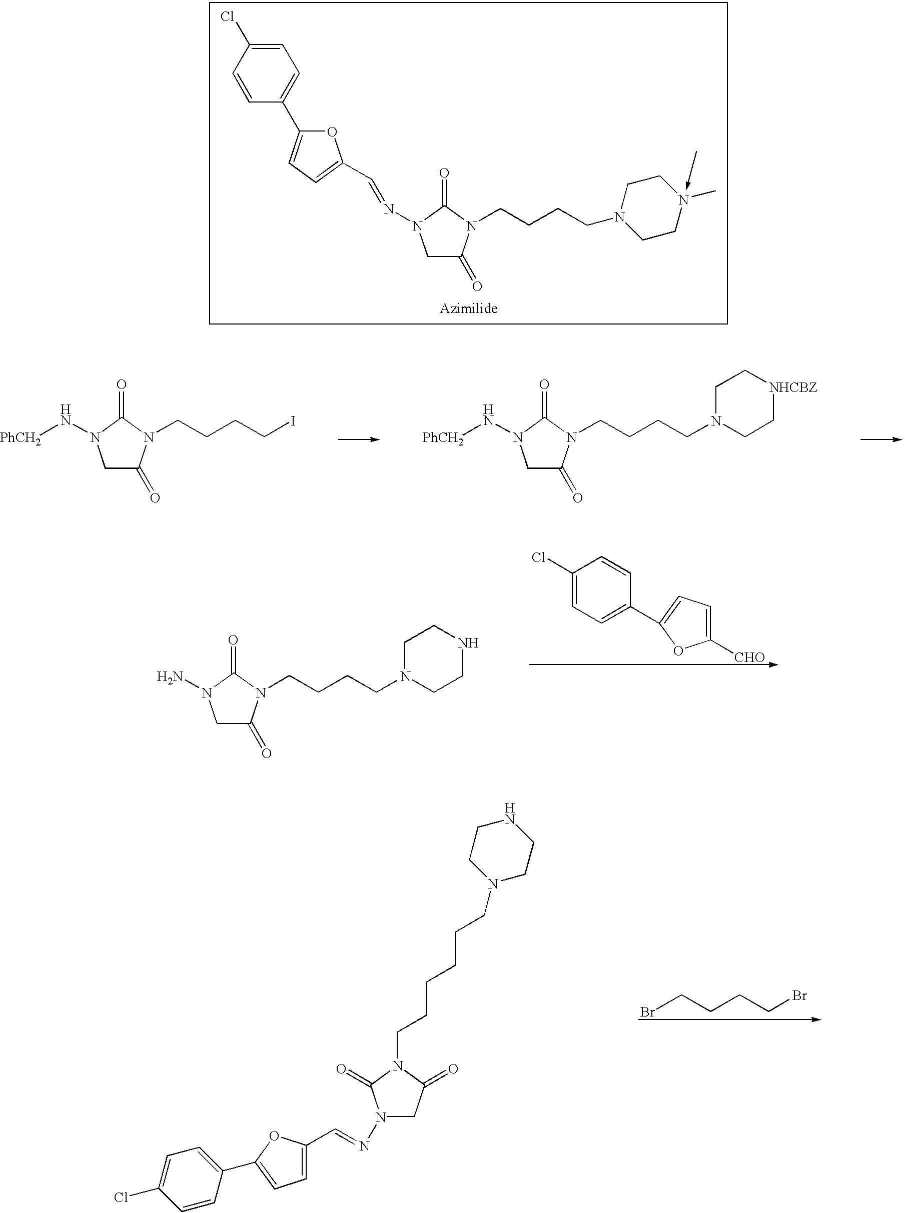

1-Benzylamino-3-(4-iodobutyl)imidazolidine-2,4-dione, prared as described in WO9304061, (5 mmol), diisopropylethylamine (10 mmol) and N-benzyloxycarbonylpiperazine (5 mmol) are dissolved in MeCN (50 mL). The progress of the reaction is monitored by tlc. When it is complete, the mixture is added to water and extracted with CH₂Cl₂. The extract is dried and evaporated, and the residue is chromatographed to afford 1-benzylamino-3-[4-(4-benzyloxycarbonyl piperazinyl)butyl]-imidazoline-2,4-dione.

The above compound (2 mmol) is dissolved in EtOH (25 mL), and to the solution are added 5% Pd/C (100 mg) and ammonium formate (250 mg). The progress of the reaction is monitored by tlc. When it is complete, the mixture is filtered and added to water, then extracted with EtOAc. The extract is dried and evaporated and the residue is chromatographed to afford 1-amino-3-[4(piperazin-1-yl)butyl]imidazoline-2,4-dione.

The above compound (1 mmol) is dissolved in EtOH (20 mL). To the solution is added 5-(4-chlorophenyl)furan-2-carboxaldehyde (1 mmol) and p-toluenesulfonic acid (10 mg). The progress of the reaction is followed by tlc. When it is complete, the mixture is added to water and extracted with CH₂Cl₂. The extract is dried and evaporated to afford 1-[5-(4-chlorophenyl-2-furanylmethyleneamino]-3-[4-(piperazin-1-yl)butyl]imidazolidine-2,4-dione.

A solution of 1,4-dibromobutane (0.5 mmol) and the above compound (1 mmol) in EtOH is maintained at room temperature, while the progress of the reaction is monitored by tlc. When it is complete, the mixture is evaporated to dryness under reduced pressure, and the residue is chromatographed to afford the desired compound.

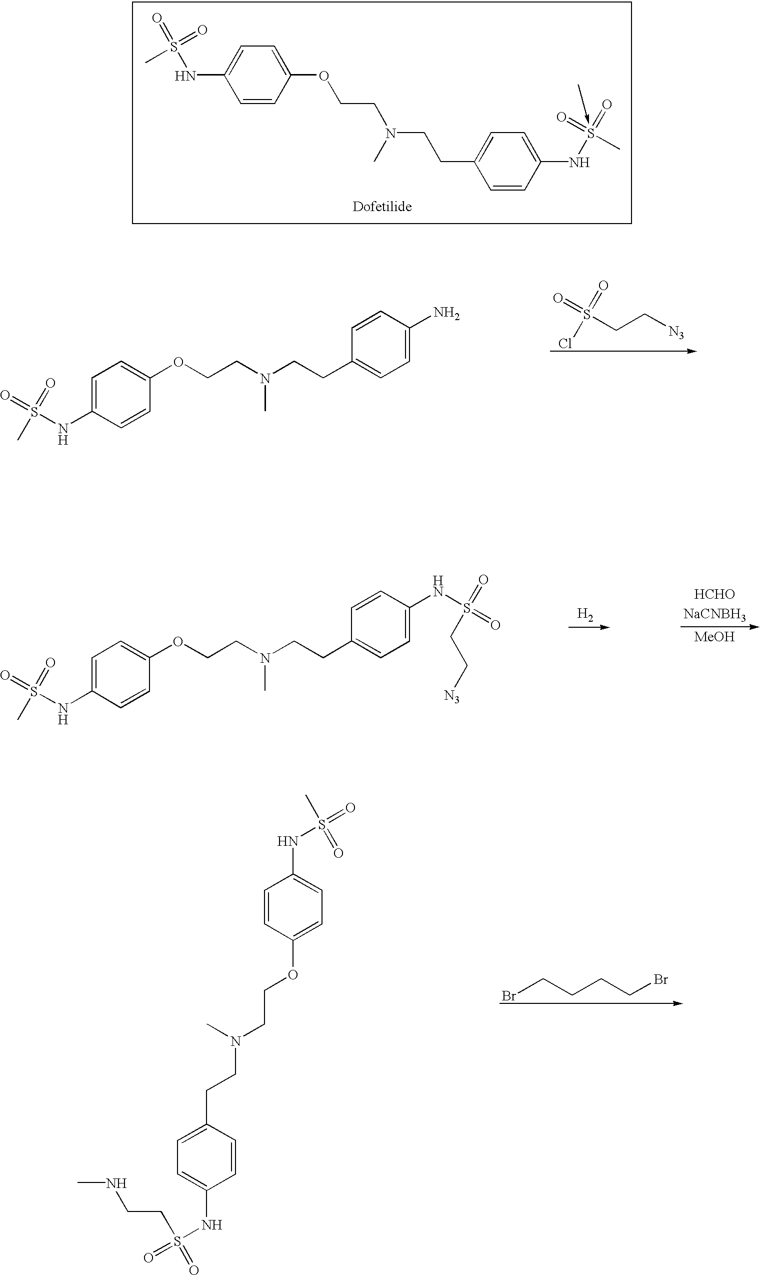

N-methyl N-(4-aminophenylethyl) 2-[4-(methylsulfonylamino)phenoxy] ethylamine, prepared as described in Example A14, (5 mmol) is dissolved in CH₂Cl₂ (25 mL) and 3-azidopropylsulfonylchloride (5 mmol) is added. The reaction is monitored by tlc. When it is complete, the mixture is added to water and extracted with EtOAc. The extract is dried and evaporated; the residue is chromatographed to afford N-methyl N-[4-(3-azidopropylsulfonyl)aminophenylethyl]2-[4-(methylsulfonylamino)phenoxy]-ethylamine .

The above compound (1 mmol) is dissolved in MeOH (20 mL) and 5% Pd/C (50 mg) is so added. The mixture is stirred in a hydrogen atmosphere. The progress of the reaction is followed by tlc. When it is complete, the solution is filtered and the solvent is removed under reduced pressure. The residue is redissolved in MeOH (20 mL) and paraformaldehyde (1 mmol) and sodium cyanoborohydride (1 mmol) are added The reaction is monitored by tlc. When it is complete, the mixture is added to water and extracted with EtOAc. The extract is dried and evaporated and the residue is chromatographed to afford N-methyl N-[4-(3-methylaminopropylsulfonyl)-aminophenylethyl]-2-[4-(methylsulfonylamino)phenoxy]ethylamine.

1,4-Dibromobutane (0.5 mmol) is dissolved in MeCN, and K₂CO₃ (0.5 g) and the above compound (1 mmol) are added. The reaction is monitored by tlc. When it is complete, the mixture is added to water and extracted with EtOAc. The extract is dried and evaporated and the residue is chromatographed to afford the desired compound.

### EXAMPLE A21

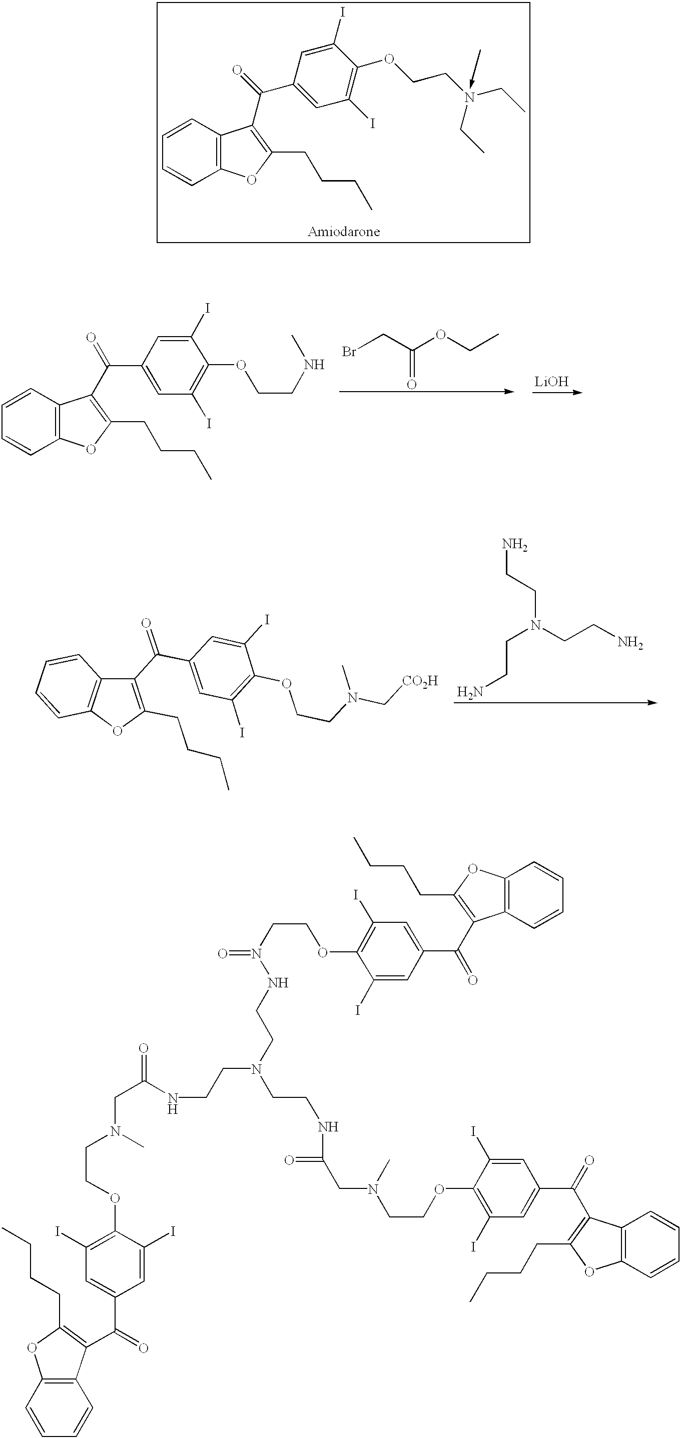

N-Methyl 2-[4-[2-butylbenzofuran-3-ylcarbonyl]-2,6-diiodophenoxy]ethylamine, prepared according to procedures described in Eur. J. Med Chem., 1974, 19-25, (5 mmol) is dissolved in EtOH (25 mL) and ethyl bromoacetate (5 mmol) and diisopropylethylamine (10 mmol) are added. The progress of the reaction is followed by tlc. When it is complete, the reaction is added to water and extracted with EtOAc. The extract is washed with dilute HCl, the dried and the solvent is evaporated under reduced pressure. The residue is dissolved in THF (15 mL), and LiOH, H₂O (5 mmol) is added. The progress of the reaction is followed by tlc. When it is complete, the pH is adjusted to 7 by addition of dilute HCl. The solvents are removed under reduced pressure and the residue is chromatographed to afford N-methyl N-[2-[4-[2-butylbenzofuran-3-ylcarbonyl]-2,6-diodophenoxy]ethyl]glycine.

The above compound (3 mmol) is dissolved in DMF (25 mL) and dicyclohexylcarbodiimide (3 mmol) and tris(2-aminoethyl)amine (1 mmol) are added. The progress of the reaction is monitored by tlc. When it is complete, the mixture is added to water and extracted with EtOAc. The extract is dried and evaporated and the residue is chromatographed to afford the triamide product.

### EXAMPLE A22

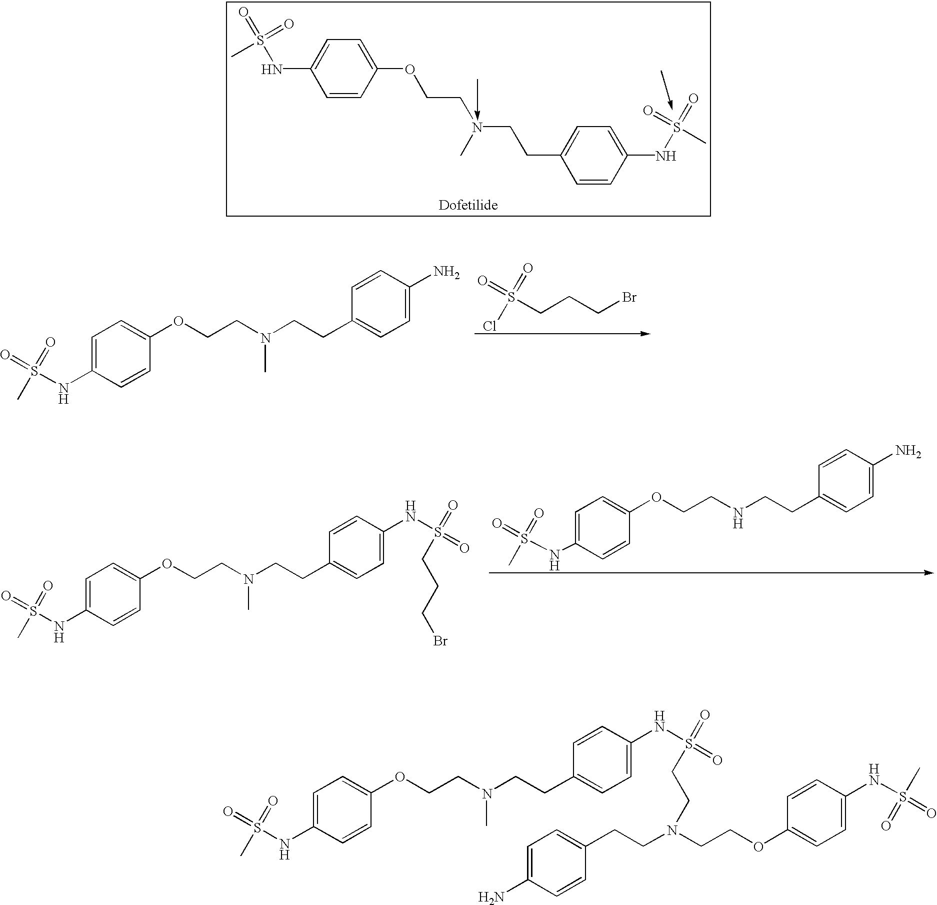

N-Methyl N-(4-aminophenylethyl) 2-[4-(methylsulfonylamino)phenoxy]-ethylamine, the preparation of which is described in Example A14, (2 mmol) is dissolved in dry CH₂Cl₂ (25 mL); diisopropylethylamine (10 mmol) and 3-bromopropanesulfonyl chloride (2 mmol) are added. The progress of the reaction is followed by tlc. When it is complete, the reaction is added to water and extracted with EtOAc. The extract is washed and dried and the solvent is evaporated under reduced pressure. The residue is chromatographed to afford 1 -bromo-3-[4-[N-methyl 2-[2-[4-methylsulfonylamino]phenoxy]ethylamino]phenylaminosulfonyl] propane.

N-2-(4-aminophenyl)ethyl 2-[4-methylsulfonylaminophenoxy]ethylamine, prepared using methods described in EP 338793, (1 mmol) and the above compound (1 mmol) are dissolved in CH₂Cl₂ (25 mL) and to the solution is added diisopropylethylamine (10 mmol) The progress of the reaction is monitored by tlc. When it is complete, the mixture is added to water and extracted with EtOAc. The extract is dried and evaporated and the residue is chromatographed to afford the desired compound.

### EXAMPLE A23

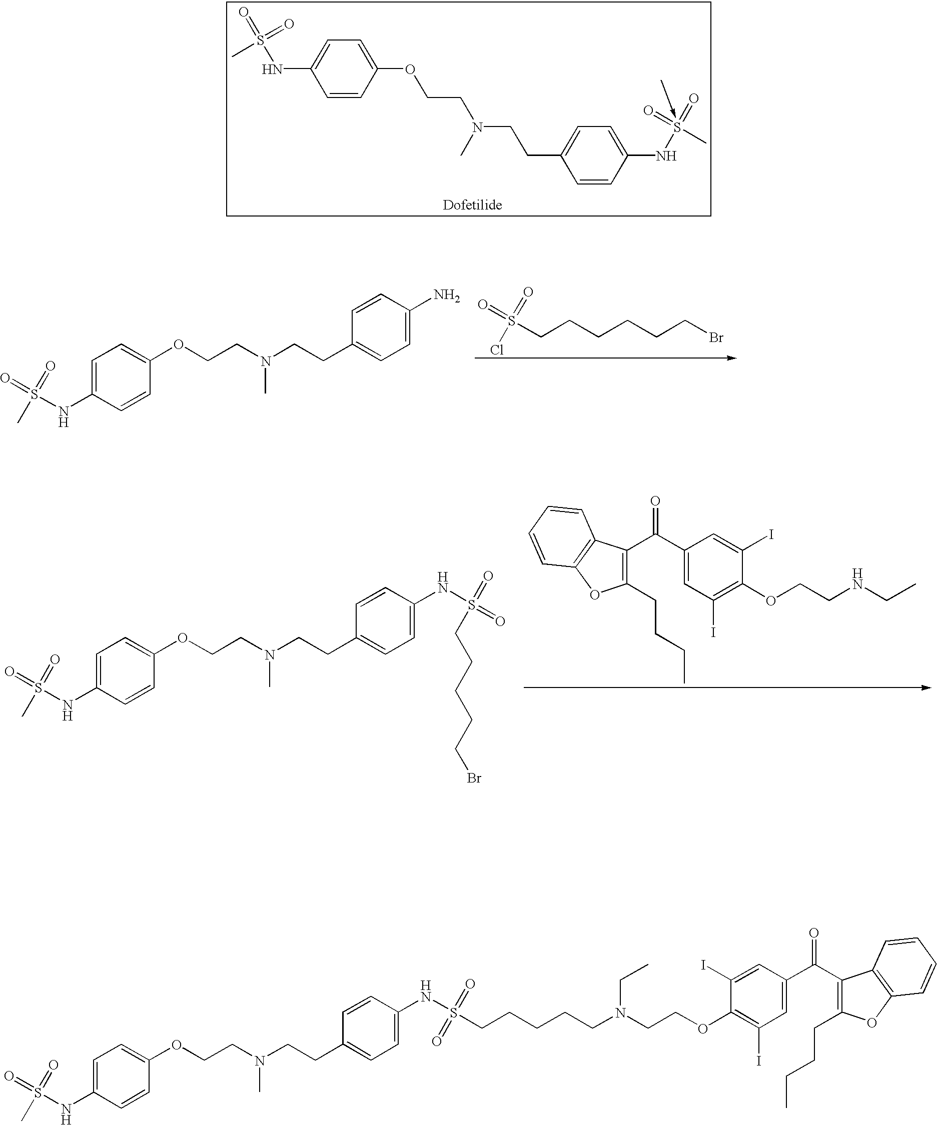

N-Methyl N-(4-aminophenylethyl) 2-[4-(methylsulfonylamino)phenoxy]-ethylamine, the preparation of which is described in Example A14, (2 mmol) is dissolved in dry CH₂Cl₂ (25 mL); diisopropylethylamine (10 mmol) and 6-bromohexanesulfonyl chloride (2 mmol) are added. The progress of the reaction is followed by tlc. When it is complete, the reaction is added to water and extracted with EtOAc. The extract is washed and dried and the solvent is evaporated under reduced pressure. The residue is chromatographed to afford 1-bromo-6-[4-[N-methyl 2-[2-[4-methylsulfonylamino]phenoxy]ethylamino]phenylaminosulfonyl]hexane.

The above compound (2 mmol) and N-ethyl 2-[4-[2-butylbenzofuran-3-ylcarbonyl]-2,6-diiodophenoxy]ethylamine (2 mmol), prepared according to procedures described in Eur. J. Med Chem., 1974, 19-25, are dissolved in MeCN (25 mL). The progress of the reaction is monitored by tlc. When it is complete, the mixture is added to dilute NaHCO₃ and extracted with EtOAc. The extract is dried and evaporated and the residue is chromatographed to afford the desired compound.

### EXAMPLE A24

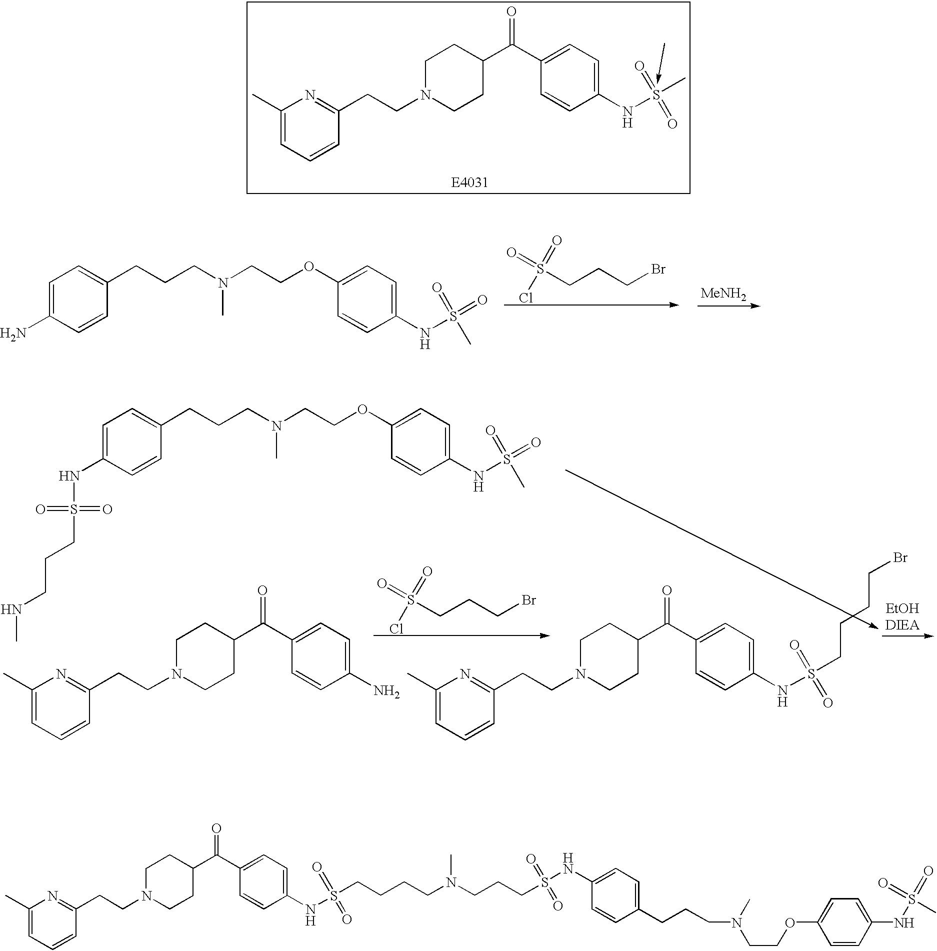

N-Methyl N-(4-aminophenylethyl) 2-[4-(methylsulfonylamino)phenoxy]ethylamine, the preparation of which is described in Example A14, (2 mmol) is dissolved in MeCN (25 mL) and to the solution is added 3-bromopropanesulfonyl chloride (2 mmol). After 6 hours, 10% methanolic methylamine (1 mL) is added. The progress of the reaction is followed by tlc. When it is complete, the mixture is added to water and extracted with CH₂Cl_{2.} The extract is dried and evaporated, and the residue is chromatographed to afford N-methyl N-[4-(3-methylaminopropylsulfonyl)-aminophenylethyl]-2-[4-(methylsulfonylamino)phenoxy]ethylamine.

The next starting material, 6-[2-[4-[4-aminobenzoyl-1 -piperidyl]ethyl]-2-methylpyridine, is prepared as follows: N-[4-[[2-(6-methyl-2-pyridinyl)ethyl]-4-piperidinyl]carbonyl]phenyl methanesulfonamide, prepared as described in J. Med. Chem., 1990, 903, (10 mmol) is dissolved in 48% HBr in AcOH (50 mL). The solution is heated to 60° and the progress of the reaction is monitored by tlc. When it is complete, the mixture is cooled and the solvent is removed under reduced pressure. The residue is taken up in water and the solution is basified with aqueous NaOH. The aqueous solution is extracted with CH₂Cl₂, and the extract is dried and evaporated. The residue is chromatographed to afford 6-[2-[4-[4-aminobenzoyl- 1-piperidyl]ethyl]-2-methylpyridine.

The above compound (2 mmol) is dissolved in CH₂Cl₂ (35 mL) and to the solution are added diilsopropylethylamine (5 mmol) and 4-bromobutanesulfonyl chloride (2 mmol). The progress of the reaction is monitored by tlc. When it is complete the mixture is added to dilute NaHCO₃ and extracted with EtOAc. The extract is dried and evaporated and the residue is chromatographed to afford the desired compound.

The product from the preceding reaction (1 mmol) and the product from the first step (1 mmol) are dissolved in EtOH (20 mL). To the mixture is added diisopropylethylamine (20 mmol). The progress of the reaction is monitored by tlc. When it is complete, the mixture is added to water and extracted with CH₂Cl₂. The extract is dried and evaporated, and the residue is chromatographed to afford the desired compound.

### EXAMPLE A25

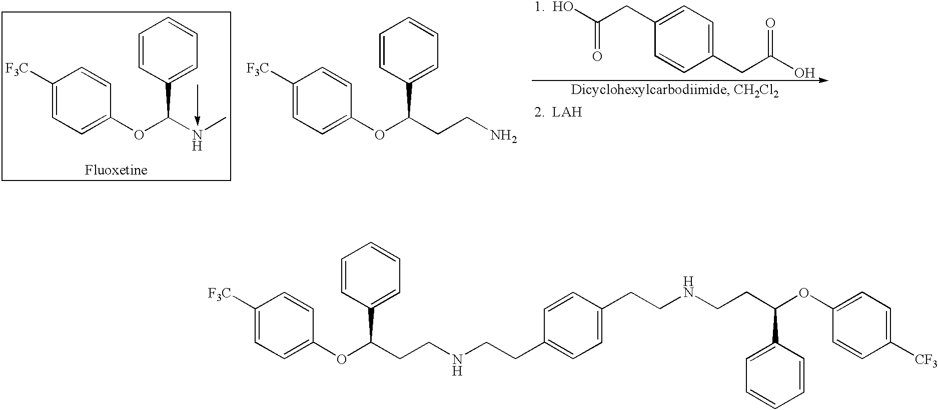

A solution of nor-fluoxetine (2 mmols), which is described in U.S. Pat. No. 4,018,895. and benzene-1,4-bisacetic acid (1 mmol) in methylene chloride is prepared under argon in a flask equipped with magnetic stirrer and drying tube. To this solution is added dicyclohexylcarbodiimide (solid, 2.1 mmols) while stirring at room temperature. The course of the reaction is followed by thin layer chromatography. When reaction has occurred, the reaction solution is diluted with ethyl acetate and washed with water and with aqueous Na₂CO₃. The organic layer is dried (Na₂SO₄), filtered and concentrated under reduced pressure to give the crude product. The desired compound is obtained by purification of the crude product by use of HPLC.

In the second step, lithium aluminum hydride (LAH) (63.3 mmol) is dissolved in THF in an ice bath under nitrogen atmosphere. The above product (12.7 mmol) is dissolved in THF and added dropwise to the LAH/THF solution. The reaction is stirred with cooling, then warmed to room temperature, placed in an oil bath and the temperature is raised by increments of 10° C. to 85° C. over a 30 minute period. The mixture is stirred at reflux for 18 hours, then cooled to room temperature and placed in an ice bath. Sodium sulfate decahydrate is slowly added to quench the excess LAH. The solids are removed by filtration and rinsing with THF. The filtrate is concentrated to a thick syrup and excess solvent removed under vacuum to afford the desired product.

### EXAMPLE A26

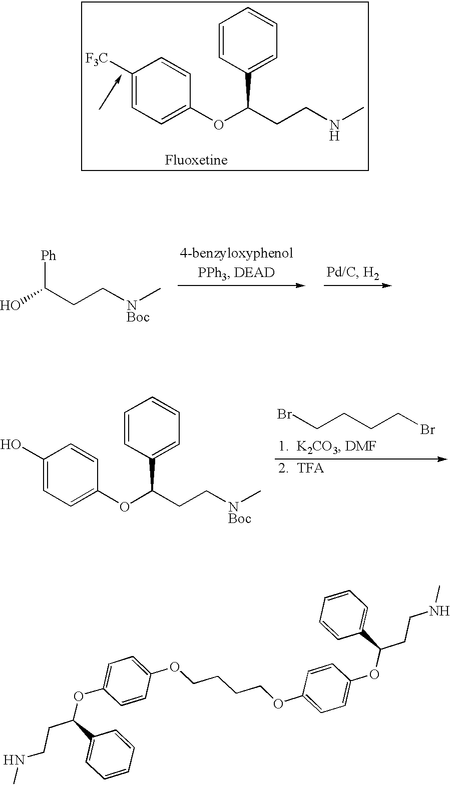

4-Benzyloxyphenol (20 mmol) is added in portions to a solution of the starting compound (20 mmol), which is described in Tetrahedron Lett. 1991, 32, 1901-1904, and triphenylphosphine (20 mmol) in anhydrous THF at 0° C. The mixture is cooled to −20° C. and diethyl azodicarboxylate (DEAD) (24 mmol) is added dropwise over 30 min. During this time, the temperature of the mixture is not allowed to rise above −10° C. When the addition is complete, the mixture is allowed to warm to room temperature and stirred for 16 h. The mixture is concentrated in vacuo and the resulting residue is purified by chromatography to afford the desired product.

A solution of the above product (11.7 mmol) in methanol is hydrogenated overnight at 35 psi, in the presence of 10% Pd/C and 1 ml of conc. HCI. The reaction mixture is filtered and the filtrate is concentrated to dryness. The resulting residue is taken into dichloromethane and washed with saturated NaHCO₃ solution, dried over MgSO₄ and concentrated. The crude product is purified by chromatography to afford the product.

A solution of 20 mmols of the above product, N-Boc-4-hydroxyfluoxetine, in DMF with 10 mmols of 1,4-dibromobutane and 20 mmols of potassium carbonate is heated as necessary and the reaction followed by TLC. When judged complete, the mixture is partitioned between ethyl acetate and water and the organic phase washed with water, dried over sodium sulfate and the solvent removed in vacua. The residue is purified by chromatography to afford the desired product. In the third step, the product (1 mmol) is dissolved in CH₂Cl₂. A solution of 10% trifluoroacetic acid in CH₂Cl₂ is added and the reaction is stirred for 1 hour at room temperature. The solvent is then removed in vacuo to provide the desired material as the TFA salt. The desired material is then purified from this mixture using HPLC to afford the desired product.

### EXAMPLE A27

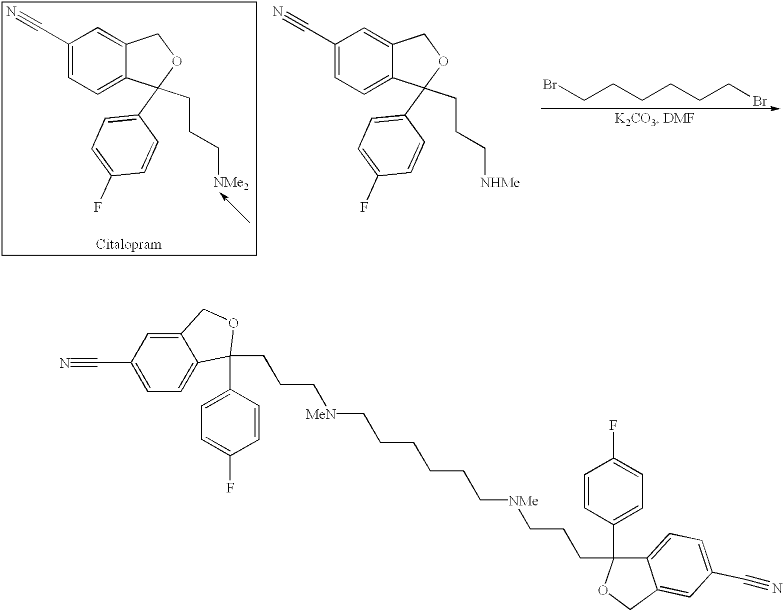

A solution of 20 mmols of nor-citalopram, which is described in Eur. J. Med. Chem. 1977, 12, 289-95, in DMF with 10 mmols of 1,6-dibromohexane and 20 mmols of potassium carbonate is heated as necessary and the reaction followed by TLC. When judged complete, the mixture is partitioned between ethyl acetate and water and the organic phase washed with water, dried over sodium sulfate and the solvent removed in vacuo. The residue is purified by chromatography to afford the title structure.

### EXAMPLE A28

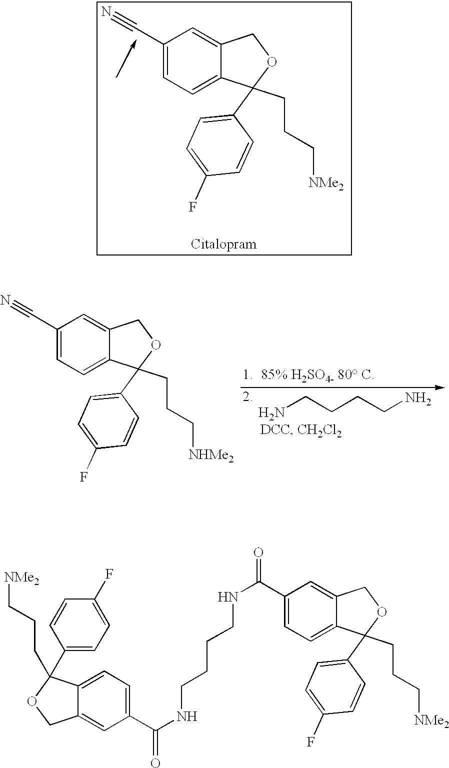

A solution of citalopram which is described in DE 02657013, in 85% H₂SO₄ is heated to 80° C. The course of the reaction is followed by TLC. The reaction is poured onto ice and the solution is neutralized to pH 7 and extracted with chloroform. The residue is purified by chromatography to afford the title product.

A solution of 5-carboxycitalopram (2 mmol), prepared as described above, and 1,4-diaminobutane (1 mmol) in methylene chloride is prepared under argon in a flask equipped with a magnetic stirrer and drying tube. To this solution is added dicyclohexylcarbodiimide (2.1 mmol) while stirring at room temperature. The course of the reaction is followed by TLC. When judged complete, the reaction solution is diluted with ethyl acetate and washed with water and with aqueous Na₂CO₃. The organic layer is dried Na₂SO₄, filtered and concentrated under reduced pressure to give the crude product. The desired compound is obtained by purification of the crude product by use of HPLC.

### EXAMPLE A29

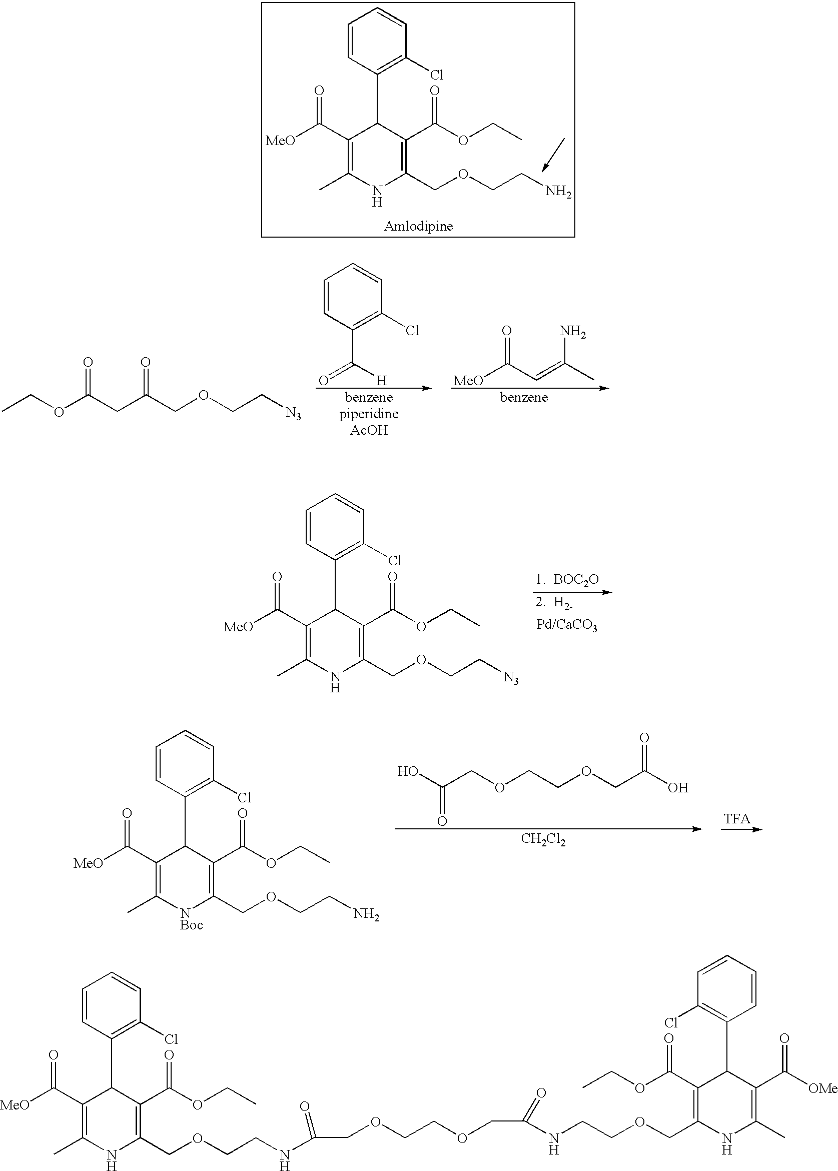

A solution of N—BOC-amlopidine (2 mmol), prepared in the first three steps shown by known technique, 3,6-dioxaoctanedioic acid (1 mmol) in CH₂Cl₂ (5 mL) is prepared under argon in a flask equipped with magnetic stirrer and a drying tube. To this solution is added dicyclohexylcarbodiimide (solid, 2.2 mmol). The progress of the reaction is followed by tlc and after reaction occurs, the reaction solution is quenched in water, aqueous sodium bicarbonate is added and the aqueous mixture is extracted with methylene chloride. The organic layer is washed with aqueous Na₂CO₃ and with H₂O, dried (Na₂SO₄), filtered and concentrated under reduced pressure to give the crude product. The desired compound is obtained by purification of the crude product with the use of HPLC.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO₄), filtered and concentrated under reduced pressure to give the crude product. The desired compound is obtained by purification of the crude product with the use of HPLC.

### EXAMPLE A30

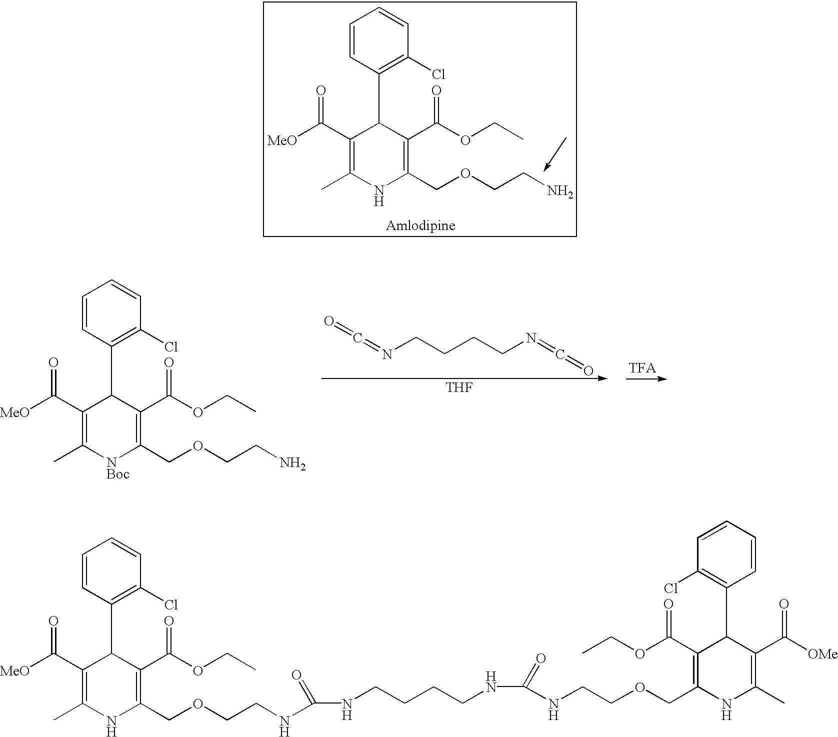

A solution of 1,4-diisocyanatobutane (1 mmol) in CH₂Cl₂ (5 mL) containing Et₃N (0.2 mL) is stirred and cooled in an ice-water bath under an inert atmosphere. To this is added dropwise a solution of N—Boc amlodipine (2 mmol) in CH₂Cl₂ (5 mL). After addition is complete, the cooling bath is removed and the reaction solution is allowed to warm to room temperature. The progress of the reaction is followed by tlc and when reaction has occurred, the reaction solution is quenched in cold 5% aqueous Na₂CO₃. The layers are separated and the organic layer is washed with aqueous Na₂CO₃, with water and is dried (Na₂SO₄), filtered and concentrated under reduced pressure to give the crude product. The desired compound is obtained by purification of the crude product with the use of HPLC.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO₄), filtered and concentrated under reduced pressure to give the crude product. The desired compound is obtained by purification of the crude product with the use of HPLC.

### EXAMPLE A31

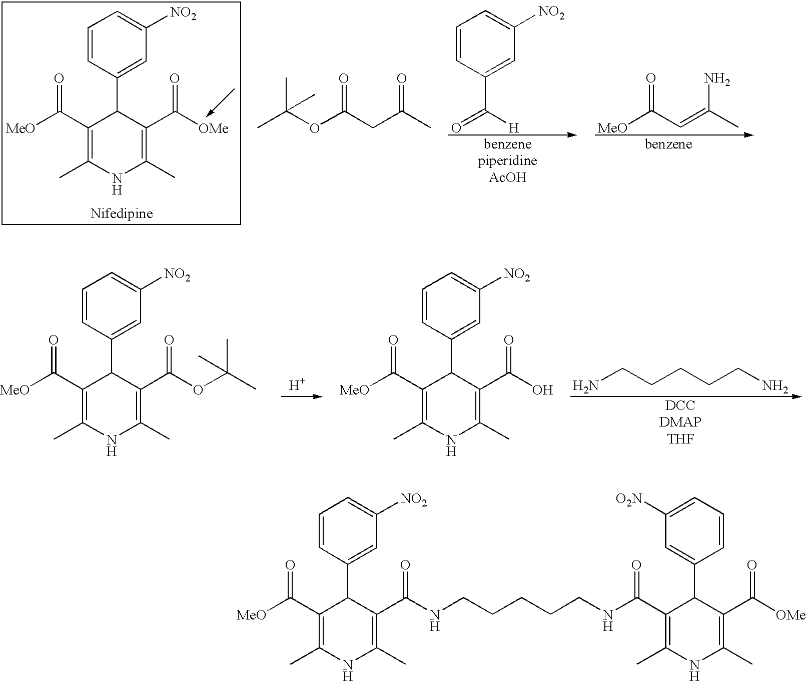

A solution of the dihydropyridine (2 mmol) (prepared as shown in the first three steps by known technique), 1,5-diaminopentane (1 mmol), and 4-dimethylaminopyridine (10 mg) in CH₂Cl₂ (5 mL) is prepared under argon in a flask equipped with magnetic stirrer and a drying tube. To this solution is added dicyclohexylcarbodiimide (solid, 2.2 mmol). The progress of the reaction is followed by tic and after reaction occurs, the reaction solution is quenched in water, aqueous sodium bicarbonate is added and the aqueous mixture is extracted with methylene chloride. The organic layer is washed with aqueous Na₂CO₃ and with H₂O, dried (Na₂SO₄), filtered and concentrated under reduced pressure to give the crude product. The desired compound is obtained by purification of the crude product with the use of HPLC.

### EXAMPLE A32

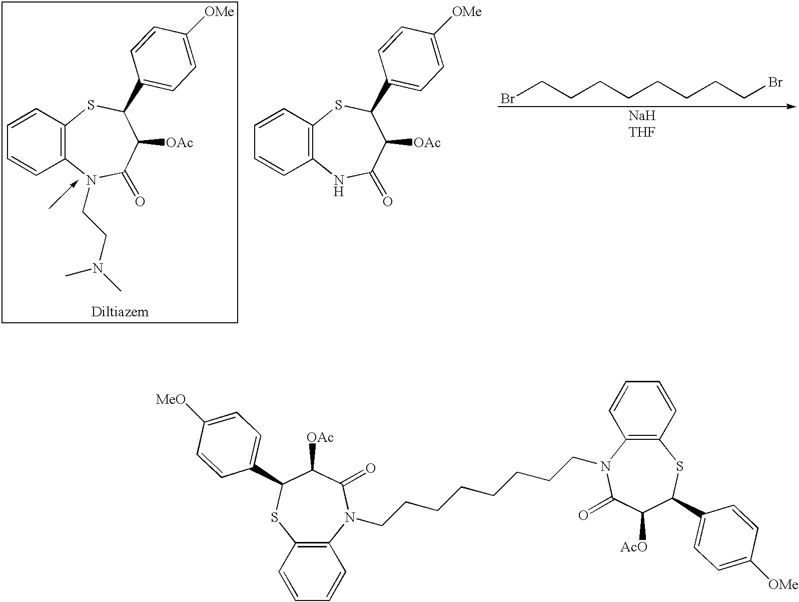

A mixture of NaH (2.1 mmol) and DMF (1 mL) is prepared under an inert atmosphere in a flask equipped with a stirring bar and a drying tube. To this is added first a solution of the benzothiazepine (CAS Reg# 87447-47-0) (2 mmol) in DMF (5 mL) and then 1,8-dibromooctane (1 mmol). The resulting mixture is stirred and the course of the reaction is followed by thin layer chromatography. After reaction occurs, the reaction is quenched with cold dilute aq. Na₂CO₃ and extracted with methylene chloride. The organic layer is dried (Na₂SO₄), filtered and concentrated under reduced pressure to give the crude product. The desired compound is obtained by purification of the crude product by use of HPLC.

### EXAMPLE A33

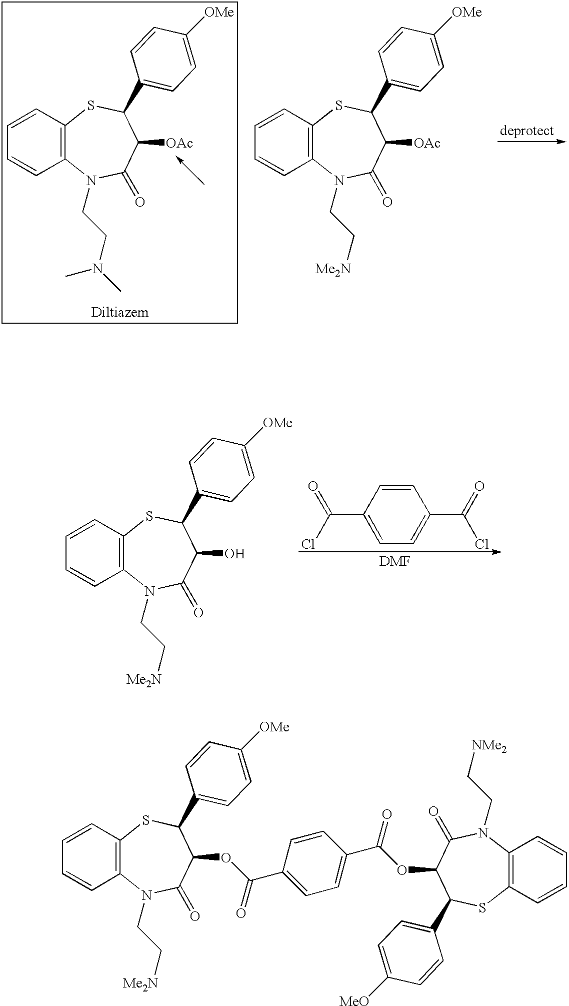

A solution of diltiazem (1 mmol) in methanol (5 mL) is stirred with potassium carbonate. The progress of the reaction is followed by tlc. After reaction occurs, the mixture is filtered to remove solids and the filtrate is concentrated to give the crude product. The desired compound is obtained by purification of the crude product with the use of HPLC.

A solution of benzene-1,4-bisacetyl chloride (1 mmol) in methylene chloride is added slowly to a solution of the compound above (2 mmols) in methylene chloride (5 mL) and pyridine (0.5 mL) in a flask equipped with a magnetic stirrer and a drying tube and which is cooled in an ice-water bath. The course of the reaction is followed by thin layer chromatography. After reaction occurs, the reaction solution is quenched in water and the aqueous mixture is extracted with ethyl acetate. The organic layer is washed with aqueous Na₂CO₃ with water, and is dried (Na₂SO₄), filtered and concentrated under reduced pressure to give the crude product. The desired compound is obtained by purification of the crude product by use of HPLC.

### EXAMPLE A34

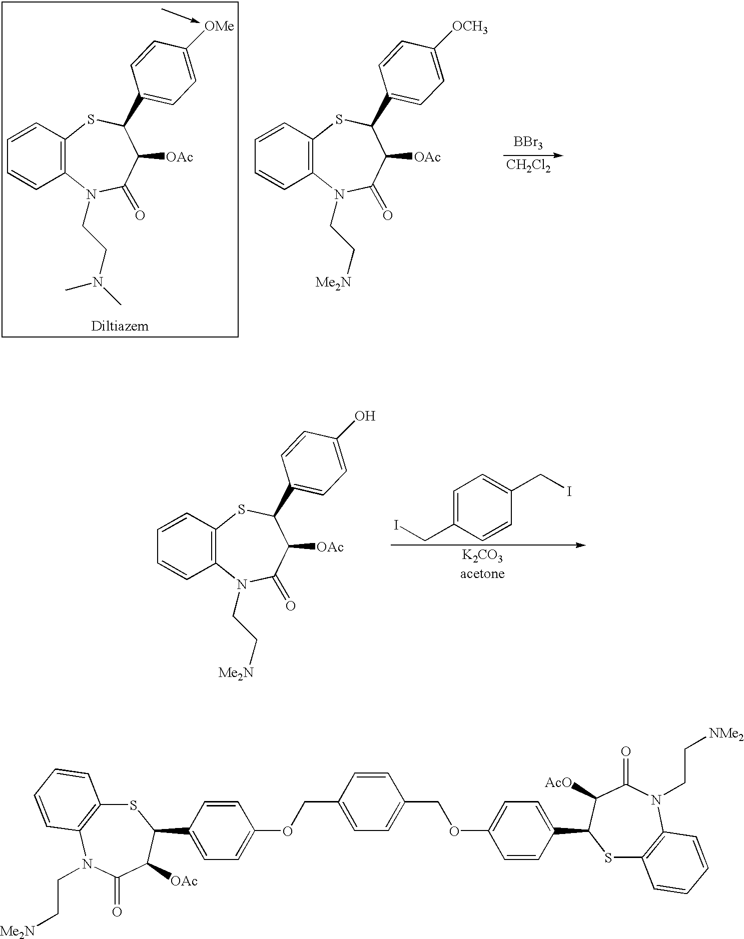

A solution of diltiazem (2 mmols) in methylene chloride (5 mL) is stirred and cooled to −78° C. under and inert atmosphere. BBr₃ (5 mmol) is added and stirring is continued as the cooling bath is removed and the temperature of the reaction solution is allowed to rise to room temperature. The course of the reaction is followed by thin layer chromatography. After reaction occurs, the reaction solution is diluted with methylene chloride and washed with cold aqueous Na₂CO₃ and with half-saturated brine. The organic layer is dried (Na₂SO₄), filtered and concentrated under reduced pressure to give the crude product. The desired compound is obtained by purification of the crude product by use of HPLC.

A solution of the compound above (2 mmols) and 1,4-bisiodomethylbenzene (1 mmol) in acetone (5 mL) containing K₂CO₃ is stirred and heated at reflux temperature under an inert atmosphere. The course of the reaction is followed by thin layer chromatography. After reaction occurs, the reaction solution is diluted with ethyl acetate and washed with water and with aqueous Na₂CO₃. The organic layer is dried (Na₂SO₄), filtered and concentrated under reduced pressure to give the crude product. The desired compound is obtained by purification of the crude product by use of HPLC.

### EXAMPLE A35

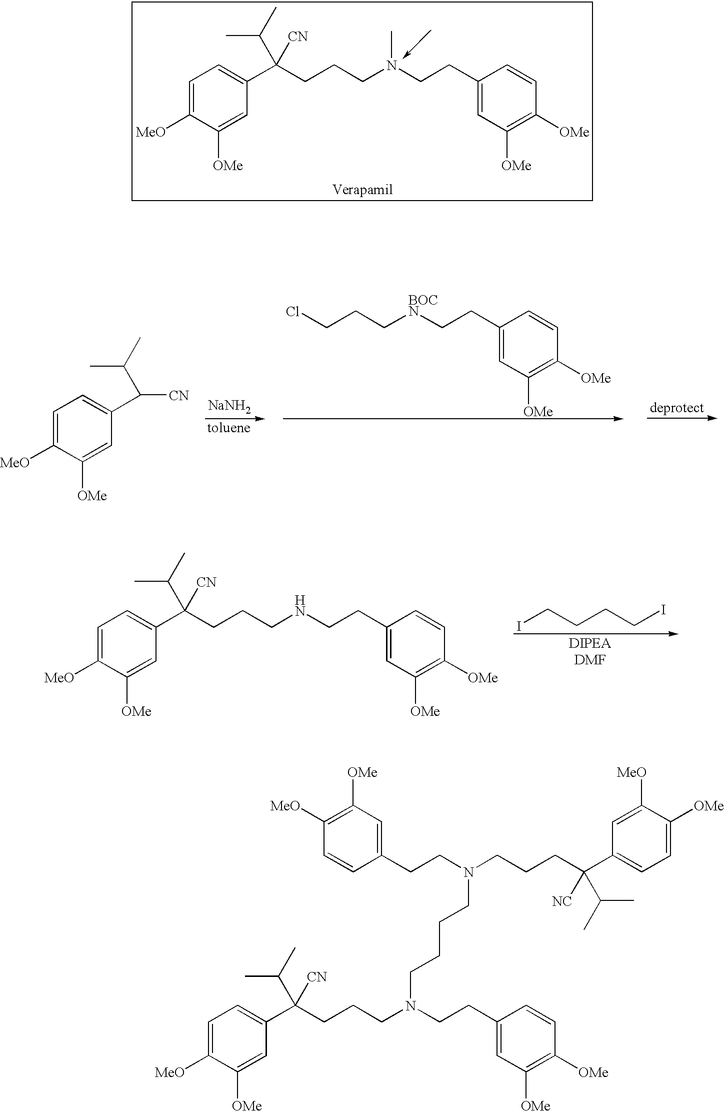

A solution of N-desmethyl-verapamil (2 mmol) (prepared as shown in the first three steps by known technique), 1,4-diiodobutane (1 mmol), and diisopropylethylamine (0.2 mL) in DMF (3 mL) is stirred and warmed under an inert atmosphere. The progress of the reaction is followed by tlc and when reaction is complete, the solution is poured into aqueous 5% NaHCO₃ and the aqueous mixture is extracted with methylene chloride. The organic extract solution is dried (Na₂SO₄), filtered and concentrated under reduced pressure to give the crude product. The desired compound is obtained by purification of the crude product by use of HPLC.

### EXAMPLE A36

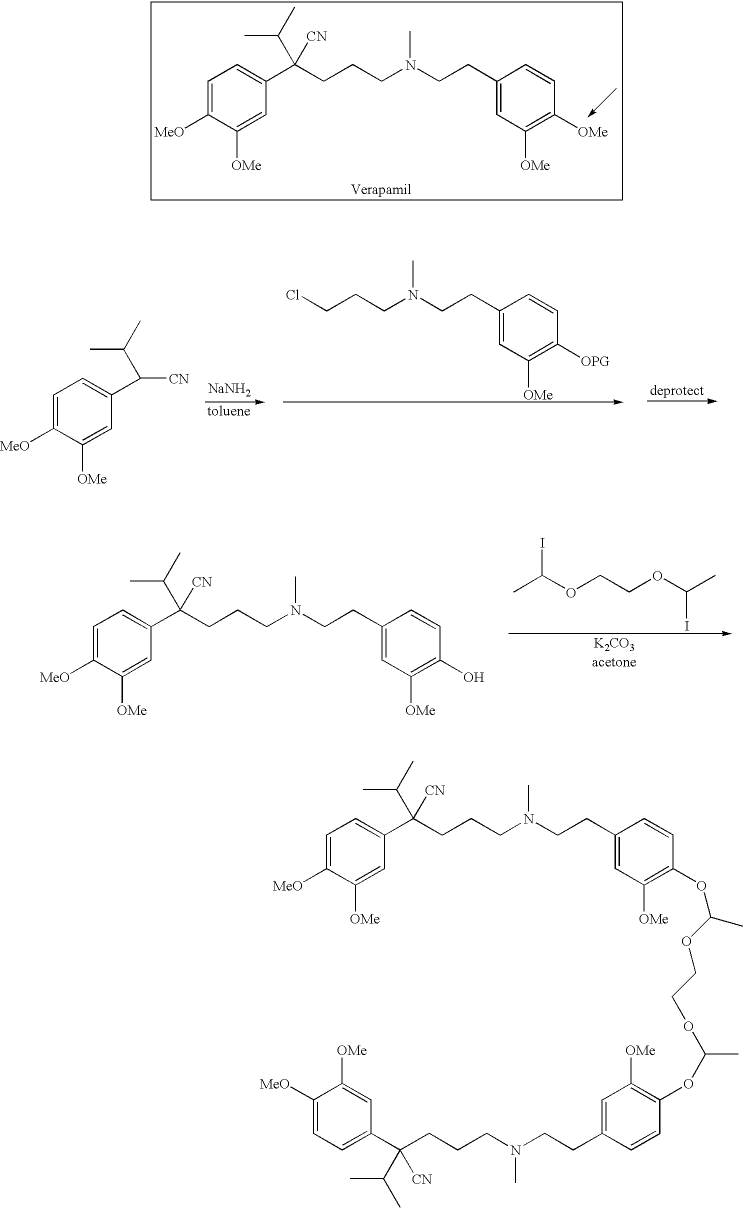

A solution of O-desmethyl-verapamil (2 mmols) (prepared as shown in the first 3 steps by known technique) and 1,2-bis-(2-iodoethoxy)ethane (1 mmol) in acetone (5 mL) containing K₂CO₃ is stirred and heated at reflux temperature under an inert atmosphere. The course of the reaction is followed by thin layer chromatography. After reaction occurs, the reaction solution is diluted with ethyl acetate and washed with water and with aqueous Na₂CO₃. The organic layer is dried (Na₂SO₄), filtered and concentrated under reduced pressure to give the crude product. The desired compound is obtained by purification of the crude product by use of HPLC.

### EXAMPLE A37

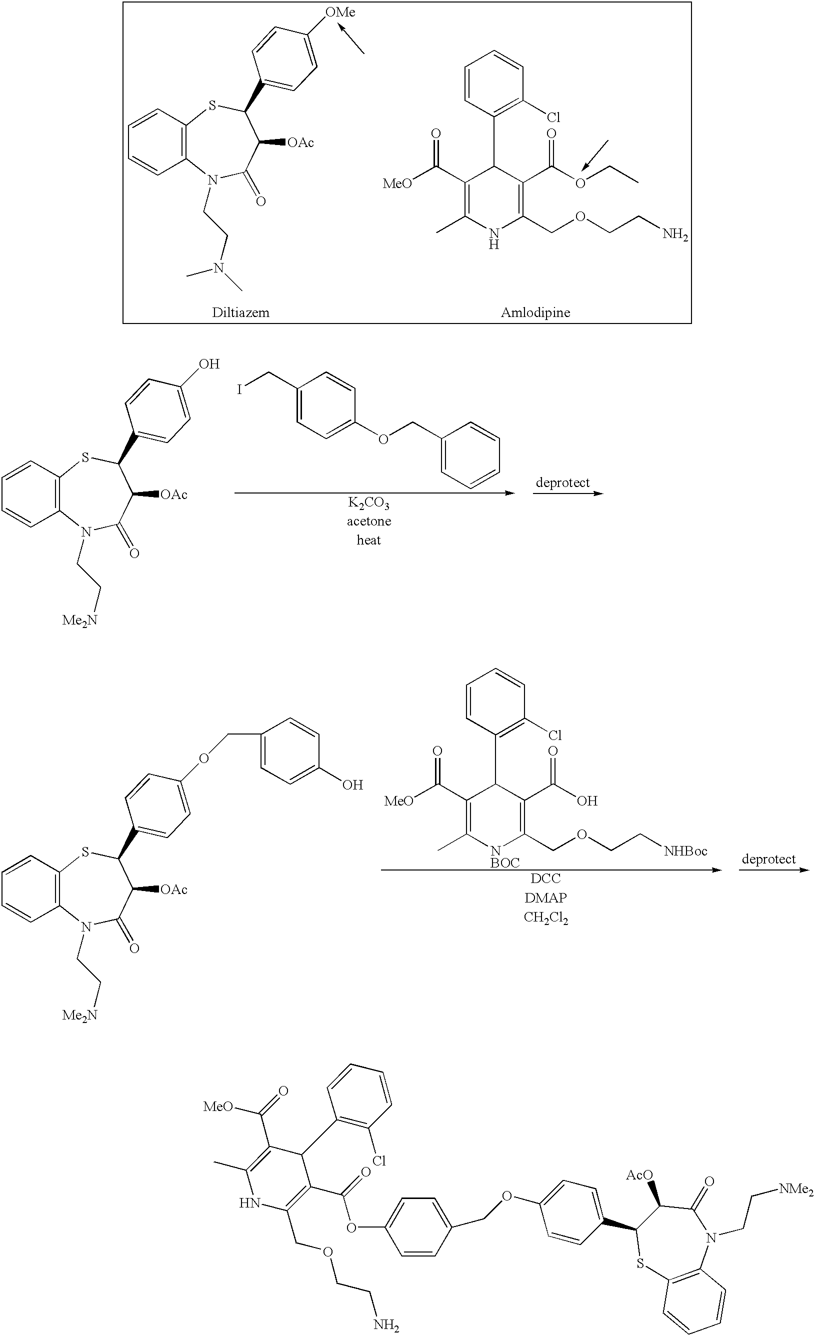

A solution of the benzothiazepine (CAS Reg# 84903-78-6) (1 mmols) and 1-isodomethyl-4-benzyloxybenzene (1 mmol) in acetone (5 mL) containing K₂CO₃ is stirred and heated at reflux temperature under an inert atmosphere. The course of the reaction is followed by thin layer chromatography. After reaction occurs, the reaction solution is diluted with ethyl acetate and washed with water and with aqueous Na₂CO₃. The organic layer is dried (Na₂SO₄), filtered and concentrated under reduced pressure to give the crude product. The desired compound is obtained by purification of the crude product by use of HPLC.

Ammonium formate (96 mg, 1.5 mmol) and 10% Pd-C (50 mg) are added to a solution of the compound obtained in the preceding reaction in methanol (3 mL) and THF (2 mL). The mixture is stirred at room temperature and the progress of the reaction is monitored by tlc. After reaction is complete, the mixture is filtered through Celite, the filter pad is rinsed with EtOAc, the combined organic layers are washed successively with aq. NaHCO₃ and with half-saturated brine, then filtered and concentrated under reduced pressure to give the crude product. The desired compound is obtained by purification of the crude product with the use of HPLC.

A solution of N,N′-di-BOC-1,4-dihydropyridine (1 mmol), the compound (1 mmol) obtained in the preceding reaction (1 mmol), and 4-dimethylaminopyridine (10 mg) in CH₂Cl₂ (5 mL) is prepared under argon in a flask equipped with magnetic stirrer and a drying tube. To this solution is added dicyclohexylcarbodiimide (solid, 2.2 mmol). The progress of the reaction is followed by tic and after reaction occurs, the reaction solution is quenched in water, aqueous sodium bicarbonate is added and the aqueous mixture is extracted with methylene chloride. The organic layer is washed with aqueous Na₂CO₃ and with H₂O, dried (Na₂SO₄), filtered and concentrated under reduced pressure to give the crude product. The desired compound is obtained by purification of the crude product with the use of HPLC.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO4), filtered and concentrated under reduced pressure to give the crude product. The desired Formula I compound is obtained by purification of the crude product with the use of HPLC.

### EXAMPLE A38

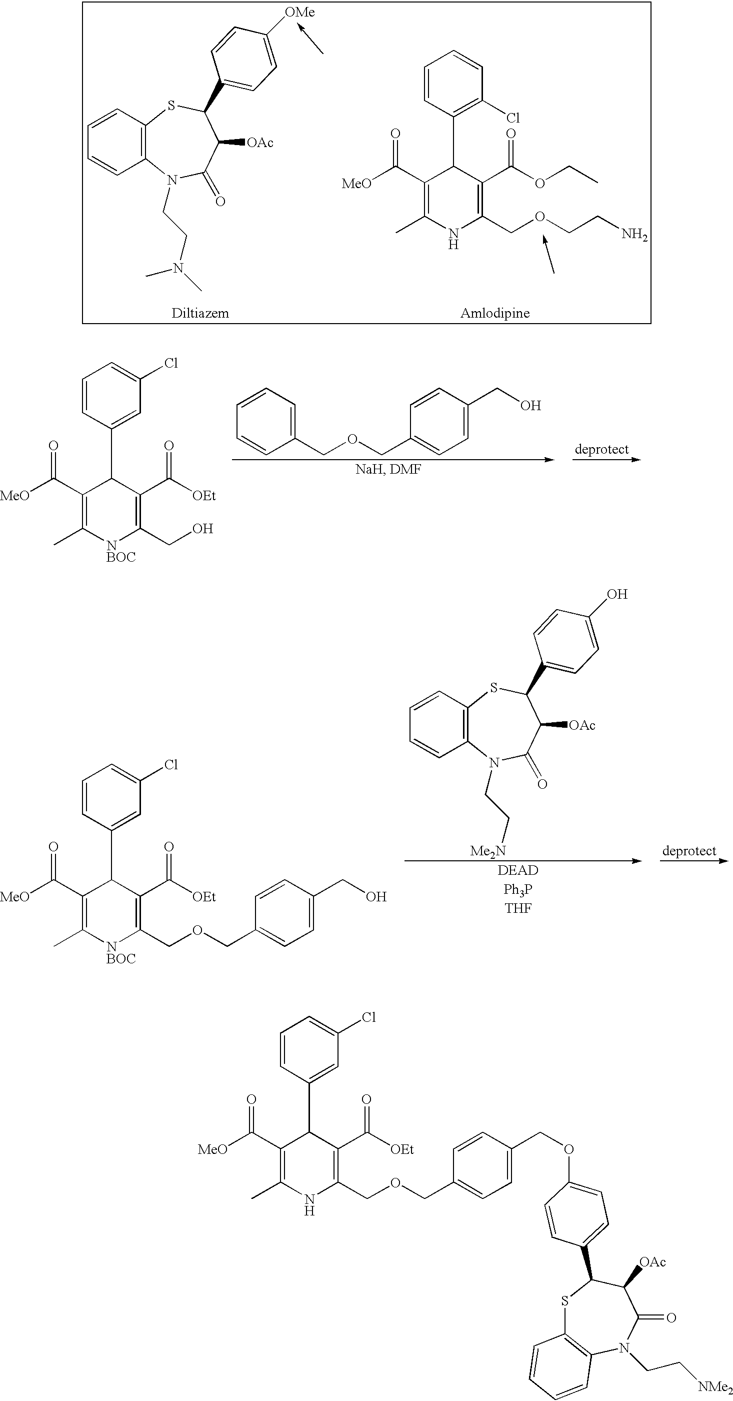

A mixture of NaH (1.1 mmol) and DMF (1 mL) is prepared under an inert atmosphere in a flask equipped with a stirring bar and a drying tube. To this is added a solution of the 1-hydroxymethyl-4-benzyloxybenzene (1 mmol) in dry DMF (5 mL) and the resulting mixture is stirred for 1 hour. Then a solution of the N—BOC-1,4-dihydropyridine (1 mmol) in dry DMF (2 mL) is added. The resulting mixture is stirred and the course of the reaction is followed by thin layer chromatography. After reaction occurs, the reaction is quenched with cold dilute aq. Na₂CO₃ and extracted with methylene chloride. The organic layer is dried (Na₂SO₄), filtered and concentrated under reduced pressure to give the crude product. The desired compound is obtained by purification of the crude product by use of HPLC.

Ammonium formate (96 mg, 1.5 mmol) and 10% Pd-C (50 mg) are added to a solution of the compound obtained in the preceding reaction in methanol (3 mL) and THF (2 mL). The mixture is stirred at room temperature and the progress of the reaction is monitored by tlc. After reaction is complete, the mixture is filtered through Celite, the filter pad is rinsed with EtOAc, the combined organic layers are washed successively with aq. NaHCO₃ and with half-saturated brine, then filtered and concentrated under reduced pressure to give the crude product. The desired compound is obtained by purification of the crude product with the use of HPLC.

Diethyl azodicarboxylate (1 mmol) is added dropwise via a syringe to a stirred solution of triphenylphosphine (1 mmol) in THF (5 mL) at room temperature. To this is added a solution of the compound obtained in the preceding reaction (1 mmol) and the benzothiazepine (CAS Reg# 84903-78-6) (1 mmol) in THF (3 mL). The resulting solution is stirred at RT and the progress of the reaction is followed by tlc. After reaction occurs, solvent is removed by evaporation under reduced pressure and the residue is purified by HPLC, giving the desired compound.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO₄), filtered and concentrated under reduced pressure to give the crude product. The desired compound is obtained by purification of the crude product with the use of HPLC.

### EXAMPLE A39

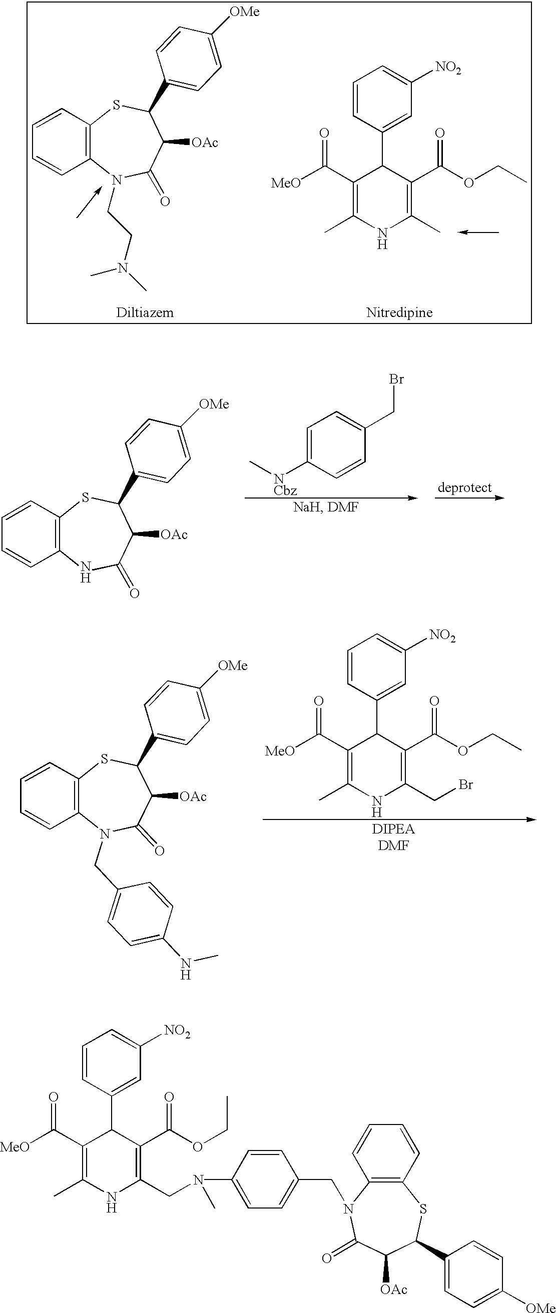

A mixture of NaH (1.1 mmol) and DMF (1 mL) is prepared under an inert atmosphere in a flask equipped with a stirring bar and a drying tube. To this is first added a solution of the benzothiazepine (1 mmol) in dry DMF (3 mL) followed by the 1-bromomethyl-4-(N-Cbz-N-methyl)aminobenzene (1 mmol) in dry DMF (1 mL). The resulting mixture is stirred and the course of the reaction is followed by thin layer chromatography. After reaction occurs, the reaction is quenched with cold dilute aq. Na₂CO₃ and extracted with methylene chloride. The organic layer is dried (Na₂SO₄), filtered and concentrated under reduced pressure to give the crude product. The desired compound is obtained by purification of the crude product by use of HPLC.

Ammonium formate (96 mg, 1.5 mmol) and 10% Pd—C (50 mg) are added to a solution of the compound obtained in the preceding reaction in methanol (3 mL) and THF (2 mL). The mixture is stirred at room temperature and the progress of the reaction is monitored by tlc. After reaction is complete, the mixture is filtered through Celite, the filter pad is rinsed with EtOAc, the combined organic layers are washed successively with aq. NaHCO₃ and with half-saturated brine, then filtered and concentrated under reduced pressure to give the crude product. The desired compound is obtained by purification of the crude product with the use of HPLC.

A solution of the compound from the preceding reaction (1 mmol) and the 1,4-dihydropyridine (described in Alker, D.; Swanson, A. G. Tetrahedron Lett. 1990, 31, 1479-1482) (1 mmol) and diisopropylethylamine (0.2 mL) in DMF (3 mL) is stirred and warmed under an inert atmosphere. The progress of the reaction is followed by tlc and when reaction is complete, the solution is poured into aqueous 5% NaHCO₃ and the aqueous mixture is extracted with methylene chloride. The organic extract solution is dried (Na₂SO₄), filtered and concentrated under reduced pressure to give the crude product. The desired compound is obtained by purification of the crude product by use of HPLC.

### EXAMPLE A40

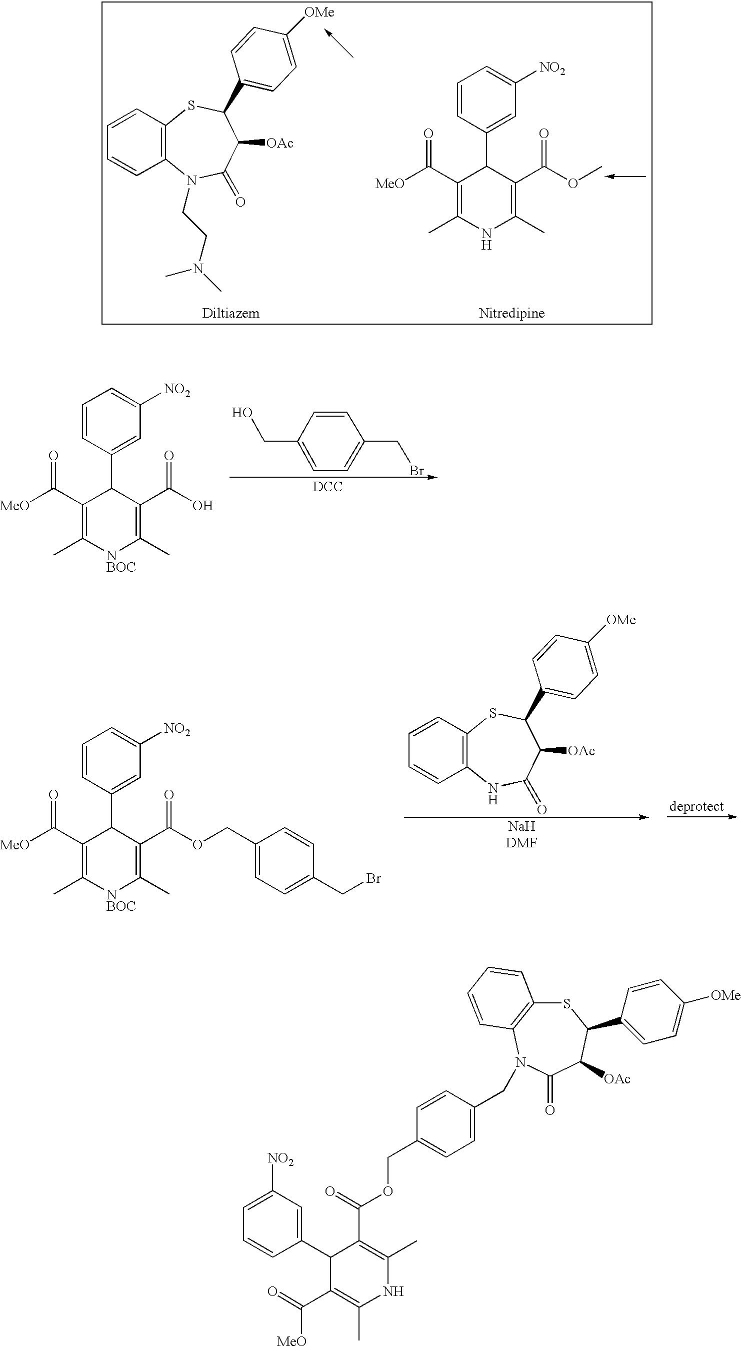

A solution of N—BOC-1,4-dihydropyridine (1 mmol), 1-hydroxymethyl-4-bromomethylbenzene (1 mmol), and 4-dimethylaminopyridine (10 mg) in CH₂Cl₂ (5 mL) is prepared under argon in a flask equipped with magnetic stirrer and a drying tube. To this solution is added dicyclohexylcarbodiimide (solid, 2.2 mmol). The progress of the reaction is followed by tic and after reaction occurs, the reaction solution is quenched in water aqueous sodium bicarbonate is added and the aqueous mixture is extracted with methylene chloride. The organic layer is washed with aqueous Na₂CO₃ and with H₂O, dried (Na₂SO₄), filtered and concentrated under reduced pressure to give the crude product. The desired compound is obtained by purification of the crude product with the use of HPLC.

A mixture of NaH (1.1 mmol) and DMF (1 mL) is prepared under an inert atmosphere in a flask equipped with a stirring bar and a drying tube. To this is added a solution of the benzothiazepine (1 mmol) in dry DMF (3 mL) followed by a solution of the N—BOC-1,4-dihydropyridine (1 mmol) in DMF (3 mL). The resulting mixture is stirred and the course of the reaction is followed by thin layer chromatography. After reaction occurs, the reaction is quenched with cold dilute aq. Na₂CO₃ and extracted with methylene chloride. The organic layer is dried (Na₂SO4), filtered and concentrated under reduced pressure to give the crude product. The desired compound is obtained by purification of the crude product by use of HPLC.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO₄), filtered and concentrated under reduced pressure to give the crude product. The desired compound is obtained by purification of the crude product with the use of HPLC.

### EXAMPLE A41

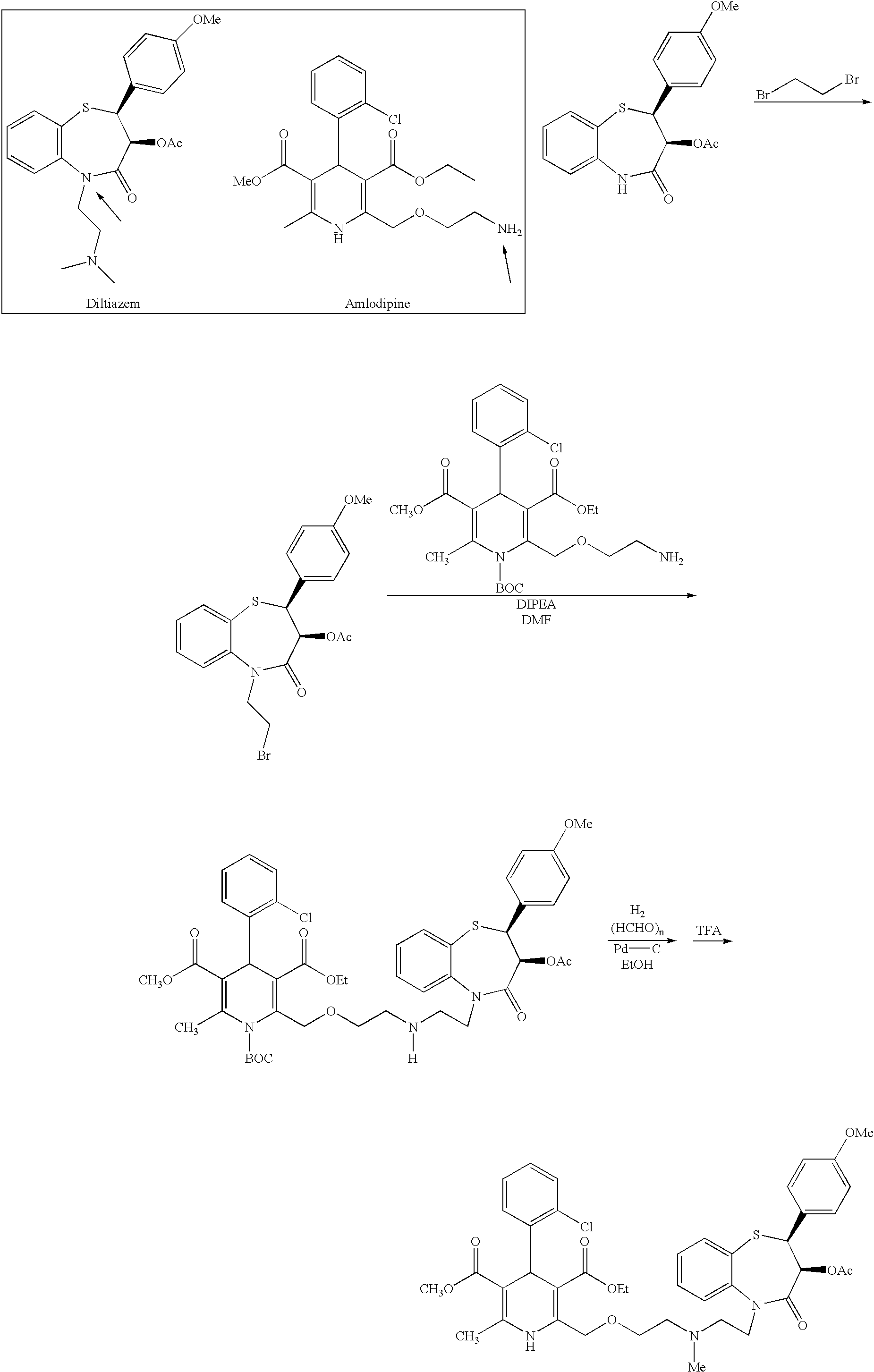

A mixture of NaH (1.1 mmol) and DMF (1 mL) is prepared under an inert atmosphere in a flask equipped with a stirring bar and a drying tube. To this is added a solution of the benzothiazepine (1 mmol) in dry DMF (3 mL) followed by 1,2-dibromoethane (10 mmol). The resulting mixture is stirred and the course of the reaction is followed by thin layer chromatography. After reaction occurs, the reaction is quenched with cold dilute aq. Na₂CO₃ and extracted with methylene chloride. The organic layer is dried (Na₂SO₄), filtered and concentrated under reduced pressure to give the crude product. The desired compound is obtained by purification of the crude product by use of HPLC.

A solution of the compound (1 mmol) prepared in the preceding reaction, N—BOC-amlodipine (1 mmol), and diisopropylethylamine (0.2 mL) in DMF (5 mL) is stirred and warmed under an inert atmosphere. The progress of the reaction is followed by tlc and when reaction is complete, the solution is poured into aqueous 5% NaHCO₃ and the aqueous mixture is extracted with methylene chloride. The organic extract solution is dried (Na₂SO₄), filtered and concentrated under reduced pressure to give the crude product. The desired compound is obtained by purification of the crude product by use of HPLC.

A solution of the product from the preceding reaction (1 mmol) and paraformaldehyde (2 mmols) in methanol (4 mL) is stirred and is acidified with acetic acid to pH 6.6 (pH meter) under a nitrogen atmosphere. Sodium cyanoborohydride (1.1 mmol) is then added and stirring is continued. The course of the reaction is followed by thin layer chromatography. After reaction occurs, the reaction solution is quenched in water and the pH of the aqueous mixture is adjusted to greater than 10 with aqueous NaOH. The mixture is extracted with ether, the organic extracts are washed with half-saturated saline, dried (Na₂SO₄), filtered and concentrated under reduced pressure to give the crude product. The desired compound is obtained by purification of the crude product by use of HPLC.

A solution of the product from the preceding reaction and trifluoroacetic acid (3 mL) in CH₂Cl₂ (5 mL) is stirred at room temperature. The progress of the reaction is followed by tlc. After reaction occurs, more CH₂Cl₂ is added and the solution is washed with aqueous Na₂CO₃ and with H₂O. The organic layer is dried (Na₂SO₄), filtered and concentrated under reduced pressure to give the crude product. The desired compound is obtained by purification of the crude product with the use of HPLC.

### EXAMPLE A42

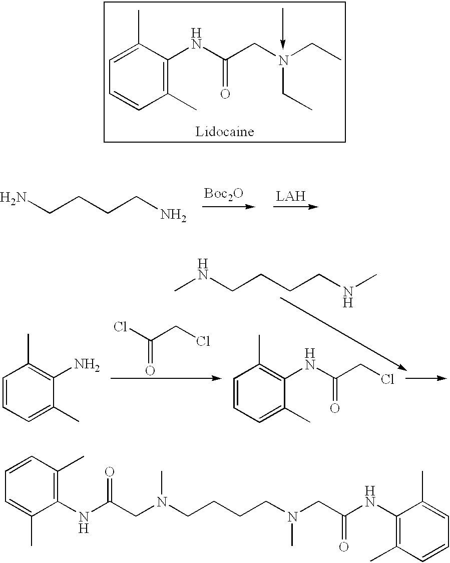

1,4-Diaminobutane (34.0 mmol) was dissolved in 100 mL dichloromethane under a nitrogen atmosphere. Di-tert-butyl dicarbonate (Boc₂O) (119.12 mmol) dissolved in 100 mL dichloromethane was added dropwise to the stirred solution and stirring was continued at room temperature until the reaction was complete (4 hours). The course of the reaction was followed by TLC (50% ethyl acetate and 50% hexanes). The reaction mixture was evaporated giving a precipitate that was collected by filtration. The precipitate was rinsed with ether to yield a white solid (9.02 grams; 92% yield).

Lithium aluminum hydride (LAH) (63.3 mmol) was dissolved in 200 mL tetrahydrofuran (THF) in an ice bath under nitrogen atmosphere. Di-Boc-protected diamine (12.7 mmol) was dissolved in 50 mLTHF and added dropwise to the LAH/THF solution. The reaction was stirred with cooling, then warmed to room temperature, placed in an oil bath and the temperature was raised by increments of 10° C. to 85° C. over a 30 minute period. The mixture was stirred at reflux for 18 hours, then cooled to room temperature and placed in an ice bath. Sodium sulfate decahydrate was slowly added to quench the excess LAH. The solids were removed by filtration and rinsing with THF. The filtrate was concentrated to a thick syrup and excess solvent removed under vacuum to yield N,N-dimethyldiaminobutane as a viscous oil (3.58 grams; 98% yield). The product was characterized by NMR (DMSO) and MS (calculated. M+H=117.2; found, 117.3).

Under nitrogen atmosphere, one equivalent of 2,6-dimethylaniline (7.2 mmoles) was dissolved in 50 mL dichloromethane. To this was added 1.3 equivalents of DIPEA (9.4 mmoles). The reaction mixture was cooled in an ice bath. Chloroacetyl chloride (7.95 mmoles) dissolved in 50 mL dichloromethane was added dropwise to the reaction mixture. The reaction was stirred at room temperature until reaction was complete (approximately 0.5 hours). The course of the reaction was followed by TLC (50% ethyl acetate and 50% hexanes). The reaction mixture was evaporated to a syrup, and was then partitioned between EtOAc and water. The organic layer was washed with saturated NaHCO₃ (2 times), saturated NaCl, then dried with MgSO₄, filtered and rinsed with EtOAc. After removal of excess solvent, 2-chloro-N-(2′,6′-dimethylphenyl)acetamide was obtained as a white solid (51% yield).

Under a nitrogen atmosphere, N,N-dimethyl-1,4-diaminobutane (1.95 mmoles), 1.9 equivalents of 2-chloro-N-(2′,6′-dimethylphenyl)acetamide (3.71 mmoles) and 2.5 equivalents of diisopropylethylamine (DIPEA) (4.89 mmoles) were dissolved in 4 mL ethanol. This was refluxed at 85 degrees C. and followed by TLC (ethyl acetate:hexanes, 1:1) until the reaction was complete (12 hours). The reaction was cooled to room temperature, inducing crystallization of the product. The product was filtered, rinsed with ether, and dried on a vacuum line to yield the desired compound as a white solid (0.88 grams (83% yield). The product was characterized by NMR (DMSO). Mass spectrometry was taken in methanol.

### EXAMPLE A43

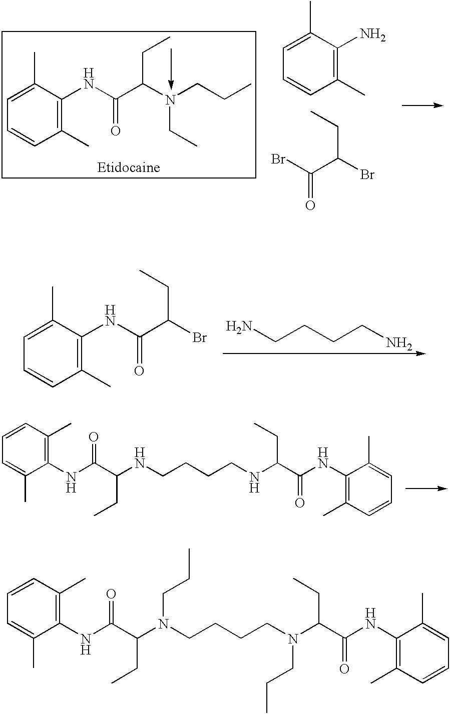

2,6-Dimethylaniline (82.5 mmols) was dissolved in 100 mL dichloromethane under a nitrogen atmosphere, and cooled in an ice bath. DIPEA (247.6 mmols) was added to the solution, and 2-bromobutyric acid (165.0 mmols) in 100 mL CH₂Cl₂ was added over one hour by dropping funnel. After the addition was complete, the solution was brought to room temperature and stirred for an additional hour. The reaction mixture was concentrated, and ether added to the residue. The ether was decanted, and the undissolved salts were rinsed with ether (×2). The combined ether solution was washed with 3 N NaOH (×2), 3 M HCl (×2), saturated NaHCO₃, saturated NaCl, and dried over MgSO₄. The crude product, 2-bromo-N-(2,6-dimethyl-phenyl)-butyramide, was concentrated and dried, and used without further purification in subsequent coupling.

Under a nitrogen atmosphere, 1,4-diaminobutane (11.34 mmols), 2-bromo-N-(2,6-dimethyl-phenyl)-butyramide (22.69 mmols), and DIPEA (28.36 mmols) were dissolved in EtOH (5 mL). The reaction was heated at reflux for 24 hours, then concentrated and chromatographed on a silica gel column to yield the desired compound.

This multibinding agent can be converted to another multibinding agent as follows: Under a nitrogen atmosphere, 1.25 mmols of the compound prepared prepared as above, and bromopropane (2.5 mmols) were dissolved in 1 mL EtOH in a sealed tube. DIPEA (2.75 mmols) was added, and the reaction refluxed for 12 hours. The reaction was concentrated, and the crude product purified by silica-gel chromatography (MeOH/CH₂Cl₂). After concentration of the product-containing fractions, the product was dissolved in MeOH (2 mL) and 4 N HCl was added until the pH was between 1 and 2. The solution was stirred for 20 minutes, and then pipetted into ether (300 mL) to precipitate the product as its dihydrochloride salt. This was filtered and dried, yielding the product in 81% yield.

### EXAMPLE A44

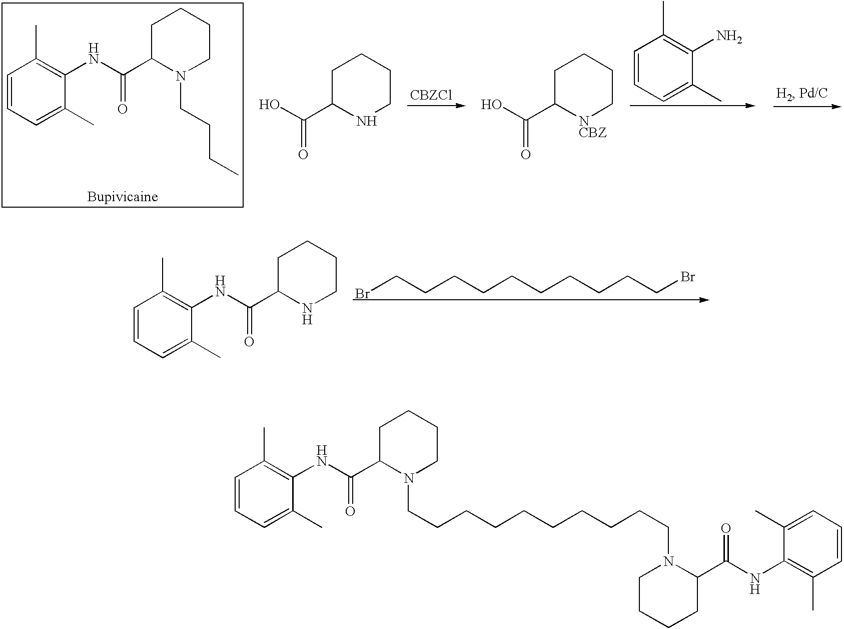

Under a nitrogen atmosphere, 77.4 mmoles of DL-pipecolinic acid (i.e., piperidine-2-carboxylic acid) was dissolved in 80 mL 3 N aqueous NaOH, and cooled in an ice bath. Benzyl chloroformate (92.3 mmoles) and 25 mL 3 N NaOH were added in alternating aliquots over 1 hour, and the reaction mixture was then stirred at room temperature for 12 hours. When the reaction was complete, the N-protected carboxylic acid (abbreviated herein as Cbz-DL-pipecolinic acid) was extracted with ether (×3). The basic layer was acidified with 6 N HCl and extracted with ether (×3). The combined ether layers were rinsed with 1 N HCl, saturated NaCl, then dried over MgSO₄, filtered (rinsing with ether) and concentrated. The yield of the desired product as a white solid was 19.65 grams (96% yield). The product was characterized by NMR (DMSO).

Under nitrogen atmosphere, Cbz-DL-pipecolinic acid (9.1 mmols) was dissolved in DMF (10 mL). DIPEA (12.4 mmols), HATU (10.7 mmols) and HOAT (0.82 mmols) were added, and the reaction was stirred at room temperature for 1 hour. 2,6-Dimethylaniline (8.25 mmoles) was added, and the reaction was stirred for 18 hours. The reaction mixture was then partitioned between water and EtOAc. The organic layer was washed with 1 N NaOH, 2 N HCl, saturated NaCl, then dried over MgSO₄, filtered (washing with EtOAc), and concentrated. The desired amide product [Cbz-piperidine-2-carboxylic acid (2,6-dimethyl-phenyl)-amide] was afforded as a white solid (3.0 grams, 99% yield). The product was characterized by NMR (DMSO).

Under nitrogen atmosphere in a Parr bottle, 8.2 mmoles of Cbz-piperidine-2-carboxylic acid (2,6-dimethyl-phenyl)-amide was dissolved in MeOH (100 mL). 10% Pd/C (0.5 g) was added, and the bottle was agitated under a hydrogen atmosphere (20 psi) for 30 minutes. The catalyst was filtered off using Millipore filter paper (washing with MeOH). The filtrate was concentrated to a thick syrup, yielding 1.75 grams (92% yield) of [piperidine-2-carboxylic acid (2,6-dimethyl-phenyl)-amide] as an off-white solid. The product was characterized by NMR (DMSO).

Under a nitrogen atmosphere, 1,10-dibromodecane (0.22 mmoles), piperidine-2-carboxylic acid (2,6-dimethyl-phenyl)-amide (0.43 mmoles), and DIPEA (0.57 mmoles) were dissolved in 2 mL ethanol, and stirred at 80° C. for 12 hours. The reaction mixture was separated by reverse-phase preparative HPLC, eluting with a gradient of 15-90% acetonitrile in water over 90 minutes. The desired peak was pooled, frozen, lyophilized, and collected as a white powder (the TFA salt). The product was dissolved in 0.1 N HCl and lyophilized to exchange to the HCl salt. The desired compound was obtained as a white solid (0.083 grams; 61% yield). The product was characterized by NMR (DMSO).

### EXAMPLE A45

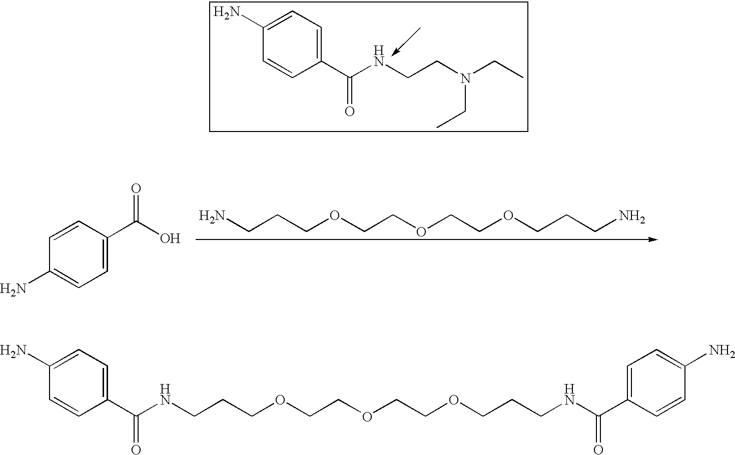

Under a nitrogen atmosphere, 4,7,10-trioxa-1,13-tridecanediamine (2.27 mmols), HATU (6.81 mmols), HOAt (0.22 mmols), and DIPEA (7.94 mmols) were added to 20 mL DMF and stirred for 20 minutes at room temperature. 4-(Butylamino) benzoic acid (5.67 mmols) was added, and the reaction was stirred overnight. The product was purified by reverse-phase preparative HPLC and characterized by NMR (DMSO).

### EXAMPLE A46

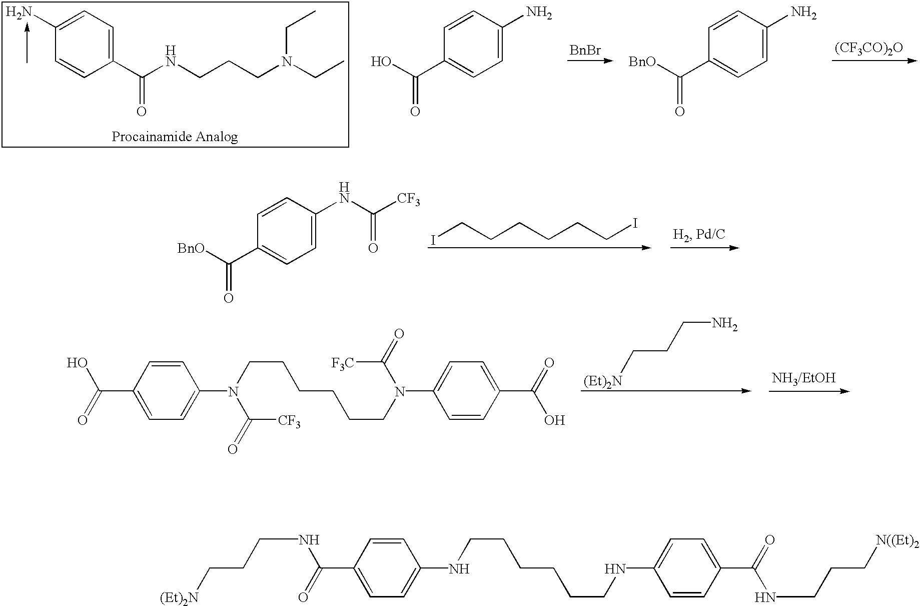

Under a nitrogen atmosphere. 4-aminobenzoic acid (52.7 mmols), benzyl bromide (67.3 mmols), benzyltriethylammonium chloride (80 mmols) and K₂CO₃ (144.7 mmols) were dissolved in acetonitrile. The reaction mixture was heated at 90° C. for 6 hours, cooled to room temperature, and filtered. The filtrate was concentrated. dissolved in CH₂Cl₂:MeOH, 9:1 v/v (100 mL), and stirred with 30 g MB amberlite for one-half hour. The residue was filtered off, and the filtrate concentrated. The crude product was purified by silica-gel chromatography (ethyl acetate/hexanes) to give 5.66 g (47% yield) of the desired product, 4-amino-benzoic acid benzyl ester. The product was characterized by NMR (CDCl₃) and MS (found, M+H=228).

Under a nitrogen atmosphere, 4-amino-benzoic acid benzyl ester (25 mmols) was dissolved in ether (400 mL). The mixture was cooled to 0° C. and trifluoroacetic anhydride (92 mmols) was added. The reaction mixture was stirred at 0° C. for 3 hours, concentrated to dryness, the residue was dissolved in ether, washed with 10% NaHCO₃ solution, then brine. The organic phase was dried over MgSO₄, filtered, and concentrated to dryness to give 4-(2,2,2-trifluoro-acetylamino)-benzoic acid benzyl ester (8.2 g, 100% yield ) as a white solid. The product was characterized by NMR (CDCl₃) and MS (found, M+H=323).

Under a nitrogen atmosphere, 4-(2,2,2-trifluoro-acetylamino)-benzoic acid benzyl ester (9.6 mmols), 1,6-diiodohexane (5 mmols), benzyltriethyl ammonium chloride (1.1 mmols) and K₂CO₃ (21.7 mmols) were dissolved in CH₃CN (150 mL). The reaction mixture was stirred under reflux for 48 hours, cooled, and filtered. The filtrate was concentrated to dryness, and purified by silica-gel chromatrography (ethyl acetate/hexanes) to yield 1.3 g (37% yield) of the desired product. The product was characterized by NMR (CDCl₃) and MS (found, MH⁺=725). MS (found, MNa⁺=747).

A solution of the above compound (1.6 mmols) in THF (200 mL) was stirred under H₂ atmosphere in the presence of 10% Pd/C (450 mg) for 12 hrs. When the reaction was complete. the catalyst was filtered off, and the filtrate was concentrated to give 1.01 g of the crude deprotected product as a white solid.

Under a nitrogen atmosphere, the above compound (1.45 mmols), was dissolved in THF (80 mL). HATU (3 mmols), HOAT (36 mmols), diethylaminopropylamine (3.55 mmols), and DIPEA (3.04 mmols) were added. The reaction mixture was stirred at room temperature for 3 days, then concentrated to dryness. The crude product was purified by silica-gel chromatography (methylene chloride/methanol/ammonia) to give 450 mg (100% yield) of the desired product. The product was characterized by NMR (CD₃OD) and MS (found, M+H=745). MS (found, MNa⁺=767).

Under a nitrogen atmosphere, the above compound (450 mg) was dissolved in a solution of 2N NH₃ in MeOH (40 mL). After addition of 25% aqueous NH₃ ammonia solution (8 mL), the mixture was heated to 62° C. overnight. When the reaction was complete as judged by TLC, the reaction mixture was cooled to room temperature, and a white precipitate was formed. The precipitate was collected, washed with H₂O, and dried under vacuum to give 230 mg of the desired compound, which was exchanged to the HCl salt. The product was characterized by NMR (CD₃OD) and MS (found, M+H=553). MS (found, MNa⁺=575).

### EXAMPLE A47

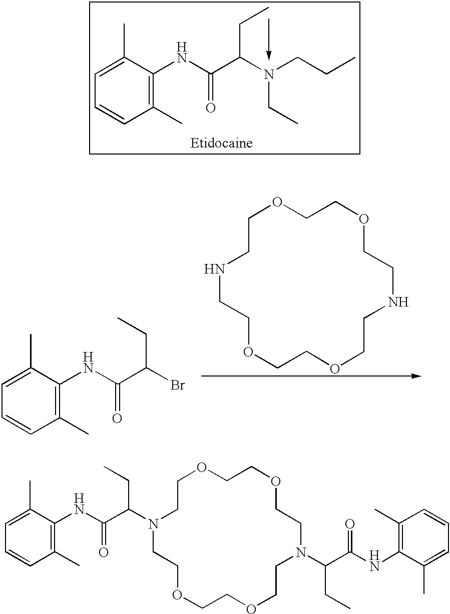

Under nitrogen atmosphere, 1,4,10,13-tetraoxa-7,16-diaza-cyclooctadecane (53.36 mmols), 2-bromo-N-(2,6-dimethyl-phenyl)-butyramide (133.41 mmols), and DIPEA (117.40 mmols) were dissolved in 20 mL ethanol. The reaction was refluxed for 6 days, then cooled and the solvent removed in vacuo. The reaction mixture was partitioned between 1N HCl (300 mL) and ethyl acetate. The acidic layer was extracted with ethyl acetate (×4), then basified with 6N NaOH (100 mL) and extracted with ethyl acetate (×3). The combined organic extracts were washed with 10% Na₂S₂O₃ (×2), brine, and dried over MgSO₄, decolorized with charcoal, filtered and concentrated The crude product (26 g) was purified using silica-gel chromatography (MeOH/CH₂Cl₂) and exchanged to the HCl salt to give the desired compound (33% yield). Characterized by NMR (DMSO).

### EXAMPLE A48

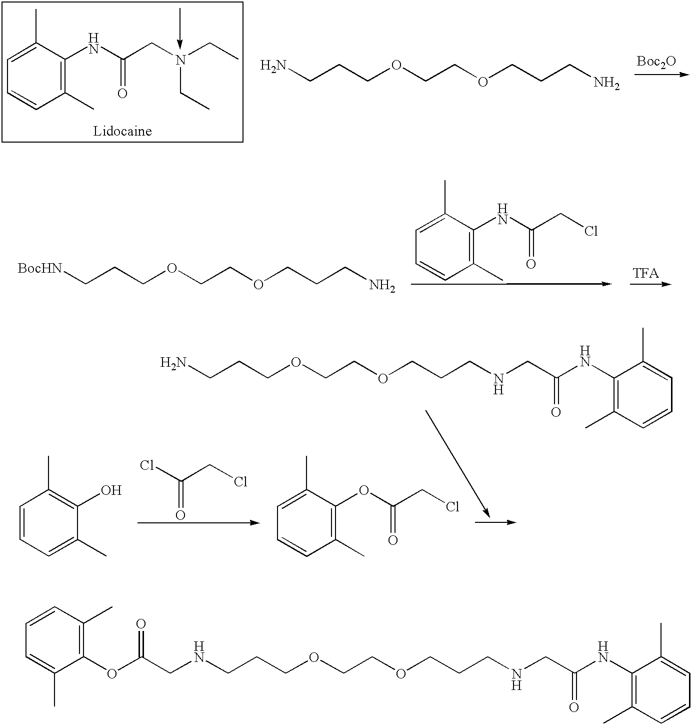

Under a nitrogen atmosphere, N-methyl-N-(3-{2[-2-(3-methylamino-propoxy)-ethoxy]-ethoxy}-propyl)-amine (130.56 mmols) was dissolved in dioxane (100 mL). Di-tert-butyl dicarbonate (Boc₂O) (97.92 mmol) in 50 mL dioxane was added over 1.5 hours. The reaction was stirred at room temperature for 21 hours, then concentrated to dryness. The mixture was partitioned between EtOAc (100 mL) and 10% KHSO₄ (50 mL), and the layers separated. The organic layer was washed with 10% KHSO₄ (3×50 mL). The aqueous layer was acidified with 6M HCl to PH 3, washed with EtOAc (50 mL), made basic with solid NaOH. Saturated with NaCl, and washed with CHCl₃. The combined organic layers were dried over Na₂SO₄, filtered, and concentrated to yield N-methyl-N-Boc-N-(3-{2[-2-(3-methylamino-propoxy)-ethoxy]-ethoxy}-propyl)-amine (18.66 g, 41% yield).

Under a nitrogen atmosphere, N-methyl-N-Boc-N-(3-{2[-2-(3-methylamino-propoxy)-ethhoxy]-ethoxy}-propyl)-amine (53.59 mmols) and 2-chloro-N-(2,6-dimethyl-phenyl)-acetamide (50.91 mmols) were added to ethanol (35 mL). DIPEA (80.39 mmols) was added, and the reaction stirred at 85 C. for 19 hours. The reaction was then concentrated to dryness. dissolved in EtOAc and filtered through silica (EtOAc). The filtrate was reconcentrated to give N-methyl-N—Boc—N-(3-{2-(3-{N-[(2,6-dimethyl-phenylaminocarbonylmethyl)-N-methylamino}propyl)amine (19.36 g, 75% yield).

Under nitrogen atmosphere, N-methyl-N—Boc—N-(3-{2-(3-{N-[(2,6-dimethyl-phenylaminocarbonylmethyl)-N-methylamino}-propoxy)-ethoxy]-ethoxy}-propyl)amine (38.04 mmoles) was dissolved in 50 mL dichloromethane Trifluoroacetic acid (50 mL) was added, and the reaction was stirred at room temperature for 0.5 hours. The reaction was concentrated to dryness, then dissolved in EtOAc (100 mL). The solution was washed with KHSO₄ (3×50Ml). The combined aqueous layers were washed with EtOAc (50Ml), made basic with solid NaOH, saturated with NaCl, and washed with chloroform (3×50Ml). The combined organic layers were dried over Na₂SO₄, filtered, and concentrated to give N-(2,6-dimethylphenyl)-2-(3-{2-[2-(3-methylaminopropoxy)-ethoxy]-ethoxy}-propylmethylamino)-acetamide (11.58 g, 74% yield). NMR was taken in DMSO.

Under a nitrogen atmosphere, 2,6-dimethylphenol (1.0 equivalent, 40 mmoles) was dissolved in dichloromethane (40 mL) and diisopropyl ethylene diamine (1.3 equivalents, 53.2 mmoles). This solution was cooled to −78° C. Chloroacetylchloride was added all at once. The reaction was followed by TLC (25 EtOAc:75 Hexanes) until reaction was complete (˜10 minutes). The reaction was extracted using water and ether. The ether layer was washed with water and then brine, dried (MgSO₄), filtered, rinsed with ether, and concentrated under vacuum. The concentrate was purified by silica gel column chromatography using hexanes as eluant, to afford 10.5 grams ( 94.6% yield) of chloro-acetic acid 2,6-dimethyl-phenyl ester product as a tan oil.

Under a nitrogen atmosphere in a sealed tube, the chloroacetic acid 2,6-dimethyl-phenyl ester product of Step 4 (1.2 equivalents, 6.72 mmoles) was reacted with 1.0 equivalent (5.6 mmoles) of the monoadduct product of Step 3 , diisopropyl ethylene diamine (1.3 equivalents, 6.72 mmoles) and ethanol (1 mL) at 85° C. for 14 hours. The reaction mixture was poured into ether with stirring, and the resulting precipitate was filtered, then dissolved in 90% water, 10% acetonitrile. The product was obtained by preparative HPLC eluting with 5% acetonitrile/95% H₂O to 90% acetonitrile/10% H₂O. MS (found, M+H 572).

### EXAMPLE A49

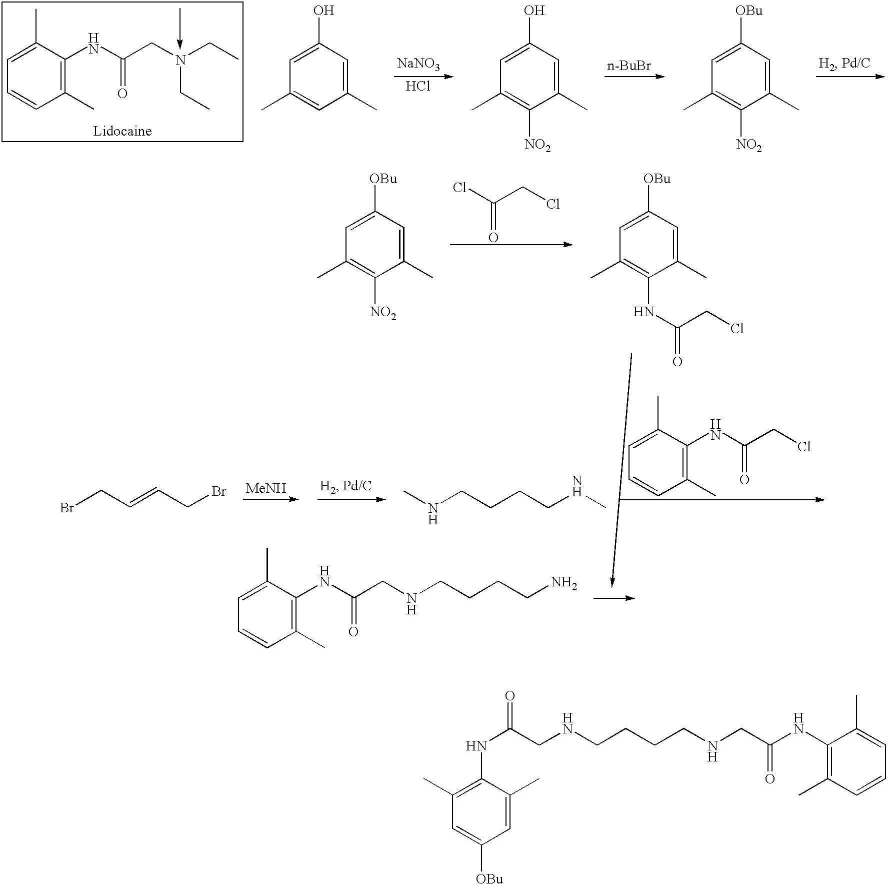

To a mixture of 3,5-dimethylphenol (25 g, 205 mmol) and NaNO₃ (26.1 g, 307 mmol) in ether (200 mL) and water (200 mL), at 0° C., 250 mL of conc.HCl solution was added dropwise within 1 hour under stirring. The reaction mixture was kept under stirring overnight and the temperature was allowed to warm to room temperature. The organic phase was seperated and the aqueous phase was extracted with ether (3×100 mL). The combined organic phase was washed with 3N HCl (2×100 mL), saturated NaHCO₃ solution (3×100 mL) and finally with brine (200 mL), dried over Na₂SO₄, filtered and concentrated to dryness. The obtained residue was purified by chromatography over silica gel by using ethyl acetate and hexane (2:8) as eluent. The collected fractions were further recrystalized in ether/hexane to afford 3.5-dimethyl-4-nitrophenol (6.7 g, 19% yield) as a yellow solid. MS: m/e 167.

A mixture of 3,5-dimethyl-4-nitrophenol (2 g, 12 mmol), bromobutane (1.6 mL, 15 mmol), K₂CO₃ (2.1 g, 15 mmol) in DMF (6 mL) was stirred at 60° C. for 6 hours. The reaction mixture was poured into 50 mL of H₂O and extracted with ether (3×50 mL). The combined ether layer was washed with H₂O and subsequently with brine, dried over Na₂SO₄, filtered and concentrated to give 4-butoxy-2.6-dimethyl-nitrobenzene (2.6 g, 97% yield) as a dark brown oil. ¹H—NMR in CDCl₃.

A solution of 4-butoxy-2,6-dimethyl-nitrobenzene (7.76 g, 34.8 mmol) in methanol (200 mL) was hydrogenated overnight at 35 psi, in the presence of 10% Pd/C (1.5 g) and 3 mL of conc.HCl. The reaction mixture was then filtered and the filtrate was concentrated to dryness. The obtained residue was partitioned into CHCl₃/i-PrOH (4:1 v/v) and saturated NaHCO₃ solution, and extracted with CHCl₃/i-PrOH (4:1 v/v). The organic phase was dried over Na₂SO₄, filtered and concentrated to give 4-butoxy-2,6-dimethyl-phenylamine, (6.5 g, 97% yield) ¹H—NMR in CDCl₃. (TLC system: Rf:0.15 with EtOAc/hexane=2/8)

To a solution of 4-butoxy-2,6-dimethyl-phenylamine (1.4 g, 7.4 mmol) and diisopropylethylamine (1.4 mL, 8.0 mmol) in CH₂Cl₂ (6 mL), 2-chloro-acetylchloride (0.88 mL, 11.0 mmol) in CH₂Cl₂ (6 mL) was added slowly at 0° C. The ice-bath was removed and the reaction mixture was kept under stirring for 0.5 hour. The reaction mixture was concentrated and the crude concentrate was partitioned between ethyl acetate and water. The organic phase was washed with water twice. The organic phase was dried over Na₂SO₄ and concentrated to dryness. The residue was was purified by flash chromatography over silica-gel using ethyl acetate/hexane (20/80 v/v) to afford 2-chloro-N-(4-butoxy-2,6-dimethyl-phenyl)-acetamide (1.95 g, 98% yield), ¹H—NMR in CDCl₃.

A solution of trans-1,4-dibromobutene (80 g, 374 mmol, Fluka 34060) in 500 mL of THF/EtOH (1/2) was added dropwise to a solution of 8N methylamine in EtOH (1L), which is kept at a temperature below 35° C. by using an ice-bath. After the addition, the reaction mixture was stirred at 38° C. for approximately 24 hours and then concentrated to yield a precipitate. The solid was filtered, washed with acetone to give N,N′-dimethyl-1,4-butenediamine dihydrobromide (53.8 g, 52% yield) as a white solid, which was hydrogenated in 350 mL MeOH/H₂O (6:1 v/v) for 20 hours at 35 psi and in the presence of 10 g of 10% Pd/C. The catalyst was filtered and washed with MeOH and H₂O. The filtrate was concentrated and precipitated by adding acetonitrile. The solid was filtered and washed with acetonitrile to give N,N′-dimethyl-1,4-butanediamine dihydrobromide as a white solid (45.5 g, 93% yield).

2-Chloro-N-(2,6-dimethylphenyl)acetamide (2.8 g, 14.2 mmol) was added to a mixture of N,N′-dimethylbutane dihydrobromide (7.68 g, 27.6 mmol) and DIPEA (8 mL, 46 mmol) in EtOH (200 mL). The mixture was stirred at 90° C. overnight, then concentrated to yield a white precipitate, which was filtered off. The filtrate was concentrated and purified by chromatography over silica-gel by using CH₂Cl₂/CH₃OH/25% aq.NH₃ (97/2.5/0.5→95/4.5/0.5→90/9/1 by volume) as eluant to obtain the desired monoadduct as a colorless foam (2.62 g, 67% yield). MS: m/e 278 (M+H)), ¹H—NMR in CD₃OD.

A mixture of the product above (2.5 g, 9.2 mmol), 2-chloro-N-(4-butoxy-2,6-dimethyl-phenyl)-acetamide (2.5 g, 9.2 mmol), and DIPEA (2.1 mL, 12 mmol) in EtOH (45 mL) was refluxed overnight. The reaction mixture was concentrated and the obtained residue was purified by chromotography over silica gel by using EtOAc/hexane/Et₃N (50:49:1), then EtOAc/Et₃N (99:1) and subsequently CH₂Cl₂/CH₃OH/25% aq.NH₃ (94/5.4/0.6) as eluant to obtain the desired product (0.28 g, 55% yield). This compound was converted to its dihydrochloride salt by treatment with 4N HCl solution in dioxane. MS: m/e 511 (M+H), and ¹H—NMR in CD₃OD.

### EXAMPLE A50

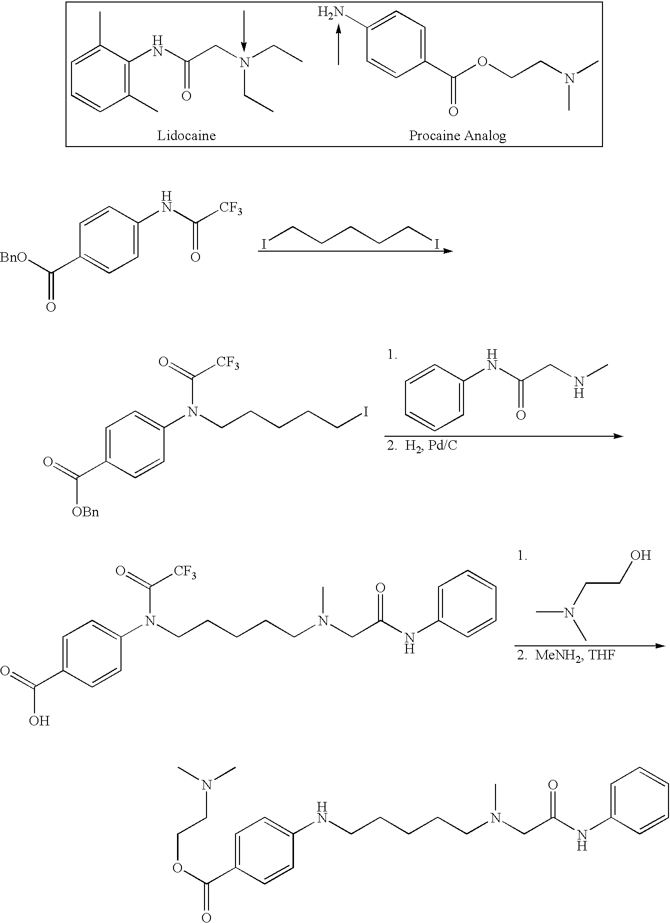

A mixture of 4-trifluoroacetylamino-benzoic acid benzyl ester (2.5 g, 7.74 mmol), 1,5-diiodopentane (12.5 g, 38.6 mmol), potassium carbonate (4.3 g, 31.1 mmol) and benzenetriethylammonium bromide (420 mg, 1.54 mmol) in acetonitrile (150 ml) was stirred under reflux overnight. After cooling to room temperature, the reaction mixture was filtered and the filtrate was concentrated. The resulting residue was purified by chromatography over silica gel using ethyl acetate/hexane (1/9 by volume) as eluent to obtain the desired compound (3.3 g, 82% yield), ¹H—NMR in CDCl₃, MS: m/e 520 (M+H).

A mixture of the compound above (3.2 g, 6.16 mmol), N-(2,6-dimethylphenyl)-2-methylamino-acetamide (2.4 g, 12.5 mmol), and potassium carbonate (4.3 g, 31.1 mmol) in acetone was stirred at 60 C. overnight. The reaction mixture was filtered and the filtrate was concentrated. The resulting residue was purified by chromatography over silica gel to obtain the desired compound (1.8 g, 50% yield), ¹H—NMR in CDCl₃, MS: m/e 584 (M+H).

A solution of compound above (1.8 g, 3.08 mmol) in tetrahydrofuran (80 ml) was stirred under H₂ atmosphere and in the presence of 10% Pd/C (1 g) for 16 hours. The reaction mixture was filtered and the filtrate was concentrated to give the desired compound as a white foam (1.35 g, 89% yield) MS: m/e 494 (M+H).

A mixture of the compound above (0.77 g, 1.56 mmol), HATU (1.8 g, 4.73 mmol), HOAT (0.065 g, 0.47 mmol), N,N-dimethylethanolamine (0.47 ml, 4.68 mmol) and DIPEA (0.82 ml, 4.7 mmol) in anhydrous tetrahydrofuran (34 ml) was stirred at room temperature overnight. The reaction mixture was concentrated and subsequently purified by chromatography over silica gel using CH₂Cl₂/CH₃OH/25%NH₄OH (98.8/1/0.2 à94/5.4/0.6 by volume) as eluant to afford the desired compound (0.5 g, 57% yield), MS: m/e 565 (M+H).

The compound above (0.5 g, 0.89 mmol) was dissolved in a solution of 2N methylamine in tetrahydrofuran. The resulting solution was stirred at room temperature overnight and then concentrated. The residue was purified by chromatography over silica gel using CH₂Cl₂/CH₃OH/25%NH₄OH (98.8/1/0.2→94/5.4/0.6 by volume) as eluant to afford the desired compound, (0.15 g, 36% yield). MS: m/e 469 (M+H). This compound was converted to the dihydrochloride salt by treatment with 4N HCl solution in dioxane.

### EXAMPLE AS51

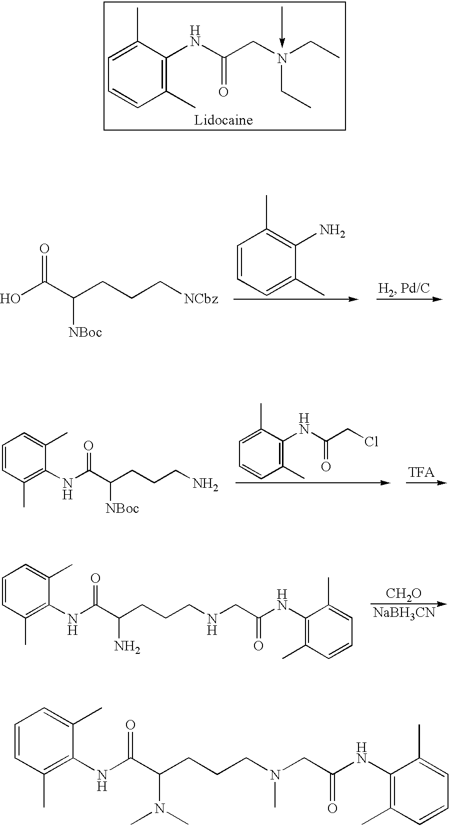

S(L)-omithine protected on the α-amino group with Boc and on the δ-amino group with Cbz (2.96 g, 8.08 mmol) was dissolved in 30 mL dry DMF. HATU (3.63 g, 9.55 mmol), HOAT (0.1 g, 0.73 mmol) and DIPEA (1.41 g, 10.9 mmol) were added and the mixture was stirred at room temperature for 40 min. 2,6-Dimethylaniline (0.886 g, 7.31 mmol) was added and the reaction mixture was stirred overnight at room temperature under N₂ atmosphere. The mixture was evaporated and resuspended in EtOAc, then washed with brine, and extracted with EtOAc. The organic phases were combined. dried(MgSO₄) evaporated then resuspended and chromatographed on a silica gel column with 2% MeOH/CH₂Cl₂ as eluant. The product was obtained as a yellow solid (4.33 g,>99% yield), and was characterized by NMR (CDCl₃).

The product of the previous step was dissolved in MeOH and 10% Pd/C (0.25 g) was added and stirred. The reaction vessel was evacuated, then H₂ was added and the solution was stirred at room temperature for 2 hours until the reaction was judged complete by TLC. The catalyst was removed by filtration and the filtrate was concentrated to afford the desired compound as a yellow oily foam (2.87 g).

The product of the previous step (2.87 g, 8.55 mmol) was reacted at 80° C. overnight with 2-chloro-N-(2′,6′-dimethylphenyl)acetamide (1.52 g, 7.69 mmol) (prepared as in Example 5 above) in the presence of DIPEA ((1.22 g, 9.47 mmol) in EtOH (25 mL). The reaction was concentrated and the residue dissolved in CH₂Cl₂. The organic phase was washed with three portions of 0.3N HCl. The aqueous layers were combined and the pH adjusted to 11 by dropwise addition of 5N NaOH. The resulting suspension was extracted with three portions of CH₂Cl₂, and these organic phases were combined, dried (MgSO₄) evaporated, then resuspended and chromatographed on silica gel (12.5×14 cm column), eluted with 2% MeOH (400 mL), 3% MeOH/CH₂Cl₂ (300 mL), and 5% MeOH (300 mL) to afford the product as a pale yellow foam (1.59 g).

The product above (0.6 g) was dissolved in CH₂Cl₂ and TFA was added with stirring for 1 hour until deprotection was complete, as judged by TLC. The reaction mixture was evaporated, and the residue redissolved in CH₂Cl₂. The organic phase was washed with three portions of 0.3N HCl. The aqueous layers were combined and the pH adjusted to 11 by dropwise addition of 5N NaOH. The resulting suspension was extracted with three portions of CH₂Cl₂. These organic layers were combined, dried (MgSO₄) and concentrated to afford the deprotected product as a viscous foam (350 mg).

The product above (0.149 g, 0.376 mmol) was dissolved in MeOH (25 mL). Formaldehyde (37% aqueous) (0.46 mL) was added, followed by NaBH₃CN (117 mg) and alkylation was carried out for 1 hour. The pH of the reaction was then adjusted with glacial acetic acid to about pH 6 and the reaction mix stirred for 30 min. The pH was then raised to pH 9 with NH₄OH and the reaction mix was concentrated by evaporation to a slurry. The slurry was redissolved in CH₂Cl₂, washed with saturated NaHCO₃ and extracted 3× with CH₂Cl₂. The organic phases were combined, dried (MgSO₄), concentrated by evaporation and the residue was purified by chromatography on silica gel eluted with 400 mL of 2%, 4%, and 5% MeOH/CH₂Cl₂ to afford the desired compound (0.135 g, 0.308 mmol; 82% yield). The compound was converted to the HCl salt (0.12 g). The product was characterized by MS (observed, M+1=439) and NMR (DMSO).

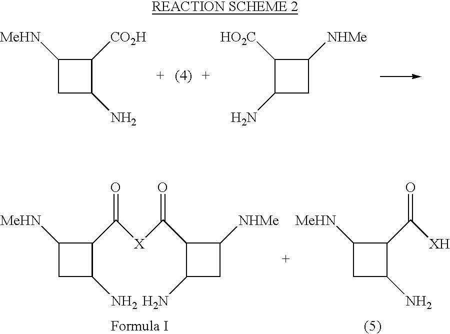

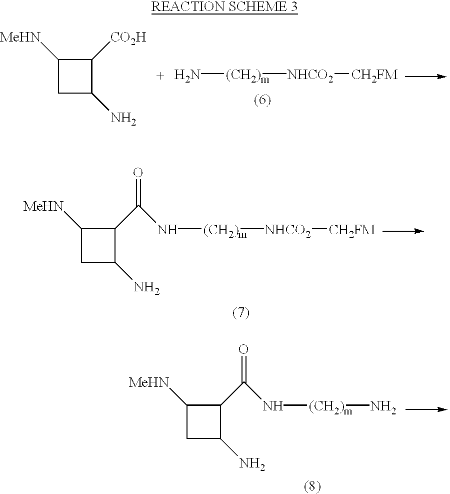

where FM represents 9-fluorenyl., and m is an integer of 1-20

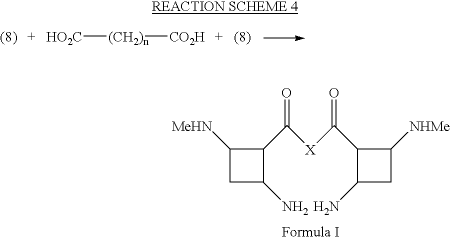

where X is a linker of formula:

—NH—(CH₂)ₘNHC(O)CH₂)ₙC(O)NH(CH₂)ₘ—NH—

in which m and n are independently integers of 1-20.

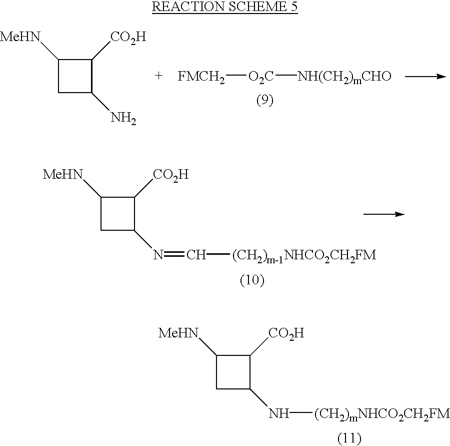

in which m is an integer of 1-20, and FM is 9-fluorenyl.

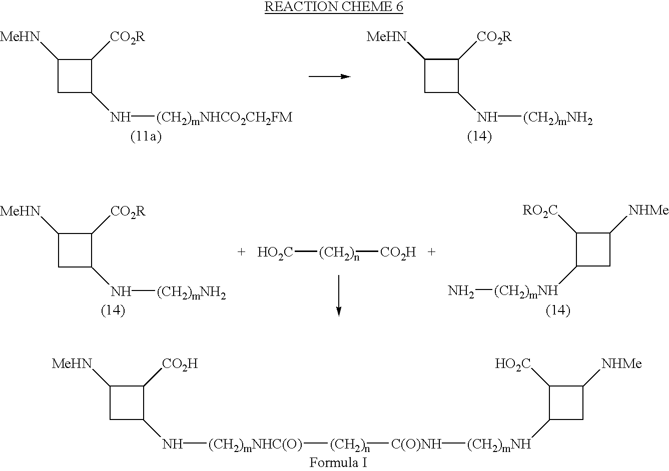

where R is a protecting group, such as an ester, m and n are as defined above, and FM is 9-fluorenyl

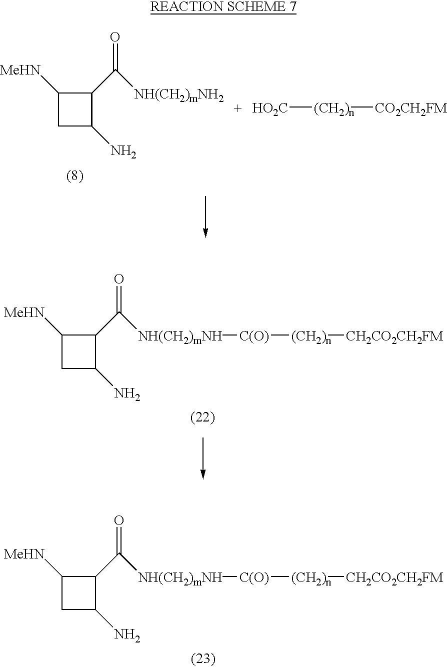

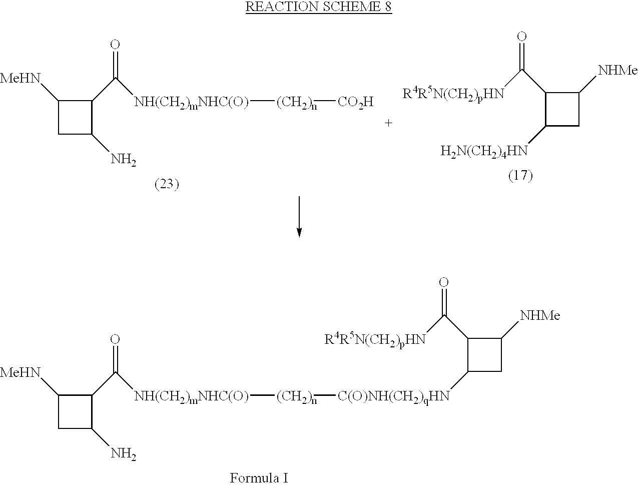

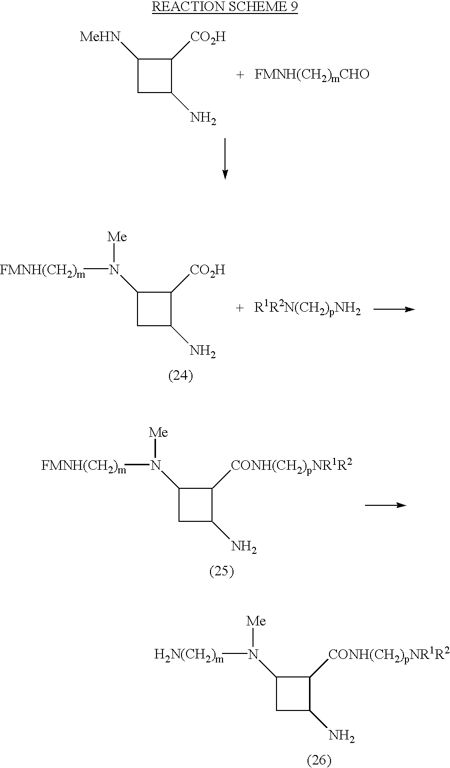

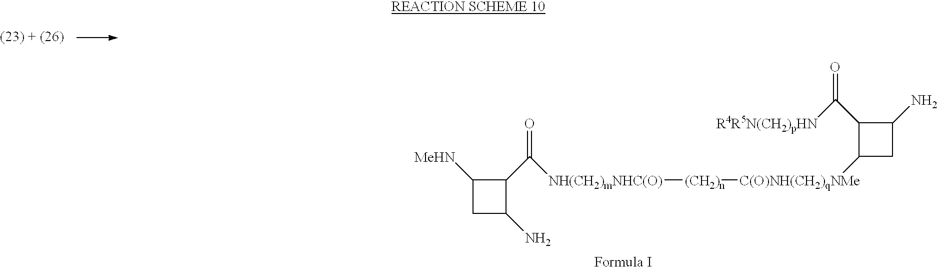

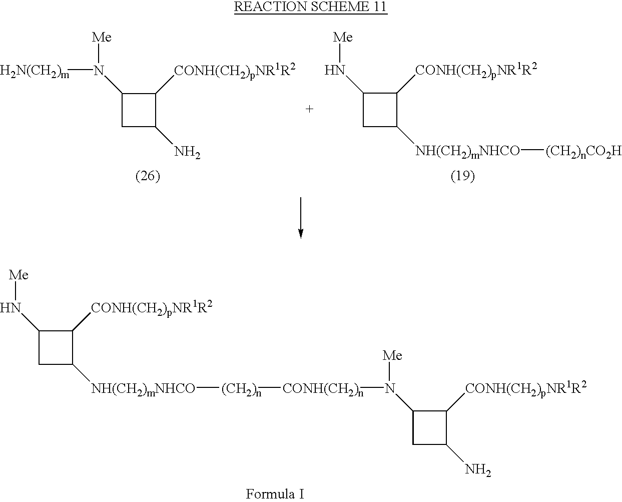

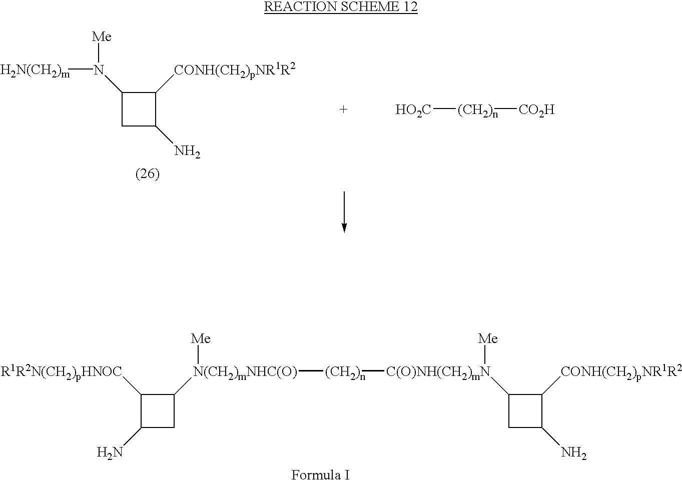

### REACTION SCHEME 13

(NONE)

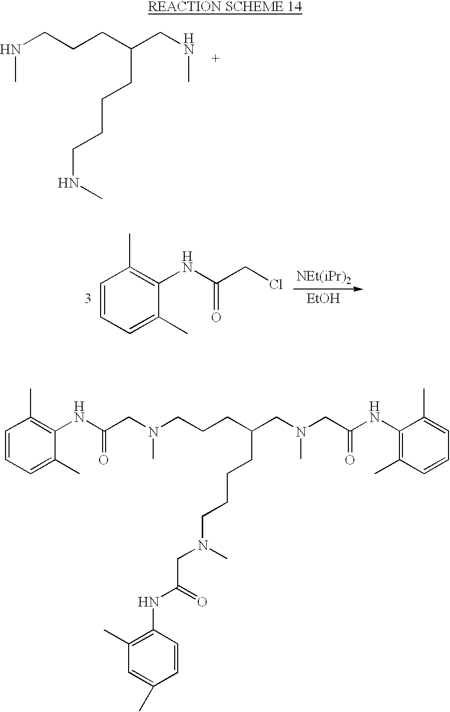

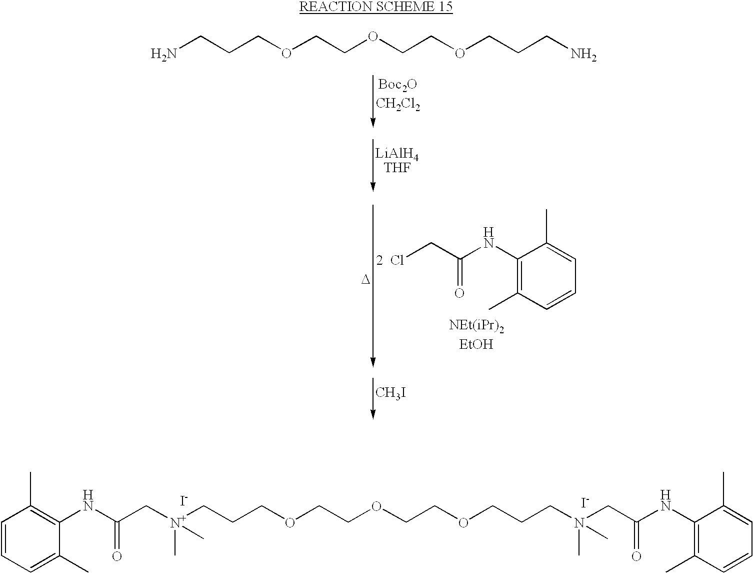

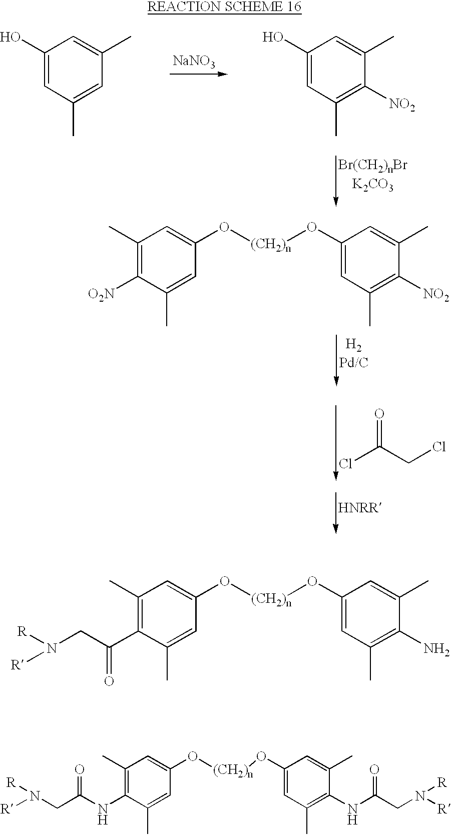

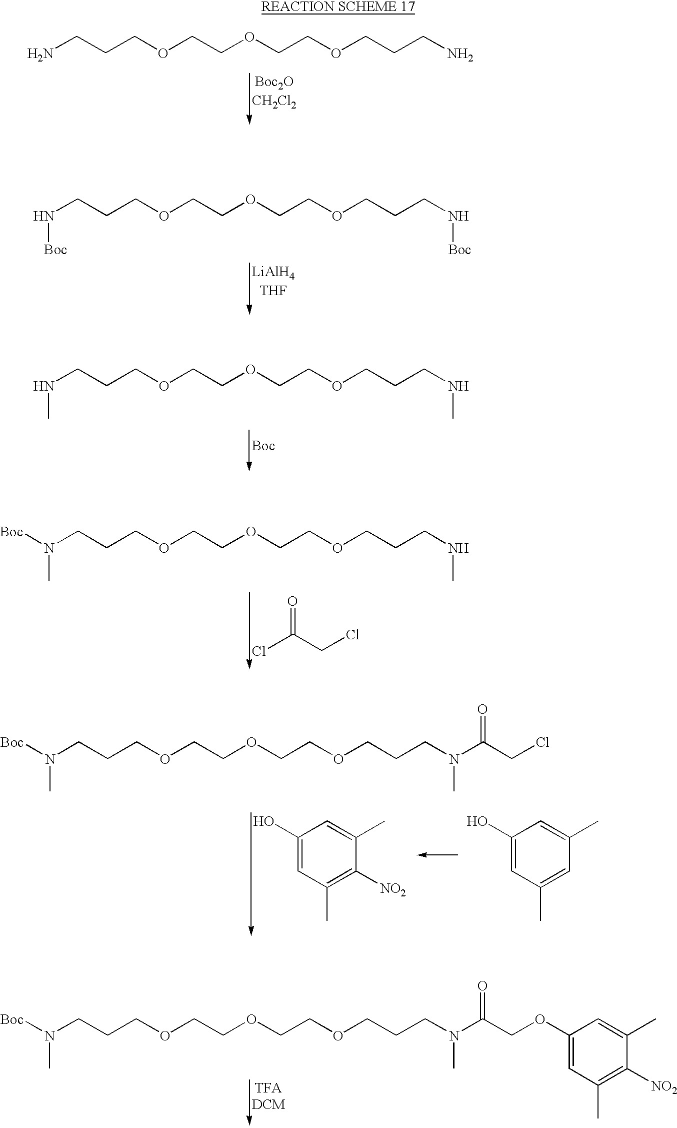

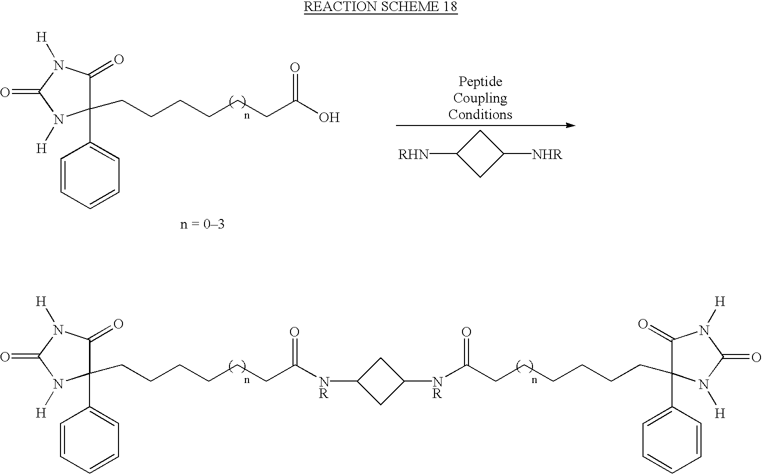

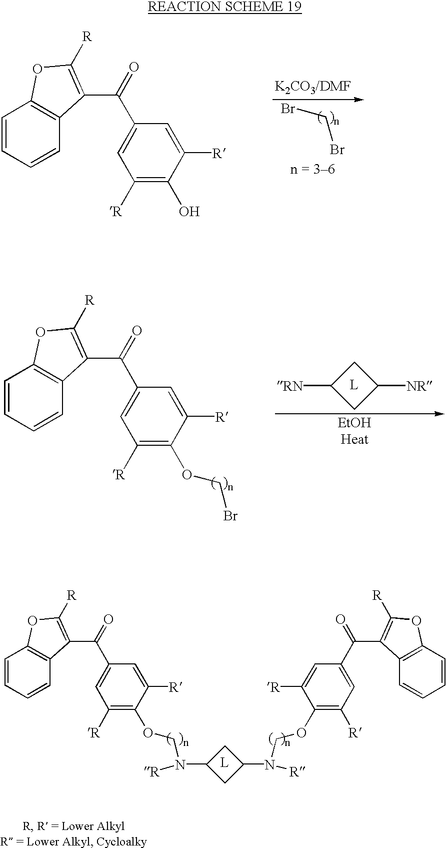

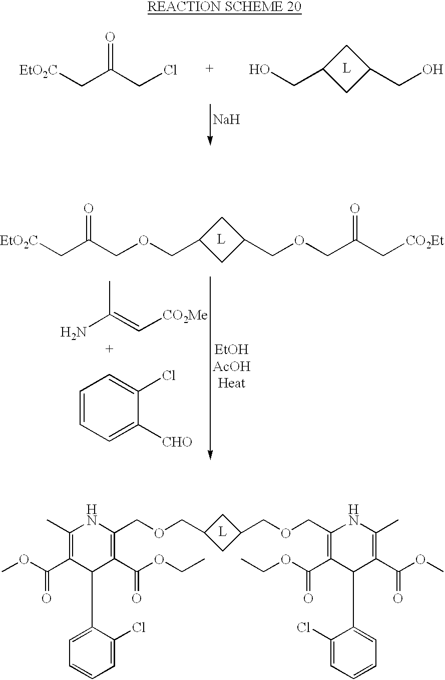

TABLE 1
Ligands Acting on Cell Membrane Transporters

| Channel | Clinical Indication | Drug | Binding Site | References |
|---|---|---|---|---|
| Shaker K+ | Arrhythmia | Amiodarone | AA residues on S6 | Grace et al. NEJM |
| | | Quinidine | segment of K+ | 338(1); 35 (1998) |
| | | | channel | |
| Na+ | Local Anesthesia | Lidocaine | AA residues on | Ragsdale et al. |
| | | Bupivacaine | IV/S6 of Na+ | PNAS 93; 9270 |
| | | Ropivacaine | channel | (1996) |
| | | Procaine | | |
| | | Chloroprocaine | | |
| | | Etidocaine | | |
| | | Mepivacaine | | |
| | | Benzocaine | | |
| | Arrhythmia | Flecainide | AA residues on | Ragsdale et al. |
| | | Mexiletine | IV/S6 of Na+ | PNAS 93; 9270 |
| | | Morcizine | Channel | (1996) |
| | | Tocainide | | |
| | Epilepsy | Phenytoin | AA residues IV/S6 | Ragsdale et al. |
| | | | of Na+ Channel | PNAS 93; 9270 |
| | | | | (1996) |
| Ca2+ | Hypertension | Amlodipine | DHP binding site- | Striessnig et al. |
| | | Felodipine | amino acids on | TiPS 19; 108 |
| | | Isradipine | IIIS5/IIIS6 and | (1998) |
| | | Nicardipine | IVS6 of Ca2+ | |
| | | Nifedipine | channel | |
| | | Nimodipine | | |
| | | Nisoldipine | | |
| | Hypertension; | Verapamil | PAA binding site- | Striessnig et al. |
| | Heart Failure | | amino acid | TiPS 19; 108 |
| | | | residues on IVS6 | (1998) |
| | | | of Ca2+ channel | |
| | Arrhythmia | Bepredil | | |
| 5HT3 | Emesis | Ondansetron | | |
| | | Granisetron | | |
| | | Dolasetron | | |
| GABA | Epilepsy | Benzodiazepines | BDZ binding site | Sigel et al. JBC |
| | | Alprazolam | on the α subunit | 258; 6965; (1983) |
| | | Brotizolam | | |
| | | Chlordiazepoxide | | |
| | | Clobazam | | |
| | | Clonazepam | | |
| | | Clorazepate | | |
| | | Demoxepam | | |
| | | Diazepam | | |
| | | Estazolam | | |
| | | Flurnazenil | | |
| | | Flurazepam | | |
| | | Halazepam | | |
| | | Lorazepam | | |
| | | Midazolam | | |
| | | Nitrazepam | | |
| | | Nordazepam | | |
| | | Oxazepam | | |
| | | Prazepam | | |
| | | Quazepam | | |
| | | Temazepam | | |
| | | Triazolam | | |
| | | Imidazopyridine | | |
| | | Zolpidem | | |
| | | Barbiturates | | |
| | | Amobarbital | | |
| | | Aprobarbital | | |
| | | Butabarbital | | |
| | | Butalbital | | |
| | | Mephobarbital | | |
| | | Methohexital | | |
| | | Pentobarbital | | |
| | | Phenobarbital | | |
| | | Secobarbital | | |
| | | Thiopental | | |
| nAChR | NMJ antagonist | Atracurium | Interface of 2 | |
| | | Doxacurium | subunits | |
| | | Mivacurium | | |
| | | Pancuronium | | |
| | | Pipecuronium | | |
| | | Rocuronium | | |
| | | Vecuronium | | |
| | | Succinylcholine | | |
| | | Tubocurarine | | |
| | Pain | ABT 594 | | |
| Pumps: | | | | |
| H+/K+ ATPase | PUD, GERD | Omeprazole | | |
| | | Lansoprazole | | |
| Na+/K+ ATPase | Heart Failure | Digitalis | Interface of 2α | Repke et al. FEBS |
| | | | subunits in the | Letter 359; 107 |
| | | | protodimer | (1995) |
| MDR | Cancer | Verapamil | | |
| | | Cyclosporin A | | |
| | | Quinine | | |
| Monoamine | Depression | Fluoxetine | | |
| Transporters | | Paroxetine | | |
| 5HT | | Fluvoxamine | | |
| | | Sertraline | | |
| | | Venlafaxine | | |

TABLE 2
Attachment Chemistry

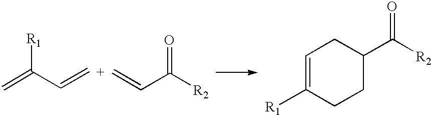

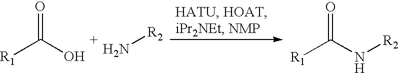

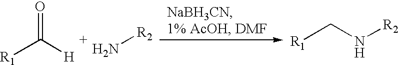

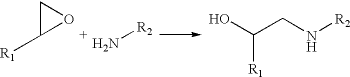

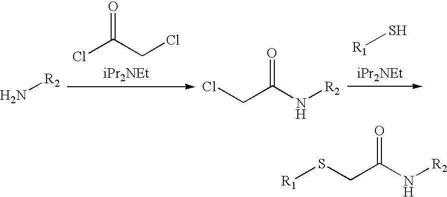

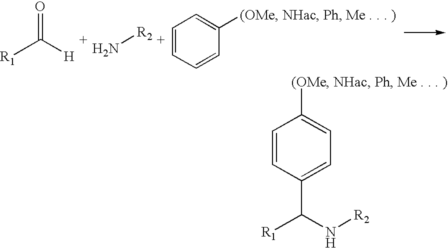

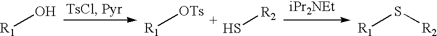

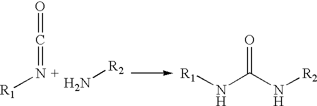

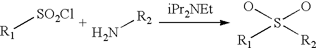

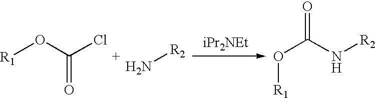

TABLE 3
Exemplary Assays

| Inhibited Transporter | Indication | Assay | Reference |
|---|---|---|---|
| Potassium channel | Arrythmias | In vitro: Patch clamp | Kamp et al, Pflügers |
| | | (cardiac myocytes); | Arch 391: 85 (1981) |
| | | In vivo: Canine model | Abdollah et al, J. |
| | | (MI, induced ventricular | Cardiovasc. Pharm. |
| | | tachycardia (VT)/ | 15(5): 799 (1990) |
| | | ventricular fibrillation | |
| | | (VF)) | |
| Sodium channel | Local Anesthesia | In vitro: Whole cell | Hamill et al, Pflügers |
| | | voltage clamp; | Arch. 391: 85 (1981); |
| | | BTX displacement | McNeal et al, J. Med. |
| | | | Chem. 28: 381 (1985) |
| | Epilepsy | In vitro: Sodium | Brown et al, J. Med. |
| | | channel binding assay | Chem. 40: 602 (1997) |
| | | In vivo: Anticonvulsant | Flaherty et al, J. Med. |
| | | activity was measured | Chem. 39: 1509 (1996) |
| | | by the maximal | |
| | | electroshock (MES) | |
| | | test. | |
| Calcium channel | Hypertension | In vitro: Relaxation of | Rovnyak et al., J. Med. |
| | | contracted rabbit | Chem. 35: 3254 (1992) |
| | | thoracic aorta strips | |
| | | In vivo: Mean arterial | |
| | | pressure in | |
| | | spontaneously | |
| | | hypertensive rats | |
| 5HT₃ receptor | Emesis | In vitro: Inhibition of | Anzinii et al, J. Med. |
| | | ¹⁴C guanidinium uptake | Chem. 38: 2692 (1995) |
| | | in NG 108-15 hybrid | |
| | | cells | |
| | | In vivo: Antagonism of | |
| | | 5HT induced reflex | |
| | | bradycardia in the rat. | |
| GABA_{A} receptor | Epilepsy | Increase in GABA- | Pomés et al, Dev. Brain |
| | | induced chloride flux | Res. 73: 85 (1993), J. |
| | | and benzodiazepine | Pharmacol Exp. Ther. |
| | | binding in primary | 271: 1616 (1994) |
| | | cultures of neocortical | |
| | | neurons and cerebellar | |
| | | granule cells. | |
| nAChR | Pain | In vitro: Displacement | Holladay et al., J. Med. |
| | | of 3[H] cytisine from rat | Chem. 41: 407 (1998) |
| | | brain membranes | |
| | | In vivo: Mouse hot plate | |
| | | assay | |
| Na+/K+ ATPase Pump | Congestive Heart | In vivo: Measurement | Yang et al., Circulation |
| | Failure | of cardiac output, stroke | 92: 262 (1995) |
| | | volume and peripheral | |
| | | vascular resistance in rat | |
| | | model of heart failure. | |
| H+/K+ ATPase Pump | Peptic Ulcer Disease | In vitro: Isolated hog | Sih et al., J. Med. |
| | (PUD); | (H+/K+ ATPase) assay | Chem. 34: 1049 (1991 |
| | Gastroesophageal | In vivo: Inhibition of | |
| | Reflux Disease (GERD) | (H+/K+) ATPase | |
| | | activity in the rat. | |
| MDR Pump (p- | Multidrug Resistance | In vitro: Accumulation | Yoshimura et al, Cancer |
| glycoprotein) | in Tumors | of rhodamine 6G in | Lett. 50: 45 (1990) |
| | | human MDR cell line, | |
| | | KB-C1 | |
| Transporters (5HT; NE; | Depression | In vitro: Inhibition of | Pristupa et al, Mol. |
| Dopamine) | | ³H-dopamine binding in | Pharm 45: 125 (1993) |
| | | cells transfected with | |
| | | human dopamine | |
| | | transporter | |
| | | In vivo: Forced | Detke et al, Exp Clin |
| | | swimming test (FST) in | Psychopharmacol 5: 107 |
| | | rodents | (1997) |

TABLE 4
Linkers

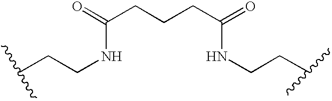

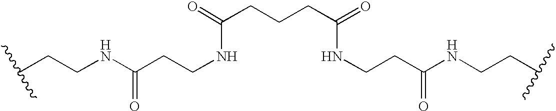

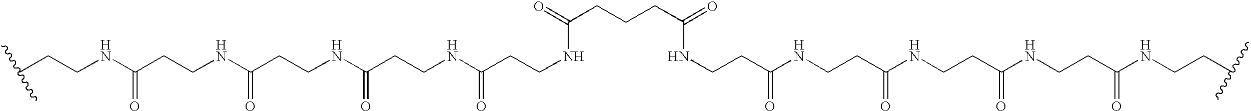

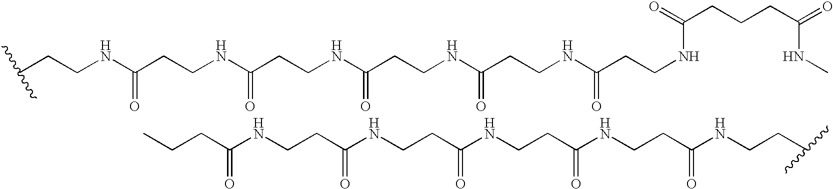

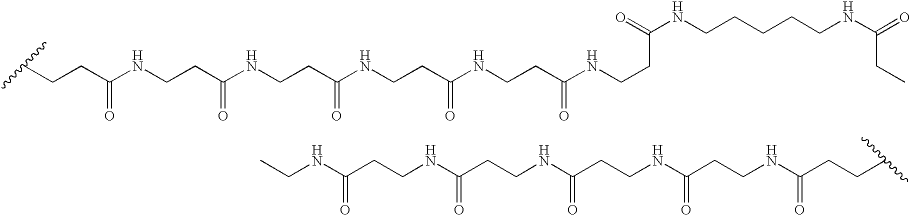

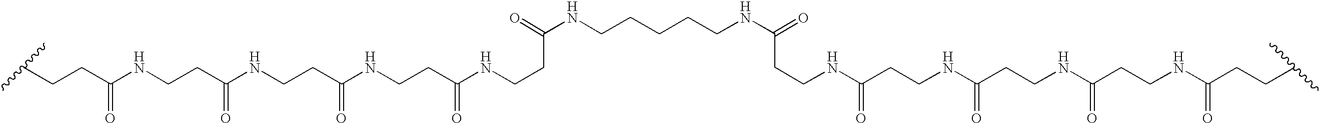

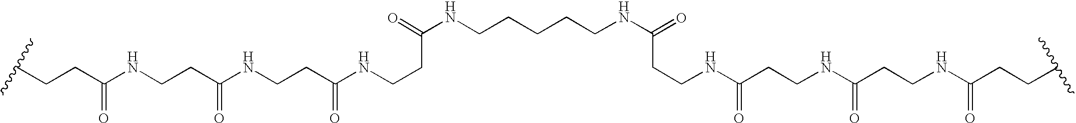

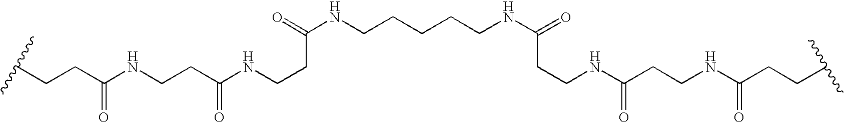

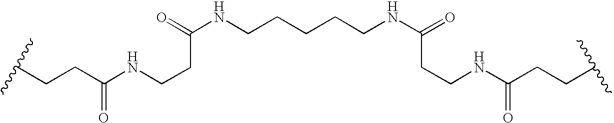

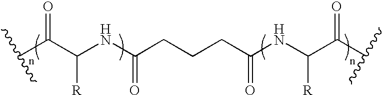

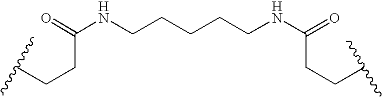

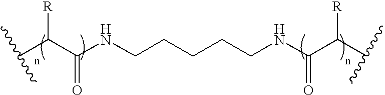

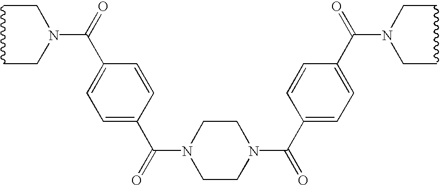

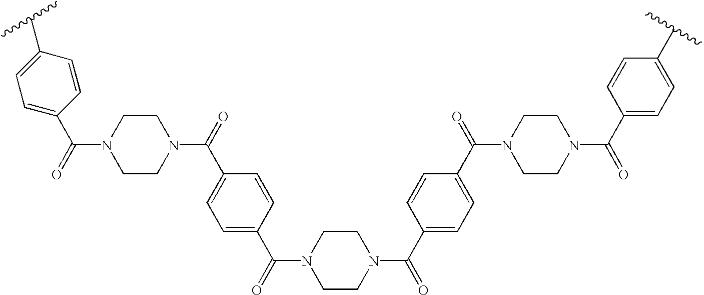

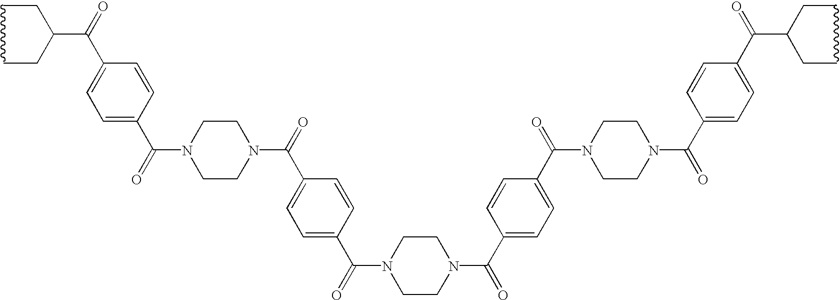

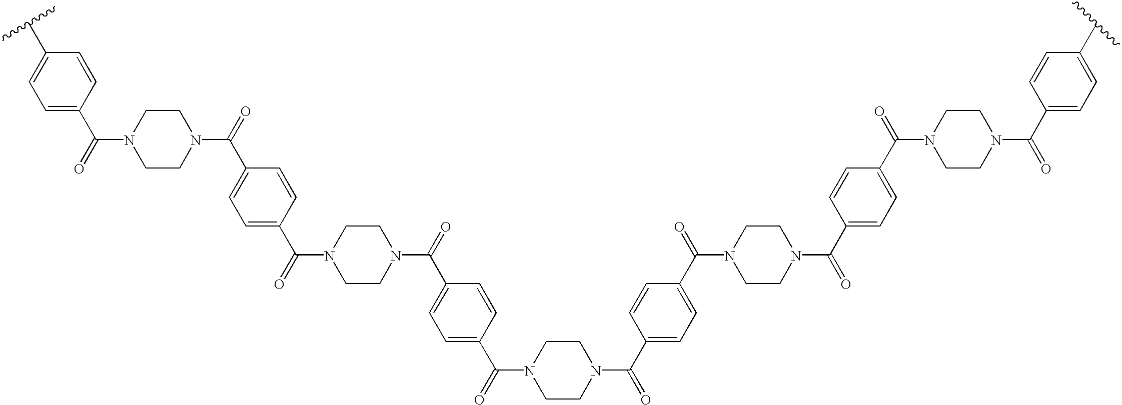

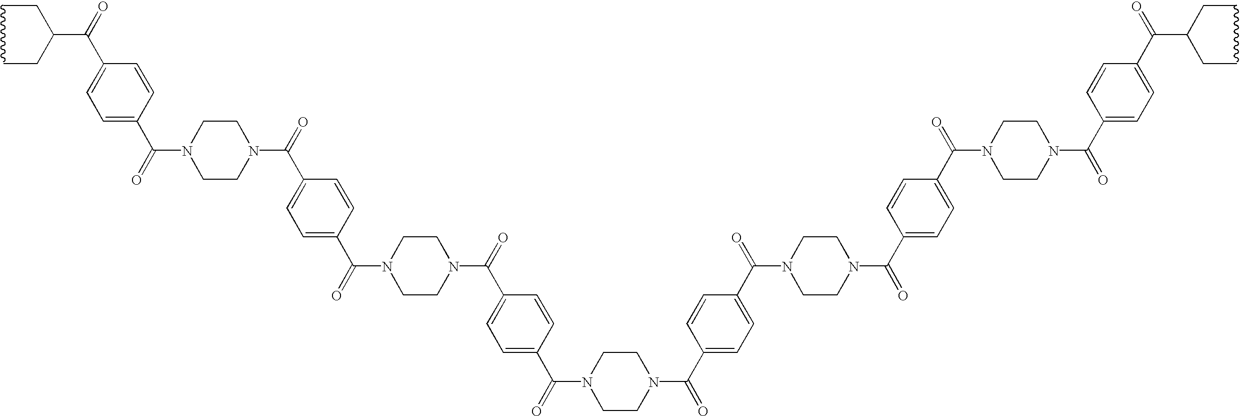

TABLE 5
Linkers

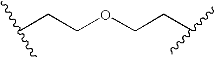

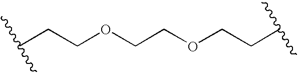

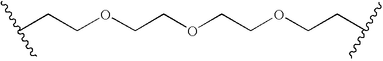

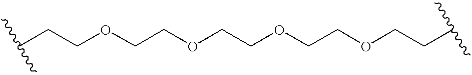

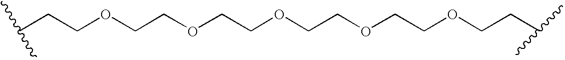

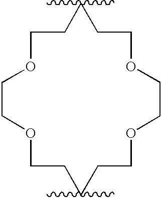

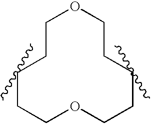

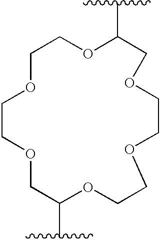

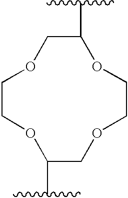

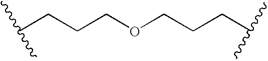

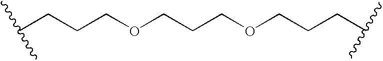

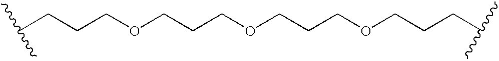

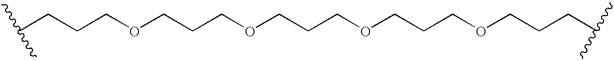

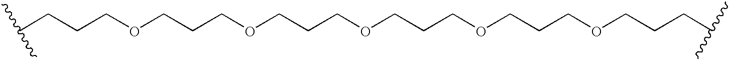

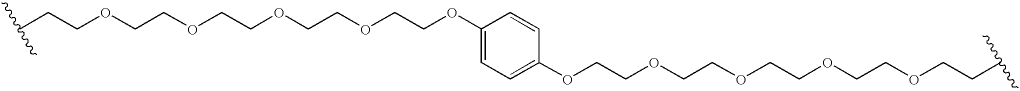

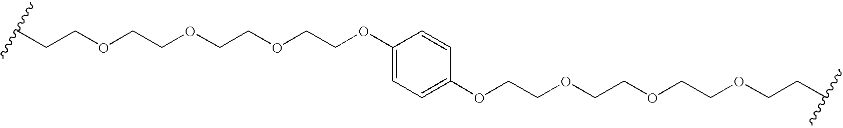

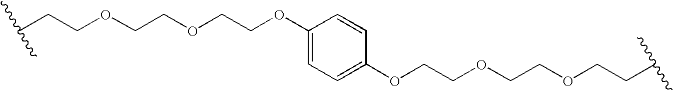

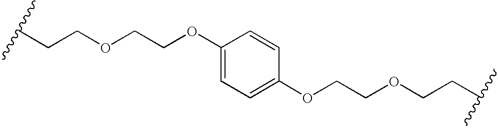

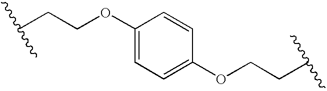

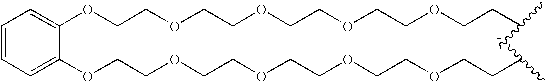

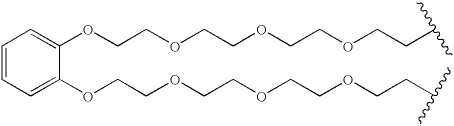

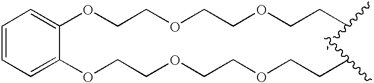

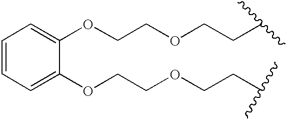

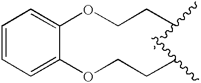

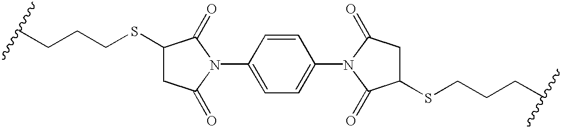

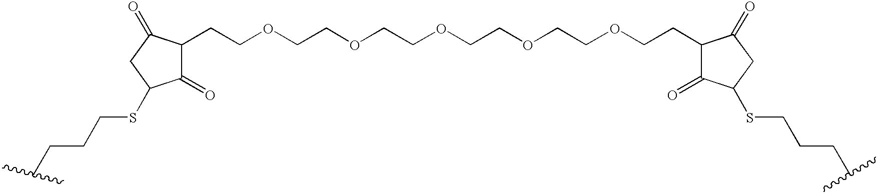

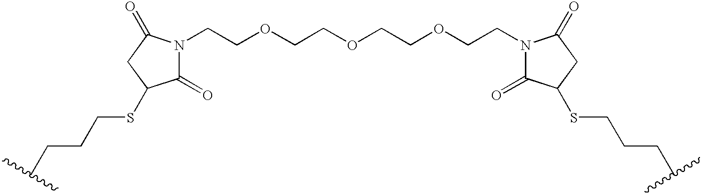

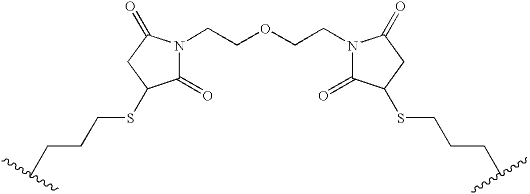

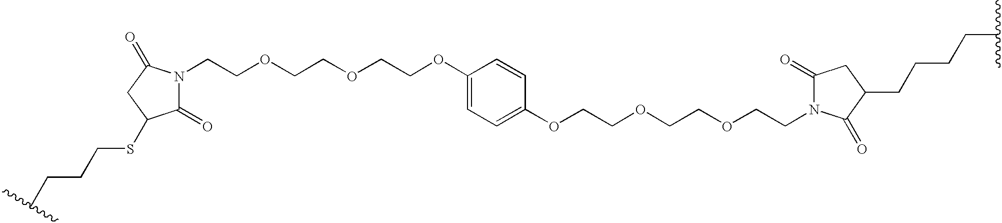

TABLE 6
Activators and Inhibitors of Membrane Transporters

| | Current and Potential | |
|---|---|---|
| Transporter | Therapeutic Indication(s) | Drugs and Other Therapies |
| Ca²⁺ | | |
| Channel | | |
| L-Type | Angina, Atherosclerosis | Amlodipine, nimodipine, |
| | Cardiac failure, | aranidipine, barnidipine, |
| | Hyperlipidemia | cilnidipine, efonidipine |
| | Hypertension, Peripheral | hydrochloride, lercanidipine, |
| | vascular disease, Alzheimers | manidipine, nilvadipine, |
| | disease, Cerebral infarction | isradipine, AE-0047, |
| | Cerebrovascular ischemia, | azelnidipine, lemildipine, |
| | Migraine, Prophylaxis of | lomerizine, pranidipine, |
| | migraine, Subarachnoid | fantofarone, oxodipine, |
| | hemorrhage, Renovascular | clevidipine, diperdipine, Bay-t- |
| | hypertension, Heart disease | 7207, AH-1058, AP-1067, CP- |
| | Central nervous system | 060S, CPC-301, CPC-317, GS- |
| | disease, Alzheimers disease, | 386, LCB-2514, LOE-908, LY- |
| | Motor neurone disease, | 042826, MR-14134, NNC-09- |
| | Parkinsons disease, | 0026, Org-13061, P-5, PCA- |
| | Reperfusion injury, Epilepsy, | 50922, PCA-50938, PCA-50941, |
| | Dementia, Depression, | RGH-2716, S-(−)-amlodipine, |
| | Epilepsy, Head Injury, | SANK-71996, semotiadil |
| | Neuropathic pain, Cardiac | analogs, SIB-1281, SNX-124, |
| | failure, Cystic fibrosis, | SNX-111 (ziconotide), SNX-325, |
| | Hypercholesterolemia, Ocular | SNX-239, SNX-236, VUF-8929, |
| | disease, Parkinsons disease, | zicontide analogs, felodipine + |
| | Neurodegenerative disease, | ramipril, vexibinol, |
| | Thromboembolism, | docosahexaenoic acid, lacidipine, |
| | Subarachnoid hemorrhage, | NS-21, bisaramil, SD-3212, |
| | Inhibition of kinetic cell | BRL-32872, nifedipine, |
| | death, Pregnancy disorder, | nifedipine, Nifelan, Verelan, |
| | Osteoporosis | semotiadil, S-312-d, CERM- |
| | | 12816, ipenoxazone, verapamil |
| | | isomers, tamolarizine, SB- |
| | | 201823A, TDN-345, atosiban, |
| | | TA-993, lifarizine, fasudil, |
| | | furnidipine, elgodipine, SKT-M- |
| | | 26, Y-22516, Verex, verapamil, |
| | | AIT-110, K-201, AIT-111, FPL- |
| | | 64176, NPS-568, L-366682, |
| | | JTV-519, SNX-482, SKF-45675 |
| T-Type | Angina, Cardiac failure | Mibefradil |
| | Hypertension, Chronic stable | U-92032 |
| | angina pectoris, Stroke, | |
| | Cerebrovascular ischemia | |
| N-Type | Cardiac failure, | SNX-111 (ziconotide), SNX-124, |
| | Cardiovascular disease, | SNX-325, SNX-239, SNX-236, |
| | Neurodegenerative disease, | zicontide analogs, conotoxins, |
| | Head injury, Brain injury, | AM-336, PD-029361, PD- |
| | Cerebrovascular ischemia, | 157667, PD-158143, A-53930A, |
| | Inflammation, Neuropathy | conopeptides |
| | Pain, neuropathic pain | |
| | Hypertension, Inflammation | |
| | Alzheimers disease, | |
| | Parkinsons disease, Motor | |
| | neuron disease, Epilepsy | |
| K⁺ | | |
| Channels | | |
| Voltage | Heart arrhythmia, | SB-237376, GYKI-16036, KCB- |
| Sensitive | Tachycardia, Ischemic heart | 295, KCB-328, KCB-345, KMC- |
| | disease, Cardiac failure, | IV-84, L-768673, PGE-8444384, |
| | Transplant rejection, | pyridotriazoles, CK-4001, |
| | Autoimmune disease, | ibutilide, d-(+)-sotalol, MS-551, |
| | Diabetes mellitus, Sickle cell | azimilide, dofetilide, sematilide, |
| | anemia, Muscular dystrophy | E-4031, nibentan, GLG-V-13, |
| | Gastrointestinal disease, | WAY-123398, ersentilide, ATI- |
| | Mental disorder, Sleep | 2001, L-735821, LY-190147, |
| | disorder, Alcoholism, | EGIS-7229, fampridine, CK- |
| | Inflammation, | 1649C, tedisamil, HMR-1883, |
| | Cerebrovascular ischemia, | L-755860, RX-871024, UCL- |
| | Myocardial infarction | 1495, UCL-1559, UCL-1684, |
| | | UK-78282 derivatives, alinidine |
| | | analogs, RSD10XX series, CPU- |
| | | 86017, TJN-505, Win-17317-3, |
| | | stobadine |
| Ca2+ | Hypertension | UCL-1530 |
| sensitive | Heart arrhythmia | |
| | CNS diseases | |
| Receptor- | Epilepsy, Parkinsons disease | Conopeptides |
| coupled | Pain, Cerebrovascular, | JTV-519 |
| | | KB-R7943, SM-20550, |
| | | cariporide, amiloride, RSD-921, |
| | | carsatrin, LY368052, BDF- |
| | | 9198, lamotrigine, stobadine, |
| | | SD-3212, conopeptides |
| Cl⁻ Channel | Cystic fibrosis, Sinusitis, | P-0822, GR-213487B, |
| | Helminth infection, Nematode | ivermectin, S-20787, cytofectins |
| | infection, | (CFTR), CFTR gene therapy; |
| | Hypercholesterolemia, | clotrimazole and analogs, AHC- |
| | Carcinoma, Diarrhea, | 93, CPC-701, CPC-702, OPC- |
| | Keratosis, Neoplasm, Sickle | 18360 |
| | cell anemia, Ischemia, | |
| | Reperfusion injury, | |
| | Hypertension, Head Injury, | |
| | Cardiac failure | |
| Monoamine | Parkinson's disease, Central | BTS-74398, NS-2389, |
| Transporters | nervous system disease, | sibutramine |
| (general) | Depression, Obesity | |
| Noradren- | Attention deficit | Tomoxetine, BW-1555U88, |
| aline | hyperactivity-disorder, | demexiptiline |
| | Depression, Nicotine use | |
| | disorder, Psychosis, | |
| | Parkinson's disease | |
| 5-HT | Anxiety disorder, Depression | Paroxetine, citalopram, |
| | Obsessive/compulsive | fluvoxamine, tianeptine, |
| | disorder, Sleep disorder, | fluoxetine, S-fluoxetine, R- |
| | Sexual dysfunction, Bulimia, | fluoxetine, sertraline, |
| | Premenstrual syndrome, | dexfenfluramine, indalpine, YM- |
| | Psychosexual disorder, | 922, cericlamine, (S)- |
| | Infarction, Antiarrhythmic, | sibutramine, DuP-631, |
| | Panic and post-traumatic | venlafaxine, paroxetine analog, |
| | stress disorder, Anorexia | roxindole, YM-992, S-9977, A- |
| | nervosa, Substance, | 80426, venlafaxine, tramadol, |
| | dependence, Migraine, | duloxetine, milnacipran |
| | Alzheimer's disease, Pain, | |
| | Incontinence, Micturition | |
| | disorder | |
| Dopamine | Schizophrenia, Cocaine use | CDTP-30640, PR-000001, PR- |
| | disorder, Parkinson's disease | 000608, PR-000609, RTI-113, |
| | Schizophrenia, Substance | RTI-177, vanoxerine, WIN- |
| | dependence | 35065 analogs, WF-23, GPI- |
| | | 2138 |
| P-Glyco- | Neoplasm, Brain tumor, | VX-710, VX-853, cinchonine, |
| protein | Breast tumor, Liver tumor, | GF-120918, LY-335979, XR- |
| | Neoplasm, Ovary tumor, | 9576, MS-209, BRI MAb MDR- |
| | Prostate tumor, Sarcoma, | 1, CP-114416, CP-117227, CR- |
| | Carcinoma, Multidrug | 10-11, GR-66234A, ISIS-7597 |
| | resistant infection, Lymphoma | analogs, KT-5822Y, MRK-16, |
| | | MRK-17, N-276-12, OC104-26, |
| | | OC42-92, OC62-805, PAK-200, |
| | | S-16317, SB-RA-31012, XR- |
| | | 1500, 10-deacetylbaccatin III |
| | | derivatives, LY-329146, KT- |
| | | 5720, SDZ-280-446 |
| Gastric | Esophagitis, Peptic ulcer, | (S)-lansoprazole, pantoprazole, |
| Proton | Duodenal ulcer, Stomach | rabeprazole, perprazole, H- |
| Pump | ulcer, Gastrointestinal disease, | 33525, IY-81149, YH-1238, |
| | Peptic ulcer, Helicobacter | YH-1885, IY-81238, (−)- |
| | pylori infection, Osteoporosis, | pantoprazole, AD-8240, |
| | Angina, Fungal infection, | bafilomycin and its derivatives, |
| | Myocardial infarction, | BY-112, FR-168888, |
| | Contraception, | scopadulcic acid B, SM-20220, |
| | Cerebrovascular ischemia | UJ-2012, YS-2012 |
| | ischemia, Ischemia, Heart | NC-1005 |
| | arrhythmia, Myocardial | |
| | infarction, Cardioprotection | |
| K_{ATP} | Angina, Asthma, | JTV-506, Y-26763, Y-27152, |
| | Hypertension, Incontinence | ZD-6169, BMS-204352, KR- |
| | Cerebrovascular ischemia, | 30450, MCC-134, ABA-267, |
| | Ischemic heart disease, | BMS-182264, BPDZ-44, |
| | Cardiovascular disease, | dehydrosoyasaponin-1, DY- |
| | Hyperinsulinemia, Asthma, | 9708, EMD-67618, KC-128, |
| | Epilepsy, Hypertension, | KC-332, KRN-4884, L-3, L- |
| | Incontinence, Urinary | 364373, LM-3339, maxikdiol, |
| | dysfunction, Micturition | NIP-121, NN-5501, NS-8, RS- |
| | disorder, Irritable bowel | 91309, S-103, SCA-40, U- |
| | syndrome, Angina, | 89232, U-99751, WAY-135201, |
| | Restenosis, Insulin dependent | ZD-0947, ZM-244085, ZM- |
| | diabetes, Non-insulin | 260384, nicorandil, KC-515, |
| | dependent diabetes, Diabetic | TAK-636, glipizide, KAD-1229, |
| | neuropathy, Anxiety disorder | DMP-543, U-37883A, PNU- |
| | Neurosis, Subarachnoid | 96293, PNU-99963, BTS-67582, |
| | hemorrhage, Alzheimers | levcromakalim, celikalim |
| | disease | |
| Na⁺ | Cardiovascular disease, Heart | Restacorin, Ro-22-9194, |
| Channel | arrhythmia, Tachycardia | alprafenone, BRB-I-28, |
| | Infarction, CNS disorders | recainam, antiarrhythmics, |
| | Pain, Asthma, Affective | Nortran, CLN-93, RSD10XX |
| | neurosis, Autism, | series, E-047/1, moracizine, |
| | Cerebrovascular ischemia, | pilsicainide, pirmenol, |
| | Depression, Epilepsy, | lamotrigine, procaine |
| | Huntingtons chorea, Seizure | hydrochloride, Steroidogenesis, |
| | Epilepsy, Convulsion, | bupivacaine, cLN-93, 4030W92, |
| | Huntingdon's chorea, Bipolar | 4991W93, transcainide, GW- |
| | disorder, Autism, Stroke, | 273293, LTA, SL-90.0571, |
| | HIV infection, Topical | AAA-241, AWD-140-190, BW- |
| | anesthesia, Migraine, | 202W92, GW-286103, |
| | Depression, Central nervous | iodoamiloride, lidocaine, PD- |
| | system disease, Anesthesia, | 85639, QX-314, ropivacaine, |
| | Urinary tract disease, | fosphenytoin, NS-7, PNU- |
| | Ulcerative colitis, local | 151774E, BW-618C89, |
| | anesthetic in surgery, Cystic | conopeptides, JTV-519, |
| | fibrosis, Parkinsons disease | lifarizine, EMD-96785, EMD- |
| | | 85131, EMD96875, FR-183998, |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective spirit and scope of the present invention. All such modifications are intended to be within the scope of the claims appended hereto.

All of the publications, patent applications and patents cited in this application are herein incorporated by reference in their entirety to the same extent as if each individual publication, patent application or patent was specifically and individually indicated to be incorporated by reference in its entirety.

What is claimed is:

1. A multi-binding compound comprising 2 to 10 ligands which may be the same or different and which are covalently attached to a linker or linkers, which may be the same or different, each of said ligands comprising a ligand domain capable of binding to a cell membrane transporter, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

2. A multi-binding compound represented by formula I:
(L)ₚ(X)_{q} I
where each L is a ligand that may be the same or different at each occurrence;
X is a linker that may be the same or different at each occurrence;
p is an integer of from 2 to 10; and
q is an integer of from 1 to 20;
wherein each of said ligands comprises a ligand domain capable of binding to a cell membrane transporter, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

3. The multi-binding compound of claim 2, wherein q is less than p.

4. The multibinding compound according to claim 1 or claim 2 wherein the compound is dimeric.

5. The multibinding compound according to claim 4 wherein the dimeric compound is heterodimeric.

6. The multibinding compound according to claim 1 or claim 2 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

7. The multibinding compound according to claim 6 wherein the linkers comprise linkers of different chain length and/or having different complementary reactive groups.

8. The multibinding compound according to claim 7 wherein the linkers are selected to have different linker lengths ranging from about 2 to 100Å.

9. The multibinding compound according to claim 8 wherein the linkers are selected to have different linker lengths ranging from about 3 to 40Å.

10. The multibinding compound according to claim 1 or 2 wherein the linker is represented by the formula::
—X′—Z—(Y′—Z)ₘ—Y″—Z—X—′—
in which:
m is an integer of from 0 to 20;
X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of 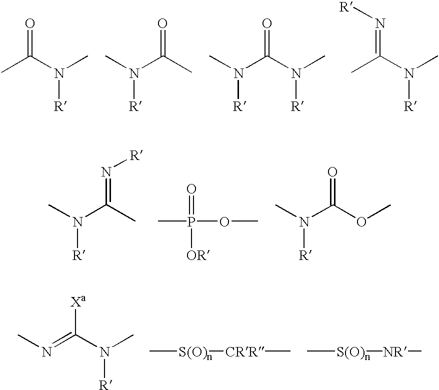
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

11. The multibinding compound according to claim 4 wherein the dimeric compound is homodimeric.

12. The multibinding compound according to claim 11 wherein the two cell membrane transporter ligands are linked to the homodimeric compound at the same point on the ligand.

13. The multibinding compound according to claim 10 wherein the two cell membrane transporter ligands are linked to the homodimeric compound at different points on the ligand.

14. A pharmaceutical composition comprising a pharmaceutically acceptable excipient and a therapeutically effective amount of one or more multi-binding compounds, or pharmaceutically acceptable salts thereof, comprising 2 to 10 ligands which may be the same or different and which are covalently attached to a linker or linkers, which may be the same or different, each of said ligands comprising a ligand domain capable of binding to a cell membrane transporter of a cell mediating mammalian or avian diseases or conditions, thereby modulating the diseases or conditions, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

15. A pharmaceutical composition comprising a pharmaceutically acceptable excipient and a therapeutically effective amount of one or more multi-binding compounds represented by formula I,
(L)ₚ(X)_{q} I
and pharmaceutically acceptable salts thereof,
where each L is a ligand that may be the same or different at each occurrence;
X is a linker that may be the same or different at each occurrence;
p is an integer of from 2 to 10; and
q is an integer of from 1 to 20;
wherein each of said ligands comprises a ligand domain capable of binding to a cell membrane transporter of a cell mediating mammalian or avian diseases or. conditions, thereby modulating the diseases or conditions, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

16. The pharmaceutical composition according to claim 15 wherein q is less than p.

17. The pharmaceutical composition according to claim 14 or claim 15 wherein the compound is dimeric.

18. The pharmaceutical composition according to claim 17 wherein the dimeric compound is heterodimeric.

19. The pharmaceutical composition according to claim 14 or claim 15 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

20. The pharmaceutical composition according to claim 19 wherein the linkers comprise linkers of different chain length and/or having different complementary reactive groups.

21. The pharmaceutical composition according to claim 20 wherein the linkers are selected to have different linker lengths ranging from about 2 to 100Å.

22. The pharmaceutical composition according to claim 21 wherein the linkers are selected to have different linker lengths ranging from about 3 to 40Å.

23. The pharmaceutical composition according to claim 14 or 15 wherein the linker is represented by the formula::
—X′—Z—(Y′—Z)ₘ—Y″—Z—X′—
in which:
m is an integer of from 0 to 20;
X′ at each separate occurrence is selected from the group consisting of —O—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of: 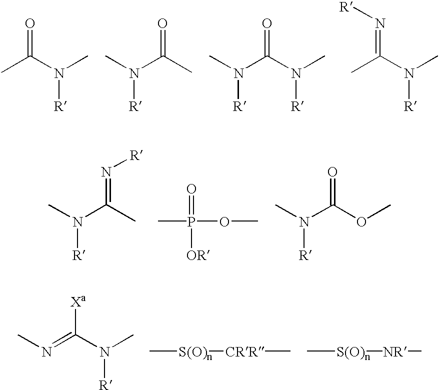
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

24. A method for modulating the activity of a cell membrane transporter in a biologic tissue, which method comprises contacting a tissue having a cell membrane transporter with a multi-binding compound, or a pharmaceutically acceptable salt thereof, under conditions sufficient to produce a change in transporter activity in said tissue, wherein the multi-binding compound comprises 2 to 10 ligands which may be the same or different and which are covalently attached to a linker or linkers, which may be the same or different, each of said ligands comprising a ligand domain capable of binding to a cell membrane transporter, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

25. A method for treating a disease or condition in a mammal or avian resulting from an activity of a cell membrane transporter, which method comprises administering to said mammal or avian a therapeutically effective amount of a pharmaceutical composition comprising a pharmaceutically acceptable excipient and one or more multi-binding compounds, or pharmaceutically acceptable salts thereof, comprising 2 to 10 ligands which may be the same or different and which are covalently attached to a linker or linkers, which may be the same or different, each of said ligands comprising a ligand domain capable of binding to a cell membrane transporter of a cell mediating mammalian or avian diseases or conditions, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

26. A method for treating a disease or condition in a mammal or avian resulting from an activity of a cell membrane transporter, which method comprises administering to said mammal or avian a therapeutically effective amount of a pharmaceutical composition comprising a pharmaceutically acceptable excipient and one or more multibinding compounds represented by formula I,
(L)ₚ(X)_{q} I
and pharmaceutically acceptable salts thereof,
where each L is a ligand that may be the same or different at each occurrence;
X is a linker that may be the same or different at each occurrence;
p is an integer of from 2 to 10; and
q is an integer of from 1 to 20;
wherein each of said ligands comprises a ligand domain capable of binding to a cell membrane transporter of a cell mediating mammalian or avian diseases or conditions, with the following provisos:
(a) when each ligand is a 1,2,3,4 tetrahydroisoquinoline, then the linker is at least two atoms;
(b) neither the compound nor the ligand is a bis-quinolinium cyclophane; and
(c) the multibinding compound does not comprise homomeric phenylsulfonamide ligands;
(d) the multibinding compound does not comprise a dimeric 1,4 dihydropyridine compound linked through carboxylic ester groups.

27. The method according to claim 26 wherein q is less than p.

28. The method according to claim 24, claim 25 or claim 26 wherein the compound is dimeric.

29. The method according to claim 28 wherein the dimeric compound is heterodimeric.

30. The method according to claim 28 wherein the dimeric compound is homodimeric.

31. The method according to claim 24, claim 25 or claim 26 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

32. The method according to claim 31 wherein the linkers comprise linkers of different chain length and/or having different complementary reactive groups.

33. The method according to claim 32 wherein the linkers are selected to have different linker lengths ranging from about 2 to 100Å.

34. The method according to claim 33 wherein the linkers are selected to have different linker lengths ranging from about 3 to 40Å.

35. The method compound according to claim 24, claim 25 or claim 26 wherein the linker is represented by the formula:
—X′—Z—(Y′—Z)ₘ—Y″—Z—X′—
in which:
m is an integer of from 0 to 20;
X′ at each separate occurrence is selected from the group consisting of —O-—, —S—, —NR—, —C(O)—, —C(O)O—, —C(O)NR—, —C(S), —C(S)O—, —C(S)NR— or a covalent bond where R is as defined below;
Z is at each separate occurrence is selected from the group consisting of alkylene, substituted alkylene, cycloalkylene, substituted cylcoalkylene, alkenylene, substituted alkenylene, alkynylene, substituted alkynylene, cycloalkenylene, substituted cycloalkenylene, arylene, heteroarylene, heterocyclene, or a covalent bond;
Y′ and Y″ at each separate occurrence are selected from the group consisting of 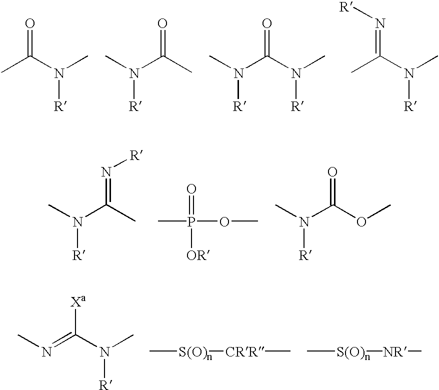
—S—S— or a covalent bond;
in which:
n is 0, 1 or 2; and
R, R′ and R″ at each separate occurrence are selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl and heterocyclic.

36. A method for identifying multimeric ligand compounds possessing multibinding properties which method comprises:
(a) identifying a ligand or a mixture of ligands wherein each ligand contains at least one reactive functionality;
(b) identifying a library of linkers wherein each linker in said library comprises at least two functional groups having complementary reactivity to at least one of the reactive functional groups of the ligand;
(c) preparing a multimeric ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands; and
(d) assaying the multimeric ligand compounds produced in the library prepared in (c) above to identify multimeric ligand compounds possessing multibinding properties.

37. A method for identifying multimeric ligand compounds possessing multibinding properties which method comprises:
(a) identifying a library of ligands wherein each ligand contains at least one reactive functionality;
(b) identifying a linker or mixture of linkers wherein each linker comprises at least two functional groups having complementary reactivity to at least one of the reactive functional groups of the ligand;
(c) preparing a multimeric ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands; and
(d) assaying the multimeric ligand compounds produced in the library prepared in (c) above to identify multimeric ligand compounds possessing multibinding properties.

38. The method according to claim 36 or 37 wherein the preparation of the multimeric ligand compound library is achieved by either the sequential or concurrent combination of the two or more stoichiometric equivalents of the ligands identified in (a) with the linkers identified in (b).

39. The method according to claim 38 wherein the multimeric ligand compounds comprising the multimeric ligand compound library are dimeric.

40. The method according to claim 39 wherein the dimeric ligand compounds comprising the dimeric ligand compound library are heterodimeric.

41. The method according to claim 39 wherein the dimeric ligand compounds comprising the dimeric ligand compound library are heterodimeric.

42. The method according to claim 39 wherein the heterodimeric ligand compound library is prepared by sequential addition of a first and second ligand.

43. The method according to claim 36 or 37 wherein, prior to procedure (d), each member of the multimeric ligand compound library is isolated from the library.

44. The method according to claim 43 wherein each member of the library is isolated by preparative liquid chromatography mass spectrometry (LCMS).

45. The method according to claim 36 or claim 37 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

46. The method according to claim 45 wherein the linkers comprise linkers of different chain length and/or having different complementary reactive groups.

47. The method according to claim 46 wherein the linkers are selected to have different linker lengths ranging from about 2 to 100Å.

48. The method according to claim 36 or 37 wherein the ligand or mixture of ligands is selected to have reactive functionality at different sites on said ligands.

49. The method according to claim 48 wherein said reactive functionality is selected from the group consisting of carboxylic acids, carboxylic acid halides, carboxyl esters, amines, halides, pseudohalides, isocyanates, vinyl unsaturation, ketones, aldehydes, thiols, alcohols, anhydrides, boronates, and precursors thereof wherein the reactive functionality on the ligand is selected to be complementary to at least one of the reactive groups on the linker so that a covalent linkage can be formed between the linker and the ligand.

50. The method according to claim 36 or claim 37 wherein the multimeric ligand compound library comprises homomeric ligand compounds.

51. The method according to claim 36 or claim 37 wherein the multimeric ligand compound library comprises heteromeric ligand compounds.

52. A library of multimeric ligand compounds which may possess multivalent properties which library is prepared by the method comprising:
(a) identifying a ligand or a mixture of ligands wherein each ligand contains at least one reactive functionality;
(b) identifying a library of linkers wherein each linker in said library comprises at least two functional groups having complementary reactivity to at least one of the reactive functional groups of the ligand; and
(c) preparing a multimeric ligand compound library by combining at least two stoichiometric equivalents of the ligand or mixture of ligands identified in (a) with the library of linkers identified in (b) under conditions wherein the complementary functional groups react to form a covalent linkage between said linker and at least two of said ligands.

53. A library of multimeric ligand compounds which may possess multivalent properties which library is prepared by the method comprising:
(a) identifying a library of ligands wherein each ligand contains at least one reactive functionality;
(b) identifying a linker or mixture of linkers wherein each linker comprises at least two functional groups having complementary reactivity to at least one of the reactive functional groups of the ligand; and
(c) preparing a multimeric ligand compound library by combining at least two stoichiometric equivalents of the library of ligands identified in (a) with the linker or mixture of linkers identified in (b) under conditions wherein the complementary functional groups react to form a covalent linkage between said linker and at least two of said ligands.

54. The library according to claim 52 or claim 53 wherein the linker or linkers employed are selected from the group comprising flexible linkers, rigid linkers, hydrophobic linkers, hydrophilic linkers, linkers of different geometry, acidic linkers, basic linkers, linkers of different polarization and/or polarizability and amphiphilic linkers.

55. The library according to claim 54 herein the linkers comprise linkers of different chain length and/or having different complementary reactive groups.

56. The library according to claim 55 herein the linkers are selected to have different linker lengths ranging from about 2 to 100Å.

57. The library according to claim 52 or 53 wherein the ligand or mixture of ligands is selected to have reactive functionality at different sites on said ligands.

58. The library according to claim 57 wherein said reactive functionality is selected from the group consisting of carboxylic acids, carboxylic acid halides, carboxyl esters, amines, halides, pseudohalides, isocyanates, vinyl unsaturation, ketones, aldehydes, thiols, alcohols, anhydrides, boronates, and precursors thereof wherein the reactive functionality on the ligand is selected to be complementary to at least one of the reactive groups on the linker so that a covalent linkage can be formed between the linker and the ligand.

59. The library according to claim 52 or claim 53 wherein the multimeric ligand compound library comprises homomeric ligand compounds.

60. The library according to claim 52 or claim 53 wherein the multimeric ligand compound library comprises heteromeric ligand compounds.

61. An iterative method for identifying multimeric ligand compounds possessing multibinding properties which method comprises:
(a) preparing a first collection or iteration of multimeric compounds which is prepared by contacting at least two stoichiometric equivalents of the ligand or mixture of ligands which target a receptor with a linker or mixture of linkers wherein said ligand or mixture of ligands comprises at least one reactive functionality and said linker or mixture of linkers comprises at least two functional groups having complementary reactivity to at least one of the reactive functional groups of the ligand wherein said contacting is conducted under conditions wherein the complementary functional groups react to form a covalent linkage between said linker and at least two of said ligands;
(b) assaying said first collection or iteration of multimeric compounds to assess which if any of said multimeric compounds possess multibinding properties;
(c) repeating the process of (a) and (b) above until at least one multimeric compound is found to possess multibinding properties;
(d) evaluating what molecular constraints imparted or are consistent with imparting multibinding properties to the multimeric compound or compounds found in the first iteration recited in (a)- (c) above;
(e) creating a second collection or iteration of multimeric compounds which elaborates upon the particular molecular constraints imparting multibinding properties to the multimeric compound or compounds found in said first iteration;
(f) evaluating what molecular constraints imparted or are consistent with imparting enhanced multibinding properties to the multimeric compound or compounds found in the second collection or iteration recited in (e) above;
(g) optionally repeating steps (e) and (f) to further elaborate upon said molecular constraints.

62. The method according to claim 61 wherein steps (e) and (f) are repeated from 2-50 times.

63. The method according to claim 62 wherein steps (e) and (f) are repeated from 5-50 times.
